(19) United States
(12) Patent Application Publication
(10) Pub. No.: US 2021/0301327 A1
(43) Pub. Date: Sep. 30, 2021

(54) METHOD FOR THE DETECTION OF MICROORGANISMS, CORRESPONDING USE AND SAMPLE CARRIER

(71) Applicant: Testo bioAnalytics GmbH, Titisee-Neustadt (DE)

(72) Inventors: Paul Quehl, Göttingen (DE); Zinaida Vasileuskaya-Schulz, Freiburg (DE); Serge Ruden, Titisee-Neustadt (DE); Daniel Braga, Freiburg (DE)

(73) Assignee: Testo bioAnalytics GmbH, Titisee-Neustadt (DE)

(21) Appl. No.: 17/175,042

(22) Filed: Feb. 12, 2021

(30) Foreign Application Priority Data
Feb. 14, 2020 (DE) 10 2020 103 961.1

(51) Int. Cl. C12Q 1/689; C12Q 1/6825

(52) U.S. Cl. CPC C12Q 1/689; C12Q 1/6825

(57) ABSTRACT

What is proposed is to provide a method for detecting microorganisms, for example of the Enterobacteriaceae family of bacteria, in a sample by means of delivery of identity-determining nucleic acid probes into the individual cell bodies, wherein the nucleic acid probes hybridize to the nucleic acids of the microorganisms, and subsequent optical detection of the hybridizations generated in the individual cell bodies, wherein a mixture of at least a first nucleic acid probe and a second nucleic acid probe is used, and wherein the first nucleic acid probe and the second nucleic acid probe respectively bind in one of two nonoverlapping regions of the target nucleic acid.

This application claims priority to DE 10 2020 103 961.1, which was filed Feb. 14, 2020 and is incorporated herein by reference as if fully set forth.

The Sequence Listing filed Feb. 12, 2021, titled Sequence Listing, prepared Feb. 11, 2021, and having a file size of 42.2 kilobytes is incorporated herein by reference as if fully set forth. The Sequence Listing filed Apr. 29, 2021, titled Sequence Listing, prepared Apr. 29, 2021, and having a file size of 283,473 bytes is incorporated herein by reference as if fully set forth. The Substitute Sequence Listing filed Jun. 1, 2021, titled Substitute Sequence Listing, prepare Jun. 1, 2021, and having a file size of 283,415 bytes is incorporated herein by reference as if fully set forth.

The invention relates to a method for detecting microorganisms, for example of the Enterobacteriaceae family of bacteria, in a sample by means of delivery of identity-determining nucleic acid probes into the individual cell bodies, wherein the nucleic acid probes hybridize to the nucleic acids of the microorganisms, and subsequent optical detection of the hybridizations generated in the individual cell bodies, wherein a mixture of at least a first nucleic acid probe and a second nucleic acid probe is used, and wherein the first nucleic acid probe and the second nucleic acid probe respectively bind in one of two nonoverlapping regions of the target nucleic acid.

Enterobacteriaceae are a group of the Gram-negative, facultatively anaerobic rod bacteria that can generally grow on simple culture media. This highly heterogeneous, phylogenetic family currently comprises about 53 genera and over 170 species. Many Enterobacteriaceae are pathogenic to humans and are therefore indicator pathogens in hygiene control.

Enterobacteriaceae are traditionally identified via specific culturing on selective culture media and biochemical secondary detection. Membership of a particular family can also be detected on the basis of identity-determining nucleic acid sequences of the microorganism to be tested. Such detection can be carried out by means of PCR or microarray. Other known methods for the specific detection of nucleic acids, i.e., DNA and/or RNA molecules, in individual cells include, for example, in-situ hybridization (ISH). This involves using short synthetic nucleic acid probes which bind to the target sequence to be detected via base pairings. A variant of ISH technology in which the nucleic acid probes are fluorescently labeled is fluorescence in-situ hybridization (FISH).

However, the known nucleic acid probes used for detection of the Enterobacteriaceae family of bacteria in the FISH method often have inadequate specificity between Enterobacteriaceae and other families of bacteria. Therefore, false-positive results frequently occur, since the nucleic acid probes do not hybridize just to the nucleic acids of the Enterobacteriaceae.

Within the Enterobacteriaceae family of bacteria and the genera, species and serotypes thereof such as especially Escherichia coli and especially EHEC and STEC and VTEC and especially E. coli 0157:H7, Salmonella and especially Salmonella enterica, Yersinia and especially Yersinia enterocolitica and Yersinia pestis, Enterobacter, antibiotic-resistant enterobacteria and other families of bacteria and the genera, species and serotypes thereof such as especially Listeria and especially Listeria monocytogenes, Bacillus and especially Bacillus cereus and Bacillus anthracis and Bacillus subtilis, Pseudomonas and especially Pseudomonas aeroginosa, Staphylococcus and especially Staphylococcus aureus and MRSA, Campylobacteraceae and especially Campylobacter, Acinetobacter and especially Acinetobacter baumannii and Acinetobacter johnsonii and especially antibiotic-resistant Acinetobacter, Gram-positive and Gram-negative antibiotic-resistant bacteria, the conventional nucleic acid probes also have inadequate sensitivity, and so false-negative results can also occur. With the individual conventional nucleic acid probes, what can often be achieved is a fluorescence intensity that is insufficient for detection of the family of bacteria to be detected. The use of multiple nucleic acid probes at the same time is generally hampered by the fact that the conventional nucleic acid probes for Enterobacteriaceae are not combinable with one another because they have overlapping target sequences.

Against this background, it is an object of the present invention to provide an improved method which allows specific detection of microorganisms, for example of the Enterobacteriaceae family of bacteria, in a sample on the basis of identity-specific nucleic acid probes.

The invention achieves this object through the features of claim 1. In particular, what is therefore proposed according to the invention to achieve the stated object in a method of the kind described at the start is that a mixture of at least a first nucleic acid probe and a second nucleic acid probe is used, and that the first nucleic acid probe and the second nucleic acid probe respectively bind in one of two nonoverlapping regions of the target nucleic acid. What is therefore easily achievable is that the first nucleic acid probe and the second nucleic acid probe (and optionally further nucleic acid probes) can bind to the same target nucleic acid at the same time. This can considerably improve the detectability of Enterobacteriaceae, since, with the combination of multiple nucleic acid probes, it is possible to achieve a higher fluorescence intensity per bacterium by multiple target sequences being able to be detected at the same time.

In an advantageous embodiment according to the invention, the mixture is formed such that, from the present combinatorics of groups 1 to 12, selection is respectively made of a representative from each group or a representative from at least two groups, especially wherein group 1 comprises some or all of SEQ ID NO: 1 to SEQ ID NO: 19, group 2 comprises some or all of SEQ ID NO: 20 to SEQ ID NO: 40, group 3 comprises some or all of SEQ ID NO: 41 to SEQ ID NO: 59, group 4 comprises some or all of SEQ ID NO: 60 to SEQ ID NO: 78, group 5 comprises some or all of SEQ ID NO: 79 to SEQ ID NO: 99, group 6 comprises some or all of SEQ ID NO: 100 to SEQ ID NO: 118, group 7 comprises some or all of SEQ ID NO: 119 to SEQ ID NO: 137, group 8 comprises some or all of SEQ ID NO: 138 to SEQ ID NO: 156, group 9 comprises some or all of SEQ ID NO: 157 to SEQ ID NO: 175, group 10 comprises some or all of SEQ ID NO: 176 to SEQ ID NO: 196, group 11 comprises some or all of SEQ ID NO: 197 to SEQ ID NO: 217, and group 12 comprises some or all of SEQ ID NO: 218 to SEQ ID NO: 236.

In an advantageous embodiment according to the invention, the mixture is formed such that, from the present combinatorics of groups 13 to 33, selection is respectively made of a representative from each group or a representative from at least two groups, especially wherein group 13 comprises some or all of SEQ ID NO: 237 to SEQ ID NO: 294, group 14 comprises some or all of SEQ ID NO: 295 to SEQ ID NO: 314, group 15 comprises some or all of SEQ ID NO: 315 to SEQ ID NO: 372, group 16 comprises some or all of SEQ ID NO: 373 to SEQ ID NO: 411, group 17 comprises some or all of SEQ ID NO: 412 to SEQ ID NO: 450, group 18 comprises some or all of SEQ ID NO: 451 to SEQ ID NO: 508, group 19 comprises some or all of SEQ ID NO: 509 to SEQ ID NO: 528, group 20 comprises some or all of SEQ ID NO: 529 to SEQ ID NO: 547, group 21 comprises some or all of SEQ ID NO: 548 to SEQ ID NO: 567, group 22 comprises some or all of SEQ ID NO: 568 to SEQ ID NO: 587, group 23 comprises some or all of SEQ ID NO: 588 to SEQ ID NO: 607, group 24 comprises some or all of SEQ ID NO: 608 to SEQ ID NO: 626, group 25 comprises some or all of SEQ ID NO: 627 to SEQ ID NO: 645, group 26 comprises some or all of SEQ ID NO: 646 to SEQ ID NO: 664, group 27 comprises some or all of SEQ ID NO: 665 to SEQ ID NO: 684, group 28 comprises some or all of SEQ ID NO: 685 to SEQ ID NO: 704, group 29 comprises some or all of SEQ ID NO: 705 to SEQ ID NO: 724, group 30 comprises some or all of SEQ ID NO: 725 to SEQ ID NO: 744, group 31 comprises some or all of SEQ ID NO: 745 to SEQ ID NO: 764, group 32 comprises some or all of SEQ ID NO: 765 to SEQ ID NO: 804, and group 33 comprises some or all of SEQ ID NO: 805 to SEQ ID NO: 824.

In an advantageous embodiment according to the invention, the mixture is formed such that, from the present combinatorics of groups 34 to 46, selection is respectively made of a representative from each group or a representative from at least two groups, especially wherein group 34 comprises some or all of SEQ ID NO: 825 to SEQ ID NO: 844, group 35 comprises some or all of SEQ ID NO: 845 to SEQ ID NO: 865, group 36 comprises some or all of SEQ ID NO: 866 to SEQ ID NO: 886, group 37 comprises some or all of SEQ ID NO: 887 to SEQ ID NO: 927, group 38 comprises some or all of SEQ ID NO: 928 to SEQ ID NO: 947, group 39 comprises some or all of SEQ ID NO: 948 to SEQ ID NO: 967, group 40 comprises some or all of SEQ ID NO: 968 to SEQ ID NO: 988, group 41 comprises some or all of SEQ ID NO: 989 to SEQ ID NO: 1009, group 42 comprises some or all of SEQ ID NO: 1010 to SEQ ID NO: 1071, group 43 comprises some or all of SEQ ID NO: 1072 to SEQ ID NO: 1133, group 44 comprises some or all of SEQ ID NO: 1134 to SEQ ID NO: 1154, group 45 comprises some or all of SEQ ID NO: 1155 to SEQ ID NO: 1196, and group 46 comprises some or all of SEQ ID NO: 1197 to SEQ ID NO: 1216.

In relation to this, the invention takes advantage of the fact that it is possible to considerably improve sensitivity (correct positive rate) and specificity (correct negative rate) with respect to Enterobacteriaceae by use of the combination of multiple nucleic acid probes from the combinatorics according to the invention of groups 1 to 12 or with respect to Listeriaceae by use of the combination of multiple nucleic acid probes from the combinatorics according to the invention of groups 13 to 33, or with respect to Listeria monocytogenes by use of the combination of multiple nucleic acid probes from the combinatorics according to the invention of groups 34 to 46. In particular, the use of more than just one nucleic acid probe can prevent the possible occurrence of mismatches in the highly variable nucleic acid probe target regions, the result being that false negative results may arise.

Furthermore, in an advantageous embodiment according to the invention, the detection reaction using nucleic acid probes is carried out by means of fluorescence in-situ hybridization (FISH), nucleic acid amplification reaction and/or microarray. An amplification reaction can, for example, be a polymerase chain reaction (“PCR”). The advantage here is that specific detection of Enterobacteriaceae can be made possible by means of different detection techniques.

In an advantageous embodiment according to the invention, the nucleic acid probes are each designed as linear probes. As an alternative or in addition, the nucleic acid probes can have secondary structure, for example can be designed as molecular beacons and/or as Scorpions probes. What is achievable as a result is a higher fluorescence intensity and also a better signal-to-noise ratio, which may be advantageous especially for an automated application.

Furthermore, in an advantageous embodiment according to the invention, optical sensitivity is set such that only those microorganisms having at least two binding events are detected. It can therefore be ensured that always at least two nucleic acid probes bind in the family of bacteria to be detected. The advantage here is that higher diagnostic sensitivity is achievable.

In an advantageous embodiment according to the invention, the first and/or second nucleic acid probe has/have at least one first dye conjugated to the 5′ end and/or at least one second dye conjugated to the 3′ end. The advantage here is that the use of various dyes can allow the detection of a particular color combination.

Furthermore, in an advantageous embodiment according to the invention, the nucleic acid probe has further nucleotides as stem sequence at the 5′ end and/or 3′ end and/or at least one functional part. In particular, the stem sequences and/or functional parts can be formed in relation to one another such that they do not mutually interact with one another. In relation to this, the invention takes advantage of the fact that the stem-forming nucleotides can form a “hairpin” structure in the absence of target sequences, thereby suppressing the fluorescence of the dye. After the functional part binds to the target sequence, said “hairpin” structure comes apart, whereupon the fluorescence of the dye, which is no longer suppressed, can be detected.

In an advantageous embodiment according to the invention, the selection of the nonoverlapping regions is chosen such that a sufficiently large spatial distance is present and the nucleic acid probes do not interfere with one another in their radiation behavior. The occurrence of incorrect measurements during signal capture can therefore be prevented.

Furthermore, in an advantageous embodiment according to the invention, the nucleic acid probes are formed such that a particular color combination is detectable. For example, for this purpose, the or a first nucleic acid probe has a first dye and the or a second nucleic acid probe has a second dye. The advantage here is that the detection of a particular color combination can allow an alternative to measurement sensitivity for specific detectability of, for example, Enterobacteriaceae.

In an advantageous embodiment according to the invention, the nucleic acid probe has at least one optically detectable label. The detectable label can, for example, be an enzymatically active group, an affinity label or a dye. The affinity label can, for example, include biotin-streptaviclin or antigen-antibody affinity binding pairs. The enzymatically active label can, for example, be peroxidase, luciferase or phosphatase. The dye can, for example, be a fluorescent label. Optical detection is therefore achievable.

Furthermore, in an advantageous embodiment according to the invention, each nucleic acid probe binds to at least 80% of the, for example, Enterobacteriaceae family of bacteria.

As an alternative or in addition, each nucleic acid probe can bind detectably just to the nucleic acids of, for example, the Enterobacteriaceae family of bacteria, and not to the nucleic acids of an organism belonging to a different family of bacteria. Highly specific detectability is therefore achievable for the example of the Enterobacteriaceae family of bacteria.

A preferred application provides for use of at least one nucleic acid probe or at least two nucleic acid probes selected from the group consisting of SEQ ID NO: 1, SEQ ID NO: 20, SEQ ID NO: 41, SEQ ID NO: 60, SEQ ID NO: 79, SEQ ID NO: 100, SEQ ID NO: 119, SEQ ID NO: 138, SEQ ID NO: 157, SEQ ID NO: 176, SEQ ID NO: 197, SEQ ID NO: 218 and/or further sequences from the present combinatorics of groups 1 to 12 for detection of Enterobacteriaceae and/or for immobilization on a carrier material, especially a fluidic channel system.

A preferred application provides for use of at least one nucleic acid probe or at least two nucleic acid probes selected from the group consisting of SEQ ID NO: 237, SEQ ID NO: 295, SEQ ID NO: 315, SEQ ID NO: 373, SEQ ID NO: 412, SEQ ID NO: 451, SEQ ID NO: 509, SEQ ID NO: 529, SEQ ID NO: 548, SEQ ID NO: 568, SEQ ID NO: 588, SEQ ID NO: 608, SEQ ID NO: 627, SEQ ID NO: 646, SEQ ID NO: 665, SEQ ID NO: 685, SEQ ID NO: 705, SEQ ID NO: 725, SEQ ID NO: 745, SEQ ID NO: 765, SEQ ID NO: 805 and/or further sequences from the present combinatorics of groups 13 to 33 for detection of Listeriaceae and/or for immobilization on a carrier material, especially a fluidic channel system.

A preferred application provides for use of at least one nucleic acid probe or at least two nucleic acid probes selected from the group consisting of SEQ ID NO: 825, SEQ ID NO: 845, SEQ ID NO: 866, SEQ ID NO: 887, SEQ ID NO: 928, SEQ ID NO: 948, SEQ ID NO: 968, SEQ ID NO: 989, SEQ ID NO: 1010, SEQ ID NO: 1072, SEQ ID NO: 1134, SEQ ID NO: 1155, SEQ ID NO: 1197 and/or further sequences from the present combinatorics of groups 34 to 46 for detection of Listeriaceae, especially Listeria monocytogenes, and/or for immobilization on a carrier material, especially a fluidic channel system.

A preferred application provides a fluidic channel system comprising means for carrying out the method, especially as described above and/or as per any of the claims directed to a method. For example, a detection zone and a preparation zone can be formed in the fluidic channel system for carrying out the method according to the invention. In particular, the cross-sections of the channels of the fluidic channel system can be matched to dimensions of the microorganisms.

The fluidic channel system can, for example, be designed as a sample carrier. The sample carrier according to the invention especially comprises at least one cavity containing at least one nucleic acid probe or at least two nucleic acid probes selected from the group consisting of SEQ ID NO: 1, SEQ ID NO: 20, SEQ ID NO: 41, SEQ ID NO: 60, SEQ ID NO: 79, SEQ ID NO: 100, SEQ ID NO: 119, SEQ ID NO: 138, SEQ ID NO: 157, SEQ ID NO: 176, SEQ ID NO: 197, SEQ ID NO: 218 and/or further sequences from the present combinatorics of groups 1 to 12. As an alternative or in addition, the sample carrier can comprise means for optical detection of labeled microorganisms.

The fluidic channel system can, for example, be designed as a sample carrier. The sample carrier according to the invention especially comprises at least one cavity containing at least one nucleic acid probe or at least two nucleic acid probes selected from the group consisting of SEQ ID NO: 237, SEQ ID NO: 295, SEQ ID NO: 315, SEQ ID NO: 373, SEQ ID NO: 412, SEQ ID NO: 451, SEQ ID NO: 509, SEQ ID NO: 529, SEQ ID NO: 548, SEQ ID NO: 568, SEQ ID NO: 588, SEQ ID NO: 608, SEQ ID NO: 627, SEQ ID NO: 646, SEQ ID NO: 665, SEQ ID NO: 685, SEQ ID NO: 705, SEQ ID NO: 725, SEQ ID NO: 745, SEQ ID NO: 765, SEQ ID NO: 805 and/or further sequences from the present combinatorics of groups 13 to 33. As an alternative or in addition, the sample carrier can comprise means for optical detection of labeled microorganisms.

The fluidic channel system can, for example, be designed as a sample carrier. The sample carrier according to the invention especially comprises at least one cavity containing at least one nucleic acid probe or at least two nucleic acid probes selected from the group consisting of SEQ ID NO: 825, SEQ ID NO: 845, SEQ ID NO: 866, SEQ ID NO: 887, SEQ ID NO: 928, SEQ ID NO: 948, SEQ ID NO: 968, SEQ ID NO: 989, SEQ ID NO: 1010, SEQ ID NO: 1072, SEQ ID NO: 1134, SEQ ID NO: 1155, SEQ ID NO: 1197 and/or further sequences from the present combinatorics of groups 34 to 46. As an alternative or in addition, the sample carrier can comprise means for optical detection of labeled microorganisms.

The sample carrier according to the invention can be designed as a disk-shaped sample carrier. For example, the sample carrier can be designed as a planar sample carrier. The advantage here is that the disk shape of the sample carrier can utilize centrifugal force for fluid conveyance. Fluid conveyance is also achievable by means of pressure or in another way. The sample carrier can alternatively have a three-dimensional extent, for example in the form of a cylinder or in the style of a cuvette.

For example, the disk-shaped nature can have rotational symmetry. This can be advantageous for centrifugation. It is also alternatively possible to form rectangular sample carriers, as in the case of a chip card, or segment-shaped sample carriers, as in the case of a pizza slice.

The invention will now be described in more detail with reference to exemplary embodiments, without however being limited to said exemplary embodiments. Further exemplary embodiments arise from combination of the features of individual or multiple claims with one another and/or with individual or multiple features of the exemplary embodiments.

In the figures:

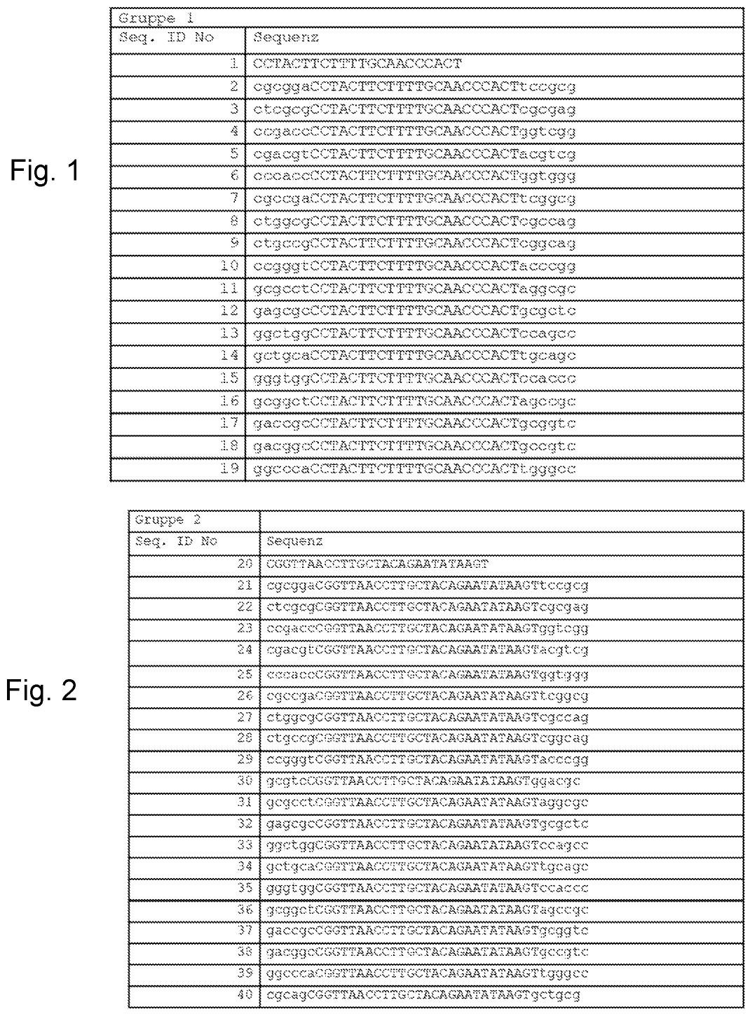

FIG. 1 shows group 1 comprising sequences SEQ ID NO: 1 to SEQ ID NO: 19 from combinatorics of the nucleic acid probes according to the invention,

FIG. 2 shows group 2 comprising sequences SEQ ID NO: 20 to SEQ ID NO: 40 from combinatorics of the nucleic acid probes according to the invention,

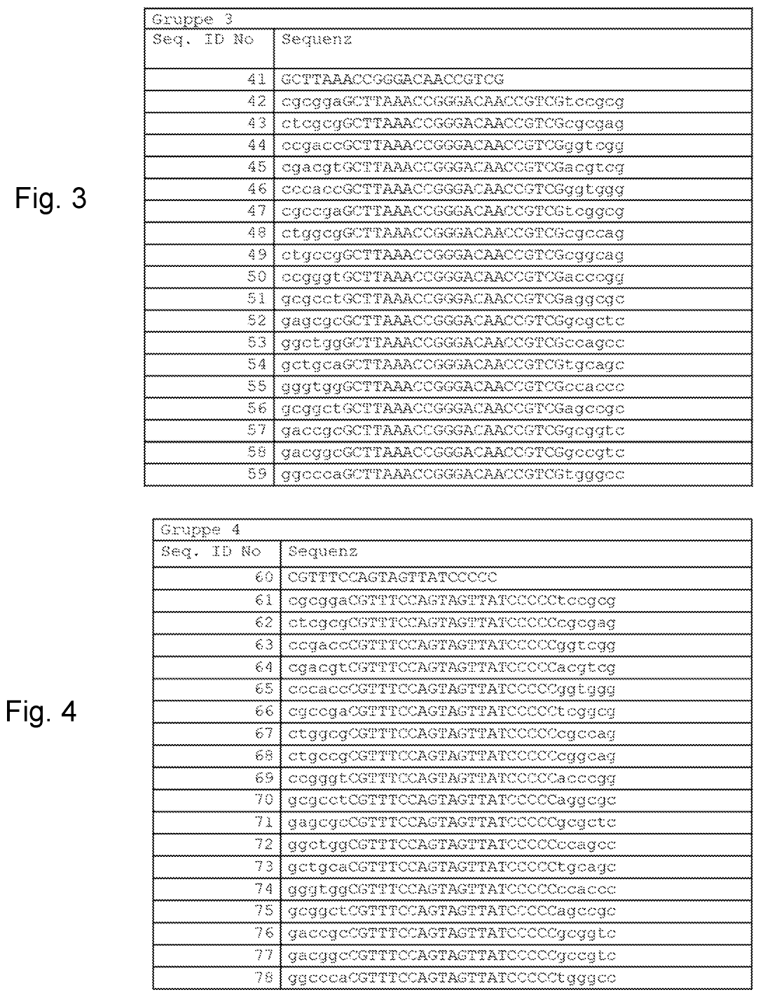

FIG. 3 shows group 3 comprising sequences SEQ ID NO: 41 to SEQ ID NO: 59 from combinatorics of the nucleic acid probes according to the invention,

FIG. 4 shows group 4 comprising sequences SEQ ID NO: 60 to SEQ ID NO: 78 from combinatorics of the nucleic acid probes according to the invention,

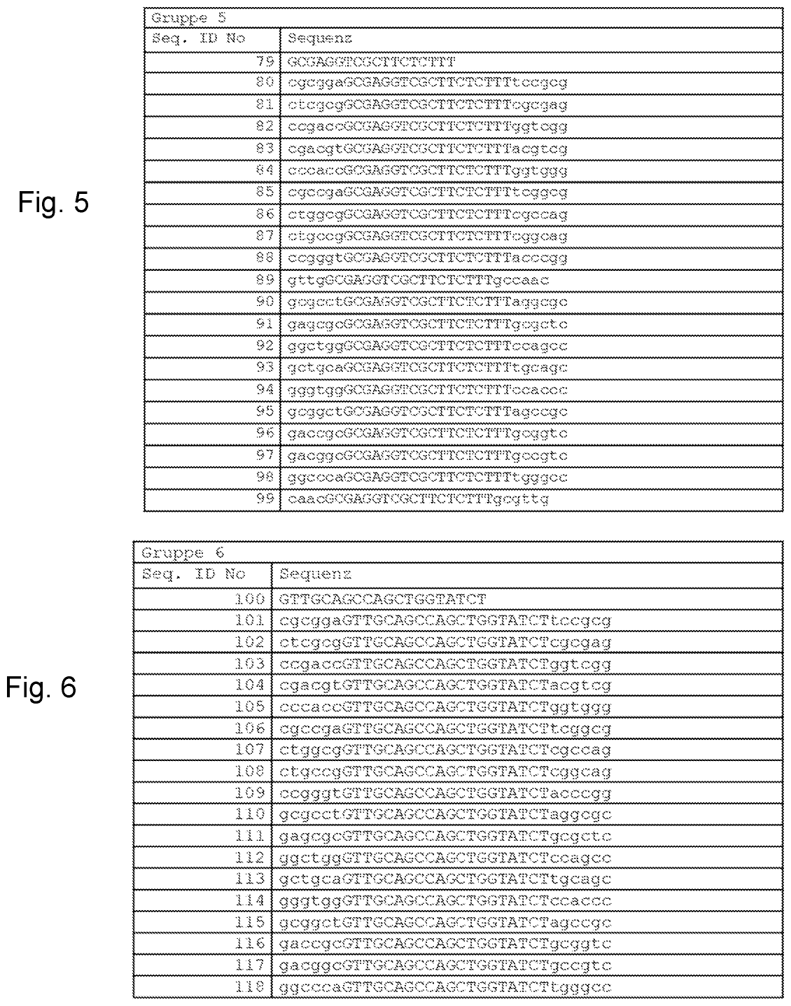

FIG. 5 shows group 5 comprising sequences SEQ ID NO: 79 to SEQ ID NO: 99 from combinatorics of the nucleic acid probes according to the invention,

FIG. 6 shows group 6 comprising sequences SEQ ID NO: 100 to SEQ ID NO: 118 from combinatorics of the nucleic acid probes according to the invention,

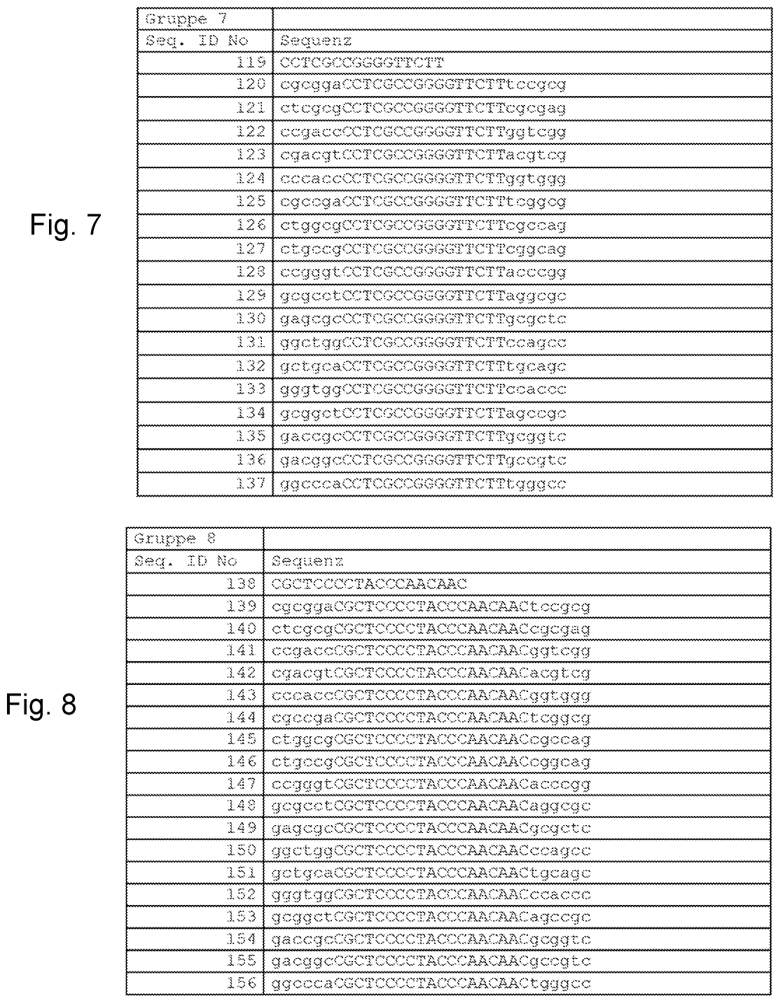

FIG. 7 shows group 7 comprising sequences SEQ ID NO: 119 to SEQ ID NO: 137 from combinatorics of the nucleic acid probes according to the invention,

FIG. 8 shows group 8 comprising sequences SEQ ID NO: 138 to SEQ ID NO: 156 from combinatorics of the nucleic acid probes according to the invention,

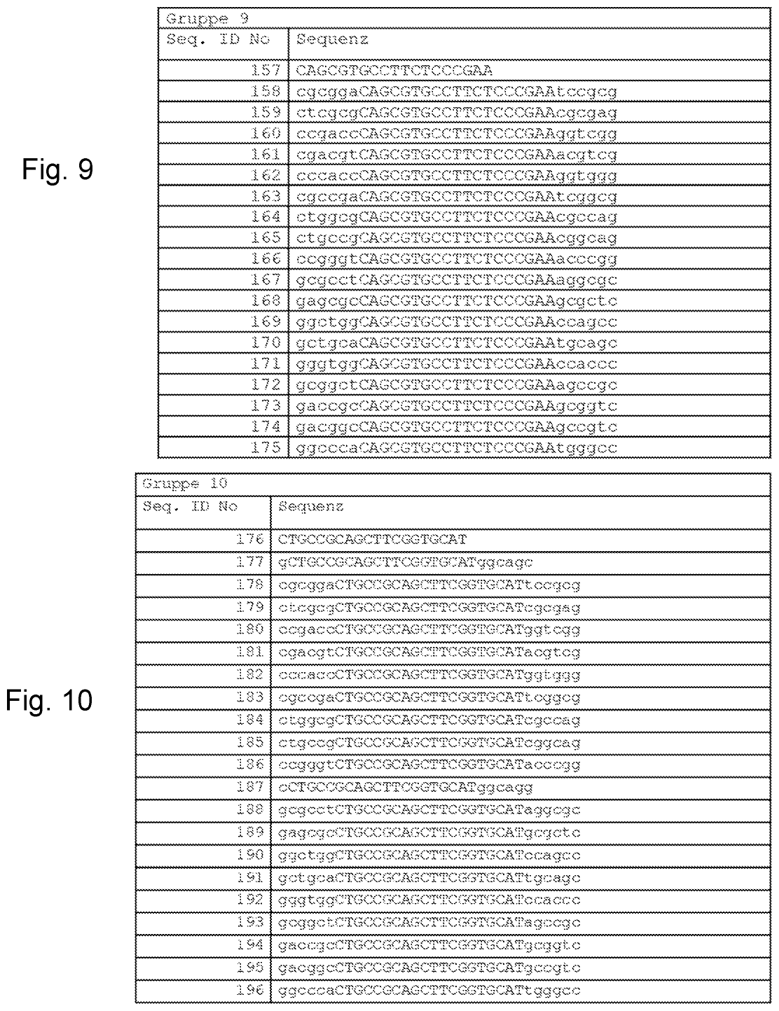

FIG. 9 shows group 9 comprising sequences SEQ ID NO: 157 to SEQ ID NO: 175 from combinatorics of the nucleic acid probes according to the invention,

FIG. 10 shows group 10 comprising sequences SEQ ID NO: 176 to SEQ ID NO: 196 from combinatorics of the nucleic acid probes according to the invention,

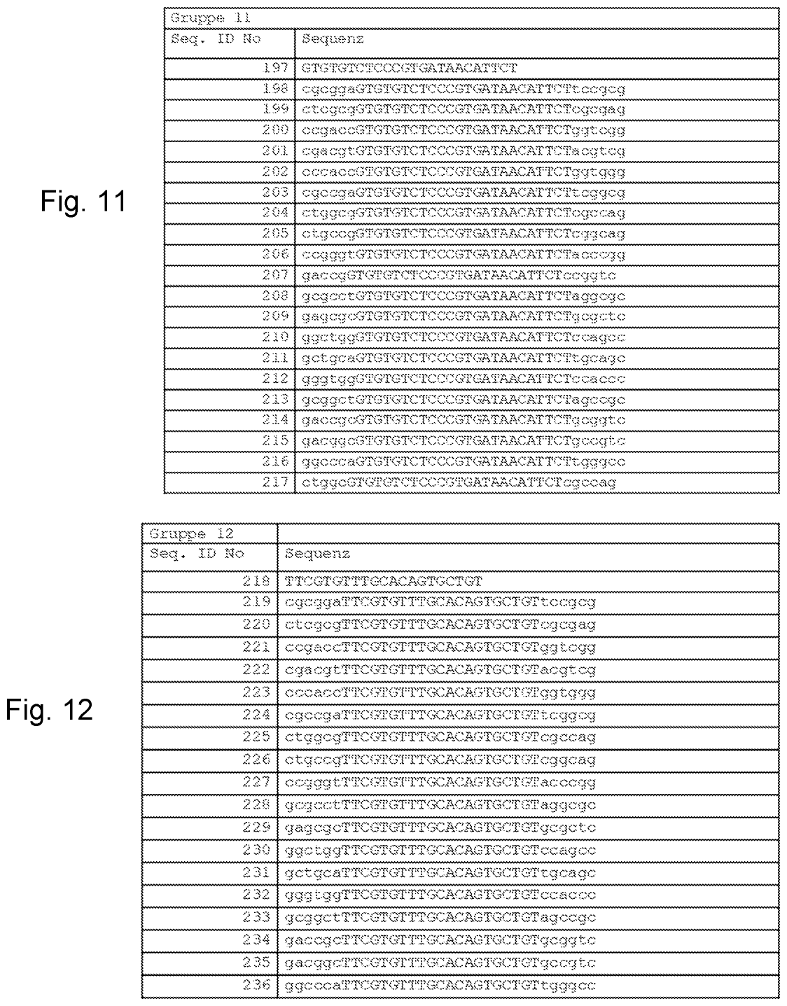

FIG. 11 shows group 11 comprising sequences SEQ ID NO: 197 to SEQ ID NO: 217 from combinatorics of the nucleic acid probes according to the invention,

FIG. 12 shows group 12 comprising sequences SEQ ID NO: 218 to SEQ ID NO: 236 from combinatorics of the nucleic acid probes according to the invention,

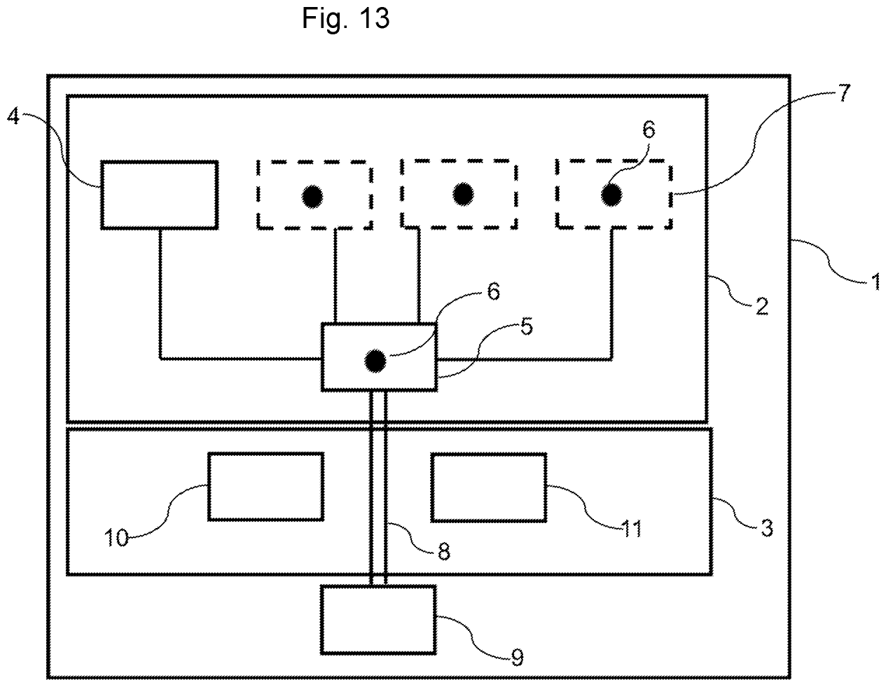

FIG. 13 shows a schematic depiction of a fluidic channel system for carrying out the method according to the invention,

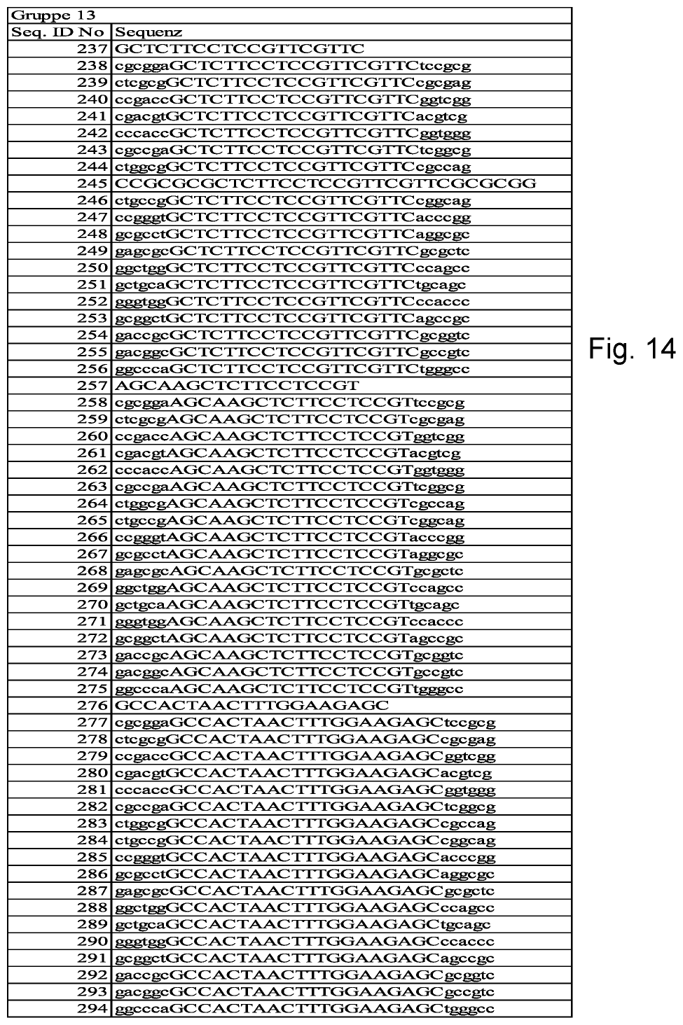

FIG. 14 shows group 13 comprising sequences SEQ ID NO: 237 to SEQ ID NO: 294 from combinatorics of the nucleic acid probes according to the invention,

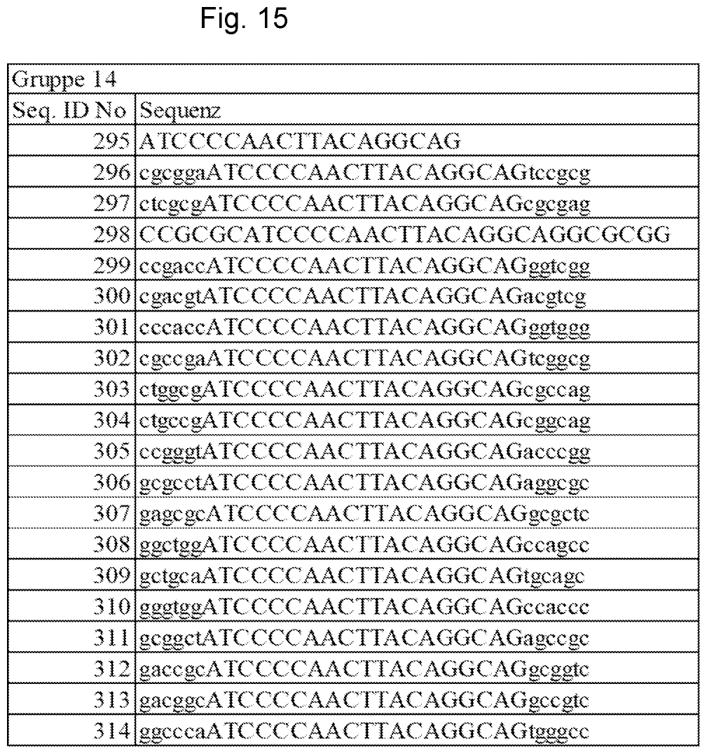

FIG. 15 shows group 14 comprising sequences SEQ ID NO: 295 to SEQ ID NO: 314 from combinatorics of the nucleic acid probes according to the invention,

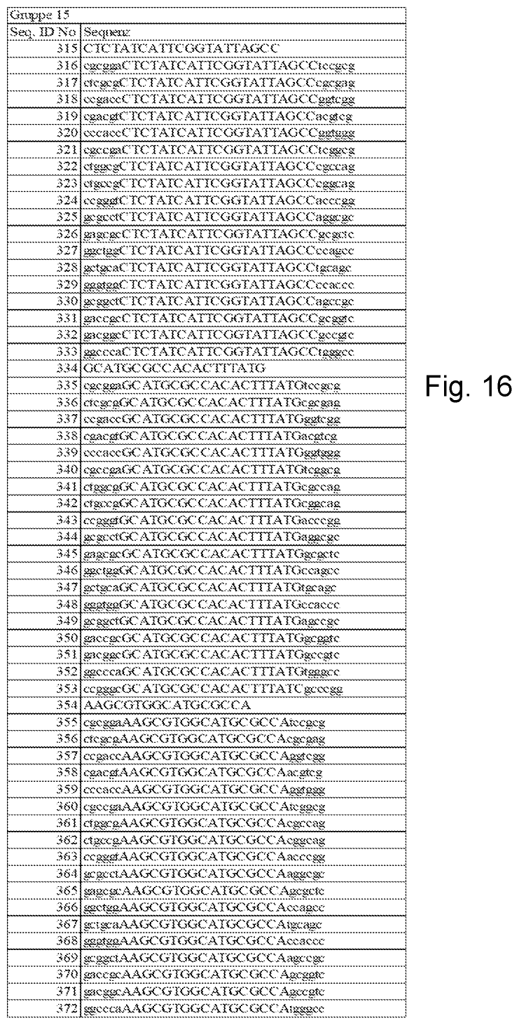

FIG. 16 shows group 15 comprising sequences SEQ ID NO: 315 to SEQ ID NO: 372 from combinatorics of the nucleic acid probes according to the invention,

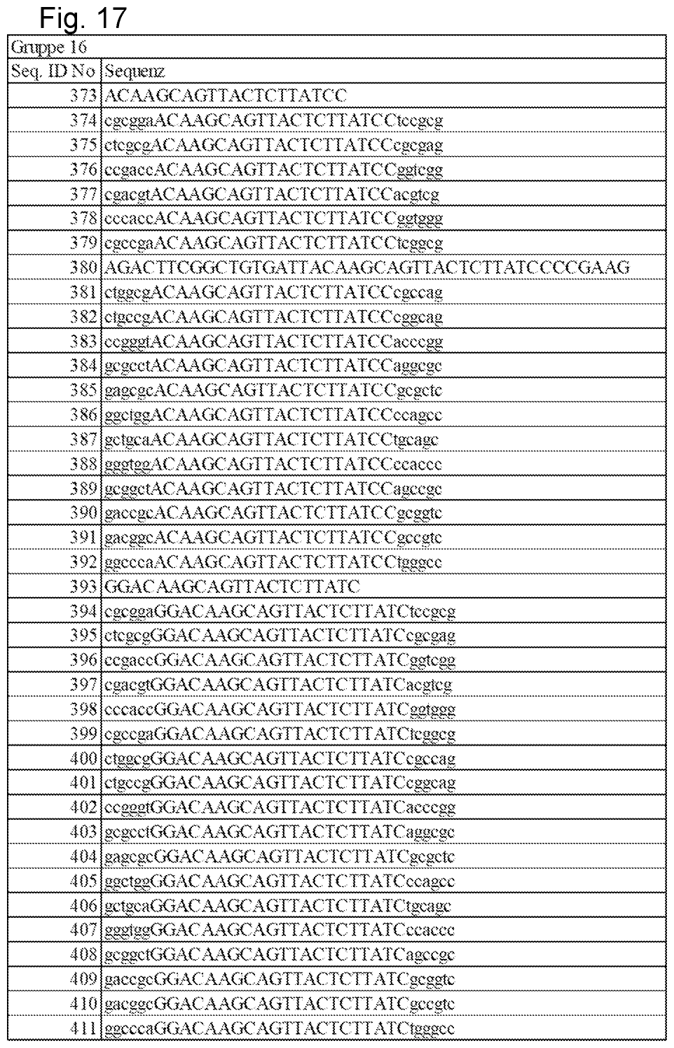

FIG. 17 shows group 16 comprising sequences SEQ ID NO: 373 to SEQ ID NO: 411 from combinatorics of the nucleic acid probes according to the invention,

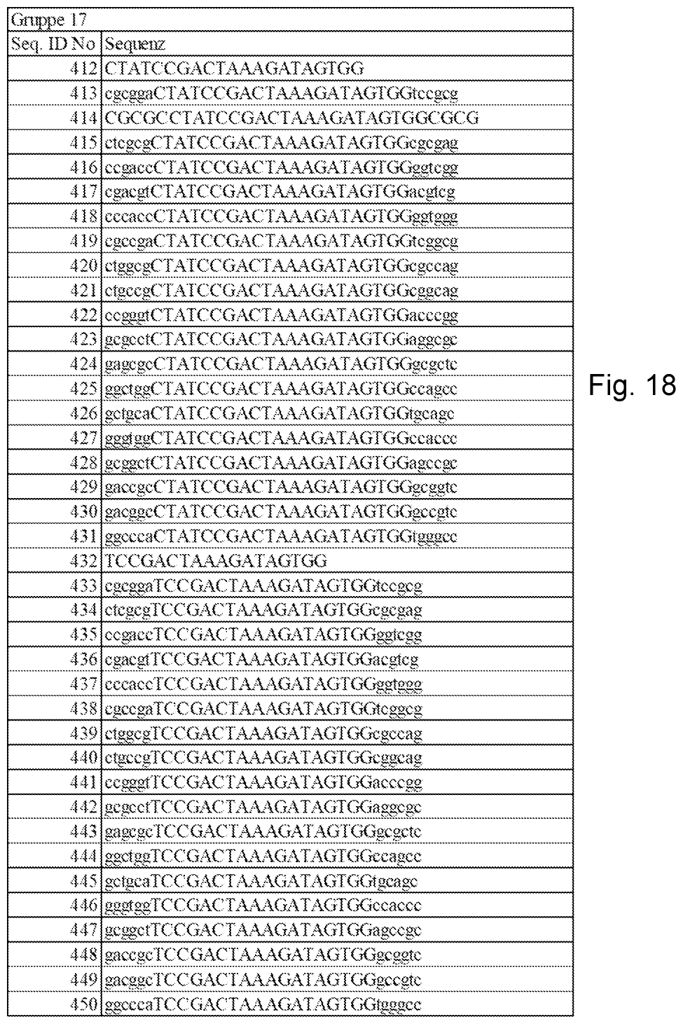

FIG. 18 shows group 17 comprising sequences SEQ ID NO: 412 to SEQ ID NO: 450 from combinatorics of the nucleic acid probes according to the invention,

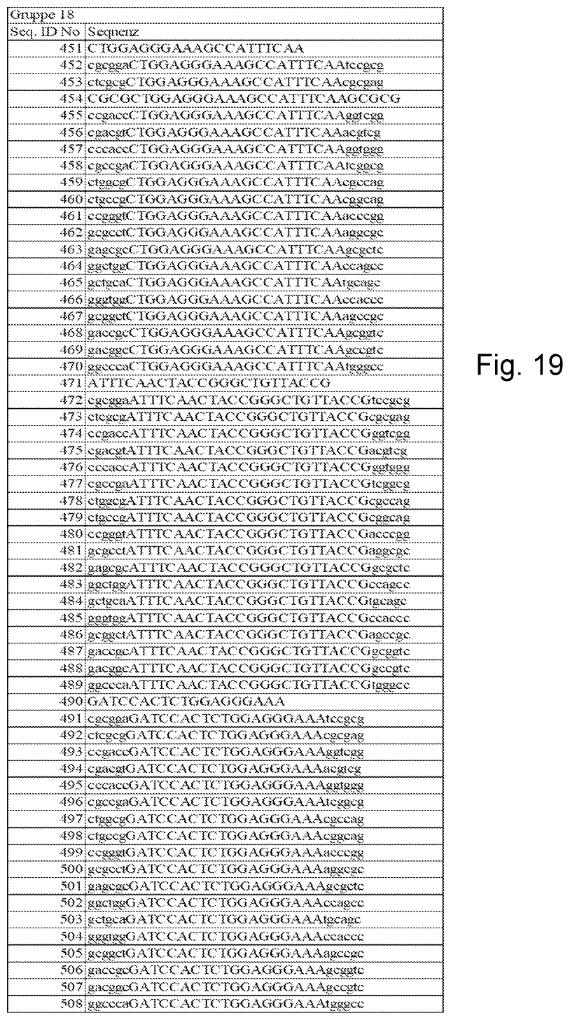

FIG. 19 shows group 18 comprising sequences SEQ ID NO: 451 to SEQ ID NO: 508 from combinatorics of the nucleic acid probes according to the invention,

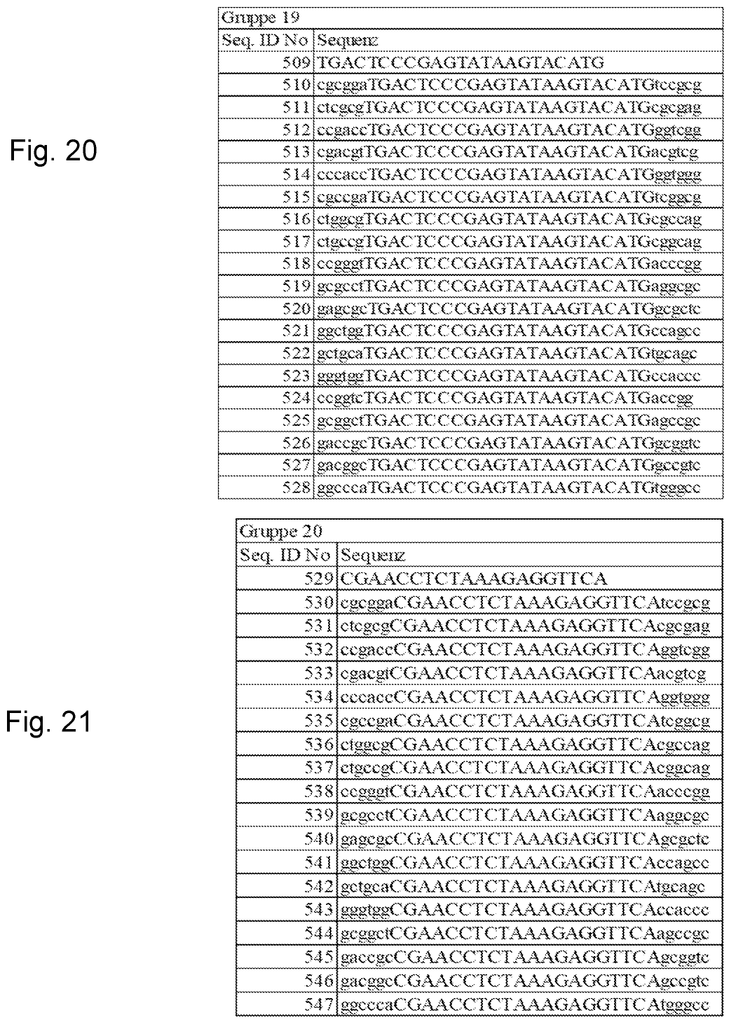

FIG. 20 shows group 19 comprising sequences SEQ ID NO: 509 to SEQ ID NO: 528 from combinatorics of the nucleic acid probes according to the invention,

FIG. 21 shows group 20 comprising sequences SEQ ID NO: 529 to SEQ ID NO: 547 from combinatorics of the nucleic acid probes according to the invention,

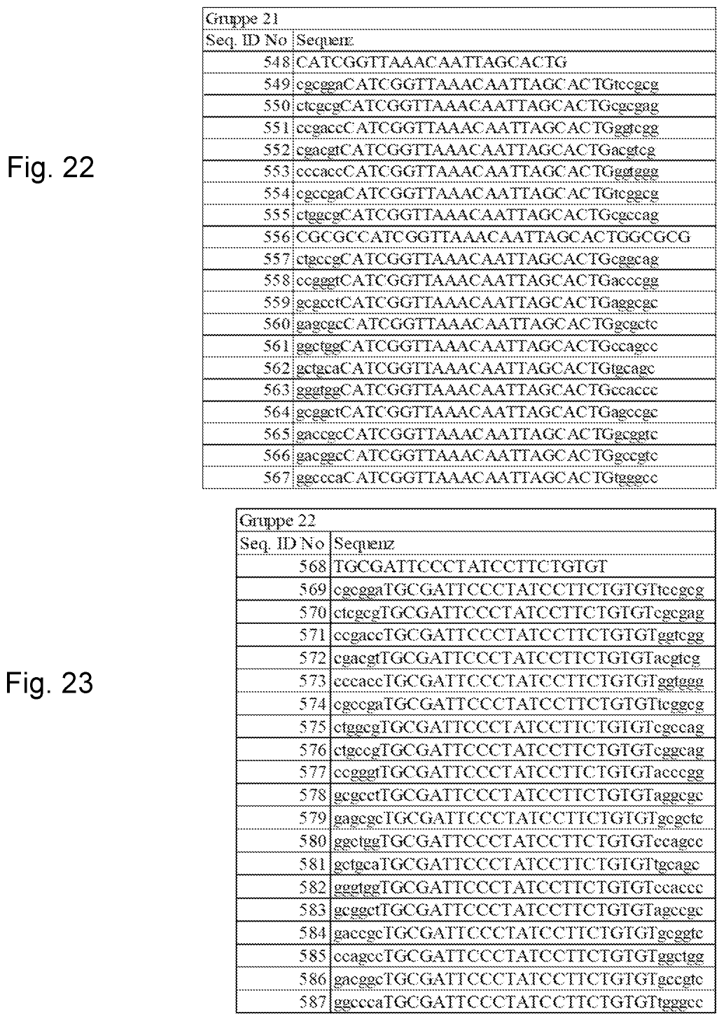

FIG. 22 shows group 21 comprising sequences SEQ ID NO: 548 to SEQ ID NO: 567 from combinatorics of the nucleic acid probes according to the invention,

FIG. 23 shows group 22 comprising sequences SEQ ID NO: 568 to SEQ ID NO: 587 from combinatorics of the nucleic acid probes according to the invention,

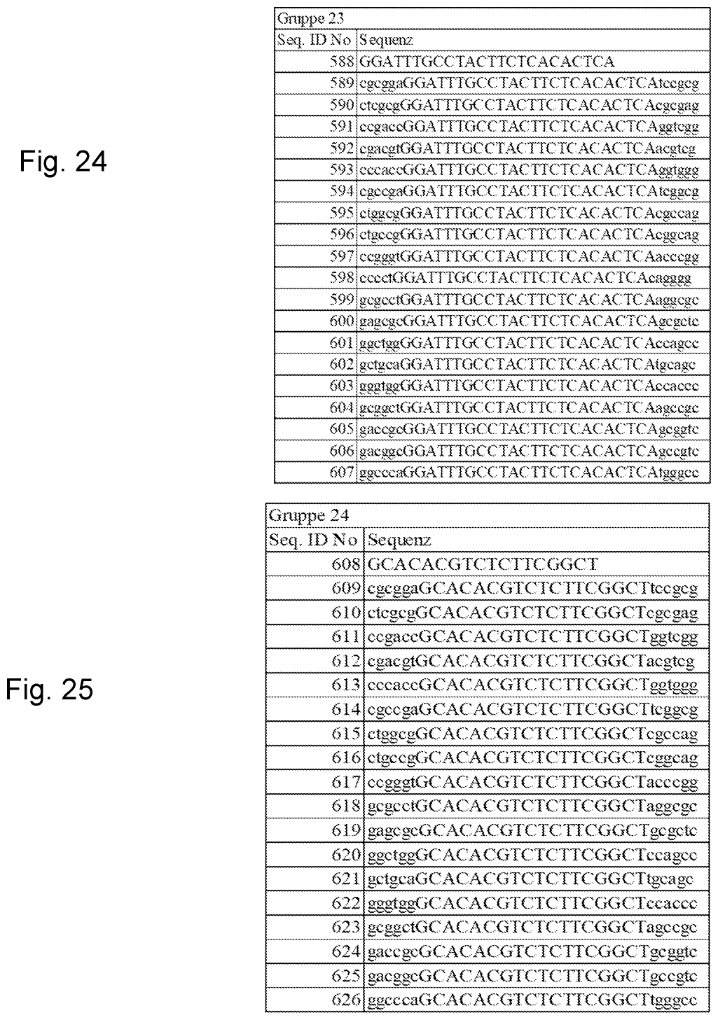

FIG. 24 shows group 23 comprising sequences SEQ ID NO: 588 to SEQ ID NO: 607 from combinatorics of the nucleic acid probes according to the invention,

FIG. 25 shows group 24 comprising sequences SEQ ID NO: 608 to SEQ ID NO: 626 from combinatorics of the nucleic acid probes according to the invention,

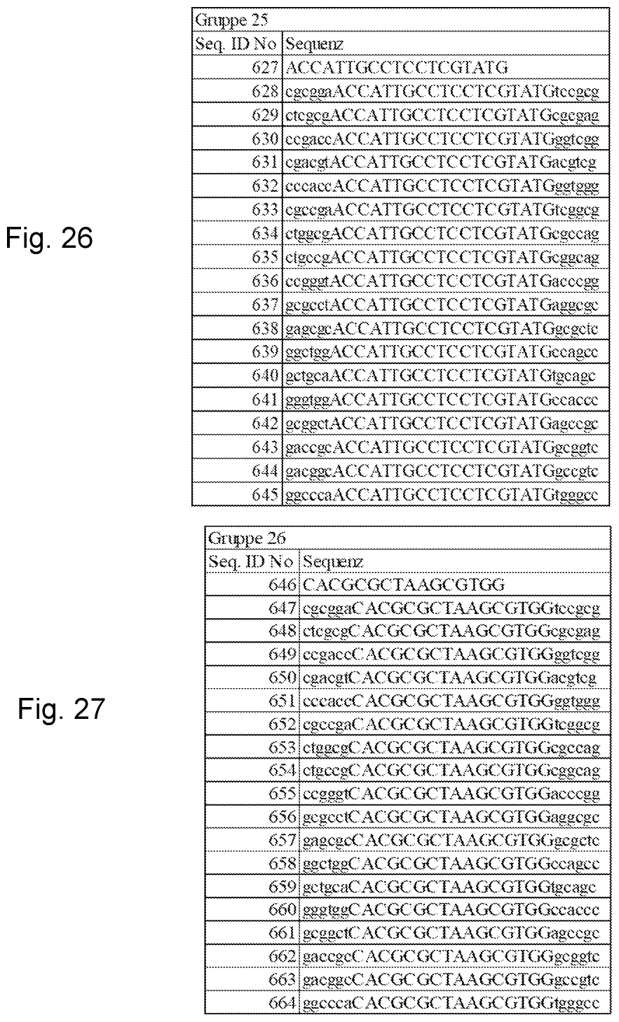

FIG. 26 shows group 25 comprising sequences SEQ ID NO: 627 to SEQ ID NO: 645 from combinatorics of the nucleic acid probes according to the invention,

FIG. 27 shows group 26 comprising sequences SEQ ID NO: 646 to SEQ ID NO: 664 from combinatorics of the nucleic acid probes according to the invention,

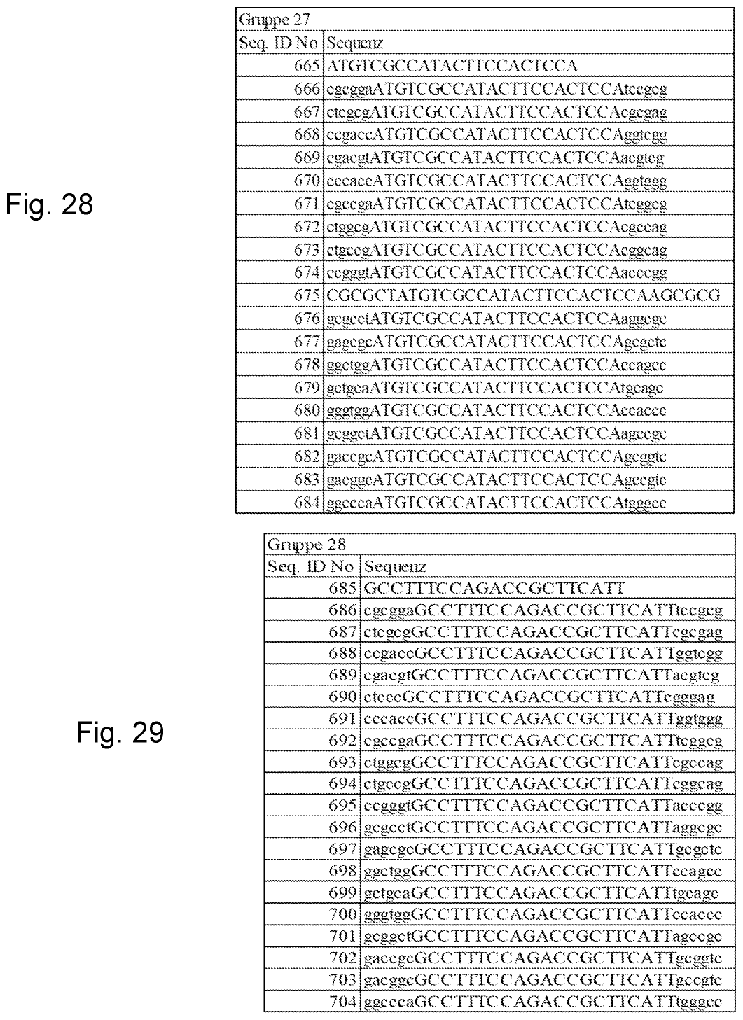

FIG. 28 shows group 27 comprising sequences SEQ ID NO: 665 to SEQ ID NO: 684 from combinatorics of the nucleic acid probes according to the invention,

FIG. 29 shows group 28 comprising sequences SEQ ID NO: 685 to SEQ ID NO: 704 from combinatorics of the nucleic acid probes according to the invention,

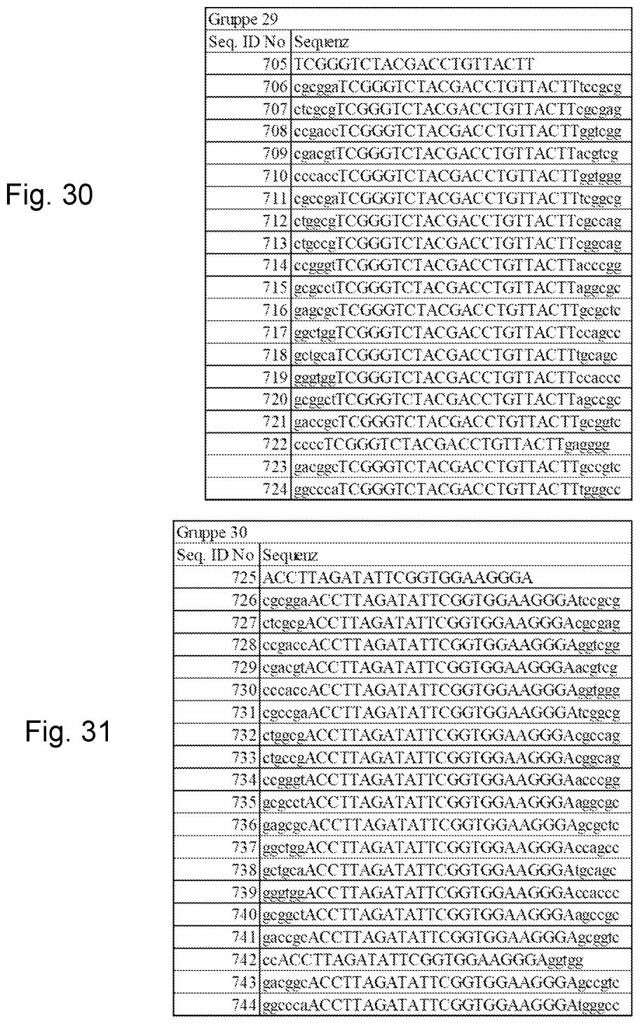

FIG. 30 shows group 29 comprising sequences SEQ ID NO: 705 to SEQ ID NO: 724 from combinatorics of the nucleic acid probes according to the invention,

FIG. 31 shows group 30 comprising sequences SEQ ID NO: 725 to SEQ ID NO: 744 from combinatorics of the nucleic acid probes according to the invention,

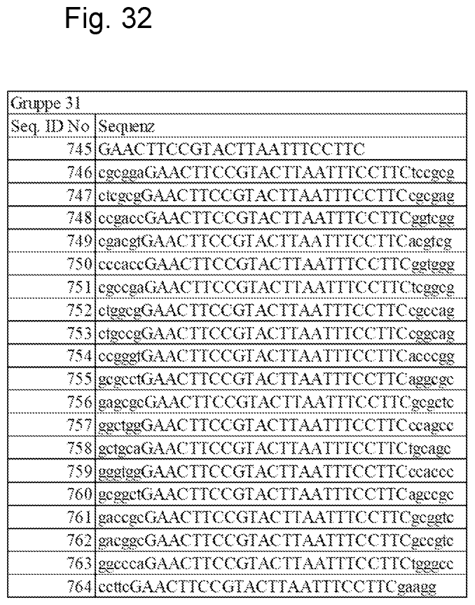

FIG. 32 shows group 31 comprising sequences SEQ ID NO: 745 to SEQ ID NO: 764 from combinatorics of the nucleic acid probes according to the invention,

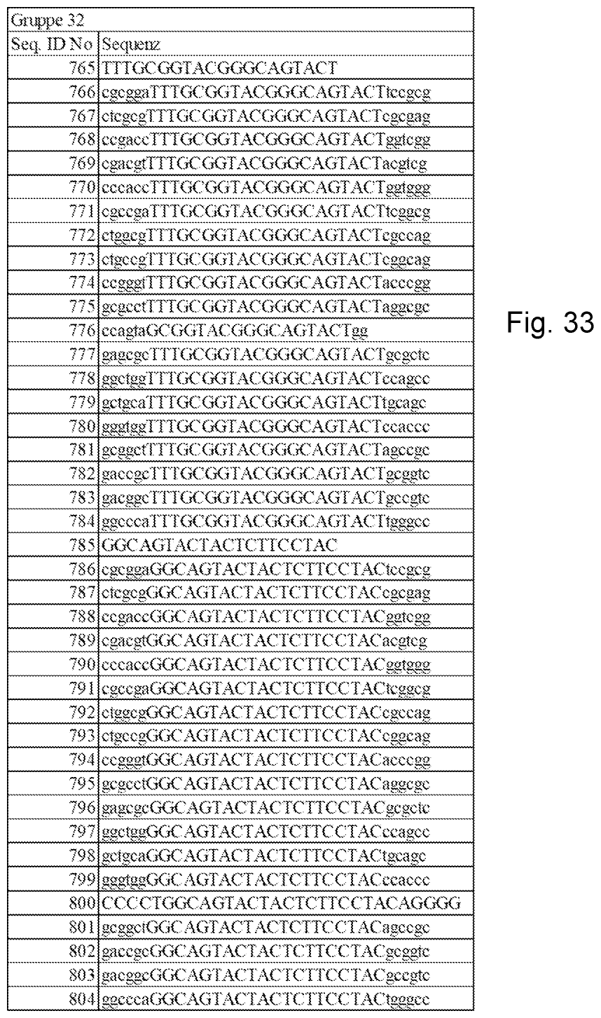

FIG. 33 shows group 32 comprising sequences SEQ ID NO: 765 to SEQ ID NO: 804 from combinatorics of the nucleic acid probes according to the invention,

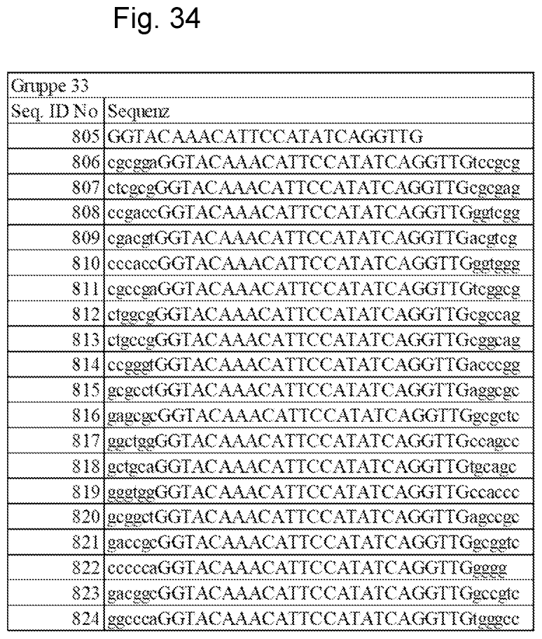

FIG. 34 shows group 33 comprising sequences SEQ ID NO: 805 to SEQ ID NO: 824 from combinatorics of the nucleic acid probes according to the invention,

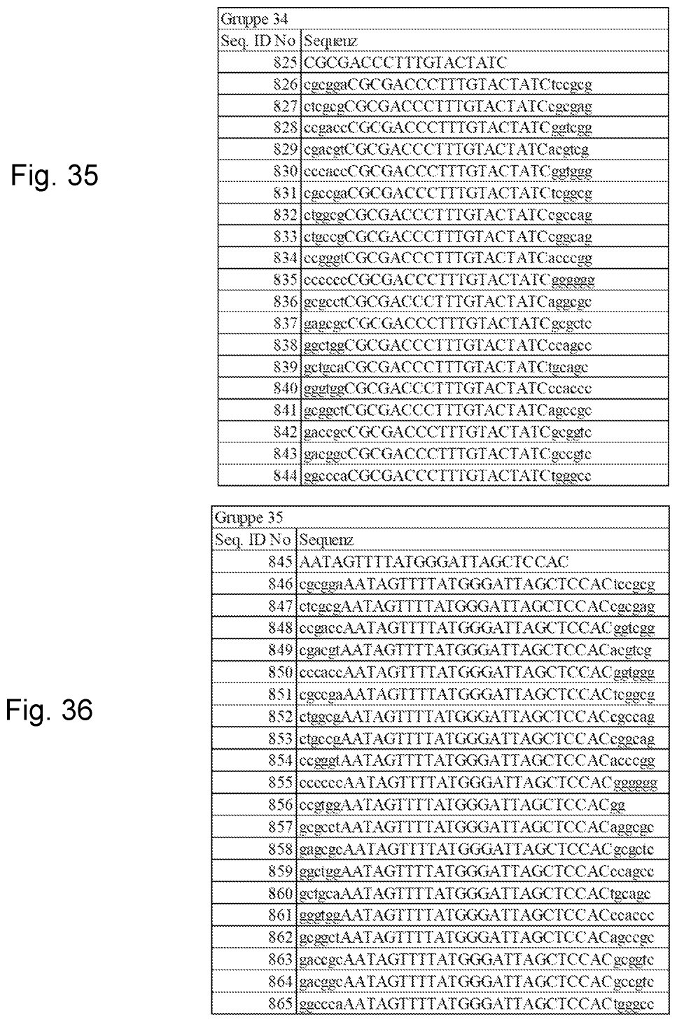

FIG. 35 shows group 34 comprising sequences SEQ ID NO: 825 to SEQ ID

i.NO: 844 from combinatorics of the
i.i.nucleic acid probes according to the invention,

FIG. 36 shows group 35 comprising sequences SEQ ID NO: 845 to SEQ ID

i.NO: 865 from combinatorics of the
i.i.nucleic acid probes according to the invention,

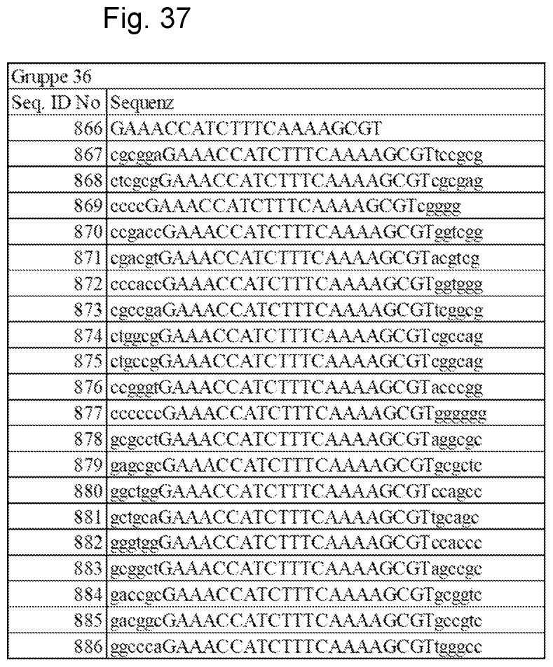

FIG. 37 shows group 36 comprising sequences SEQ ID NO: 866 to SEQ ID

i.NO: 886 from combinatorics of the
i.i.nucleic acid probes according to the invention,

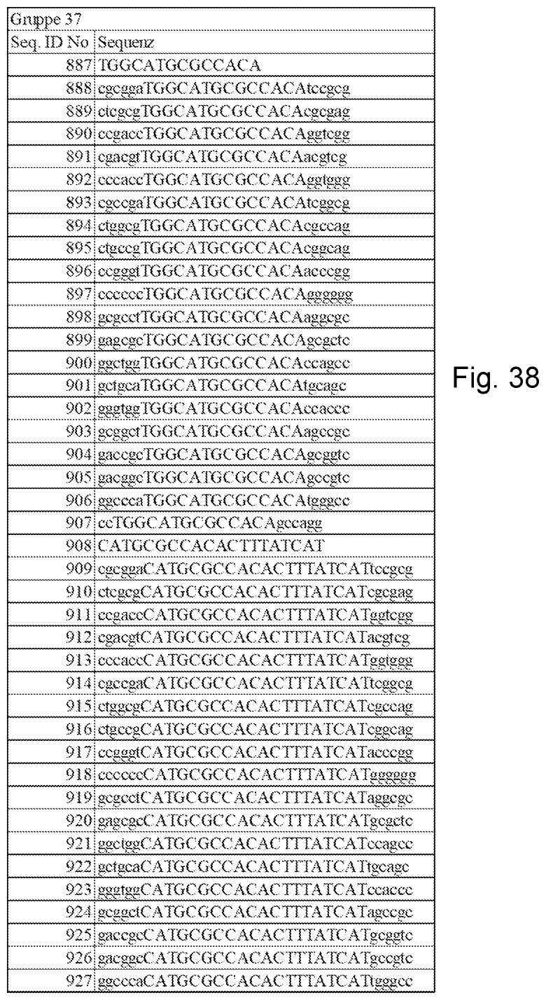

FIG. 38 shows group 37 comprising sequences SEQ ID NO: 887 to SEQ ID

i.NO: 927 from combinatorics of the
i.i.nucleic acid probes according to the invention,

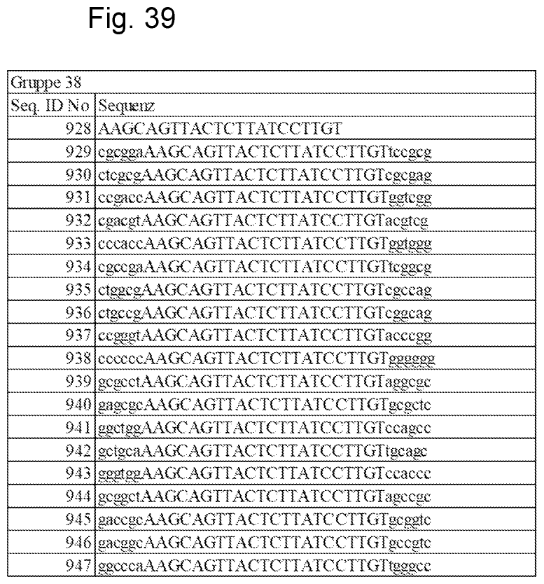

FIG. 39 shows group 38 comprising sequences SEQ ID NO: 928 to SEQ ID

i.NO: 947 from combinatorics of the
i.i.nucleic acid probes according to the invention,

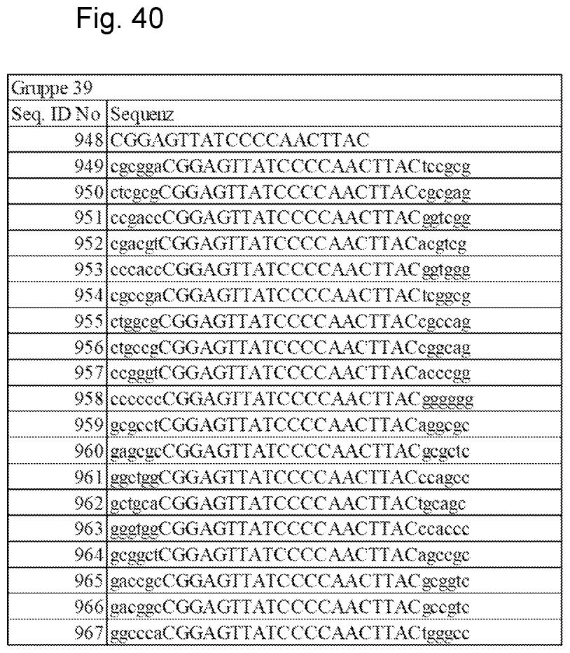

FIG. 40 shows group 39 comprising sequences SEQ ID NO: 948 to SEQ ID

i.NO: 967 from combinatorics of the
i.i.nucleic acid probes according to the invention,

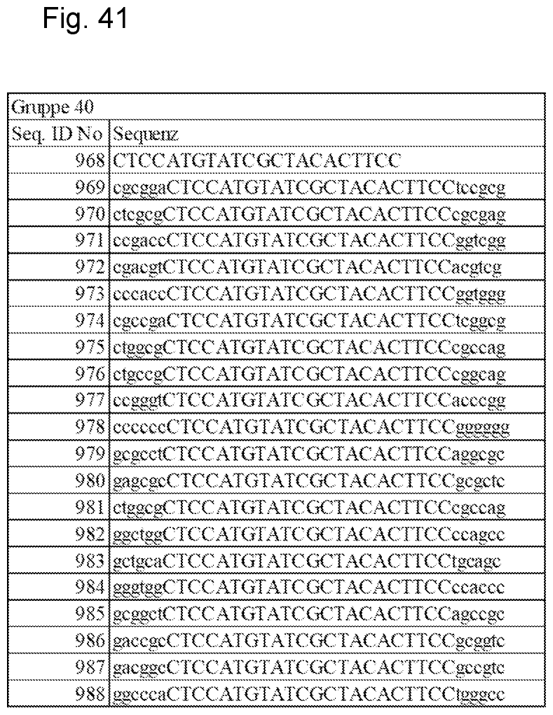

FIG. 41 shows group 40 comprising sequences SEQ ID NO: 968 to SEQ ID

i.NO: 988 from combinatorics of the
i.i.nucleic acid probes according to the invention,

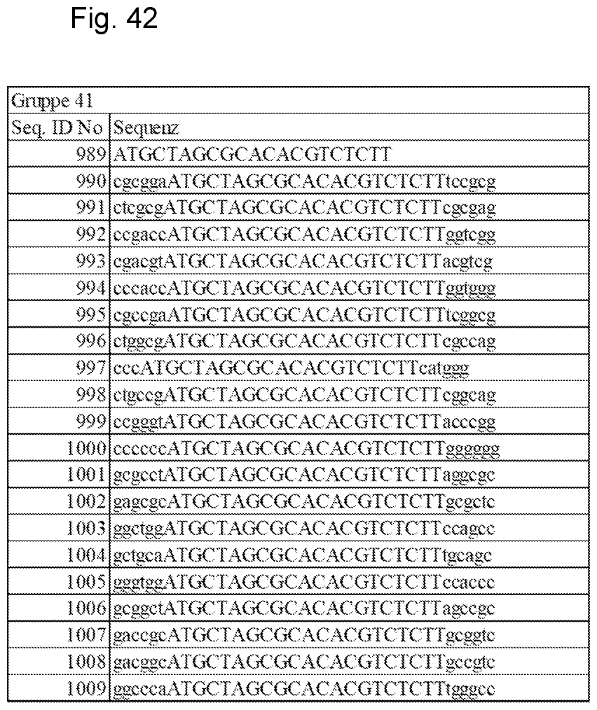

FIG. 42 shows group 41 comprising sequences SEQ ID NO: 989 to SEQ ID

i.NO: 1009 from combinatorics of the
i.i.nucleic acid probes according to the invention,

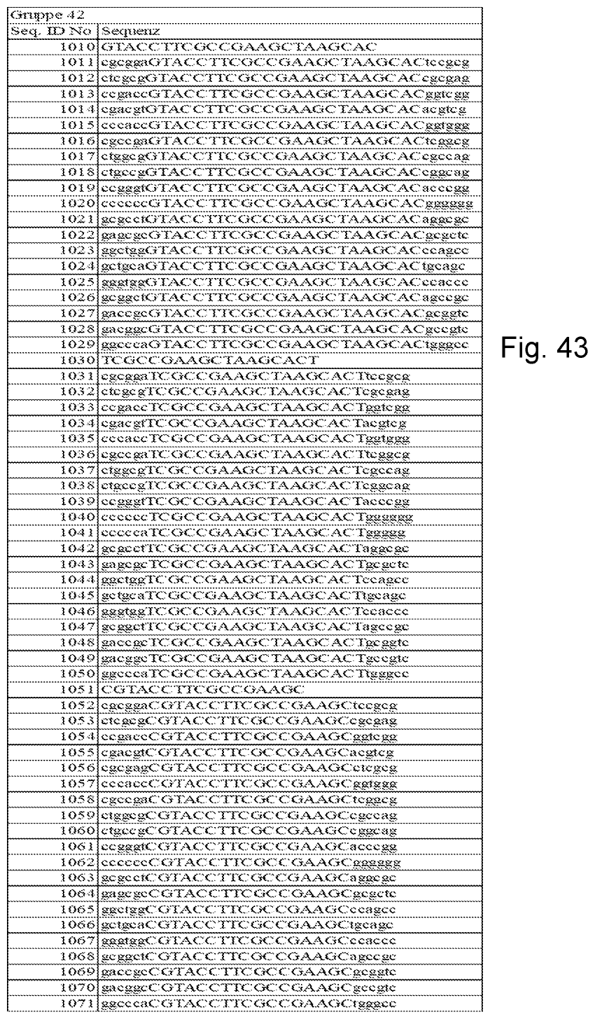

FIG. 43 shows group 42 comprising sequences SEQ ID NO: 1010 to SEQ ID NO: 1071 from combinatorics of the nucleic acid probes according to the invention,

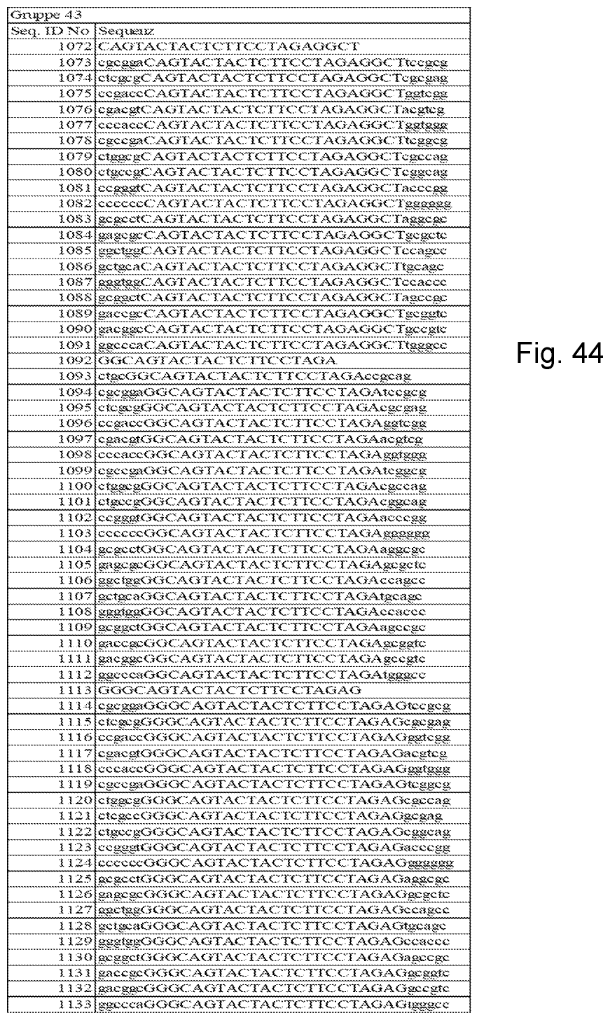

FIG. 44 shows group 43 comprising sequences SEQ ID NO: 1072 to SEQ ID NO: 1133 from combinatorics of the nucleic acid probes according to the invention,

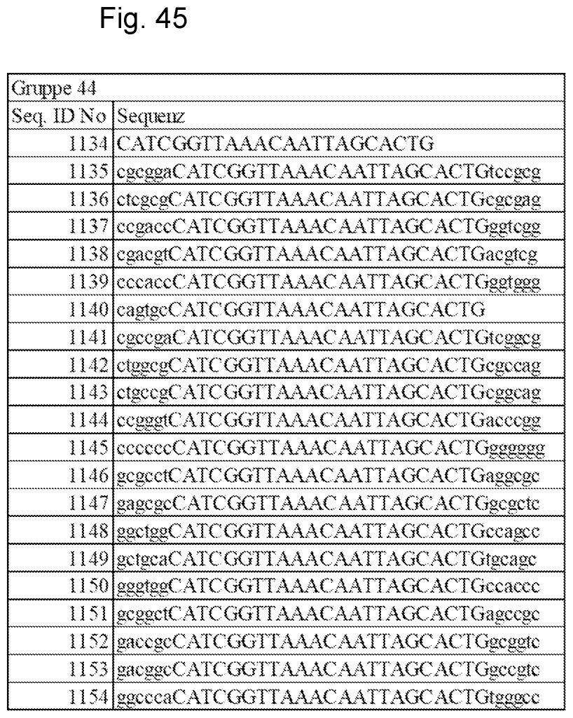

FIG. 45 shows group 44 comprising sequences SEQ ID NO: 1134 to SEQ ID NO: 1154 from combinatorics of the nucleic acid probes according to the invention,

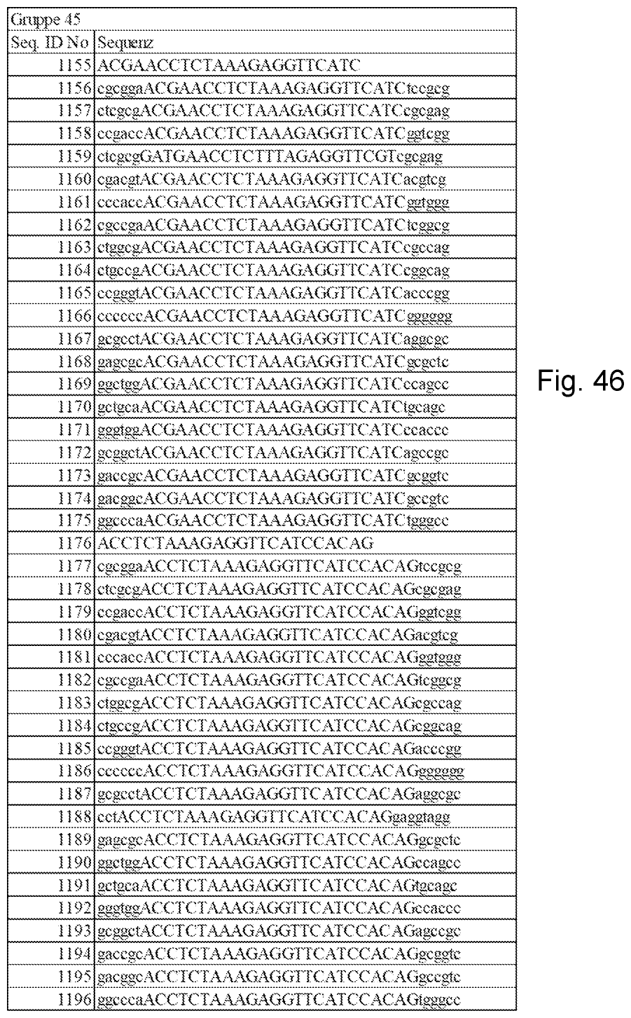

FIG. 46 shows group 45 comprising sequences SEQ ID NO: 1155 to SEQ ID NO: 1196 from combinatorics of the nucleic acid probes according to the invention,

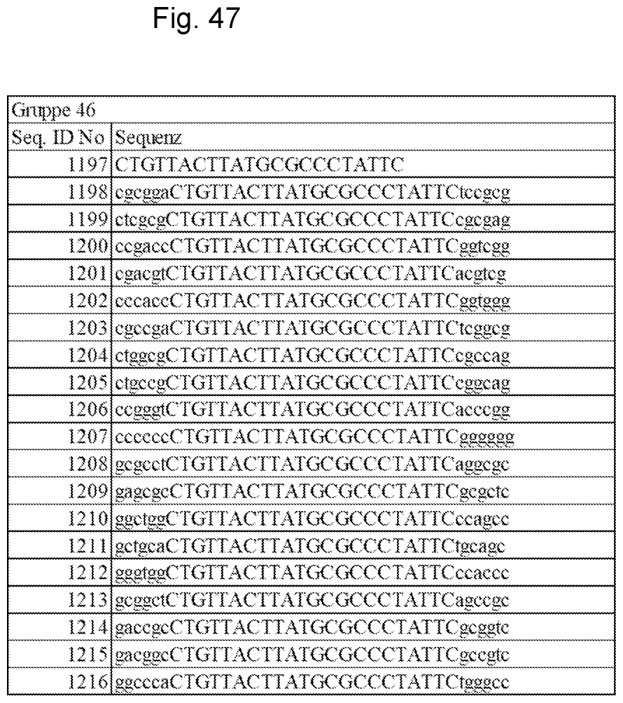

FIG. 47 shows group 46 comprising sequences SEQ ID NO: 1197 to SEQ ID NO: 1216 from combinatorics of the nucleic acid probes according to the invention.

FIGS. 1 to 12 show combinatorics of the nucleic acid probes according to the invention, consisting of groups 1 to 12. Selection is made of a representative from each group or a representative from at least two groups for a mixture of nucleic acid probes and use is made thereof in a method according to the invention for detecting microorganisms, for example of the Enterobacteriaceae family of bacteria.

The nucleic acid probes each have a stem sequence at the 5′ end and/or at the 3′ end, which is depicted in lowercase letters in the examples according to FIGS. 1 to 12. Formed in each case between the stem sequences of the nucleic acid probes is a functional part, which is depicted in uppercase letters in the examples according to FIGS. 1 to 12. The functional part is the complementary region, i.e., binding region, in relation to the target sequence. The stem sequences within each nucleic acid probe can bind to one another in the absence of target sequences and thus form a “hairpin” structure, thereby suppressing the fluorescence of the dye. After the functional part binds to the target sequence, said “hairpin” structure comes apart, whereupon the fluorescence of the dye can be detected. To realize the method, a fluidic channel system, for example a disk-shaped sample carrier, which can, but need not, have rotational symmetry, can be provided with means for carrying out said method. In particular, the channel system can comprise a cavity containing at least one nucleic acid probe or at least two nucleic acid probes selected from the mentioned groups and/or further sequences from the present combinatorics of groups 1 to 12 and/or be provided with means for optical detection of labeled microorganisms.

FIGS. 14 to 34 show combinatorics of the nucleic acid probes according to the invention, consisting of groups 13 to 33. Selection is made of a representative from each group or a representative from at least two groups for a mixture of nucleic acid probes and use is made thereof in a method according to the invention for detecting microorganisms, especially of the Listeriaceae family of bacteria.

FIGS. 35 to 47 show combinatorics of the nucleic acid probes according to the invention, consisting of groups 34 to 46. Selection is made of a representative from each group or a representative from at least two groups for a mixture of nucleic acid probes and use is made thereof in a method according to the invention for detecting microorganisms, especially of the Listeria monocytogenes species of bacteria.

FIG. 13 shows a fluidic channel system 1 for detection of microorganisms, for example of the Enterobacteriaceae family of bacteria, comprising a preparation zone 2 and a detection zone 3. The fluidic channel system has a sampling chamber 4 into which a test sample is directly introducible. The sampling chamber 4 is connected to a reaction chamber 5 which contains reagents 6 kept in reserve.

It is apparent from FIG. 13 that reaction chamber 5 is connected to at least one reagent chamber 7 which contains reagents 6 kept in reserve. In the channel system design depicted here in FIG. 13, the channel system 1 is formed with three reagent chambers 7 which are connected to one another in parallel.

In further exemplary embodiments, a different number of reagent chambers 7 is present, for example more than three or less than three. In a further exemplary embodiment, a channel system 1 is free of reagent chambers 7.

The reaction chamber 5 is connected to a detection chamber 9 via a detection channel 8. The channel system 1 has a light source 10 and a detector 11 that are formed in the detection zone 3.

In a preferred application, the test sample is first introduced into the fluidic channel system 1. In the preparation zone 2, the test sample is conducted to the reaction chamber 5. At the same time or afterwards, the reagents 6 are conducted from the reagent chambers 7 to the reaction chamber 5, though this step can be omitted if reagents are already kept in reserve in the reaction chamber 5.

After the reaction with the reagents 6 kept in reserve, the test sample is conducted into the detection chamber 9 via the detection channel 8. Thereafter, the detection reaction is carried out by optical signal capture by means of photometry. For example, the detection reaction is carried out by means of turbidometry, cytometry or fluorescence measurement.

According to the invention, what is therefore proposed is to provide a method for detecting microorganisms, for example of the Enterobacteriaceae family of bacteria, in a sample by means of delivery of identity-determining nucleic acid probes into the individual cell bodies, wherein the nucleic acid probes hybridize to the nucleic acids of the microorganisms, and subsequent optical detection of the hybridizations generated in the individual cell bodies, wherein a mixture of at least a first nucleic acid probe and a second nucleic acid probe is used, and wherein the first nucleic acid probe and the second nucleic acid probe respectively bind in one of two nonoverlapping regions of the target nucleic acid.

### LIST OF REFERENCE SIGNS

1 Fluidic channel system

2 Preparation zone

3 Detection zone

4 Sampling chamber

5 Reaction chamber (containing reagents kept in reserve)

6 Reagents kept in reserve

7 Optionally at least one reagent chamber (containing reagents kept in reserve)

8 Detection channel

9 Detection chamber

10 Light source

11 Detector

Group 13

| Seq. ID No | Sequence |
|---|---|
| 237 | GCTCTTCCTCCGTTCGTTC |
| 238 | cgcggaGCTCTTCCTCCGTTCGTTCtccgcg |
| 239 | ctcgcgGCTCTTCCTCCGTTCGTTCcgcgag |
| 240 | ccgaccGCTCTTCCTCCGTTCGTTCggtcgg |
| 241 | cgacgtGCTCTTCCTCCGTTCGTTCacgtcg |
| 242 | cccaccGCTCTTCCTCCGTTCGTTCggtggg |
| 243 | cgccgaGCTCTTCCTCCGTTCGTTCtcggcg |
| 244 | ctggcgGCTCTTCCTCCGTTCGTTCcgccag |
| 245 | CCGCGCGCTCTTCCTCCGTTCGTTCGCGCGG |
| 246 | ctgccgGCTCTTCCTCCGTTCGTTCcggcag |
| 247 | ccgggtGCTCTTCCTCCGTTCGTTCacccgg |
| 248 | gcgcctGCTCTTCCTCCGTTCGTTCaggcgc |
| 249 | gagcgcGCTCTTCCTCCGTTCGTTCgcgctc |
| 250 | ggctggGCTCTTCCTCCGTTCGTTCccagcc |
| 251 | gctgcaGCTCTTCCTCCGTTCGTTCtgcagc |
| 252 | gggtggGCTCTTCCTCCGTTCGTTCccaccc |
| 253 | gcggctGCTCTTCCTCCGTTCGTTCagccgc |
| 254 | gaccgcGCTCTTCCTCCGTTCGTTCgcggtc |
| 255 | gacggcGCTCTTCCTCCGTTCGTTCgccgtc |
| 256 | ggcccaGCTCTTCCTCCGTTCGTTCtgggcc |
| 257 | AGCAAGCTCTTCCTCCGT |
| 258 | cgcggaAGCAAGCTCTTCCTCCGTtccgcg |
| 259 | ctcgcgAGCAAGCTCTTCCTCCGTcgcgag |
| 260 | ccgaccAGCAAGCTCTTCCTCCGTggtcgg |
| 261 | cgacgtAGCAAGCTCTTCCTCCGTacgtcg |
| 262 | cccaccAGCAAGCTCTTCCTCCGTggtggg |
| 263 | cgccgaAGCAAGCTCTTCCTCCGTtcggcg |
| 264 | ctggcgAGCAAGCTCTTCCTCCGTcgccag |
| 265 | ctgccgAGCAAGCTCTTCCTCCGTcggcag |
| 266 | ccgggtAGCAAGCTCTTCCTCCGTacccgg |
| 267 | gcgcctAGCAAGCTCTTCCTCCGTaggcgc |
| 268 | gagcgcAGCAAGCTCTTCCTCCGTgcgctc |
| 269 | ggctggAGCAAGCTCTTCCTCCGTccagcc |
| 270 | gctgcaAGCAAGCTCTTCCTCCGTtgcagc |
| 271 | gggtggAGCAAGCTCTTCCTCCGTccaccc |
| 272 | gcggctAGCAAGCTCTTCCTCCGTagccgc |
| 273 | gaccgcAGCAAGCTCTTCCTCCGTgcggtc |
| 274 | gacggcAGCAAGCTCTTCCTCCGTgccgtc |
| 275 | ggcccaAGCAAGCTCTTCCTCCGTtgggcc |
| 276 | GCCACTAACTTTGGAAGAGC |
| 277 | cgcggaGCCACTAACTTTGGAAGAGCtccgcg |
| 278 | ctcgcgGCCACTAACTTTGGAAGAGCcgcgag |
| 279 | ccgaccGCCACTAACTTTGGAAGAGCggtcgg |
| 280 | cgacgtGCCACTAACTTTGGAAGAGCacgtcg |
| 281 | cccaccGCCACTAACTTTGGAAGAGCggtggg |
| 282 | cgccgaGCCACTAACTTTGGAAGAGCtcggcg |
| 283 | ctggcgGCCACTAACTTTGGAAGAGCcgccag |
| 284 | ctgccgGCCACTAACTTTGGAAGAGCcggcag |
| 285 | ccgggtGCCACTAACTTTGGAAGAGCacccgg |
| 286 | gcgcctGCCACTAACTTTGGAAGAGCaggcgc |
| 287 | gagcgcGCCACTAACTTTGGAAGAGCgcgctc |
| 288 | ggctggGCCACTAACTTTGGAAGAGCccagcc |
| 289 | gctgcaGCCACTAACTTTGGAAGAGCtgcagc |
| 290 | gggtggGCCACTAACTTTGGAAGAGCccaccc |
| 291 | gcggctGCCACTAACTTTGGAAGAGCagccgc |
| 292 | gaccgcGCCACTAACTTTGGAAGAGCgcggtc |
| 293 | gacggcGCCACTAACTTTGGAAGAGCgccgtc |
| 294 | ggcccaGCCACTAACTTTGGAAGAGCtgggcc |

Group 14

| Seq. ID No | Sequence |
|---|---|
| 295 | ATCCCCAACTTACAGGCAG |
| 296 | cgcggaATCCCCAACTTACAGGCAGtccgcg |
| 297 | ctcgcgATCCCCAACTTACAGGCAGcgcgag |
| 298 | CCGCGCATCCCCAACTTACAGGCAGGCGCGG |
| 299 | ccgaccATCCCCAACTTACAGGCAGggtcgg |
| 300 | cgacgtATCCCCAACTTACAGGCAGacgtcg |
| 301 | cccaccATCCCCAACTTACAGGCAGggtggg |
| 302 | cgccgaATCCCCAACTTACAGGCAGtcggcg |
| 303 | ctggcgATCCCCAACTTACAGGCAGcgccag |
| 304 | ctgccgATCCCCAACTTACAGGCAGcggcag |
| 305 | ccgggtATCCCCAACTTACAGGCAGacccgg |
| 306 | gcgcctATCCCCAACTTACAGGCAGaggcgc |
| 307 | gagcgcATCCCCAACTTACAGGCAGgcgctc |
| 308 | ggctggATCCCCAACTTACAGGCAGccagcc |
| 309 | gctgcaATCCCCAACTTACAGGCAGtgcagc |
| 310 | gggtggATCCCCAACTTACAGGCAGccaccc |
| 311 | gcggctATCCCCAACTTACAGGCAGagccgc |
| 312 | gaccgcATCCCCAACTTACAGGCAGgcggtc |
| 313 | gacggcATCCCCAACTTACAGGCAGgccgtc |
| 314 | ggcccaATCCCCAACTTACAGGCAGtgggcc |

Group 15

| Seq. ID No | Sequence |
|---|---|
| 315 | CTCTATCATTCGGTATTAGCC |
| 316 | cgcggaCTCTATCATTCGGTATTAGCCtccgcg |
| 317 | ctcgcgCTCTATCATTCGGTATTAGCCcgcgag |
| 318 | ccgaccCTCTATCATTCGGTATTAGCCggtcgg |
| 319 | cgacgtCTCTATCATTCGGTATTAGCCacgtcg |
| 320 | cccaccCTCTATCATTCGGTATTAGCCggtggg |
| 321 | cgccgaCTCTATCATTCGGTATTAGCCtcggcg |
| 322 | ctggcgCTCTATCATTCGGTATTAGCCcgccag |
| 323 | ctgccgCTCTATCATTCGGTATTAGCCcggcag |
| 324 | ccgggtCTCTATCATTCGGTATTAGCCacccgg |
| 325 | gcgcctCTCTATCATTCGGTATTAGCCaggcgc |
| 326 | gagcgcCTCTATCATTCGGTATTAGCCgcgctc |
| 327 | ggctggCTCTATCATTCGGTATTAGCCccagcc |
| 328 | gctgcaCTCTATCATTCGGTATTAGCCtgcagc |
| 329 | gggtggCTCTATCATTCGGTATTAGCCccaccc |
| 330 | gcggctCTCTATCATTCGGTATTAGCCagccgc |
| 331 | gaccgcCTCTATCATTCGGTATTAGCCgcggtc |
| 332 | gacggcCTCTATCATTCGGTATTAGCCgccgtc |
| 333 | ggcccaCTCTATCATTCGGTATTAGCCtgggcc |
| 334 | GCATGCGCCACACTTTATG |
| 335 | cgcggaGCATGCGCCACACTTTATGtccgcg |
| 336 | ctcgcgGCATGCGCCACACTTTATGcgcgag |
| 337 | ccgaccGCATGCGCCACACTTTATGggtcgg |
| 338 | cgacgtGCATGCGCCACACTTTATGacgtcg |
| 339 | cccaccGCATGCGCCACACTTTATGggtggg |
| 340 | cgccgaGCATGCGCCACACTTTATGtcggcg |
| 341 | ctggcgGCATGCGCCACACTTTATGcgccag |
| 342 | ctgccgGCATGCGCCACACTTTATGcggcag |
| 343 | ccgggtGCATGCGCCACACTTTATGacccgg |
| 344 | gcgcctGCATGCGCCACACTTTATGaggcgc |
| 345 | gagcgcGCATGCGCCACACTTTATGgcgctc |
| 346 | ggctggGCATGCGCCACACTTTATGccagcc |
| 347 | gctgcaGCATGCGCCACACTTTATGtgcagc |
| 348 | gggtggGCATGCGCCACACTTTATGccaccc |
| 349 | gcggctGCATGCGCCACACTTTATGagccgc |
| 350 | gaccgcGCATGCGCCACACTTTATGgcggtc |
| 351 | gacggcGCATGCGCCACACTTTATGgccgtc |
| 352 | ggcccaGCATGCGCCACACTTTATGtgggcc |
| 353 | ccgggcGCATGCGCCACACTTTATCgcccgg |
| 354 | AAGCGTGGCATGCGCCA |
| 355 | cgcggaAAGCGTGGCATGCGCCAtccgcg |
| 356 | ctcgcgAAGCGTGGCATGCGCCAcgcgag |
| 357 | ccgaccAAGCGTGGCATGCGCCAggtcgg |
| 358 | cgacgtAAGCGTGGCATGCGCCAacgtcg |
| 359 | cccaccAAGCGTGGCATGCGCCAggtggg |
| 360 | cgccgaAAGCGTGGCATGCGCCAtcggcg |
| 361 | ctggcgAAGCGTGGCATGCGCCAcgccag |
| 362 | ctgccgAAGCGTGGCATGCGCCAcggcag |
| 363 | ccgggtAAGCGTGGCATGCGCCAacccgg |
| 364 | gcgcctAAGCGTGGCATGCGCCAaggcgc |
| 365 | gagcgcAAGCGTGGCATGCGCCAgcgctc |
| 366 | ggctggAAGCGTGGCATGCGCCAccagcc |
| 367 | gctgcaAAGCGTGGCATGCGCCAtgcagc |
| 368 | gggtggAAGCGTGGCATGCGCCAccaccc |
| 369 | gcggctAAGCGTGGCATGCGCCAagccgc |
| 370 | gaccgcAAGCGTGGCATGCGCCAgcggtc |
| 371 | gacggcAAGCGTGGCATGCGCCAgccgtc |
| 372 | ggcccaAAGCGTGGCATGCGCCAtgggcc |

Group 16

| Seq. | |
|---|---|
| ID No | Sequence |
| 373 | ACAAGCAGTTACTCTTATCC |
| 374 | cgcggaACAAGCAGTTACTCTTATCCtccgcg |
| 375 | ctcgcgACAAGCAGTTACTCTTATCCcgcgag |
| 376 | ccgaccACAAGCAGTTACTCTTATCCggtcgg |
| 377 | cgacgtACAAGCAGTTACTCTTATCCacgtcg |
| 378 | cccaccACAAGCAGTTACTCTTATCCggtggg |
| 379 | cgccgaACAAGCAGTTACTCTTATCCtcggcg |
| 380 | AGACTTCGGCTGTGATTACAAGCAGTTACTCTTATCCCCGAAG |
| 381 | ctggcgACAAGCAGTTACTCTTATCCcgccag |
| 382 | ctgccgACAAGCAGTTACTCTTATCCcggcag |
| 383 | ccgggtACAAGCAGTTACTCTTATCCacccgg |
| 384 | gcgcctACAAGCAGTTACTCTTATCCaggcgc |
| 385 | gagcgcACAAGCAGTTACTCTTATCCgcgctc |
| 386 | ggctggACAAGCAGTTACTCTTATCCccagcc |
| 387 | gctgcaACAAGCAGTTACTCTTATCCtgcagc |
| 388 | gggtggACAAGCAGTTACTCTTATCCccaccc |
| 389 | gcggctACAAGCAGTTACTCTTATCCagccgc |
| 390 | gaccgcACAAGCAGTTACTCTTATCCgcggtc |
| 391 | gacggcACAAGCAGTTACTCTTATCCgccgtc |
| 392 | ggcccaACAAGCAGTTACTCTTATCCtgggcc |
| 393 | GGACAAGCAGTTACTCTTATC |
| 394 | cgcggaGGACAAGCAGTTACTCTTATCtccgcg |
| 395 | ctcgcgGGACAAGCAGTTACTCTTATCcgcgag |
| 396 | ccgaccGGACAAGCAGTTACTCTTATCggtcgg |
| 397 | cgacgtGGACAAGCAGTTACTCTTATCacgtcg |
| 398 | cccaccGGACAAGCAGTTACTCTTATCggtggg |
| 399 | cgccgaGGACAAGCAGTTACTCTTATCtcggcg |
| 400 | ctggcgGGACAAGCAGTTACTCTTATCcgccag |
| 401 | ctgccgGGACAAGCAGTTACTCTTATCcggcag |
| 402 | ccgggtGGACAAGCAGTTACTCTTATCacccgg |
| 403 | gcgcctGGACAAGCAGTTACTCTTATCaggcgc |
| 404 | gagcgcGGACAAGCAGTTACTCTTATCgcgctc |
| 405 | ggctggGGACAAGCAGTTACTCTTATCccagcc |
| 406 | gctgcaGGACAAGCAGTTACTCTTATCtgcagc |
| 407 | gggtggGGACAAGCAGTTACTCTTATCccaccc |
| 408 | gcggctGGACAAGCAGTTACTCTTATCagccgc |
| 409 | gaccgcGGACAAGCAGTTACTCTTATCgcggtc |
| 410 | gacggcGGACAAGCAGTTACTCTTATCgccgtc |
| 411 | ggcccaGGACAAGCAGTTACTCTTATCtgggcc |

Group 17

| Seq. ID No | Sequence |
|---|---|
| 412 | CTATCCGACTAAAGATAGTGG |
| 413 | cgcggaCTATCCGACTAAAGATAGTGGtccgcg |
| 414 | CGCGCCTATCCGACTAAAGATAGTGGCGCG |
| 415 | ctcgcgCTATCCGACTAAAGATAGTGGcgcgag |
| 416 | ccgaccCTATCCGACTAAAGATAGTGGggtcgg |
| 417 | cgacgtCTATCCGACTAAAGATAGTGGacgtcg |
| 418 | cccaccCTATCCGACTAAAGATAGTGGggtggg |
| 419 | cgccgaCTATCCGACTAAAGATAGTGGtcggcg |
| 420 | ctggcgCTATCCGACTAAAGATAGTGGcgccag |
| 421 | ctgccgCTATCCGACTAAAGATAGTGGcggcag |
| 422 | ccgggtCTATCCGACTAAAGATAGTGGacccgg |
| 423 | gcgcctCTATCCGACTAAAGATAGTGGaggcgc |
| 424 | gagcgcCTATCCGACTAAAGATAGTGGgcgctc |
| 425 | ggctggCTATCCGACTAAAGATAGTGGccagcc |
| 426 | gctgcaCTATCCGACTAAAGATAGTGGtgcagc |
| 427 | gggtggCTATCCGACTAAAGATAGTGGccaccc |
| 428 | gcggctCTATCCGACTAAAGATAGTGGagccgc |
| 429 | gaccgcCTATCCGACTAAAGATAGTGGgcggtc |
| 430 | gacggcCTATCCGACTAAAGATAGTGGgccgtc |
| 431 | ggcccaCTATCCGACTAAAGATAGTGGtgggcc |
| 432 | TCCGACTAAAGATAGTGG |
| 433 | cgcggaTCCGACTAAAGATAGTGGtccgcg |
| 434 | ctcgcgTCCGACTAAAGATAGTGGcgcgag |
| 435 | ccgaccTCCGACTAAAGATAGTGGggtcgg |
| 436 | cgacgtTCCGACTAAAGATAGTGGacgtcg |
| 437 | cccaccTCCGACTAAAGATAGTGGggtggg |
| 438 | cgccgaTCCGACTAAAGATAGTGGtcggcg |
| 439 | ctggcgTCCGACTAAAGATAGTGGcgccag |
| 440 | ctgccgTCCGACTAAAGATAGTGGcggcag |
| 441 | ccgggtTCCGACTAAAGATAGTGGacccgg |
| 442 | gcgcctTCCGACTAAAGATAGTGGaggcgc |
| 443 | gagcgcTCCGACTAAAGATAGTGGgcgctc |
| 444 | ggctggTCCGACTAAAGATAGTGGccagcc |
| 445 | gctgcaTCCGACTAAAGATAGTGGtgcagc |
| 446 | gggtggTCCGACTAAAGATAGTGGccaccc |
| 447 | gcggctTCCGACTAAAGATAGTGGagccgc |
| 448 | gaccgcTCCGACTAAAGATAGTGGgcggtc |
| 449 | gacggcTCCGACTAAAGATAGTGGgccgtc |
| 450 | ggcccaTCCGACTAAAGATAGTGGtgggcc |

Group 18

| Seq. ID No | Sequence |
|---|---|
| 451 | CTGGAGGGAAAGCCATTTCAA |
| 452 | cgcggaCTGGAGGGAAAGCCATTTCAAtccgcg |
| 453 | ctcgcgCTGGAGGGAAAGCCATTTCAAcgcgag |
| 454 | CGCGCTGGAGGGAAAGCCATTTCAAGCGCG |
| 455 | ccgaccCTGGAGGGAAAGCCATTTCAAggtcgg |
| 456 | cgacgtCTGGAGGGAAAGCCATTTCAAacgtcg |
| 457 | cccaccCTGGAGGGAAAGCCATTTCAAggtggg |
| 458 | cgccgaCTGGAGGGAAAGCCATTTCAAtcggcg |
| 459 | ctggcgCTGGAGGGAAAGCCATTTCAAcgccag |
| 460 | ctgccgCTGGAGGGAAAGCCATTTCAAcggcag |
| 461 | ccgggtCTGGAGGGAAAGCCATTTCAAacccgg |
| 462 | gcgcctCTGGAGGGAAAGCCATTTCAAaggcgc |
| 463 | gagcgcCTGGAGGGAAAGCCATTTCAAgcgctc |
| 464 | ggctggCTGGAGGGAAAGCCATTTCAAccagcc |
| 465 | gctgcaCTGGAGGGAAAGCCATTTCAAtgcagc |
| 466 | gggtggCTGGAGGGAAAGCCATTTCAAccaccc |
| 467 | gcggctCTGGAGGGAAAGCCATTTCAAagccgc |
| 468 | gaccgcCTGGAGGGAAAGCCATTTCAAgcggtc |
| 469 | gacggcCTGGAGGGAAAGCCATTTCAAgccgtc |
| 470 | ggcccaCTGGAGGGAAAGCCATTTCAAtgggcc |
| 471 | ATTTCAACTACCGGGCTGTTACCG |
| 472 | cgcggaATTTCAACTACCGGGCTGTTACCGtccgcg |
| 473 | ctcgcgATTTCAACTACCGGGCTGTTACCGcgcgag |
| 474 | ccgaccATTTCAACTACCGGGCTGTTACCGggtcgg |
| 475 | cgacgtATTTCAACTACCGGGCTGTTACCGacgtcg |
| 476 | cccaccATTTCAACTACCGGGCTGTTACCGggtggg |
| 477 | cgccgaATTTCAACTACCGGGCTGTTACCGtcggcg |
| 478 | ctggcgATTTCAACTACCGGGCTGTTACCGcgccag |
| 479 | ctgccgATTTCAACTACCGGGCTGTTACCGcggcag |
| 480 | ccgggtATTTCAACTACCGGGCTGTTACCGacccgg |
| 481 | gcgcctATTTCAACTACCGGGCTGTTACCGaggcgc |
| 482 | gagcgcATTTCAACTACCGGGCTGTTACCGgcgctc |
| 483 | ggctggATTTCAACTACCGGGCTGTTACCGccagcc |
| 484 | gctgcaATTTCAACTACCGGGCTGTTACCGtgcagc |
| 485 | gggtggATTTCAACTACCGGGCTGTTACCGccaccc |
| 486 | gcggctATTTCAACTACCGGGCTGTTACCGagccgc |
| 487 | gaccgcATTTCAACTACCGGGCTGTTACCGgcggtc |
| 488 | gacggcATTTCAACTACCGGGCTGTTACCGgccgtc |
| 489 | ggcccaATTTCAACTACCGGGCTGTTACCGtgggcc |
| 490 | GATCCACTCTGGAGGGAAA |
| 491 | cgcggaGATCCACTCTGGAGGGAAAtccgcg |
| 492 | ctcgcgGATCCACTCTGGAGGGAAAcgcgag |
| 493 | ccgaccGATCCACTCTGGAGGGAAAggtcgg |
| 494 | cgacgtGATCCACTCTGGAGGGAAAacgtcg |
| 495 | cccaccGATCCACTCTGGAGGGAAAggtggg |
| 496 | cgccgaGATCCACTCTGGAGGGAAAtcggcg |
| 497 | ctggcgGATCCACTCTGGAGGGAAAcgccag |
| 498 | ctgccgGATCCACTCTGGAGGGAAAcggcag |
| 499 | ccgggtGATCCACTCTGGAGGGAAAacccgg |
| 500 | gcgcctGATCCACTCTGGAGGGAAAaggcgc |
| 501 | gagcgcGATCCACTCTGGAGGGAAAgcgctc |
| 502 | ggctggGATCCACTCTGGAGGGAAAccagcc |
| 503 | gctgcaGATCCACTCTGGAGGGAAAtgcagc |
| 504 | gggtggGATCCACTCTGGAGGGAAAccaccc |
| 505 | gcggctGATCCACTCTGGAGGGAAAagccgc |
| 506 | gaccgcGATCCACTCTGGAGGGAAAgcggtc |
| 507 | gacggcGATCCACTCTGGAGGGAAAgccgtc |
| 508 | ggcccaGATCCACTCTGGAGGGAAAtgggcc |

Group 19

| Seq. ID No | Sequence |
|---|---|
| 509 | TGACTCCCGAGTATAAGTACATG |
| 510 | cgcggaTGACTCCCGAGTATAAGTACATGtccgcg |
| 511 | ctcgcgTGACTCCCGAGTATAAGTACATGcgcgag |
| 512 | ccgaccTGACTCCCGAGTATAAGTACATGggtcgg |
| 513 | cgacgtTGACTCCCGAGTATAAGTACATGacgtcg |
| 514 | cccaccTGACTCCCGAGTATAAGTACATGggtggg |
| 515 | cgccgaTGACTCCCGAGTATAAGTACATGtcggcg |
| 516 | ctggcgTGACTCCCGAGTATAAGTACATGcgccag |
| 517 | ctgccgTGACTCCCGAGTATAAGTACATGcggcag |
| 518 | ccgggtTGACTCCCGAGTATAAGTACATGacccgg |
| 519 | gcgcctTGACTCCCGAGTATAAGTACATGaggcgc |
| 520 | gagcgcTGACTCCCGAGTATAAGTACATGgcgctc |
| 521 | ggctggTGACTCCCGAGTATAAGTACATGccagcc |
| 522 | gctgcaTGACTCCCGAGTATAAGTACATGtgcagc |
| 523 | gggtggTGACTCCCGAGTATAAGTACATGccaccc |
| 524 | ccggtcTGACTCCCGAGTATAAGTACATGaccgg |
| 525 | gcggctTGACTCCCGAGTATAAGTACATGagccgc |
| 526 | gaccgcTGACTCCCGAGTATAAGTACATGgcggtc |
| 527 | gacggcTGACTCCCGAGTATAAGTACATGgccgtc |
| 528 | ggcccaTGACTCCCGAGTATAAGTACATGtgggcc |

Group 20

| Seq. ID No | Sequence |
|---|---|
| 529 | CGAACCTCTAAAGAGGTTCA |
| 530 | cgcggaCGAACCTCTAAAGAGGTTCAtccgcg |
| 531 | ctcgcgCGAACCTCTAAAGAGGTTCAcgcgag |
| 532 | ccgaccCGAACCTCTAAAGAGGTTCAggtcgg |
| 533 | cgacgtCGAACCTCTAAAGAGGTTCAacgtcg |
| 534 | cccaccCGAACCTCTAAAGAGGTTCAggtggg |
| 535 | cgccgaCGAACCTCTAAAGAGGTTCAtcggcg |
| 536 | ctggcgCGAACCTCTAAAGAGGTTCAcgccag |
| 537 | ctgccgCGAACCTCTAAAGAGGTTCAcggcag |
| 538 | ccgggtCGAACCTCTAAAGAGGTTCAacccgg |
| 539 | gcgcctCGAACCTCTAAAGAGGTTCAaggcgc |
| 540 | gagcgcCGAACCTCTAAAGAGGTTCAgcgctc |
| 541 | ggctggCGAACCTCTAAAGAGGTTCAccagcc |
| 542 | gctgcaCGAACCTCTAAAGAGGTTCAtgcagc |
| 543 | gggtggCGAACCTCTAAAGAGGTTCAccaccc |
| 544 | gcggctCGAACCTCTAAAGAGGTTCAagccgc |
| 545 | gaccgcCGAACCTCTAAAGAGGTTCAgcggtc |
| 546 | gacggcCGAACCTCTAAAGAGGTTCAgccgtc |
| 547 | ggcccaCGAACCTCTAAAGAGGTTCAtgggcc |

Group 21

| Seq. ID No | Sequence |
|---|---|
| 548 | CATCGGTTAAACAATTAGCACTG |
| 549 | cgcggaCATCGGTTAAACAATTAGCACTGtccgcg |
| 550 | ctcgcgCATCGGTTAAACAATTAGCACTGcgcgag |
| 551 | ccgaccCATCGGTTAAACAATTAGCACTGggtcgg |
| 552 | cgacgtCATCGGTTAAACAATTAGCACTGacgtcg |
| 553 | cccaccCATCGGTTAAACAATTAGCACTGggtggg |
| 554 | cgccgaCATCGGTTAAACAATTAGCACTGtcggcg |
| 555 | ctggcgCATCGGTTAAACAATTAGCACTGcgccag |
| 556 | CGCGCCATCGGTTAAACAATTAGCACTGGCGCG |
| 557 | ctgccgCATCGGTTAAACAATTAGCACTGcggcag |
| 558 | ccgggtCATCGGTTAAACAATTAGCACTGacccgg |
| 559 | gcgcctCATCGGTTAAACAATTAGCACTGaggcgc |
| 560 | gagcgcCATCGGTTAAACAATTAGCACTGgcgctc |
| 561 | ggctggCATCGGTTAAACAATTAGCACTGccagcc |
| 562 | gctgcaCATCGGTTAAACAATTAGCACTGtgcagc |
| 563 | gggtggCATCGGTTAAACAATTAGCACTGccaccc |
| 564 | gcggctCATCGGTTAAACAATTAGCACTGagccgc |
| 565 | gaccgcCATCGGTTAAACAATTAGCACTGgcggtc |
| 566 | gacggcCATCGGTTAAACAATTAGCACTGgccgtc |
| 567 | ggcccaCATCGGTTAAACAATTAGCACTGtgggcc |

Group 22

| Seq. ID No | Sequence |
|---|---|
| 568 | TGCGATTCCCTATCCTTCTGTGT |
| 569 | cgcggaTGCGATTCCCTATCCTTCTGTGTtccgcg |
| 570 | ctcgcgTGCGATTCCCTATCCTTCTGTGTcgcgag |
| 571 | ccgaccTGCGATTCCCTATCCTTCTGTGTggtcgg |
| 572 | cgacgtTGCGATTCCCTATCCTTCTGTGTacgtcg |
| 573 | cccaccTGCGATTCCCTATCCTTCTGTGTggtggg |
| 574 | cgccgaTGCGATTCCCTATCCTTCTGTGTtcggcg |
| 575 | ctggcgTGCGATTCCCTATCCTTCTGTGTcgccag |
| 576 | ctgccgTGCGATTCCCTATCCTTCTGTGTcggcag |
| 577 | ccgggtTGCGATTCCCTATCCTTCTGTGTacccgg |
| 578 | gcgcctTGCGATTCCCTATCCTTCTGTGTaggcgc |
| 579 | gagcgcTGCGATTCCCTATCCTTCTGTGTgcgctc |
| 580 | ggctggTGCGATTCCCTATCCTTCTGTGTccagcc |
| 581 | gctgcaTGCGATTCCCTATCCTTCTGTGTtgcagc |
| 582 | gggtggTGCGATTCCCTATCCTTCTGTGTccaccc |
| 583 | gcggctTGCGATTCCCTATCCTTCTGTGTagccgc |
| 584 | gaccgcTGCGATTCCCTATCCTTCTGTGTgcggtc |
| 585 | ccagccTGCGATTCCCTATCCTTCTGTGTggctgg |
| 586 | gacggcTGCGATTCCCTATCCTTCTGTGTgccgtc |
| 587 | ggcccaTGCGATTCCCTATCCTTCTGTGTtgggcc |

Group 23

| Seq. ID No | Sequence |
|---|---|
| 588 | GGATTTGCCTACTTCTCACACTCA |
| 589 | cgcggaGGATTTGCCTACTTCTCACACTCAtccgcg |
| 590 | ctcgcgGGATTTGCCTACTTCTCACACTCAcgcgag |
| 591 | ccgaccGGATTTGCCTACTTCTCACACTCAggtcgg |
| 592 | cgacgtGGATTTGCCTACTTCTCACACTCAacgtcg |
| 593 | cccaccGGATTTGCCTACTTCTCACACTCAggtggg |
| 594 | cgccgaGGATTTGCCTACTTCTCACACTCAtcggcg |
| 595 | ctggcgGGATTTGCCTACTTCTCACACTCAcgccag |
| 596 | ctgccgGGATTTGCCTACTTCTCACACTCAcggcag |
| 597 | ccgggtGGATTTGCCTACTTCTCACACTCAacccgg |
| 598 | cccctGGATTTGCCTACTTCTCACACTCAcagggg |
| 599 | gcgcctGGATTTGCCTACTTCTCACACTCAaggcgc |
| 600 | gagcgcGGATTTGCCTACTTCTCACACTCAgcgctc |
| 601 | ggctggGGATTTGCCTACTTCTCACACTCAccagcc |
| 602 | gctgcaGGATTTGCCTACTTCTCACACTCAtgcagc |
| 603 | gggtggGGATTTGCCTACTTCTCACACTCAccaccc |
| 604 | gcggctGGATTTGCCTACTTCTCACACTCAagccgc |
| 605 | gaccgcGGATTTGCCTACTTCTCACACTCAgcggtc |
| 606 | gacggcGGATTTGCCTACTTCTCACACTCAgccgtc |
| 607 | ggcccaGGATTTGCCTACTTCTCACACTCAtgggcc |

Group 24

| Seq. ID No | Sequence |
|---|---|
| 608 | GCACACGTCTCTTCGGCT |
| 609 | cgcggaGCACACGTCTCTTCGGCTtccgcg |
| 610 | ctcgcgGCACACGTCTCTTCGGCTcgcgag |
| 611 | ccgaccGCACACGTCTCTTCGGCTggtcgg |
| 612 | cgacgtGCACACGTCTCTTCGGCTacgtcg |
| 613 | cccaccGCACACGTCTCTTCGGCTggtggg |
| 614 | cgccgaGCACACGTCTCTTCGGCTtcggcg |
| 615 | ctggcgGCACACGTCTCTTCGGCTcgccag |
| 616 | ctgccgGCACACGTCTCTTCGGCTcggcag |
| 617 | ccgggtGCACACGTCTCTTCGGCTacccgg |
| 618 | gcgcctGCACACGTCTCTTCGGCTaggcgc |
| 619 | gagcgcGCACACGTCTCTTCGGCTgcgctc |
| 620 | ggctggGCACACGTCTCTTCGGCTccagcc |
| 621 | gctgcaGCACACGTCTCTTCGGCTtgcagc |
| 622 | gggtggGCACACGTCTCTTCGGCTccaccc |
| 623 | gcggctGCACACGTCTCTTCGGCTagccgc |
| 624 | gaccgcGCACACGTCTCTTCGGCTgcggtc |
| 625 | gacggcGCACACGTCTCTTCGGCTgccgtc |
| 626 | ggcccaGCACACGTCTCTTCGGCTtgggcc |

Group 25

| Seq. ID No | Sequence |
|---|---|
| 627 | ACCATTGCCTCCTCGTATG |
| 628 | cgcggaACCATTGCCTCCTCGTATGtccgcg |
| 629 | ctcgcgACCATTGCCTCCTCGTATGcgcgag |
| 630 | ccgaccACCATTGCCTCCTCGTATGggtcgg |
| 631 | cgacgtACCATTGCCTCCTCGTATGacgtcg |
| 632 | cccaccACCATTGCCTCCTCGTATGggtggg |
| 633 | cgccgaACCATTGCCTCCTCGTATGtcggcg |
| 634 | ctggcgACCATTGCCTCCTCGTATGcgccag |
| 635 | ctgccgACCATTGCCTCCTCGTATGcggcag |
| 636 | ccgggtACCATTGCCTCCTCGTATGacccgg |
| 637 | gcgcctACCATTGCCTCCTCGTATGaggcgc |
| 638 | gagcgcACCATTGCCTCCTCGTATGgcgctc |
| 639 | ggctggACCATTGCCTCCTCGTATGccagcc |
| 640 | gctgcaACCATTGCCTCCTCGTATGtgcagc |
| 641 | gggtggACCATTGCCTCCTCGTATGccaccc |
| 642 | gcggctACCATTGCCTCCTCGTATGagccgc |
| 643 | gaccgcACCATTGCCTCCTCGTATGgcggtc |
| 644 | gacggcACCATTGCCTCCTCGTATGgccgtc |
| 645 | ggcccaACCATTGCCTCCTCGTATGtgggcc |

Group 26

| Seq. ID No | Sequence |
|---|---|
| 646 | CACGCGCTAAGCGTGG |
| 647 | cgcggaCACGCGCTAAGCGTGGtccgcg |
| 648 | ctcgcgCACGCGCTAAGCGTGGcgcgag |
| 649 | ccgaccCACGCGCTAAGCGTGGggtcgg |
| 650 | cgacgtCACGCGCTAAGCGTGGacgtcg |
| 651 | cccaccCACGCGCTAAGCGTGGggtggg |
| 652 | cgccgaCACGCGCTAAGCGTGGtcggcg |
| 653 | ctggcgCACGCGCTAAGCGTGGcgccag |
| 654 | ctgccgCACGCGCTAAGCGTGGcggcag |
| 655 | ccgggtCACGCGCTAAGCGTGGacccgg |
| 656 | gcgcctCACGCGCTAAGCGTGGaggcgc |
| 657 | gagcgcCACGCGCTAAGCGTGGgcgctc |
| 658 | ggctggCACGCGCTAAGCGTGGccagcc |
| 659 | gctgcaCACGCGCTAAGCGTGGtgcagc |
| 660 | gggtggCACGCGCTAAGCGTGGccaccc |
| 661 | gcggctCACGCGCTAAGCGTGGagccgc |
| 662 | gaccgcCACGCGCTAAGCGTGGgcggtc |
| 663 | gacggcCACGCGCTAAGCGTGGgccgtc |
| 664 | ggcccaCACGCGCTAAGCGTGGtgggcc |

Group 27

| Seq. ID No | Sequence |
|---|---|
| 665 | ATGTCGCCATACTTCCACTCCA |
| 666 | cgcggaATGTCGCCATACTTCCACTCCAtccgcg |
| 667 | ctcgcgATGTCGCCATACTTCCACTCCAcgcgag |
| 668 | ccgaccATGTCGCCATACTTCCACTCCAggtcgg |
| 669 | cgacgtATGTCGCCATACTTCCACTCCAacgtcg |
| 670 | cccaccATGTCGCCATACTTCCACTCCAggtggg |
| 671 | cgccgaATGTCGCCATACTTCCACTCCAtcggcg |
| 672 | ctggcgATGTCGCCATACTTCCACTCCAcgccag |
| 673 | ctgccgATGTCGCCATACTTCCACTCCAcggcag |
| 674 | ccgggtATGTCGCCATACTTCCACTCCAacccgg |
| 675 | CGCGCTATGTCGCCATACTTCCACTCCAAGCGCG |
| 676 | gcgcctATGTCGCCATACTTCCACTCCAaggcgc |
| 677 | gagcgcATGTCGCCATACTTCCACTCCAgcgctc |
| 678 | ggctggATGTCGCCATACTTCCACTCCAccagcc |
| 679 | gctgcaATGTCGCCATACTTCCACTCCAtgcagc |
| 680 | gggtggATGTCGCCATACTTCCACTCCAccaccc |
| 681 | gcggctATGTCGCCATACTTCCACTCCAagccgc |
| 682 | gaccgcATGTCGCCATACTTCCACTCCAgcggtc |
| 683 | gacggcATGTCGCCATACTTCCACTCCAgccgtc |
| 684 | ggcccaATGTCGCCATACTTCCACTCCAtgggcc |

Group 28

| Seq. ID No | Sequence |
|---|---|
| 685 | GCCTTTCCAGACCGCTTCATT |
| 686 | cgcggaGCCTTTCCAGACCGCTTCATTtccgcg |
| 687 | ctcgcgGCCTTTCCAGACCGCTTCATTcgcgag |
| 688 | ccgaccGCCTTTCCAGACCGCTTCATTggtcgg |
| 689 | cgacgtGCCTTTCCAGACCGCTTCATTacgtcg |
| 690 | ctcccGCCTTTCCAGACCGCTTCATTcgggag |
| 691 | cccaccGCCTTTCCAGACCGCTTCATTggtggg |
| 692 | cgccgaGCCTTTCCAGACCGCTTCATTtcggcg |
| 693 | ctggcgGCCTTTCCAGACCGCTTCATTcgccag |
| 694 | ctgccgGCCTTTCCAGACCGCTTCATTcggcag |
| 695 | ccgggtGCCTTTCCAGACCGCTTCATTacccgg |
| 696 | gcgcctGCCTTTCCAGACCGCTTCATTaggcgc |
| 697 | gagcgcGCCTTTCCAGACCGCTTCATTgcgctc |
| 698 | ggctggGCCTTTCCAGACCGCTTCATTccagcc |
| 699 | gctgcaGCCTTTCCAGACCGCTTCATTtgcagc |
| 700 | gggtggGCCTTTCCAGACCGCTTCATTccaccc |
| 701 | gcggctGCCTTTCCAGACCGCTTCATTagccgc |
| 702 | gaccgcGCCTTTCCAGACCGCTTCATTgcggtc |
| 703 | gacggcGCCTTTCCAGACCGCTTCATTgccgtc |
| 704 | ggcccaGCCTTTCCAGACCGCTTCATTtgggcc |

Group 29

| Seq. ID No | Sequence |
|---|---|
| 705 | TCGGGTCTACGACCTGTTACTT |
| 706 | cgcggaTCGGGTCTACGACCTGTTACTTtccgcg |
| 707 | ctcgcgTCGGGTCTACGACCTGTTACTTcgcgag |
| 708 | ccgaccTCGGGTCTACGACCTGTTACTTggtcgg |
| 709 | cgacgtTCGGGTCTACGACCTGTTACTTacgtcg |
| 710 | cccaccTCGGGTCTACGACCTGTTACTTggtggg |
| 711 | cgccgaTCGGGTCTACGACCTGTTACTTtcggcg |
| 712 | ctggcgTCGGGTCTACGACCTGTTACTTcgccag |
| 713 | ctgccgTCGGGTCTACGACCTGTTACTTcggcag |
| 714 | ccgggtTCGGGTCTACGACCTGTTACTTacccgg |
| 715 | gcgcctTCGGGTCTACGACCTGTTACTTaggcgc |
| 716 | gagcgcTCGGGTCTACGACCTGTTACTTgcgctc |
| 717 | ggctggTCGGGTCTACGACCTGTTACTTccagcc |
| 718 | gctgcaTCGGGTCTACGACCTGTTACTTtgcagc |
| 719 | gggtggTCGGGTCTACGACCTGTTACTTccaccc |
| 720 | gcggctTCGGGTCTACGACCTGTTACTTagccgc |
| 721 | gaccgcTCGGGTCTACGACCTGTTACTTgcggtc |
| 722 | ccccTCGGGTCTACGACCTGTTACTTgagggg |
| 723 | gacggcTCGGGTCTACGACCTGTTACTTgccgtc |
| 724 | ggcccaTCGGGTCTACGACCTGTTACTTtgggcc |

Group 30

| Seq. ID No | Sequence |
|---|---|
| 725 | ACCTTAGATATTCGGTGGAAGGGA |
| 726 | cgcggaACCTTAGATATTCGGTGGAAGGGAtccgcg |
| 727 | ctcgcgACCTTAGATATTCGGTGGAAGGGAcgcgag |
| 728 | ccgaccACCTTAGATATTCGGTGGAAGGGAggtcgg |
| 729 | cgacgtACCTTAGATATTCGGTGGAAGGGAacgtcg |
| 730 | cccaccACCTTAGATATTCGGTGGAAGGGAggtggg |
| 731 | cgccgaACCTTAGATATTCGGTGGAAGGGAtcggcg |
| 732 | ctggcgACCTTAGATATTCGGTGGAAGGGAcgccag |
| 733 | ctgccgACCTTAGATATTCGGTGGAAGGGAcggcag |
| 734 | ccgggtACCTTAGATATTCGGTGGAAGGGAacccgg |
| 735 | gcgcctACCTTAGATATTCGGTGGAAGGGAaggcgc |
| 736 | gagcgcACCTTAGATATTCGGTGGAAGGGAgcgctc |
| 737 | ggctggACCTTAGATATTCGGTGGAAGGGAccagcc |
| 738 | gctgcaACCTTAGATATTCGGTGGAAGGGAtgcagc |
| 739 | gggtggACCTTAGATATTCGGTGGAAGGGAccaccc |
| 740 | gcggctACCTTAGATATTCGGTGGAAGGGAagccgc |
| 741 | gaccgcACCTTAGATATTCGGTGGAAGGGAgcggtc |
| 742 | ccACCTTAGATATTCGGTGGAAGGGAggtgg |
| 743 | gacggcACCTTAGATATTCGGTGGAAGGGAgccgtc |
| 744 | ggcccaACCTTAGATATTCGGTGGAAGGGAtgggcc |

Group 31

| Seq. ID No | Sequence |
|---|---|
| 745 | GAACTTCCGTACTTAATTTCCTTC |
| 746 | cgcggaGAACTTCCGTACTTAATTTCCTTCtccgcg |
| 747 | ctcgcgGAACTTCCGTACTTAATTTCCTTCcgcgag |
| 748 | ccgaccGAACTTCCGTACTTAATTTCCTTCggtcgg |
| 749 | cgacgtGAACTTCCGTACTTAATTTCCTTCacgtcg |
| 750 | cccaccGAACTTCCGTACTTAATTTCCTTCggtggg |
| 751 | cgccgaGAACTTCCGTACTTAATTTCCTTCtcggcg |
| 752 | ctggcgGAACTTCCGTACTTAATTTCCTTCcgccag |
| 753 | ctgccgGAACTTCCGTACTTAATTTCCTTCcggcag |
| 754 | ccgggtGAACTTCCGTACTTAATTTCCTTCacccgg |
| 755 | gcgcctGAACTTCCGTACTTAATTTCCTTCaggcgc |
| 756 | gagcgcGAACTTCCGTACTTAATTTCCTTCgcgctc |
| 757 | ggctggGAACTTCCGTACTTAATTTCCTTCccagcc |
| 758 | gctgcaGAACTTCCGTACTTAATTTCCTTCtgcagc |
| 759 | gggtggGAACTTCCGTACTTAATTTCCTTCccaccc |
| 760 | gcggctGAACTTCCGTACTTAATTTCCTTCagccgc |
| 761 | gaccgcGAACTTCCGTACTTAATTTCCTTCgcggtc |
| 762 | gacggcGAACTTCCGTACTTAATTTCCTTCgccgtc |
| 763 | ggcccaGAACTTCCGTACTTAATTTCCTTCtgggcc |
| 764 | ccttcGAACTTCCGTACTTAATTTCCTTCgaagg |

Group 32

| Seq. ID No | Sequence |
|---|---|
| 765 | TTTGCGGTACGGGCAGTACT |
| 766 | cgcggaTTTGCGGTACGGGCAGTACTtccgcg |
| 767 | ctcgcgTTTGCGGTACGGGCAGTACTcgcgag |
| 768 | ccgaccTTTGCGGTACGGGCAGTACTggtcgg |
| 769 | cgacgtTTTGCGGTACGGGCAGTACTacgtcg |
| 770 | cccaccTTTGCGGTACGGGCAGTACTggtggg |
| 771 | cgccgaTTTGCGGTACGGGCAGTACTtcggcg |
| 772 | ctggcgTTTGCGGTACGGGCAGTACTcgccag |
| 773 | ctgccgTTTGCGGTACGGGCAGTACTcggcag |
| 774 | ccgggtTTTGCGGTACGGGCAGTACTacccgg |
| 775 | gcgcctTTTGCGGTACGGGCAGTACTaggcgc |
| 776 | ccagtaGCGGTACGGGCAGTACTgg |
| 777 | gagcgcTTTGCGGTACGGGCAGTACTgcgctc |
| 778 | ggctggTTTGCGGTACGGGCAGTACTccagcc |
| 779 | gctgcaTTTGCGGTACGGGCAGTACTtgcagc |
| 780 | gggtggTTTGCGGTACGGGCAGTACTccaccc |
| 781 | gcggctTTTGCGGTACGGGCAGTACTagccgc |
| 782 | gaccgcTTTGCGGTACGGGCAGTACTgcggtc |
| 783 | gacggcTTTGCGGTACGGGCAGTACTgccgtc |
| 784 | ggcccaTTTGCGGTACGGGCAGTACTtgggcc |
| 785 | GGCAGTACTACTCTTCCTAC |
| 786 | cgcggaGGCAGTACTACTCTTCCTACtccgcg |
| 787 | ctcgcgGGCAGTACTACTCTTCCTACcgcgag |
| 788 | ccgaccGGCAGTACTACTCTTCCTACggtcgg |
| 789 | cgacgtGGCAGTACTACTCTTCCTACacgtcg |
| 790 | cccaccGGCAGTACTACTCTTCCTACggtggg |
| 791 | cgccgaGGCAGTACTACTCTTCCTACtcggcg |
| 792 | ctggcgGGCAGTACTACTCTTCCTACcgccag |
| 793 | ctgccgGGCAGTACTACTCTTCCTACcggcag |
| 794 | ccgggtGGCAGTACTACTCTTCCTACacccgg |
| 795 | gcgcctGGCAGTACTACTCTTCCTACaggcgc |
| 796 | gagcgcGGCAGTACTACTCTTCCTACgcgctc |
| 797 | ggctggGGCAGTACTACTCTTCCTACccagcc |
| 798 | gctgcaGGCAGTACTACTCTTCCTACtgcagc |
| 799 | gggtggGGCAGTACTACTCTTCCTACccaccc |
| 800 | CCCCTGGCAGTACTACTCTTCCTACAGGGG |
| 801 | gcggctGGCAGTACTACTCTTCCTACagccgc |
| 802 | gaccgcGGCAGTACTACTCTTCCTACgcggtc |
| 803 | gacggcGGCAGTACTACTCTTCCTACgccgtc |
| 804 | ggcccaGGCAGTACTACTCTTCCTACtgggcc |

Group 33

| Seq. ID No | Sequence |
|---|---|
| 805 | GGTACAAACATTCCATATCAGGTTG |
| 806 | cgcggaGGTACAAACATTCCATATCAGGTTGtccgcg |
| 807 | ctcgcgGGTACAAACATTCCATATCAGGTTGcgcgag |
| 808 | ccgaccGGTACAAACATTCCATATCAGGTTGggtcgg |
| 809 | cgacgtGGTACAAACATTCCATATCAGGTTGacgtcg |
| 810 | cccaccGGTACAAACATTCCATATCAGGTTGggtggg |
| 811 | cgccgaGGTACAAACATTCCATATCAGGTTGtcggcg |
| 812 | ctggcgGGTACAAACATTCCATATCAGGTTGcgccag |
| 813 | ctgccgGGTACAAACATTCCATATCAGGTTGcggcag |
| 814 | ccgggtGGTACAAACATTCCATATCAGGTTGacccgg |
| 815 | gcgcctGGTACAAACATTCCATATCAGGTTGaggcgc |
| 816 | gagcgcGGTACAAACATTCCATATCAGGTTGgcgctc |
| 817 | ggctggGGTACAAACATTCCATATCAGGTTGccagcc |
| 818 | gctgcaGGTACAAACATTCCATATCAGGTTGtgcagc |
| 819 | gggtggGGTACAAACATTCCATATCAGGTTGccaccc |
| 820 | gcggctGGTACAAACATTCCATATCAGGTTGagccgc |
| 821 | gaccgcGGTACAAACATTCCATATCAGGTTGgcggtc |
| 822 | cccccaGGTACAAACATTCCATATCAGGTTGgggg |
| 823 | gacggcGGTACAAACATTCCATATCAGGTTGgccgtc |
| 824 | ggcccaGGTACAAACATTCCATATCAGGTTGtgggcc |

Group 34

| Seq. ID No | Sequence |
|---|---|
| 825 | CGCGACCCTTTGTACTATC |
| 826 | cgcggaCGCGACCCTTTGTACTATCtccgcg |
| 827 | ctcgcgCGCGACCCTTTGTACTATCcgcgag |
| 828 | ccgaccCGCGACCCTTTGTACTATCggtcgg |
| 829 | cgacgtCGCGACCCTTTGTACTATCacgtcg |
| 830 | cccaccCGCGACCCTTTGTACTATCggtggg |
| 831 | cgccgaCGCGACCCTTTGTACTATCtcggcg |
| 832 | ctggcgCGCGACCCTTTGTACTATCcgccag |
| 833 | ctgccgCGCGACCCTTTGTACTATCcggcag |
| 834 | ccgggtCGCGACCCTTTGTACTATCacccgg |
| 835 | ccccccCGCGACCCTTTGTACTATCgggggg |
| 836 | gcgcctCGCGACCCTTTGTACTATCaggcgc |
| 837 | gagcgcCGCGACCCTTTGTACTATCgcgctc |
| 838 | ggctggCGCGACCCTTTGTACTATCccagcc |
| 839 | gctgcaCGCGACCCTTTGTACTATCtgcagc |
| 840 | gggtggCGCGACCCTTTGTACTATCccaccc |
| 841 | gcggctCGCGACCCTTTGTACTATCagccgc |
| 842 | gaccgcCGCGACCCTTTGTACTATCgcggtc |
| 843 | gacggcCGCGACCCTTTGTACTATCgccgtc |
| 844 | ggcccaCGCGACCCTTTGTACTATCtgggcc |

Group 35

| Seq. ID No | Sequence |
|---|---|
| 845 | AATAGTTTTATGGGATTAGCTCCAC |
| 846 | cgcggaAATAGTTTTATGGGATTAGCTCCACtccgcg |
| 847 | ctcgcgAATAGTTTTATGGGATTAGCTCCACcgcgag |
| 848 | ccgaccAATAGTTTTATGGGATTAGCTCCACggtcgg |
| 849 | cgacgtAATAGTTTTATGGGATTAGCTCCACacgtcg |
| 850 | cccaccAATAGTTTTATGGGATTAGCTCCACggtggg |
| 851 | cgccgaAATAGTTTTATGGGATTAGCTCCACtcggcg |
| 852 | ctggcgAATAGTTTTATGGGATTAGCTCCACcgccag |
| 853 | ctgccgAATAGTTTTATGGGATTAGCTCCACcggcag |
| 854 | ccgggtAATAGTTTTATGGGATTAGCTCCACacccgg |
| 855 | ccccccAATAGTTTTATGGGATTAGCTCCACgggggg |
| 856 | ccgtggAATAGTTTTATGGGATTAGCTCCACgg |
| 857 | gcgcctAATAGTTTTATGGGATTAGCTCCACaggcgc |
| 858 | gagcgcAATAGTTTTATGGGATTAGCTCCACgcgctc |
| 859 | ggctggAATAGTTTTATGGGATTAGCTCCACccagcc |
| 860 | gctgcaAATAGTTTTATGGGATTAGCTCCACtgcagc |
| 861 | gggtggAATAGTTTTATGGGATTAGCTCCACccaccc |
| 862 | gcggctAATAGTTTTATGGGATTAGCTCCACagccgc |
| 863 | gaccgcAATAGTTTTATGGGATTAGCTCCACgcggtc |
| 864 | gacggcAATAGTTTTATGGGATTAGCTCCACgccgtc |
| 865 | ggcccaAATAGTTTTATGGGATTAGCTCCACtgggcc |

Group 36

| Seq. ID No | Sequence |
|---|---|
| 866 | GAAACCATCTTTCAAAAGCGT |
| 867 | cgcggaGAAACCATCTTTCAAAAGCGTtccgcg |
| 868 | ctcgcgGAAACCATCTTTCAAAAGCGTcgcgag |
| 869 | ccccGAAACCATCTTTCAAAAGCGTcgggg |
| 870 | ccgaccGAAACCATCTTTCAAAAGCGTggtcgg |
| 871 | cgacgtGAAACCATCTTTCAAAAGCGTacgtcg |
| 872 | cccaccGAAACCATCTTTCAAAAGCGTggtggg |
| 873 | cgccgaGAAACCATCTTTCAAAAGCGTtcggcg |
| 874 | ctggcgGAAACCATCTTTCAAAAGCGTcgccag |
| 875 | ctgccgGAAACCATCTTTCAAAAGCGTcggcag |
| 876 | ccgggtGAAACCATCTTTCAAAAGCGTacccgg |
| 877 | ccccccGAAACCATCTTTCAAAAGCGTgggggg |
| 878 | gcgcctGAAACCATCTTTCAAAAGCGTaggcgc |
| 879 | gagcgcGAAACCATCTTTCAAAAGCGTgcgctc |
| 880 | ggctggGAAACCATCTTTCAAAAGCGTccagcc |
| 881 | gctgcaGAAACCATCTTTCAAAAGCGTtgcagc |
| 882 | gggtggGAAACCATCTTTCAAAAGCGTccaccc |
| 883 | gcggctGAAACCATCTTTCAAAAGCGTagccgc |
| 884 | gaccgcGAAACCATCTTTCAAAAGCGTgcggtc |
| 885 | gacggcGAAACCATCTTTCAAAAGCGTgccgtc |
| 886 | ggcccaGAAACCATCTTTCAAAAGCGTtgggcc |

Group 37

| Seq. ID No | Sequence |
|---|---|
| 887 | TGGCATGCGCCACA |
| 888 | cgcggaTGGCATGCGCCACAtccgcg |
| 889 | ctcgcgTGGCATGCGCCACAcgcgag |
| 890 | ccgaccTGGCATGCGCCACAggtcgg |
| 891 | cgacgtTGGCATGCGCCACAacgtcg |
| 892 | cccaccTGGCATGCGCCACAggtggg |
| 893 | cgccgaTGGCATGCGCCACAtcggcg |
| 894 | ctggcgTGGCATGCGCCACAcgccag |
| 895 | ctgccgTGGCATGCGCCACAcggcag |
| 896 | ccgggtTGGCATGCGCCACAacccgg |
| 897 | ccccccTGGCATGCGCCACAgggggg |
| 898 | gcgcctTGGCATGCGCCACAaggcgc |
| 899 | gagcgcTGGCATGCGCCACAgcgctc |
| 900 | ggctggTGGCATGCGCCACAccagcc |
| 901 | gctgcaTGGCATGCGCCACAtgcagc |
| 902 | gggtggTGGCATGCGCCACAccaccc |
| 903 | gcggctTGGCATGCGCCACAagccgc |
| 904 | gaccgcTGGCATGCGCCACAgcggtc |
| 905 | gacggcTGGCATGCGCCACAgccgtc |
| 906 | ggcccaTGGCATGCGCCACAtgggcc |
| 907 | ccTGGCATGCGCCACAgccagg |
| 908 | CATGCGCCACACTTTATCAT |
| 909 | cgcggaCATGCGCCACACTTTATCATtccgcg |
| 910 | ctcgcgCATGCGCCACACTTTATCATcgcgag |
| 911 | ccgaccCATGCGCCACACTTTATCATggtcgg |
| 912 | cgacgtCATGCGCCACACTTTATCATacgtcg |
| 913 | cccaccCATGCGCCACACTTTATCATggtggg |
| 914 | cgccgaCATGCGCCACACTTTATCATtcggcg |
| 915 | ctggcgCATGCGCCACACTTTATCATcgccag |
| 916 | ctgccgCATGCGCCACACTTTATCATcggcag |
| 917 | ccgggtCATGCGCCACACTTTATCATacccgg |
| 918 | ccccccCATGCGCCACACTTTATCATgggggg |
| 919 | gcgcctCATGCGCCACACTTTATCATaggcgc |
| 920 | gagcgcCATGCGCCACACTTTATCATgcgctc |
| 921 | ggctggCATGCGCCACACTTTATCATccagcc |
| 922 | gctgcaCATGCGCCACACTTTATCATtgcagc |
| 923 | gggtggCATGCGCCACACTTTATCATccaccc |
| 924 | gcggctCATGCGCCACACTTTATCATagccgc |
| 925 | gaccgcCATGCGCCACACTTTATCATgcggtc |
| 926 | gacggcCATGCGCCACACTTTATCATgccgtc |
| 927 | ggcccaCATGCGCCACACTTTATCATtgggcc |

Group 38

| Seq. ID No | Sequence |
|---|---|
| 928 | AAGCAGTTACTCTTATCCTTGT |
| 929 | cgcggaAAGCAGTTACTCTTATCCTTGTtccgcg |
| 930 | ctcgcgAAGCAGTTACTCTTATCCTTGTcgcgag |
| 931 | ccgaccAAGCAGTTACTCTTATCCTTGTggtcgg |
| 932 | cgacgtAAGCAGTTACTCTTATCCTTGTacgtcg |
| 933 | cccaccAAGCAGTTACTCTTATCCTTGTggtggg |
| 934 | cgccgaAAGCAGTTACTCTTATCCTTGTtcggcg |
| 935 | ctggcgAAGCAGTTACTCTTATCCTTGTcgccag |
| 936 | ctgccgAAGCAGTTACTCTTATCCTTGTcggcag |
| 937 | ccgggtAAGCAGTTACTCTTATCCTTGTacccgg |
| 938 | ccccccAAGCAGTTACTCTTATCCTTGTgggggg |
| 939 | gcgcctAAGCAGTTACTCTTATCCTTGTaggcgc |
| 940 | gagcgcAAGCAGTTACTCTTATCCTTGTgcgctc |
| 941 | ggctggAAGCAGTTACTCTTATCCTTGTccagcc |
| 942 | gctgcaAAGCAGTTACTCTTATCCTTGTtgcagc |
| 943 | gggtggAAGCAGTTACTCTTATCCTTGTccaccc |
| 944 | gcggctAAGCAGTTACTCTTATCCTTGTagccgc |
| 945 | gaccgcAAGCAGTTACTCTTATCCTTGTgcggtc |
| 946 | gacggcAAGCAGTTACTCTTATCCTTGTgccgtc |
| 947 | ggcccaAAGCAGTTACTCTTATCCTTGTtgggcc |

Group 39

| Seq. ID No | Sequence |
|---|---|
| 948 | CGGAGTTATCCCCAACTTAC |
| 949 | cgcggaCGGAGTTATCCCCAACTTACtccgcg |
| 950 | ctcgcgCGGAGTTATCCCCAACTTACcgcgag |
| 951 | ccgaccCGGAGTTATCCCCAACTTACggtcgg |
| 952 | cgacgtCGGAGTTATCCCCAACTTACacgtcg |
| 953 | cccaccCGGAGTTATCCCCAACTTACggtggg |
| 954 | cgccgaCGGAGTTATCCCCAACTTACtcggcg |
| 955 | ctggcgCGGAGTTATCCCCAACTTACcgccag |
| 956 | ctgccgCGGAGTTATCCCCAACTTACcggcag |
| 957 | ccgggtCGGAGTTATCCCCAACTTACacccgg |
| 958 | ccccccCGGAGTTATCCCCAACTTACgggggg |
| 959 | gcgcctCGGAGTTATCCCCAACTTACaggcgc |
| 960 | gagcgcCGGAGTTATCCCCAACTTACgcgctc |
| 961 | ggctggCGGAGTTATCCCCAACTTACccagcc |
| 962 | gctgcaCGGAGTTATCCCCAACTTACtgcagc |
| 963 | gggtggCGGAGTTATCCCCAACTTACccaccc |
| 964 | gcggctCGGAGTTATCCCCAACTTACagccgc |
| 965 | gaccgcCGGAGTTATCCCCAACTTACgcggtc |
| 966 | gacggcCGGAGTTATCCCCAACTTACgccgtc |
| 967 | ggcccaCGGAGTTATCCCCAACTTACtgggcc |

Group 40

| Seq. ID No | Sequence |
|---|---|
| 968 | CTCCATGTATCGCTACACTTCC |
| 969 | cgcggaCTCCATGTATCGCTACACTTCCtccgcg |
| 970 | ctcgcgCTCCATGTATCGCTACACTTCCcgcgag |
| 971 | ccgaccCTCCATGTATCGCTACACTTCCggtcgg |
| 972 | cgacgtCTCCATGTATCGCTACACTTCCacgtcg |
| 973 | cccaccCTCCATGTATCGCTACACTTCCggtggg |
| 974 | cgccgaCTCCATGTATCGCTACACTTCCtcggcg |
| 975 | ctggcgCTCCATGTATCGCTACACTTCCcgccag |
| 976 | ctgccgCTCCATGTATCGCTACACTTCCcggcag |
| 977 | ccgggtCTCCATGTATCGCTACACTTCCacccgg |
| 978 | ccccccCTCCATGTATCGCTACACTTCCgggggg |
| 979 | gcgcctCTCCATGTATCGCTACACTTCCaggcgc |
| 980 | gagcgcCTCCATGTATCGCTACACTTCCgcgctc |
| 981 | ctggcgCTCCATGTATCGCTACACTTCCcgccag |
| 982 | ggctggCTCCATGTATCGCTACACTTCCccagcc |
| 983 | gctgcaCTCCATGTATCGCTACACTTCCtgcagc |
| 984 | gggtggCTCCATGTATCGCTACACTTCCccaccc |
| 985 | gcggctCTCCATGTATCGCTACACTTCCagccgc |
| 986 | gaccgcCTCCATGTATCGCTACACTTCCgcggtc |
| 987 | gacggcCTCCATGTATCGCTACACTTCCgccgtc |
| 988 | ggcccaCTCCATGTATCGCTACACTTCCtgggcc |

Group 41

| Seq. ID No | Sequence |
|---|---|
| 989 | ATGCTAGCGCACACGTCTCTT |
| 990 | cgcggaATGCTAGCGCACACGTCTCTTtccgcg |
| 991 | ctcgcgATGCTAGCGCACACGTCTCTTcgcgag |
| 992 | ccgaccATGCTAGCGCACACGTCTCTTggtcgg |
| 993 | cgacgtATGCTAGCGCACACGTCTCTTacgtcg |
| 994 | cccaccATGCTAGCGCACACGTCTCTTggtggg |
| 995 | cgccgaATGCTAGCGCACACGTCTCTTtcggcg |
| 996 | ctggcgATGCTAGCGCACACGTCTCTTcgccag |
| 997 | cccATGCTAGCGCACACGTCTCTTcatggg |
| 998 | ctgccgATGCTAGCGCACACGTCTCTTcggcag |
| 999 | ccgggtATGCTAGCGCACACGTCTCTTacccgg |
| 1000 | ccccccATGCTAGCGCACACGTCTCTTgggggg |
| 1001 | gcgcctATGCTAGCGCACACGTCTCTTaggcgc |
| 1002 | gagcgcATGCTAGCGCACACGTCTCTTgcgctc |
| 1003 | ggctggATGCTAGCGCACACGTCTCTTccagcc |
| 1004 | gctgcaATGCTAGCGCACACGTCTCTTtgcagc |
| 1005 | gggtggATGCTAGCGCACACGTCTCTTccaccc |
| 1006 | gcggctATGCTAGCGCACACGTCTCTTagccgc |
| 1007 | gaccgcATGCTAGCGCACACGTCTCTTgcggtc |
| 1008 | gacggcATGCTAGCGCACACGTCTCTTgccgtc |
| 1009 | ggcccaATGCTAGCGCACACGTCTCTTtgggcc |

Group 42

| Seq. ID No | Sequence |
|---|---|
| 1010 | GTACCTTCGCCGAAGCTAAGCAC |
| 1011 | cgcggaGTACCTTCGCCGAAGCTAAGCACtccgcg |
| 1012 | ctcgcgGTACCTTCGCCGAAGCTAAGCACcgcgag |
| 1013 | ccgaccGTACCTTCGCCGAAGCTAAGCACggtcgg |
| 1014 | cgacgtGTACCTTCGCCGAAGCTAAGCACacgtcg |
| 1015 | cccaccGTACCTTCGCCGAAGCTAAGCACggtggg |
| 1016 | cgccgaGTACCTTCGCCGAAGCTAAGCACtcggcg |
| 1017 | ctggcgGTACCTTCGCCGAAGCTAAGCACcgccag |
| 1018 | ctgccgGTACCTTCGCCGAAGCTAAGCACcggcag |
| 1019 | ccgggtGTACCTTCGCCGAAGCTAAGCACacccgg |
| 1020 | ccccccGTACCTTCGCCGAAGCTAAGCACgggggg |
| 1021 | gcgcctGTACCTTCGCCGAAGCTAAGCACaggcgc |
| 1022 | gagcgcGTACCTTCGCCGAAGCTAAGCACgcgctc |
| 1023 | ggctggGTACCTTCGCCGAAGCTAAGCACccagcc |
| 1024 | gctgcaGTACCTTCGCCGAAGCTAAGCACtgcagc |
| 1025 | gggtggGTACCTTCGCCGAAGCTAAGCACccaccc |
| 1026 | gcggctGTACCTTCGCCGAAGCTAAGCACagccgc |
| 1027 | gaccgcGTACCTTCGCCGAAGCTAAGCACgcggtc |
| 1028 | gacggcGTACCTTCGCCGAAGCTAAGCACgccgtc |
| 1029 | ggcccaGTACCTTCGCCGAAGCTAAGCACtgggcc |
| 1030 | TCGCCGAAGCTAAGCACT |
| 1031 | cgcggaTCGCCGAAGCTAAGCACTtccgcg |
| 1032 | ctcgcgTCGCCGAAGCTAAGCACTcgcgag |
| 1033 | ccgaccTCGCCGAAGCTAAGCACTggtcgg |
| 1034 | cgacgtTCGCCGAAGCTAAGCACTacgtcg |
| 1035 | cccaccTCGCCGAAGCTAAGCACTggtggg |
| 1036 | cgccgaTCGCCGAAGCTAAGCACTtcggcg |
| 1037 | ctggcgTCGCCGAAGCTAAGCACTcgccag |
| 1038 | ctgccgTCGCCGAAGCTAAGCACTcggcag |
| 1039 | ccgggtTCGCCGAAGCTAAGCACTacccgg |
| 1040 | ccccccTCGCCGAAGCTAAGCACTgggggg |
| 1041 | cccccaTCGCCGAAGCTAAGCACTggggg |
| 1042 | gcgcctTCGCCGAAGCTAAGCACTaggcgc |
| 1043 | gagcgcTCGCCGAAGCTAAGCACTgcgctc |
| 1044 | ggctggTCGCCGAAGCTAAGCACTccagcc |
| 1045 | gctgcaTCGCCGAAGCTAAGCACTtgcagc |
| 1046 | gggtggTCGCCGAAGCTAAGCACTccaccc |
| 1047 | gcggctTCGCCGAAGCTAAGCACTagccgc |
| 1048 | gaccgcTCGCCGAAGCTAAGCACTgcggtc |
| 1049 | gacggcTCGCCGAAGCTAAGCACTgccgtc |
| 1050 | ggcccaTCGCCGAAGCTAAGCACTtgggcc |
| 1051 | CGTACCTTCGCCGAAGC |
| 1052 | cgcggaCGTACCTTCGCCGAAGCtccgcg |
| 1053 | ctcgcgCGTACCTTCGCCGAAGCcgcgag |
| 1054 | ccgaccCGTACCTTCGCCGAAGCggtcgg |
| 1055 | cgacgtCGTACCTTCGCCGAAGCacgtcg |
| 1056 | cgcgagCGTACCTTCGCCGAAGCctcgcg |
| 1057 | cccaccCGTACCTTCGCCGAAGCggtggg |
| 1058 | cgccgaCGTACCTTCGCCGAAGCtcggcg |
| 1059 | ctggcgCGTACCTTCGCCGAAGCcgccag |
| 1060 | ctgccgCGTACCTTCGCCGAAGCcggcag |
| 1061 | ccgggtCGTACCTTCGCCGAAGCacccgg |
| 1062 | ccccccCGTACCTTCGCCGAAGCgggggg |
| 1063 | gcgcctCGTACCTTCGCCGAAGCaggcgc |
| 1064 | gagcgcCGTACCTTCGCCGAAGCgcgctc |
| 1065 | ggctggCGTACCTTCGCCGAAGCccagcc |
| 1066 | gctgcaCGTACCTTCGCCGAAGCtgcagc |
| 1067 | gggtggCGTACCTTCGCCGAAGCccaccc |
| 1068 | gcggctCGTACCTTCGCCGAAGCagccgc |
| 1069 | gaccgcCGTACCTTCGCCGAAGCgcggtc |
| 1070 | gacggcCGTACCTTCGCCGAAGCgccgtc |
| 1071 | ggcccaCGTACCTTCGCCGAAGCtgggcc |

Group 43

| Seq. ID No | Sequence |
|---|---|
| 1072 | CAGTACTACTCTTCCTAGAGGCT |
| 1073 | cgcggaCAGTACTACTCTTCCTAGAGGCTtccgcg |
| 1074 | ctcgcgCAGTACTACTCTTCCTAGAGGCTcgcgag |
| 1075 | ccgaccCAGTACTACTCTTCCTAGAGGCTggtcgg |
| 1076 | cgacgtCAGTACTACTCTTCCTAGAGGCTacgtcg |
| 1077 | cccaccCAGTACTACTCTTCCTAGAGGCTggtggg |
| 1078 | cgccgaCAGTACTACTCTTCCTAGAGGCTtcggcg |
| 1079 | ctggcgCAGTACTACTCTTCCTAGAGGCTcgccag |
| 1080 | ctgccgCAGTACTACTCTTCCTAGAGGCTcggcag |
| 1081 | ccgggtCAGTACTACTCTTCCTAGAGGCTacccgg |
| 1082 | ccccccCAGTACTACTCTTCCTAGAGGCTgggggg |
| 1083 | gcgcctCAGTACTACTCTTCCTAGAGGCTaggcgc |
| 1084 | gagcgcCAGTACTACTCTTCCTAGAGGCTgcgctc |
| 1085 | ggctggCAGTACTACTCTTCCTAGAGGCTccagcc |
| 1086 | gctgcaCAGTACTACTCTTCCTAGAGGCTtgcagc |
| 1087 | gggtggCAGTACTACTCTTCCTAGAGGCTccaccc |
| 1088 | gcggctCAGTACTACTCTTCCTAGAGGCTagccgc |
| 1089 | gaccgcCAGTACTACTCTTCCTAGAGGCTgcggtc |
| 1090 | gacggcCAGTACTACTCTTCCTAGAGGCTgccgtc |
| 1091 | ggcccaCAGTACTACTCTTCCTAGAGGCTtgggcc |
| 1092 | GGCAGTACTACTCTTCCTAGA |
| 1093 | ctgcGGCAGTACTACTCTTCCTAGAccgcag |
| 1094 | cgcggaGGCAGTACTACTCTTCCTAGAtccgcg |
| 1095 | ctcgcgGGCAGTACTACTCTTCCTAGAcgcgag |
| 1096 | ccgaccGGCAGTACTACTCTTCCTAGAggtcgg |
| 1097 | cgacgtGGCAGTACTACTCTTCCTAGAacgtcg |
| 1098 | cccaccGGCAGTACTACTCTTCCTAGAggtggg |
| 1099 | cgccgaGGCAGTACTACTCTTCCTAGAtcggcg |
| 1100 | ctggcgGGCAGTACTACTCTTCCTAGAcgccag |
| 1101 | ctgccgGGCAGTACTACTCTTCCTAGAcggcag |
| 1102 | ccgggtGGCAGTACTACTCTTCCTAGAacccgg |
| 1103 | ccccccGGCAGTACTACTCTTCCTAGAgggggg |
| 1104 | gcgcctGGCAGTACTACTCTTCCTAGAaggcgc |
| 1105 | gagcgcGGCAGTACTACTCTTCCTAGAgcgctc |
| 1106 | ggctggGGCAGTACTACTCTTCCTAGAccagcc |
| 1107 | gctgcaGGCAGTACTACTCTTCCTAGAtgcagc |
| 1108 | gggtggGGCAGTACTACTCTTCCTAGAccaccc |
| 1109 | gcggctGGCAGTACTACTCTTCCTAGAagccgc |
| 1110 | gaccgcGGCAGTACTACTCTTCCTAGAgcggtc |
| 1111 | gacggcGGCAGTACTACTCTTCCTAGAgccgtc |
| 1112 | ggcccaGGCAGTACTACTCTTCCTAGAtgggcc |
| 1113 | GGGCAGTACTACTCTTCCTAGAG |
| 1114 | cgcggaGGGCAGTACTACTCTTCCTAGAGtccgcg |
| 1115 | ctcgcgGGGCAGTACTACTCTTCCTAGAGcgcgag |
| 1116 | ccgaccGGGCAGTACTACTCTTCCTAGAGggtcgg |
| 1117 | cgacgtGGGCAGTACTACTCTTCCTAGAGacgtcg |
| 1118 | cccaccGGGCAGTACTACTCTTCCTAGAGggtggg |
| 1119 | cgccgaGGGCAGTACTACTCTTCCTAGAGtcggcg |
| 1120 | ctggcgGGGCAGTACTACTCTTCCTAGAGcgccag |
| 1121 | ctcgccGGGCAGTACTACTCTTCCTAGAGgcgag |
| 1122 | ctgccgGGGCAGTACTACTCTTCCTAGAGcggcag |
| 1123 | ccgggtGGGCAGTACTACTCTTCCTAGAGacccgg |
| 1124 | ccccccGGGCAGTACTACTCTTCCTAGAGgggggg |
| 1125 | gcgcctGGGCAGTACTACTCTTCCTAGAGaggcgc |
| 1126 | gagcgcGGGCAGTACTACTCTTCCTAGAGgcgctc |
| 1127 | ggctggGGGCAGTACTACTCTTCCTAGAGccagcc |
| 1128 | gctgcaGGGCAGTACTACTCTTCCTAGAGtgcagc |
| 1129 | gggtggGGGCAGTACTACTCTTCCTAGAGccaccc |
| 1130 | gcggctGGGCAGTACTACTCTTCCTAGAGagccgc |
| 1131 | gaccgcGGGCAGTACTACTCTTCCTAGAGgcggtc |
| 1132 | gacggcGGGCAGTACTACTCTTCCTAGAGgccgtc |
| 1133 | ggcccaGGGCAGTACTACTCTTCCTAGAGtgggcc |

Group 44

| Seq. ID No | Sequence |
|---|---|
| 1134 | CATCGGTTAAACAATTAGCACTG |
| 1135 | cgcggaCATCGGTTAAACAATTAGCACTGtccgcg |
| 1136 | ctcgcgCATCGGTTAAACAATTAGCACTGcgcgag |
| 1137 | ccgaccCATCGGTTAAACAATTAGCACTGggtcgg |
| 1138 | cgacgtCATCGGTTAAACAATTAGCACTGacgtcg |
| 1139 | cccaccCATCGGTTAAACAATTAGCACTGggtggg |
| 1140 | cagtgcCATCGGTTAAACAATTAGCACTG |
| 1141 | cgccgaCATCGGTTAAACAATTAGCACTGtcggcg |
| 1142 | ctggcgCATCGGTTAAACAATTAGCACTGcgccag |
| 1143 | ctgccgCATCGGTTAAACAATTAGCACTGcggcag |
| 1144 | ccgggtCATCGGTTAAACAATTAGCACTGacccgg |
| 1145 | ccccccCATCGGTTAAACAATTAGCACTGgggggg |
| 1146 | gcgcctCATCGGTTAAACAATTAGCACTGaggcgc |
| 1147 | gagcgcCATCGGTTAAACAATTAGCACTGgcgctc |
| 1148 | ggctggCATCGGTTAAACAATTAGCACTGccagcc |
| 1149 | gctgcaCATCGGTTAAACAATTAGCACTGtgcagc |
| 1150 | gggtggCATCGGTTAAACAATTAGCACTGccaccc |
| 1151 | gcggctCATCGGTTAAACAATTAGCACTGagccgc |
| 1152 | gaccgcCATCGGTTAAACAATTAGCACTGgcggtc |
| 1153 | gacggcCATCGGTTAAACAATTAGCACTGgccgtc |
| 1154 | ggcccaCATCGGTTAAACAATTAGCACTGtgggcc |

Group 45

| Seq. ID No | Sequence |
|---|---|
| 1155 | ACGAACCTCTAAAGAGGTTCATC |
| 1156 | cgcggaACGAACCTCTAAAGAGGTTCATCtccgcg |
| 1157 | ctcgcgACGAACCTCTAAAGAGGTTCATCcgcgag |
| 1158 | ccgaccACGAACCTCTAAAGAGGTTCATCggtcgg |
| 1159 | ctcgcgGATGAACCTCTTTAGAGGTTCGTcgcgag |
| 1160 | cgacgtACGAACCTCTAAAGAGGTTCATCacgtcg |
| 1161 | cccaccACGAACCTCTAAAGAGGTTCATCggtggg |
| 1162 | cgccgaACGAACCTCTAAAGAGGTTCATCtcggcg |
| 1163 | ctggcgACGAACCTCTAAAGAGGTTCATCcgccag |
| 1164 | ctgccgACGAACCTCTAAAGAGGTTCATCcggcag |
| 1165 | ccgggtACGAACCTCTAAAGAGGTTCATCacccgg |
| 1166 | ccccccACGAACCTCTAAAGAGGTTCATCgggggg |
| 1167 | gcgcctACGAACCTCTAAAGAGGTTCATCaggcgc |
| 1168 | gagcgcACGAACCTCTAAAGAGGTTCATCgcgctc |
| 1169 | ggctggACGAACCTCTAAAGAGGTTCATCccagcc |
| 1170 | gctgcaACGAACCTCTAAAGAGGTTCATCtgcagc |
| 1171 | gggtggACGAACCTCTAAAGAGGTTCATCccaccc |
| 1172 | gcggctACGAACCTCTAAAGAGGTTCATCagccgc |
| 1173 | gaccgcACGAACCTCTAAAGAGGTTCATCgcggtc |
| 1174 | gacggcACGAACCTCTAAAGAGGTTCATCgccgtc |
| 1175 | ggcccaACGAACCTCTAAAGAGGTTCATCtgggcc |
| 1176 | ACCTCTAAAGAGGTTCATCCACAG |
| 1177 | cgcggaACCTCTAAAGAGGTTCATCCACAGtccgcg |
| 1178 | ctcgcgACCTCTAAAGAGGTTCATCCACAGcgcgag |
| 1179 | ccgaccACCTCTAAAGAGGTTCATCCACAGggtcgg |
| 1180 | cgacgtACCTCTAAAGAGGTTCATCCACAGacgtcg |
| 1181 | cccaccACCTCTAAAGAGGTTCATCCACAGggtggg |
| 1182 | cgccgaACCTCTAAAGAGGTTCATCCACAGtcggcg |
| 1183 | ctggcgACCTCTAAAGAGGTTCATCCACAGcgccag |
| 1184 | ctgccgACCTCTAAAGAGGTTCATCCACAGcggcag |
| 1185 | ccgggtACCTCTAAAGAGGTTCATCCACAGacccgg |
| 1186 | ccccccACCTCTAAAGAGGTTCATCCACAGgggggg |
| 1187 | gcgcctACCTCTAAAGAGGTTCATCCACAGaggcgc |
| 1188 | cctACCTCTAAAGAGGTTCATCCACAGgaggtagg |
| 1189 | gagcgcACCTCTAAAGAGGTTCATCCACAGgcgctc |
| 1190 | ggctggACCTCTAAAGAGGTTCATCCACAGccagcc |
| 1191 | gctgcaACCTCTAAAGAGGTTCATCCACAGtgcagc |
| 1192 | gggtggACCTCTAAAGAGGTTCATCCACAGccaccc |
| 1193 | gcggctACCTCTAAAGAGGTTCATCCACAGagccgc |
| 1194 | gaccgcACCTCTAAAGAGGTTCATCCACAGgcggtc |
| 1195 | gacggcACCTCTAAAGAGGTTCATCCACAGgccgtc |
| 1196 | ggcccaACCTCTAAAGAGGTTCATCCACAGtgggcc |

Group 46

| Seq. ID No | Sequence |
|---|---|
| 1197 | CTGTTACTTATGCGCCCTATTC |
| 1198 | cgcggaCTGTTACTTATGCGCCCTATTCtccgcg |
| 1199 | ctcgcgCTGTTACTTATGCGCCCTATTCcgcgag |
| 1200 | ccgaccCTGTTACTTATGCGCCCTATTCggtcgg |
| 1201 | cgacgtCTGTTACTTATGCGCCCTATTCacgtcg |
| 1202 | cccaccCTGTTACTTATGCGCCCTATTCggtggg |
| 1203 | cgccgaCTGTTACTTATGCGCCCTATTCtcggcg |
| 1204 | ctggcgCTGTTACTTATGCGCCCTATTCcgccag |
| 1205 | ctgccgCTGTTACTTATGCGCCCTATTCcggcag |
| 1206 | ccgggtCTGTTACTTATGCGCCCTATTCacccgg |
| 1207 | ccccccCTGTTACTTATGCGCCCTATTCgggggg |
| 1208 | gcgcctCTGTTACTTATGCGCCCTATTCaggcgc |
| 1209 | gagcgcCTGTTACTTATGCGCCCTATTCgcgctc |
| 1210 | ggctggCTGTTACTTATGCGCCCTATTCccagcc |
| 1211 | gctgcaCTGTTACTTATGCGCCCTATTCtgcagc |
| 1212 | gggtggCTGTTACTTATGCGCCCTATTCccaccc |
| 1213 | gcggctCTGTTACTTATGCGCCCTATTCagccgc |
| 1214 | gaccgcCTGTTACTTATGCGCCCTATTCgcggtc |
| 1215 | gacggcCTGTTACTTATGCGCCCTATTCgccgtc |
| 1216 | ggcccaCTGTTACTTATGCGCCCTATTCtgggcc |

1. A method for detecting microorganisms, for example of the Enterobacteriaceae family of bacteria, in a sample by means of delivery of identity-determining nucleic acid probes into the individual cell bodies, wherein the nucleic acid probes hybridize to the nucleic acids of the microorganisms, and subsequent optical detection of the hybridizations generated in the individual cell bodies, wherein a mixture of at least a first nucleic acid probe and a second nucleic acid probe is used, and in that the first nucleic acid probe and the second nucleic acid probe respectively bind in one of two nonoverlapping regions of the target nucleic acid.

2. The method as claimed in claim 1, wherein the mixture is formed such that, from the present combinatorics of groups 1 to 12, selection is respectively made of a representative from each group or a representative from at least two groups, especially wherein group 1 comprises some or all of SEQ ID NO: 1 to SEQ ID NO: 19, group 2 comprises some or all of SEQ ID NO: 20 to SEQ ID NO: 40, group 3 comprises some or all of SEQ ID NO: 41 to SEQ ID NO: 59, group 4 comprises some or all of SEQ ID NO: 60 to SEQ ID NO: 78, group 5 comprises some or all of SEQ ID NO: 79 to SEQ ID NO: 99, group 6 comprises some or all of SEQ ID NO: 100 to SEQ ID NO: 118, group 7 comprises some or all of SEQ ID NO: 119 to SEQ ID NO: 137, group 8 comprises some or all of SEQ ID NO: 138 to SEQ ID NO: 156, group 9 comprises some or all of SEQ ID NO: 157 to SEQ ID NO: 175, group 10 comprises some or all of SEQ ID NO: 176 to SEQ ID NO: 196, group 11 comprises some or all of SEQ ID NO: 197 to SEQ ID NO: 217, and group 12 comprises some or all of SEQ ID NO: 218 to SEQ ID NO: 236.

3. The method as claimed in claim 1, wherein the mixture is formed such that, from the present combinatorics of groups 13 to 33, selection is respectively made of a representative from each group or a representative from at least two groups, especially wherein group 13 comprises some or all of SEQ ID NO: 237 to SEQ ID NO: 294, group 14 comprises some or all of SEQ ID NO: 295 to SEQ ID NO: 314, group 15 comprises some or all of SEQ ID NO: 315 to SEQ ID NO: 372, group 16 comprises some or all of SEQ ID NO: 373 to SEQ ID NO: 411, group 17 comprises some or all of SEQ ID NO: 412 to SEQ ID NO: 450, group 18 comprises some or all of SEQ ID NO: 451 to SEQ ID NO: 508, group 19 comprises some or all of SEQ ID NO: 509 to SEQ ID NO: 528, group 20 comprises some or all of SEQ ID NO: 529 to SEQ ID NO: 547, group 21 comprises some or all of SEQ ID NO: 548 to SEQ ID NO: 567, group 22 comprises some or all of SEQ ID NO: 568 to SEQ ID NO: 587, group 23 comprises some or all of SEQ ID NO: 588 to SEQ ID NO: 607, group 24 comprises some or all of SEQ ID NO: 608 to SEQ ID NO: 626, group 25 comprises some or all of SEQ ID NO: 627 to SEQ ID NO: 645, group 26 comprises some or all of SEQ ID NO: 646 to SEQ ID NO: 664, group 27 comprises some or all of SEQ ID NO: 665 to SEQ ID NO: 684, group 28 comprises some or all of SEQ ID NO: 685 to SEQ ID NO: 704, group 29 comprises some or all of SEQ ID NO: 705 to SEQ ID NO: 724, group 30 comprises some or all of SEQ ID NO: 725 to SEQ ID NO: 744, group 31 comprises some or all of SEQ ID NO: 745 to SEQ ID NO: 764, group 32 comprises some or all of SEQ ID NO: 765 to SEQ ID NO: 804, and group 33 comprises some or all of SEQ ID NO: 805 to SEQ ID NO: 824.

4. The method as claimed in claim 1, wherein the mixture is formed such that, from the present combinatorics of groups 34 to 46, selection is respectively made of a representative from each group or a representative from at least two groups, especially wherein group 34 comprises some or all of SEQ ID NO: 825 to SEQ ID NO: 844, group 35 comprises some or all of SEQ ID NO: 845 to SEQ ID NO: 865, group 36 comprises some or all of SEQ ID NO: 866 to SEQ ID NO: 886, group 37 comprises some or all of SEQ ID NO: 887 to SEQ ID NO: 927, group 38 comprises some or all of SEQ ID NO: 928 to SEQ ID NO: 947, group 39 comprises some or all of SEQ ID NO: 948 to SEQ ID NO: 967, group 40 comprises some or all of SEQ ID NO: 968 to SEQ ID NO: 988, group 41 comprises some or all of SEQ ID NO: 989 to SEQ ID NO: 1009, group 42 comprises some or all of SEQ ID NO: 1010 to SEQ ID NO: 1071, group 43 comprises some or all of SEQ ID NO: 1072 to SEQ ID NO: 1133, group 44 comprises some or all of SEQ ID NO: 1134 to SEQ ID NO: 1154, group 45 comprises some or all of SEQ ID NO: 1155 to SEQ ID NO: 1196, and group 46 comprises some or all of SEQ ID NO: 1197 to SEQ ID NO: 1216.

5. The method as claimed in claim 1, wherein the detection reaction using nucleic acid probes is carried out by means of in-situ hybridization and/or fluorescence in-situ hybridization (FISH), nucleic acid amplification reaction and/or microarray.

6. The method as claimed in claim 1, wherein the nucleic acid probes are each designed as linear probes and/or probes having secondary structure, preferably molecular beacons.

7. The method as claimed in claim 1, wherein optical sensitivity is set such that only those microorganisms having at least two binding events are detected.

8. The method as claimed in claim 1, wherein the first and/or second nucleic acid probe has/have at least one first dye conjugated to the 5′ end and/or at least one second dye conjugated to the 3′ end and/or intramolecularly bound dyes.

9. The method as claimed in claim 1, wherein the nucleic acid probe has further nucleotides as stem sequence at the 5′ end and/or 3′ end and/or at least one functional part, especially wherein the stem sequences and/or functional parts can be formed in relation to one another such that they do not mutually interact with one another.

10. The method as claimed in claim 1, wherein the selection of the nonoverlapping regions is chosen such that the nucleic acid probes do not interfere with one another in their radiation behavior.

11. The method as claimed in claim 1, wherein the nucleic acid probes are formed such that a particular color combination is detectable, especially wherein the or a first nucleic acid probe has a first dye and the or a second nucleic acid probe has a second dye.

12. The method as claimed in claim 1, wherein the nucleic acid probe has at least one optically detectable label.

13. The method as claimed in claim 1, wherein the detectable label is an enzyme label, affinity label and/or a dye, especially wherein the dye is a fluorescent dye and/or the affinity label includes biotin-streptavidin or antigen-antibody affinity binding pairs.

14. The method as claimed in claim 1, wherein each nucleic acid probe binds to at least 80% of the Enterobacteriaceae family of bacteria and/or each nucleic acid probe binds detectably just to the nucleic acids of the Enterobacteriaceae family of bacteria, and not to the nucleic acids of an organism belonging to a different family of bacteria.

15. The use of at least one nucleic acid probe or at least two nucleic acid probes selected from the group consisting of SEQ ID NO: 1, SEQ ID NO: 20, SEQ ID NO: 41, SEQ ID NO: 60, SEQ ID NO: 79, SEQ ID NO: 100, SEQ ID NO: 119, SEQ ID NO: 138, SEQ ID NO: 157, SEQ ID NO: 176, SEQ ID NO: 197, SEQ ID NO: 218 and/or further sequences from the present combinatorics of groups 1 to 12 for detection of Enterobacteriaceae and/or for immobilization on a carrier material, especially a fluidic channel system.

16. The use of at least one nucleic acid probe or at least two nucleic acid probes selected from the group consisting of SEQ ID NO: 237, SEQ ID NO: 295, SEQ ID NO: 315, SEQ ID NO: 373, SEQ ID NO: 412, SEQ ID NO: 451, SEQ ID NO: 509, SEQ ID NO: 529, SEQ ID NO: 548, SEQ ID NO: 568, SEQ ID NO: 588, SEQ ID NO: 608, SEQ ID NO: 627, SEQ ID NO: 646, SEQ ID NO: 665, SEQ ID NO: 685, SEQ ID NO: 705, SEQ ID NO: 725, SEQ ID NO: 745, SEQ ID NO: 765, SEQ ID NO: 805 and/or further sequences from the present combinatorics of groups 13 to 33 for detection of Listeriaceae and/or for immobilization on a carrier material, especially a fluidic channel system.

17. The use of at least one nucleic acid probe or at least two nucleic acid probes selected from the group consisting of SEQ ID NO: 825, SEQ ID NO: 845, SEQ ID NO: 866, SEQ ID NO: 887, SEQ ID NO: 928, SEQ ID NO: 948, SEQ ID NO: 968, SEQ ID NO: 989, SEQ ID NO: 1010, SEQ ID NO: 1072, SEQ ID NO: 1134, SEQ ID NO: 1155, SEQ ID NO: 1197 and/or further sequences from the present combinatorics of groups 34 to 46 for detection of Listeriaceae, especially Listeria monocytogenes, and/or for immobilization on a carrier material, especially a fluidic channel system.

18. A fluidic channel system, preferably disk-shaped sample carrier, comprising means for carrying out the method as claimed in claim 1, especially comprising at least one cavity containing at least one nucleic acid probe or at least two nucleic acid probes selected from the group consisting of SEQ ID NO: 1, SEQ ID NO: 20, SEQ ID NO: 41, SEQ ID NO: 60, SEQ ID NO: 79, SEQ ID NO: 100, SEQ ID NO: 119, SEQ ID NO: 138, SEQ ID NO: 157, SEQ ID NO: 176, SEQ ID NO: 197, SEQ ID NO: 218 and/or further sequences from the present combinatorics of groups 1 to 12 and/or at least one nucleic acid probe or at least two nucleic acid probes selected from the group consisting of SEQ ID NO: 237, SEQ ID NO: 295, SEQ ID NO: 315, SEQ ID NO: 373, SEQ ID NO: 412, SEQ ID NO: 451, SEQ ID NO: 509, SEQ ID NO: 529, SEQ ID NO: 548, SEQ ID NO: 568, SEQ ID NO: 588, SEQ ID NO: 608, SEQ ID NO: 627, SEQ ID NO: 646, SEQ ID NO: 665, SEQ ID NO: 685, SEQ ID NO: 705, SEQ ID NO: 725, SEQ ID NO: 745, SEQ ID NO: 765, SEQ ID NO: 805 and/or further sequences from the present combinatorics of groups 13 to 33 and/or at least one nucleic acid probe or at least two nucleic acid probes selected from the group consisting of SEQ ID NO: 825, SEQ ID NO: 845, SEQ ID NO: 866, SEQ ID NO: 887, SEQ ID NO: 928, SEQ ID NO: 948, SEQ ID NO: 968, SEQ ID NO: 989, SEQ ID NO: 1010, SEQ ID NO: 1072, SEQ ID NO: 1134, SEQ ID NO: 1155, SEQ ID NO: 1197 and/or further sequences from the present combinatorics of groups 34 to 46 and/or comprising means for optical detection of labeled microorganisms.


SEQUENCE LISTING (1216 sequences)

SEQ ID NO: 1 - DNA, 22 bp, Artificial Sequence
  synthesized nucleic acid probe to detect Enterobacteriaceae 1
cctacttcttttgcaacccact

SEQ ID NO: 2 - DNA, 34 bp, Artificial Sequence
  synthesized nucleic acid probe to detect Enterobacteriaceae 2
cgcggacctacttcttttgcaacccacttccgcg

SEQ ID NO: 3 - DNA, 34 bp, Artificial Sequence
  synthesized nucleic acid probe to detect Enterobacteriaceae 3
ctcgcgcctacttcttttgcaacccactcgcgag

SEQ ID NO: 4 - DNA, 34 bp, Artificial Sequence
  synthesized nucleic acid probe to detect Enterobacteriaceae 4
ccgacccctacttcttttgcaacccactggtcgg

SEQ ID NO: 5 - DNA, 34 bp, Artificial Sequence
  synthesized nucleic acid probe to detect Enterobacteriaceae 5
cgacgtcctacttcttttgcaacccactacgtcg

SEQ ID NO: 6 - DNA, 34 bp, Artificial Sequence
  synthesized nucleic acid probe to detect Enterobacteriaceae 6
cccacccctacttcttttgcaacccactggtggg

SEQ ID NO: 7 - DNA, 34 bp, Artificial Sequence
  synthesized nucleic acid probe to detect Enterobacteriaceae 7
cgccgacctacttcttttgcaacccacttcggcg

SEQ ID NO: 8 - DNA, 34 bp, Artificial Sequence
  synthesized nucleic acid probe to detect Enterobacteriaceae 8
ctggcgcctacttcttttgcaacccactcgccag

SEQ ID NO: 9 - DNA, 34 bp, Artificial Sequence
  synthesized nucleic acid probe to detect Enterobacteriaceae 9
ctgccgcctacttcttttgcaacccactcggcag

SEQ ID NO: 10 - DNA, 34 bp, Artificial Sequence
  synthesized nucleic acid probe to detect Enterobacteriaceae 10
ccgggtcctacttcttttgcaacccactacccgg

SEQ ID NO: 11 - DNA, 34 bp, Artificial Sequence
  synthesized nucleic acid probe to detect Enterobacteriaceae 11
gcgcctcctacttcttttgcaacccactaggcgc

SEQ ID NO: 12 - DNA, 34 bp, Artificial Sequence
  synthesized nucleic acid probe to detect Enterobacteriaceae 12
gagcgccctacttcttttgcaacccactgcgctc

SEQ ID NO: 13 - DNA, 34 bp, Artificial Sequence
  Seq. IDF NO 13
ggctggcctacttcttttgcaacccactccagcc

SEQ ID NO: 14 - DNA, 34 bp, Artificial Sequence
  synthesized nucleic acid probe to detect Enterobacteriaceae 14
gctgcacctacttcttttgcaacccacttgcagc

SEQ ID NO: 15 - DNA, 34 bp, Artificial Sequence
  synthesized nucleic acid probe to detect Enterobacteriaceae 15
gggtggcctacttcttttgcaacccactccaccc

SEQ ID NO: 16 - DNA, 34 bp, Artificial Sequence
  synthesized nucleic acid probe to detect Enterobacteriaceae 16
gcggctcctacttcttttgcaacccactagccgc

SEQ ID NO: 17 - DNA, 34 bp, Artificial Sequence
  synthesized nucleic acid probe to detect Enterobacteriaceae 17
gaccgccctacttcttttgcaacccactgcggtc

SEQ ID NO: 18 - DNA, 34 bp, Artificial Sequence
  synthesized nucleic acid probe to detect Enterobacteriaceae 18
gacggccctacttcttttgcaacccactgccgtc

SEQ ID NO: 19 - DNA, 34 bp, Artificial Sequence
  synthesized nucleic acid probe to detect Enterobacteriaceae 19
ggcccacctacttcttttgcaacccacttgggcc

SEQ ID NO: 20 - DNA, 27 bp, Artificial Sequence
  synthesized nucleic acid probe to detect Enterobacteriaceae 20
cggttaaccttgctacagaatataagt

SEQ ID NO: 21 - DNA, 39 bp, Artificial Sequence
  synthesized nucleic acid probe to detect Enterobacteriaceae 21
cgcggacggttaaccttgctacagaatataagttccgcg

SEQ ID NO: 22 - DNA, 39 bp, Artificial Sequence
  synthesized nucleic acid probe to detect Enterobacteriaceae 22
ctcgcgcggttaaccttgctacagaatataagtcgcgag

SEQ ID NO: 23 - DNA, 39 bp, Artificial Sequence
  synthesized nucleic acid probe to detect Enterobacteriaceae 23
ccgacccggttaaccttgctacagaatataagtggtcgg

SEQ ID NO: 24 - DNA, 39 bp, Artificial Sequence
  synthesized nucleic acid probe to detect Enterobacteriaceae 24
cgacgtcggttaaccttgctacagaatataagtacgtcg

SEQ ID NO: 25 - DNA, 39 bp, Artificial Sequence
  synthesized nucleic acid probe to detect Enterobacteriaceae 25
cccacccggttaaccttgctacagaatataagtggtggg

SEQ ID NO: 26 - DNA, 39 bp, Artificial Sequence
  synthesized nucleic acid probe to detect Enterobacteriaceae 26
cgccgacggttaaccttgctacagaatataagttcggcg

SEQ ID NO: 27 - DNA, 39 bp, Artificial Sequence
  synthesized nucleic acid probe to detect Enterobacteriaceae 27
ctggcgcggttaaccttgctacagaatataagtcgccag

SEQ ID NO: 28 - DNA, 39 bp, Artificial Sequence
  synthesized nucleic acid probe to detect Enterobacteriaceae 28
ctgccgcggttaaccttgctacagaatataagtcggcag

SEQ ID NO: 29 - DNA, 39 bp, Artificial Sequence
  synthesized nucleic acid probe to detect Enterobacteriaceae 29
ccgggtcggttaaccttgctacagaatataagtacccgg

SEQ ID NO: 30 - DNA, 38 bp, Artificial Sequence
  synthesized nucleic acid probe to detect Enterobacteriaceae 30
gcgtccggttaaccttgctacagaatataagtggacgc

SEQ ID NO: 31 - DNA, 39 bp, Artificial Sequence
  synthesized nucleic acid probe to detect Enterobacteriaceae 31
gcgcctcggttaaccttgctacagaatataagtaggcgc

SEQ ID NO: 32 - DNA, 39 bp, Artificial Sequence
  synthesized nucleic acid probe to detect Enterobacteriaceae 32
gagcgccggttaaccttgctacagaatataagtgcgctc

SEQ ID NO: 33 - DNA, 39 bp, Artificial Sequence
  synthesized nucleic acid probe to detect Enterobacteriaceae 33
ggctggcggttaaccttgctacagaatataagtccagcc

SEQ ID NO: 34 - DNA, 39 bp, Artificial Sequence
  synthesized nucleic acid probe to detect Enterobacteriaceae 34
gctgcacggttaaccttgctacagaatataagttgcagc

SEQ ID NO: 35 - DNA, 39 bp, Artificial Sequence
  synthesized nucleic acid probe to detect Enterobacteriaceae 35
gggtggcggttaaccttgctacagaatataagtccaccc

SEQ ID NO: 36 - DNA, 39 bp, Artificial Sequence
  synthesized nucleic acid probe to detect Enterobacteriaceae 36
gcggctcggttaaccttgctacagaatataagtagccgc

SEQ ID NO: 37 - DNA, 39 bp, Artificial Sequence
  synthesized nucleic acid probe to detect Enterobacteriaceae 37
gaccgccggttaaccttgctacagaatataagtgcggtc

SEQ ID NO: 38 - DNA, 39 bp, Artificial Sequence
  synthesized nucleic acid probe to detect Enterobacteriaceae 38
gacggccggttaaccttgctacagaatataagtgccgtc

SEQ ID NO: 39 - DNA, 39 bp, Artificial Sequence
  synthesized nucleic acid probe to detect Enterobacteriaceae 39
ggcccacggttaaccttgctacagaatataagttgggcc

SEQ ID NO: 40 - DNA, 38 bp, Artificial Sequence
  synthesized nucleic acid probe to detect Enterobacteriaceae 40
cgcagcggttaaccttgctacagaatataagtgctgcg

SEQ ID NO: 41 - DNA, 22 bp, Artificial Sequence
  synthesized nucleic acid probe to detect Enterobacteriaceae 41
gcttaaaccgggacaaccgtcg

SEQ ID NO: 42 - DNA, 34 bp, Artificial Sequence
  synthesized nucleic acid probe to detect Enterobacteriaceae 42
cgcggagcttaaaccgggacaaccgtcgtccgcg

SEQ ID NO: 43 - DNA, 34 bp, Artificial Sequence
  synthesized nucleic acid probe to detect Enterobacteriaceae 43
ctcgcggcttaaaccgggacaaccgtcgcgcgag

SEQ ID NO: 44 - DNA, 34 bp, Artificial Sequence
  synthesized nucleic acid probe to detect Enterobacteriaceae 44
ccgaccgcttaaaccgggacaaccgtcgggtcgg

SEQ ID NO: 45 - DNA, 34 bp, Artificial Sequence
  synthesized nucleic acid probe to detect Enterobacteriaceae 45
cgacgtgcttaaaccgggacaaccgtcgacgtcg

SEQ ID NO: 46 - DNA, 34 bp, Artificial Sequence
  synthesized nucleic acid probe to detect Enterobacteriaceae 46
cccaccgcttaaaccgggacaaccgtcgggtggg

SEQ ID NO: 47 - DNA, 34 bp, Artificial Sequence
  synthesized nucleic acid probe to detect Enterobacteriaceae 47
cgccgagcttaaaccgggacaaccgtcgtcggcg

SEQ ID NO: 48 - DNA, 34 bp, Artificial Sequence
  synthesized nucleic acid probe to detect Enterobacteriaceae 48
ctggcggcttaaaccgggacaaccgtcgcgccag

SEQ ID NO: 49 - DNA, 34 bp, Artificial Sequence
  synthesized nucleic acid probe to detect Enterobacteriaceae 49
ctgccggcttaaaccgggacaaccgtcgcggcag

SEQ ID NO: 50 - DNA, 34 bp, Artificial Sequence
  synthesized nucleic acid probe to detect Enterobacteriaceae 50
ccgggtgcttaaaccgggacaaccgtcgacccgg

SEQ ID NO: 51 - DNA, 34 bp, Artificial Sequence
  synthesized nucleic acid probe to detect Enterobacteriaceae 51
gcgcctgcttaaaccgggacaaccgtcgaggcgc

SEQ ID NO: 52 - DNA, 34 bp, Artificial Sequence
  synthesized nucleic acid probe to detect Enterobacteriaceae 52
gagcgcgcttaaaccgggacaaccgtcggcgctc

SEQ ID NO: 53 - DNA, 34 bp, Artificial Sequence
  synthesized nucleic acid probe to detect Enterobacteriaceae 53
ggctgggcttaaaccgggacaaccgtcgccagcc

SEQ ID NO: 54 - DNA, 34 bp, Artificial Sequence
  synthesized nucleic acid probe to detect Enterobacteriaceae 54
gctgcagcttaaaccgggacaaccgtcgtgcagc

SEQ ID NO: 55 - DNA, 34 bp, Artificial Sequence
  synthesized nucleic acid probe to detect Enterobacteriaceae 55
gggtgggcttaaaccgggacaaccgtcgccaccc

SEQ ID NO: 56 - DNA, 34 bp, Artificial Sequence
  synthesized nucleic acid probe to detect Enterobacteriaceae 56
gcggctgcttaaaccgggacaaccgtcgagccgc

SEQ ID NO: 57 - DNA, 34 bp, Artificial Sequence
  synthesized nucleic acid probe to detect Enterobacteriaceae 57
gaccgcgcttaaaccgggacaaccgtcggcggtc

SEQ ID NO: 58 - DNA, 34 bp, Artificial Sequence
  synthesized nucleic acid probe to detect Enterobacteriaceae 58
gacggcgcttaaaccgggacaaccgtcggccgtc

SEQ ID NO: 59 - DNA, 34 bp, Artificial Sequence
  synthesized nucleic acid probe to detect Enterobacteriaceae 59
ggcccagcttaaaccgggacaaccgtcgtgggcc

SEQ ID NO: 60 - DNA, 21 bp, Artificial Sequence
  synthesized nucleic acid probe to detect Enterobacteriaceae 60
cgtttccagtagttatccccc

SEQ ID NO: 61 - DNA, 33 bp, Artificial Sequence
  synthesized nucleic acid probe to detect Enterobacteriaceae 61
cgcggacgtttccagtagttatccccctccgcg

SEQ ID NO: 62 - DNA, 33 bp, Artificial Sequence
  synthesized nucleic acid probe to detect Enterobacteriaceae 62
ctcgcgcgtttccagtagttatccccccgcgag

SEQ ID NO: 63 - DNA, 33 bp, Artificial Sequence
  synthesized nucleic acid probe to detect Enterobacteriaceae 63
ccgacccgtttccagtagttatcccccggtcgg

SEQ ID NO: 64 - DNA, 33 bp, Artificial Sequence
  synthesized nucleic acid probe to detect Enterobacteriaceae 64
cgacgtcgtttccagtagttatcccccacgtcg

SEQ ID NO: 65 - DNA, 33 bp, Artificial Sequence
  synthesized nucleic acid probe to detect Enterobacteriaceae 65
cccacccgtttccagtagttatcccccggtggg

SEQ ID NO: 66 - DNA, 33 bp, Artificial Sequence
  synthesized nucleic acid probe to detect Enterobacteriaceae 66
cgccgacgtttccagtagttatccccctcggcg

SEQ ID NO: 67 - DNA, 33 bp, Artificial Sequence
  synthesized nucleic acid probe to detect Enterobacteriaceae 67
ctggcgcgtttccagtagttatccccccgccag

SEQ ID NO: 68 - DNA, 33 bp, Artificial Sequence
  synthesized nucleic acid probe to detect Enterobacteriaceae 68
ctgccgcgtttccagtagttatccccccggcag

SEQ ID NO: 69 - DNA, 33 bp, Artificial Sequence
  synthesized nucleic acid probe to detect Enterobacteriaceae 69
ccgggtcgtttccagtagttatcccccacccgg

SEQ ID NO: 70 - DNA, 33 bp, Artificial Sequence
  synthesized nucleic acid probe to detect Enterobacteriaceae 70
gcgcctcgtttccagtagttatcccccaggcgc

SEQ ID NO: 71 - DNA, 33 bp, Artificial Sequence
  synthesized nucleic acid probe to detect Enterobacteriaceae 71
gagcgccgtttccagtagttatcccccgcgctc

SEQ ID NO: 72 - DNA, 33 bp, Artificial Sequence
  synthesized nucleic acid probe to detect Enterobacteriaceae 72
ggctggcgtttccagtagttatcccccccagcc

SEQ ID NO: 73 - DNA, 33 bp, Artificial Sequence
  synthesized nucleic acid probe to detect Enterobacteriaceae 73
gctgcacgtttccagtagttatccccctgcagc

SEQ ID NO: 74 - DNA, 33 bp, Artificial Sequence
  synthesized nucleic acid probe to detect Enterobacteriaceae 74
gggtggcgtttccagtagttatcccccccaccc

SEQ ID NO: 75 - DNA, 33 bp, Artificial Sequence
  synthesized nucleic acid probe to detect Enterobacteriaceae 75
gcggctcgtttccagtagttatcccccagccgc

SEQ ID NO: 76 - DNA, 33 bp, Artificial Sequence
  synthesized nucleic acid probe to detect Enterobacteriaceae 76
gaccgccgtttccagtagttatcccccgcggtc

SEQ ID NO: 77 - DNA, 33 bp, Artificial Sequence
  synthesized nucleic acid probe to detect Enterobacteriaceae 77
gacggccgtttccagtagttatcccccgccgtc

SEQ ID NO: 78 - DNA, 33 bp, Artificial Sequence
  synthesized nucleic acid probe to detect Enterobacteriaceae 78
ggcccacgtttccagtagttatccccctgggcc

SEQ ID NO: 79 - DNA, 18 bp, Artificial Sequence
  synthesized nucleic acid probe to detect Enterobacteriaceae 79
gcgaggtcgcttctcttt

SEQ ID NO: 80 - DNA, 30 bp, Artificial Sequence
  synthesized nucleic acid probe to detect Enterobacteriaceae 80
cgcggagcgaggtcgcttctcttttccgcg

SEQ ID NO: 81 - DNA, 30 bp, Artificial Sequence
  synthesized nucleic acid probe to detect Enterobacteriaceae 81
ctcgcggcgaggtcgcttctctttcgcgag

SEQ ID NO: 82 - DNA, 30 bp, Artificial Sequence
  synthesized nucleic acid probe to detect Enterobacteriaceae 82
ccgaccgcgaggtcgcttctctttggtcgg

SEQ ID NO: 83 - DNA, 30 bp, Artificial Sequence
  synthesized nucleic acid probe to detect Enterobacteriaceae 83
cgacgtgcgaggtcgcttctctttacgtcg

SEQ ID NO: 84 - DNA, 30 bp, Artificial Sequence
  synthesized nucleic acid probe to detect Enterobacteriaceae 84
cccaccgcgaggtcgcttctctttggtggg

SEQ ID NO: 85 - DNA, 30 bp, Artificial Sequence
  synthesized nucleic acid probe to detect Enterobacteriaceae 85
cgccgagcgaggtcgcttctcttttcggcg

SEQ ID NO: 86 - DNA, 30 bp, Artificial Sequence
  synthesized nucleic acid probe to detect Enterobacteriaceae 86
ctggcggcgaggtcgcttctctttcgccag

SEQ ID NO: 87 - DNA, 30 bp, Artificial Sequence
  synthesized nucleic acid probe to detect Enterobacteriaceae 87
ctgccggcgaggtcgcttctctttcggcag

SEQ ID NO: 88 - DNA, 30 bp, Artificial Sequence
  synthesized nucleic acid probe to detect Enterobacteriaceae 88
ccgggtgcgaggtcgcttctctttacccgg

SEQ ID NO: 89 - DNA, 28 bp, Artificial Sequence
  synthesized nucleic acid probe to detect Enterobacteriaceae 89
gttggcgaggtcgcttctctttgccaac

SEQ ID NO: 90 - DNA, 30 bp, Artificial Sequence
  synthesized nucleic acid probe to detect Enterobacteriaceae 90
gcgcctgcgaggtcgcttctctttaggcgc

SEQ ID NO: 91 - DNA, 30 bp, Artificial Sequence
  synthesized nucleic acid probe to detect Enterobacteriaceae 91
gagcgcgcgaggtcgcttctctttgcgctc

SEQ ID NO: 92 - DNA, 30 bp, Artificial Sequence
  synthesized nucleic acid probe to detect Enterobacteriaceae 92
ggctgggcgaggtcgcttctctttccagcc

SEQ ID NO: 93 - DNA, 30 bp, Artificial Sequence
  synthesized nucleic acid probe to detect Enterobacteriaceae 93
gctgcagcgaggtcgcttctcttttgcagc

SEQ ID NO: 94 - DNA, 30 bp, Artificial Sequence
  synthesized nucleic acid probe to detect Enterobacteriaceae 94
gggtgggcgaggtcgcttctctttccaccc

SEQ ID NO: 95 - DNA, 30 bp, Artificial Sequence
  synthesized nucleic acid probe to detect Enterobacteriaceae 95
gcggctgcgaggtcgcttctctttagccgc

SEQ ID NO: 96 - DNA, 30 bp, Artificial Sequence
  synthesized nucleic acid probe to detect Enterobacteriaceae 96
gaccgcgcgaggtcgcttctctttgcggtc

SEQ ID NO: 97 - DNA, 30 bp, Artificial Sequence
  synthesized nucleic acid probe to detect Enterobacteriaceae 97
gacggcgcgaggtcgcttctctttgccgtc

SEQ ID NO: 98 - DNA, 30 bp, Artificial Sequence
  synthesized nucleic acid probe to detect Enterobacteriaceae 98
ggcccagcgaggtcgcttctcttttgggcc

SEQ ID NO: 99 - DNA, 28 bp, Artificial Sequence
  synthesized nucleic acid probe to detect Enterobacteriaceae 99
caacgcgaggtcgcttctctttgcgttg

SEQ ID NO: 100 - DNA, 20 bp, Artificial Sequence
  synthesized nucleic acid probe to detect Enterobacteriaceae 100
gttgcagccagctggtatct

SEQ ID NO: 101 - DNA, 32 bp, Artificial Sequence
  synthesized nucleic acid probe to detect Enterobacteriaceae 101
cgcggagttgcagccagctggtatcttccgcg

SEQ ID NO: 102 - DNA, 32 bp, Artificial Sequence
  synthesized nucleic acid probe to detect Enterobacteriaceae 102
ctcgcggttgcagccagctggtatctcgcgag

SEQ ID NO: 103 - DNA, 32 bp, Artificial Sequence
  synthesized nucleic acid probe to detect Enterobacteriaceae 103
ccgaccgttgcagccagctggtatctggtcgg

SEQ ID NO: 104 - DNA, 32 bp, Artificial Sequence
  synthesized nucleic acid probe to detect Enterobacteriaceae 104
cgacgtgttgcagccagctggtatctacgtcg

SEQ ID NO: 105 - DNA, 32 bp, Artificial Sequence
  synthesized nucleic acid probe to detect Enterobacteriaceae 105
cccaccgttgcagccagctggtatctggtggg

SEQ ID NO: 106 - DNA, 32 bp, Artificial Sequence
  synthesized nucleic acid probe to detect Enterobacteriaceae 106
cgccgagttgcagccagctggtatcttcggcg

SEQ ID NO: 107 - DNA, 32 bp, Artificial Sequence
  synthesized nucleic acid probe to detect Enterobacteriaceae 107
ctggcggttgcagccagctggtatctcgccag

SEQ ID NO: 108 - DNA, 32 bp, Artificial Sequence
  synthesized nucleic acid probe to detect Enterobacteriaceae 108
ctgccggttgcagccagctggtatctcggcag

SEQ ID NO: 109 - DNA, 32 bp, Artificial Sequence
  synthesized nucleic acid probe to detect Enterobacteriaceae 109
ccgggtgttgcagccagctggtatctacccgg

SEQ ID NO: 110 - DNA, 32 bp, Artificial Sequence
  synthesized nucleic acid probe to detect Enterobacteriaceae 110
gcgcctgttgcagccagctggtatctaggcgc

SEQ ID NO: 111 - DNA, 32 bp, Artificial Sequence
  synthesized nucleic acid probe to detect Enterobacteriaceae 111
gagcgcgttgcagccagctggtatctgcgctc

SEQ ID NO: 112 - DNA, 32 bp, Artificial Sequence
  synthesized nucleic acid probe to detect Enterobacteriaceae 112
ggctgggttgcagccagctggtatctccagcc

SEQ ID NO: 113 - DNA, 32 bp, Artificial Sequence
  synthesized nucleic acid probe to detect Enterobacteriaceae 113
gctgcagttgcagccagctggtatcttgcagc

SEQ ID NO: 114 - DNA, 32 bp, Artificial Sequence
  synthesized nucleic acid probe to detect Enterobacteriaceae 114
gggtgggttgcagccagctggtatctccaccc

SEQ ID NO: 115 - DNA, 32 bp, Artificial Sequence
  synthesized nucleic acid probe to detect Enterobacteriaceae 115
gcggctgttgcagccagctggtatctagccgc

SEQ ID NO: 116 - DNA, 32 bp, Artificial Sequence
  synthesized nucleic acid probe to detect Enterobacteriaceae 116
gaccgcgttgcagccagctggtatctgcggtc

SEQ ID NO: 117 - DNA, 32 bp, Artificial Sequence
  synthesized nucleic acid probe to detect Enterobacteriaceae 117
gacggcgttgcagccagctggtatctgccgtc

SEQ ID NO: 118 - DNA, 32 bp, Artificial Sequence
  synthesized nucleic acid probe to detect Enterobacteriaceae 118
ggcccagttgcagccagctggtatcttgggcc

SEQ ID NO: 119 - DNA, 16 bp, Artificial Sequence
  synthesized nucleic acid probe to detect Enterobacteriaceae 119
cctcgccggggttctt

SEQ ID NO: 120 - DNA, 28 bp, Artificial Sequence
  synthesized nucleic acid probe to detect Enterobacteriaceae 120
cgcggacctcgccggggttctttccgcg

SEQ ID NO: 121 - DNA, 28 bp, Artificial Sequence
  synthesized nucleic acid probe to detect Enterobacteriaceae 121
ctcgcgcctcgccggggttcttcgcgag

SEQ ID NO: 122 - DNA, 28 bp, Artificial Sequence
  synthesized nucleic acid probe to detect Enterobacteriaceae 122
ccgacccctcgccggggttcttggtcgg

SEQ ID NO: 123 - DNA, 28 bp, Artificial Sequence
  synthesized nucleic acid probe to detect Enterobacteriaceae 123
cgacgtcctcgccggggttcttacgtcg

SEQ ID NO: 124 - DNA, 28 bp, Artificial Sequence
  synthesized nucleic acid probe to detect Enterobacteriaceae 124
cccacccctcgccggggttcttggtggg

SEQ ID NO: 125 - DNA, 28 bp, Artificial Sequence
  synthesized nucleic acid probe to detect Enterobacteriaceae 125
cgccgacctcgccggggttctttcggcg

SEQ ID NO: 126 - DNA, 28 bp, Artificial Sequence
  synthesized nucleic acid probe to detect Enterobacteriaceae 126
ctggcgcctcgccggggttcttcgccag

SEQ ID NO: 127 - DNA, 28 bp, Artificial Sequence
  synthesized nucleic acid probe to detect Enterobacteriaceae 127
ctgccgcctcgccggggttcttcggcag

SEQ ID NO: 128 - DNA, 28 bp, Artificial Sequence
  synthesized nucleic acid probe to detect Enterobacteriaceae 128
ccgggtcctcgccggggttcttacccgg

SEQ ID NO: 129 - DNA, 28 bp, Artificial Sequence
  synthesized nucleic acid probe to detect Enterobacteriaceae 129
gcgcctcctcgccggggttcttaggcgc

SEQ ID NO: 130 - DNA, 28 bp, Artificial Sequence
  synthesized nucleic acid probe to detect Enterobacteriaceae 130
gagcgccctcgccggggttcttgcgctc

SEQ ID NO: 131 - DNA, 28 bp, Artificial Sequence
  synthesized nucleic acid probe to detect Enterobacteriaceae 131
ggctggcctcgccggggttcttccagcc

SEQ ID NO: 132 - DNA, 28 bp, Artificial Sequence
  synthesized nucleic acid probe to detect Enterobacteriaceae 132
gctgcacctcgccggggttctttgcagc

SEQ ID NO: 133 - DNA, 28 bp, Artificial Sequence
  synthesized nucleic acid probe to detect Enterobacteriaceae 133
gggtggcctcgccggggttcttccaccc

SEQ ID NO: 134 - DNA, 28 bp, Artificial Sequence
  synthesized nucleic acid probe to detect Enterobacteriaceae 134
gcggctcctcgccggggttcttagccgc

SEQ ID NO: 135 - DNA, 28 bp, Artificial Sequence
  synthesized nucleic acid probe to detect Enterobacteriaceae 135
gaccgccctcgccggggttcttgcggtc

SEQ ID NO: 136 - DNA, 28 bp, Artificial Sequence
  synthesized nucleic acid probe to detect Enterobacteriaceae 136
gacggccctcgccggggttcttgccgtc

SEQ ID NO: 137 - DNA, 28 bp, Artificial Sequence
  synthesized nucleic acid probe to detect Enterobacteriaceae 137
ggcccacctcgccggggttctttgggcc

SEQ ID NO: 138 - DNA, 19 bp, Artificial Sequence
  synthesized nucleic acid probe to detect Enterobacteriaceae 138
cgctcccctacccaacaac

SEQ ID NO: 139 - DNA, 31 bp, Artificial Sequence
  synthesized nucleic acid probe to detect Enterobacteriaceae 139
cgcggacgctcccctacccaacaactccgcg

SEQ ID NO: 140 - DNA, 31 bp, Artificial Sequence
  synthesized nucleic acid probe to detect Enterobacteriaceae 140
ctcgcgcgctcccctacccaacaaccgcgag

SEQ ID NO: 141 - DNA, 31 bp, Artificial Sequence
  synthesized nucleic acid probe to detect Enterobacteriaceae 141
ccgacccgctcccctacccaacaacggtcgg

SEQ ID NO: 142 - DNA, 31 bp, Artificial Sequence
  synthesized nucleic acid probe to detect Enterobacteriaceae 142
cgacgtcgctcccctacccaacaacacgtcg

SEQ ID NO: 143 - DNA, 31 bp, Artificial Sequence
  synthesized nucleic acid probe to detect Enterobacteriaceae 143
cccacccgctcccctacccaacaacggtggg

SEQ ID NO: 144 - DNA, 31 bp, Artificial Sequence
  synthesized nucleic acid probe to detect Enterobacteriaceae 144
cgccgacgctcccctacccaacaactcggcg

SEQ ID NO: 145 - DNA, 31 bp, Artificial Sequence
  synthesized nucleic acid probe to detect Enterobacteriaceae 145
ctggcgcgctcccctacccaacaaccgccag

SEQ ID NO: 146 - DNA, 31 bp, Artificial Sequence
  synthesized nucleic acid probe to detect Enterobacteriaceae 146
ctgccgcgctcccctacccaacaaccggcag

SEQ ID NO: 147 - DNA, 31 bp, Artificial Sequence
  synthesized nucleic acid probe to detect Enterobacteriaceae 147
ccgggtcgctcccctacccaacaacacccgg

SEQ ID NO: 148 - DNA, 31 bp, Artificial Sequence
  synthesized nucleic acid probe to detect Enterobacteriaceae 148
gcgcctcgctcccctacccaacaacaggcgc

SEQ ID NO: 149 - DNA, 31 bp, Artificial Sequence
  synthesized nucleic acid probe to detect Enterobacteriaceae 149
gagcgccgctcccctacccaacaacgcgctc

SEQ ID NO: 150 - DNA, 31 bp, Artificial Sequence
  synthesized nucleic acid probe to detect Enterobacteriaceae 150
ggctggcgctcccctacccaacaacccagcc

SEQ ID NO: 151 - DNA, 31 bp, Artificial Sequence
  synthesized nucleic acid probe to detect Enterobacteriaceae 151
gctgcacgctcccctacccaacaactgcagc

SEQ ID NO: 152 - DNA, 31 bp, Artificial Sequence
  synthesized nucleic acid probe to detect Enterobacteriaceae 152
gggtggcgctcccctacccaacaacccaccc

SEQ ID NO: 153 - DNA, 31 bp, Artificial Sequence
  synthesized nucleic acid probe to detect Enterobacteriaceae 153
gcggctcgctcccctacccaacaacagccgc

SEQ ID NO: 154 - DNA, 31 bp, Artificial Sequence
  synthesized nucleic acid probe to detect Enterobacteriaceae 154
gaccgccgctcccctacccaacaacgcggtc

SEQ ID NO: 155 - DNA, 31 bp, Artificial Sequence
  synthesized nucleic acid probe to detect Enterobacteriaceae 155
gacggccgctcccctacccaacaacgccgtc

SEQ ID NO: 156 - DNA, 31 bp, Artificial Sequence
  synthesized nucleic acid probe to detect Enterobacteriaceae 156
ggcccacgctcccctacccaacaactgggcc

SEQ ID NO: 157 - DNA, 19 bp, Artificial Sequence
  synthesized nucleic acid probe to detect Enterobacteriaceae 157
cagcgtgccttctcccgaa

SEQ ID NO: 158 - DNA, 31 bp, Artificial Sequence
  synthesized nucleic acid probe to detect Enterobacteriaceae 158
cgcggacagcgtgccttctcccgaatccgcg

SEQ ID NO: 159 - DNA, 31 bp, Artificial Sequence
  synthesized nucleic acid probe to detect Enterobacteriaceae 159
ctcgcgcagcgtgccttctcccgaacgcgag

SEQ ID NO: 160 - DNA, 31 bp, Artificial Sequence
  synthesized nucleic acid probe to detect Enterobacteriaceae 160
ccgacccagcgtgccttctcccgaaggtcgg

SEQ ID NO: 161 - DNA, 31 bp, Artificial Sequence
  synthesized nucleic acid probe to detect Enterobacteriaceae 161
cgacgtcagcgtgccttctcccgaaacgtcg

SEQ ID NO: 162 - DNA, 31 bp, Artificial Sequence
  synthesized nucleic acid probe to detect Enterobacteriaceae 162
cccacccagcgtgccttctcccgaaggtggg

SEQ ID NO: 163 - DNA, 31 bp, Artificial Sequence
  synthesized nucleic acid probe to detect Enterobacteriaceae 163
cgccgacagcgtgccttctcccgaatcggcg

SEQ ID NO: 164 - DNA, 31 bp, Artificial Sequence
  synthesized nucleic acid probe to detect Enterobacteriaceae 164
ctggcgcagcgtgccttctcccgaacgccag

SEQ ID NO: 165 - DNA, 31 bp, Artificial Sequence
  synthesized nucleic acid probe to detect Enterobacteriaceae 165
ctgccgcagcgtgccttctcccgaacggcag

SEQ ID NO: 166 - DNA, 31 bp, Artificial Sequence
  synthesized nucleic acid probe to detect Enterobacteriaceae 166
ccgggtcagcgtgccttctcccgaaacccgg

SEQ ID NO: 167 - DNA, 31 bp, Artificial Sequence
  synthesized nucleic acid probe to detect Enterobacteriaceae 167
gcgcctcagcgtgccttctcccgaaaggcgc

SEQ ID NO: 168 - DNA, 31 bp, Artificial Sequence
  synthesized nucleic acid probe to detect Enterobacteriaceae 168
gagcgccagcgtgccttctcccgaagcgctc

SEQ ID NO: 169 - DNA, 31 bp, Artificial Sequence
  synthesized nucleic acid probe to detect Enterobacteriaceae 169
ggctggcagcgtgccttctcccgaaccagcc

SEQ ID NO: 170 - DNA, 31 bp, Artificial Sequence
  synthesized nucleic acid probe to detect Enterobacteriaceae 170
gctgcacagcgtgccttctcccgaatgcagc

SEQ ID NO: 171 - DNA, 31 bp, Artificial Sequence
  synthesized nucleic acid probe to detect Enterobacteriaceae 171
gggtggcagcgtgccttctcccgaaccaccc

SEQ ID NO: 172 - DNA, 31 bp, Artificial Sequence
  synthesized nucleic acid probe to detect Enterobacteriaceae 172
gcggctcagcgtgccttctcccgaaagccgc

SEQ ID NO: 173 - DNA, 31 bp, Artificial Sequence
  synthesized nucleic acid probe to detect Enterobacteriaceae 173
gaccgccagcgtgccttctcccgaagcggtc

SEQ ID NO: 174 - DNA, 31 bp, Artificial Sequence
  synthesized nucleic acid probe to detect Enterobacteriaceae 174
gacggccagcgtgccttctcccgaagccgtc

SEQ ID NO: 175 - DNA, 31 bp, Artificial Sequence
  synthesized nucleic acid probe to detect Enterobacteriaceae 175
ggcccacagcgtgccttctcccgaatgggcc

SEQ ID NO: 176 - DNA, 20 bp, Artificial Sequence
  synthesized nucleic acid probe to detect Enterobacteriaceae 176
ctgccgcagcttcggtgcat

SEQ ID NO: 177 - DNA, 27 bp, Artificial Sequence
  synthesized nucleic acid probe to detect Enterobacteriaceae 177
gctgccgcagcttcggtgcatggcagc

SEQ ID NO: 178 - DNA, 32 bp, Artificial Sequence
  synthesized nucleic acid probe to detect Enterobacteriaceae 178
cgcggactgccgcagcttcggtgcattccgcg

SEQ ID NO: 179 - DNA, 32 bp, Artificial Sequence
  synthesized nucleic acid probe to detect Enterobacteriaceae 179
ctcgcgctgccgcagcttcggtgcatcgcgag

SEQ ID NO: 180 - DNA, 32 bp, Artificial Sequence
  synthesized nucleic acid probe to detect Enterobacteriaceae 180
ccgaccctgccgcagcttcggtgcatggtcgg

SEQ ID NO: 181 - DNA, 32 bp, Artificial Sequence
  synthesized nucleic acid probe to detect Enterobacteriaceae 181
cgacgtctgccgcagcttcggtgcatacgtcg

SEQ ID NO: 182 - DNA, 32 bp, Artificial Sequence
  synthesized nucleic acid probe to detect Enterobacteriaceae 182
cccaccctgccgcagcttcggtgcatggtggg

SEQ ID NO: 183 - DNA, 32 bp, Artificial Sequence
  synthesized nucleic acid probe to detect Enterobacteriaceae 183
cgccgactgccgcagcttcggtgcattcggcg

SEQ ID NO: 184 - DNA, 32 bp, Artificial Sequence
  synthesized nucleic acid probe to detect Enterobacteriaceae 184
ctggcgctgccgcagcttcggtgcatcgccag

SEQ ID NO: 185 - DNA, 32 bp, Artificial Sequence
  synthesized nucleic acid probe to detect Enterobacteriaceae 185
ctgccgctgccgcagcttcggtgcatcggcag

SEQ ID NO: 186 - DNA, 32 bp, Artificial Sequence
  synthesized nucleic acid probe to detect Enterobacteriaceae 186
ccgggtctgccgcagcttcggtgcatacccgg

SEQ ID NO: 187 - DNA, 27 bp, Artificial Sequence
  synthesized nucleic acid probe to detect Enterobacteriaceae 187
cctgccgcagcttcggtgcatggcagg

SEQ ID NO: 188 - DNA, 32 bp, Artificial Sequence
  synthesized nucleic acid probe to detect Enterobacteriaceae 188
gcgcctctgccgcagcttcggtgcataggcgc

SEQ ID NO: 189 - DNA, 32 bp, Artificial Sequence
  synthesized nucleic acid probe to detect Enterobacteriaceae 189
gagcgcctgccgcagcttcggtgcatgcgctc

SEQ ID NO: 190 - DNA, 32 bp, Artificial Sequence
  synthesized nucleic acid probe to detect Enterobacteriaceae 190
ggctggctgccgcagcttcggtgcatccagcc

SEQ ID NO: 191 - DNA, 32 bp, Artificial Sequence
  synthesized nucleic acid probe to detect Enterobacteriaceae 191
gctgcactgccgcagcttcggtgcattgcagc

SEQ ID NO: 192 - DNA, 32 bp, Artificial Sequence
  synthesized nucleic acid probe to detect Enterobacteriaceae 192
gggtggctgccgcagcttcggtgcatccaccc

SEQ ID NO: 193 - DNA, 32 bp, Artificial Sequence
  synthesized nucleic acid probe to detect Enterobacteriaceae 193
gcggctctgccgcagcttcggtgcatagccgc

SEQ ID NO: 194 - DNA, 32 bp, Artificial Sequence
  synthesized nucleic acid probe to detect Enterobacteriaceae 194
gaccgcctgccgcagcttcggtgcatgcggtc

SEQ ID NO: 195 - DNA, 32 bp, Artificial Sequence
  synthesized nucleic acid probe to detect Enterobacteriaceae 195
gacggcctgccgcagcttcggtgcatgccgtc

SEQ ID NO: 196 - DNA, 32 bp, Artificial Sequence
  synthesized nucleic acid probe to detect Enterobacteriaceae 196
ggcccactgccgcagcttcggtgcattgggcc

SEQ ID NO: 197 - DNA, 24 bp, Artificial Sequence
  synthesized nucleic acid probe to detect Enterobacteriaceae 197
gtgtgtctcccgtgataacattct

SEQ ID NO: 198 - DNA, 36 bp, Artificial Sequence
  synthesized nucleic acid probe to detect Enterobacteriaceae 198
cgcggagtgtgtctcccgtgataacattcttccgcg

SEQ ID NO: 199 - DNA, 36 bp, Artificial Sequence
  synthesized nucleic acid probe to detect Enterobacteriaceae 199
ctcgcggtgtgtctcccgtgataacattctcgcgag

SEQ ID NO: 200 - DNA, 36 bp, Artificial Sequence
  synthesized nucleic acid probe to detect Enterobacteriaceae 200
ccgaccgtgtgtctcccgtgataacattctggtcgg

SEQ ID NO: 201 - DNA, 36 bp, Artificial Sequence
  synthesized nucleic acid probe to detect Enterobacteriaceae 201
cgacgtgtgtgtctcccgtgataacattctacgtcg

SEQ ID NO: 202 - DNA, 36 bp, Artificial Sequence
  synthesized nucleic acid probe to detect Enterobacteriaceae 202
cccaccgtgtgtctcccgtgataacattctggtggg

SEQ ID NO: 203 - DNA, 36 bp, Artificial Sequence
  synthesized nucleic acid probe to detect Enterobacteriaceae 203
cgccgagtgtgtctcccgtgataacattcttcggcg

SEQ ID NO: 204 - DNA, 36 bp, Artificial Sequence
  synthesized nucleic acid probe to detect Enterobacteriaceae 204
ctggcggtgtgtctcccgtgataacattctcgccag

SEQ ID NO: 205 - DNA, 36 bp, Artificial Sequence
  synthesized nucleic acid probe to detect Enterobacteriaceae 205
ctgccggtgtgtctcccgtgataacattctcggcag

SEQ ID NO: 206 - DNA, 36 bp, Artificial Sequence
  synthesized nucleic acid probe to detect Enterobacteriaceae 206
ccgggtgtgtgtctcccgtgataacattctacccgg

SEQ ID NO: 207 - DNA, 35 bp, Artificial Sequence
  synthesized nucleic acid probe to detect Enterobacteriaceae 207
gaccggtgtgtctcccgtgataacattctccggtc

SEQ ID NO: 208 - DNA, 36 bp, Artificial Sequence
  synthesized nucleic acid probe to detect Enterobacteriaceae 208
gcgcctgtgtgtctcccgtgataacattctaggcgc

SEQ ID NO: 209 - DNA, 36 bp, Artificial Sequence
  synthesized nucleic acid probe to detect Enterobacteriaceae 209
gagcgcgtgtgtctcccgtgataacattctgcgctc

SEQ ID NO: 210 - DNA, 36 bp, Artificial Sequence
  synthesized nucleic acid probe to detect Enterobacteriaceae 210
ggctgggtgtgtctcccgtgataacattctccagcc

SEQ ID NO: 211 - DNA, 36 bp, Artificial Sequence
  synthesized nucleic acid probe to detect Enterobacteriaceae 211
gctgcagtgtgtctcccgtgataacattcttgcagc

SEQ ID NO: 212 - DNA, 36 bp, Artificial Sequence
  synthesized nucleic acid probe to detect Enterobacteriaceae 212
gggtgggtgtgtctcccgtgataacattctccaccc

SEQ ID NO: 213 - DNA, 36 bp, Artificial Sequence
  synthesized nucleic acid probe to detect Enterobacteriaceae 213
gcggctgtgtgtctcccgtgataacattctagccgc

SEQ ID NO: 214 - DNA, 36 bp, Artificial Sequence
  synthesized nucleic acid probe to detect Enterobacteriaceae 214
gaccgcgtgtgtctcccgtgataacattctgcggtc

SEQ ID NO: 215 - DNA, 36 bp, Artificial Sequence
  synthesized nucleic acid probe to detect Enterobacteriaceae 215
gacggcgtgtgtctcccgtgataacattctgccgtc

SEQ ID NO: 216 - DNA, 36 bp, Artificial Sequence
  synthesized nucleic acid probe to detect Enterobacteriaceae 216
ggcccagtgtgtctcccgtgataacattcttgggcc

SEQ ID NO: 217 - DNA, 35 bp, Artificial Sequence
  synthesized nucleic acid probe to detect Enterobacteriaceae 217
ctggcgtgtgtctcccgtgataacattctcgccag

SEQ ID NO: 218 - DNA, 21 bp, Artificial Sequence
  synthesized nucleic acid probe to detect Enterobacteriaceae 218
ttcgtgtttgcacagtgctgt

SEQ ID NO: 219 - DNA, 33 bp, Artificial Sequence
  synthesized nucleic acid probe to detect Enterobacteriaceae 219
cgcggattcgtgtttgcacagtgctgttccgcg

SEQ ID NO: 220 - DNA, 33 bp, Artificial Sequence
  synthesized nucleic acid probe to detect Enterobacteriaceae 220
ctcgcgttcgtgtttgcacagtgctgtcgcgag

SEQ ID NO: 221 - DNA, 33 bp, Artificial Sequence
  synthesized nucleic acid probe to detect Enterobacteriaceae 221
ccgaccttcgtgtttgcacagtgctgtggtcgg

SEQ ID NO: 222 - DNA, 33 bp, Artificial Sequence
  synthesized nucleic acid probe to detect Enterobacteriaceae 222
cgacgtttcgtgtttgcacagtgctgtacgtcg

SEQ ID NO: 223 - DNA, 33 bp, Artificial Sequence
  synthesized nucleic acid probe to detect Enterobacteriaceae 223
cccaccttcgtgtttgcacagtgctgtggtggg

SEQ ID NO: 224 - DNA, 33 bp, Artificial Sequence
  synthesized nucleic acid probe to detect Enterobacteriaceae 224
cgccgattcgtgtttgcacagtgctgttcggcg

SEQ ID NO: 225 - DNA, 33 bp, Artificial Sequence
  synthesized nucleic acid probe to detect Enterobacteriaceae 225
ctggcgttcgtgtttgcacagtgctgtcgccag

SEQ ID NO: 226 - DNA, 33 bp, Artificial Sequence
  synthesized nucleic acid probe to detect Enterobacteriaceae 226
ctgccgttcgtgtttgcacagtgctgtcggcag

SEQ ID NO: 227 - DNA, 33 bp, Artificial Sequence
  synthesized nucleic acid probe to detect Enterobacteriaceae 227
ccgggtttcgtgtttgcacagtgctgtacccgg

SEQ ID NO: 228 - DNA, 33 bp, Artificial Sequence
  synthesized nucleic acid probe to detect Enterobacteriaceae 228
gcgcctttcgtgtttgcacagtgctgtaggcgc

SEQ ID NO: 229 - DNA, 33 bp, Artificial Sequence
  synthesized nucleic acid probe to detect Enterobacteriaceae 229
gagcgcttcgtgtttgcacagtgctgtgcgctc

SEQ ID NO: 230 - DNA, 33 bp, Artificial Sequence
  synthesized nucleic acid probe to detect Enterobacteriaceae 230
ggctggttcgtgtttgcacagtgctgtccagcc

SEQ ID NO: 231 - DNA, 33 bp, Artificial Sequence
  synthesized nucleic acid probe to detect Enterobacteriaceae 231
gctgcattcgtgtttgcacagtgctgttgcagc

SEQ ID NO: 232 - DNA, 33 bp, Artificial Sequence
  synthesized nucleic acid probe to detect Enterobacteriaceae 232
gggtggttcgtgtttgcacagtgctgtccaccc

SEQ ID NO: 233 - DNA, 33 bp, Artificial Sequence
  synthesized nucleic acid probe to detect Enterobacteriaceae 233
gcggctttcgtgtttgcacagtgctgtagccgc

SEQ ID NO: 234 - DNA, 33 bp, Artificial Sequence
  synthesized nucleic acid probe to detect Enterobacteriaceae 234
gaccgcttcgtgtttgcacagtgctgtgcggtc

SEQ ID NO: 235 - DNA, 33 bp, Artificial Sequence
  synthesized nucleic acid probe to detect Enterobacteriaceae 235
gacggcttcgtgtttgcacagtgctgtgccgtc

SEQ ID NO: 236 - DNA, 33 bp, Artificial Sequence
  synthesized nucleic acid probe to detect Enterobacteriaceae 236
ggcccattcgtgtttgcacagtgctgttgggcc

SEQ ID NO: 237 - DNA, 19 bp, Artificial Sequence
  synthesized nucleic acid probe to detect Enterobacteriaceae 237
gctcttcctccgttcgttc

SEQ ID NO: 238 - DNA, 31 bp, Artificial Sequence
  synthesized nucleic acid probe to detect Enterobacteriaceae_238
cgcggagctcttcctccgttcgttctccgcg

SEQ ID NO: 239 - DNA, 31 bp, Artificial Sequence
  synthesized nucleic acid probe to detect Enterobacteriaceae_239
ctcgcggctcttcctccgttcgttccgcgag

SEQ ID NO: 240 - DNA, 31 bp, Artificial Sequence
  synthesized nucleic acid probe to detect Enterobacteriaceae_240
ccgaccgctcttcctccgttcgttcggtcgg

SEQ ID NO: 241 - DNA, 31 bp, Artificial Sequence
  synthesized nucleic acid probe to detect Enterobacteriaceae_241
cgacgtgctcttcctccgttcgttcacgtcg

SEQ ID NO: 242 - DNA, 31 bp, Artificial Sequence
  synthesized nucleic acid probe to detect Enterobacteriaceae_242
cccaccgctcttcctccgttcgttcggtggg

SEQ ID NO: 243 - DNA, 31 bp, Artificial Sequence
  synthesized nucleic acid probe to detect Enterobacteriaceae_243
cgccgagctcttcctccgttcgttctcggcg

SEQ ID NO: 244 - DNA, 31 bp, Artificial Sequence
  synthesized nucleic acid probe to detect Enterobacteriaceae_244
ctggcggctcttcctccgttcgttccgccag

SEQ ID NO: 245 - DNA, 31 bp, Artificial Sequence
  synthesized nucleic acid probe to detect Enterobacteriaceae_245
ccgcgcgctcttcctccgttcgttcgcgcgg

SEQ ID NO: 246 - DNA, 31 bp, Artificial Sequence
  synthesized nucleic acid probe to detect Enterobacteriaceae_246
ctgccggctcttcctccgttcgttccggcag

SEQ ID NO: 247 - DNA, 31 bp, Artificial Sequence
  synthesized nucleic acid probe to detect Enterobacteriaceae_247
ccgggtgctcttcctccgttcgttcacccgg

SEQ ID NO: 248 - DNA, 31 bp, Artificial Sequence
  synthesized nucleic acid probe to detect Enterobacteriaceae_248
gcgcctgctcttcctccgttcgttcaggcgc

SEQ ID NO: 249 - DNA, 31 bp, Artificial Sequence
  synthesized nucleic acid probe to detect Enterobacteriaceae_249
gagcgcgctcttcctccgttcgttcgcgctc

SEQ ID NO: 250 - DNA, 31 bp, Artificial Sequence
  synthesized nucleic acid probe to detect Enterobacteriaceae_250
ggctgggctcttcctccgttcgttcccagcc

SEQ ID NO: 251 - DNA, 31 bp, Artificial Sequence
  synthesized nucleic acid probe to detect Enterobacteriaceae_251
gctgcagctcttcctccgttcgttctgcagc

SEQ ID NO: 252 - DNA, 31 bp, Artificial Sequence
  synthesized nucleic acid probe to detect Enterobacteriaceae_252
gggtgggctcttcctccgttcgttcccaccc

SEQ ID NO: 253 - DNA, 31 bp, Artificial Sequence
  synthesized nucleic acid probe to detect Enterobacteriaceae_253
gcggctgctcttcctccgttcgttcagccgc

SEQ ID NO: 254 - DNA, 31 bp, Artificial Sequence
  synthesized nucleic acid probe to detect Enterobacteriaceae_254
gaccgcgctcttcctccgttcgttcgcggtc

SEQ ID NO: 255 - DNA, 31 bp, Artificial Sequence
  synthesized nucleic acid probe to detect Enterobacteriaceae_255
gacggcgctcttcctccgttcgttcgccgtc

SEQ ID NO: 256 - DNA, 31 bp, Artificial Sequence
  synthesized nucleic acid probe to detect Enterobacteriaceae_256
ggcccagctcttcctccgttcgttctgggcc

SEQ ID NO: 257 - DNA, 18 bp, Artificial Sequence
  synthesized nucleic acid probe to detect Enterobacteriaceae_257
agcaagctcttcctccgt

SEQ ID NO: 258 - DNA, 30 bp, Artificial Sequence
  synthesized nucleic acid probe to detect Enterobacteriaceae_258
cgcggaagcaagctcttcctccgttccgcg

SEQ ID NO: 259 - DNA, 30 bp, Artificial Sequence
  synthesized nucleic acid probe to detect Enterobacteriaceae_259
ctcgcgagcaagctcttcctccgtcgcgag

SEQ ID NO: 260 - DNA, 30 bp, Artificial Sequence
  synthesized nucleic acid probe to detect Enterobacteriaceae_260
ccgaccagcaagctcttcctccgtggtcgg

SEQ ID NO: 261 - DNA, 30 bp, Artificial Sequence
  synthesized nucleic acid probe to detect Enterobacteriaceae_261
cgacgtagcaagctcttcctccgtacgtcg

SEQ ID NO: 262 - DNA, 30 bp, Artificial Sequence
  synthesized nucleic acid probe to detect Enterobacteriaceae_262
cccaccagcaagctcttcctccgtggtggg

SEQ ID NO: 263 - DNA, 30 bp, Artificial Sequence
  synthesized nucleic acid probe to detect Enterobacteriaceae_263
cgccgaagcaagctcttcctccgttcggcg

SEQ ID NO: 264 - DNA, 30 bp, Artificial Sequence
  synthesized nucleic acid probe to detect Enterobacteriaceae_264
ctggcgagcaagctcttcctccgtcgccag

SEQ ID NO: 265 - DNA, 30 bp, Artificial Sequence
  synthesized nucleic acid probe to detect Enterobacteriaceae_265
ctgccgagcaagctcttcctccgtcggcag

SEQ ID NO: 266 - DNA, 30 bp, Artificial Sequence
  synthesized nucleic acid probe to detect Enterobacteriaceae_266
ccgggtagcaagctcttcctccgtacccgg

SEQ ID NO: 267 - DNA, 30 bp, Artificial Sequence
  synthesized nucleic acid probe to detect Enterobacteriaceae_267
gcgcctagcaagctcttcctccgtaggcgc

SEQ ID NO: 268 - DNA, 30 bp, Artificial Sequence
  synthesized nucleic acid probe to detect Enterobacteriaceae_268
gagcgcagcaagctcttcctccgtgcgctc

SEQ ID NO: 269 - DNA, 30 bp, Artificial Sequence
  synthesized nucleic acid probe to detect Enterobacteriaceae_269
ggctggagcaagctcttcctccgtccagcc

SEQ ID NO: 270 - DNA, 30 bp, Artificial Sequence
  synthesized nucleic acid probe to detect Enterobacteriaceae_270
gctgcaagcaagctcttcctccgttgcagc

SEQ ID NO: 271 - DNA, 30 bp, Artificial Sequence
  synthesized nucleic acid probe to detect Enterobacteriaceae_271
gggtggagcaagctcttcctccgtccaccc

SEQ ID NO: 272 - DNA, 30 bp, Artificial Sequence
  synthesized nucleic acid probe to detect Enterobacteriaceae_272
gcggctagcaagctcttcctccgtagccgc

SEQ ID NO: 273 - DNA, 30 bp, Artificial Sequence
  synthesized nucleic acid probe to detect Enterobacteriaceae_273
gaccgcagcaagctcttcctccgtgcggtc

SEQ ID NO: 274 - DNA, 30 bp, Artificial Sequence
  synthesized nucleic acid probe to detect Enterobacteriaceae_274
gacggcagcaagctcttcctccgtgccgtc

SEQ ID NO: 275 - DNA, 30 bp, Artificial Sequence
  synthesized nucleic acid probe to detect Enterobacteriaceae_275
ggcccaagcaagctcttcctccgttgggcc

SEQ ID NO: 276 - DNA, 20 bp, Artificial Sequence
  synthesized nucleic acid probe to detect Enterobacteriaceae_276
gccactaactttggaagagc

SEQ ID NO: 277 - DNA, 32 bp, Artificial Sequence
  synthesized nucleic acid probe to detect Enterobacteriaceae_277
cgcggagccactaactttggaagagctccgcg

SEQ ID NO: 278 - DNA, 32 bp, Artificial Sequence
  synthesized nucleic acid probe to detect Enterobacteriaceae_278
ctcgcggccactaactttggaagagccgcgag

SEQ ID NO: 279 - DNA, 32 bp, Artificial Sequence
  synthesized nucleic acid probe to detect Enterobacteriaceae_279
ccgaccgccactaactttggaagagcggtcgg

SEQ ID NO: 280 - DNA, 32 bp, Artificial Sequence
  synthesized nucleic acid probe to detect Enterobacteriaceae_280
cgacgtgccactaactttggaagagcacgtcg

SEQ ID NO: 281 - DNA, 32 bp, Artificial Sequence
  synthesized nucleic acid probe to detect Enterobacteriaceae_281
cccaccgccactaactttggaagagcggtggg

SEQ ID NO: 282 - DNA, 32 bp, Artificial Sequence
  synthesized nucleic acid probe to detect Enterobacteriaceae_282
cgccgagccactaactttggaagagctcggcg

SEQ ID NO: 283 - DNA, 32 bp, Artificial Sequence
  synthesized nucleic acid probe to detect Enterobacteriaceae_283
ctggcggccactaactttggaagagccgccag

SEQ ID NO: 284 - DNA, 32 bp, Artificial Sequence
  synthesized nucleic acid probe to detect Enterobacteriaceae_284
ctgccggccactaactttggaagagccggcag

SEQ ID NO: 285 - DNA, 32 bp, Artificial Sequence
  synthesized nucleic acid probe to detect Enterobacteriaceae_285
ccgggtgccactaactttggaagagcacccgg

SEQ ID NO: 286 - DNA, 32 bp, Artificial Sequence
  synthesized nucleic acid probe to detect Enterobacteriaceae_286
gcgcctgccactaactttggaagagcaggcgc

SEQ ID NO: 287 - DNA, 32 bp, Artificial Sequence
  synthesized nucleic acid probe to detect Enterobacteriaceae_287
gagcgcgccactaactttggaagagcgcgctc

SEQ ID NO: 288 - DNA, 32 bp, Artificial Sequence
  synthesized nucleic acid probe to detect Enterobacteriaceae_288
ggctgggccactaactttggaagagcccagcc

SEQ ID NO: 289 - DNA, 32 bp, Artificial Sequence
  synthesized nucleic acid probe to detect Enterobacteriaceae_289
gctgcagccactaactttggaagagctgcagc

SEQ ID NO: 290 - DNA, 32 bp, Artificial Sequence
  synthesized nucleic acid probe to detect Enterobacteriaceae_290
gggtgggccactaactttggaagagcccaccc

SEQ ID NO: 291 - DNA, 32 bp, Artificial Sequence
  synthesized nucleic acid probe to detect Enterobacteriaceae_291
gcggctgccactaactttggaagagcagccgc

SEQ ID NO: 292 - DNA, 32 bp, Artificial Sequence
  synthesized nucleic acid probe to detect Enterobacteriaceae_292
gaccgcgccactaactttggaagagcgcggtc

SEQ ID NO: 293 - DNA, 32 bp, Artificial Sequence
  synthesized nucleic acid probe to detect Enterobacteriaceae_293
gacggcgccactaactttggaagagcgccgtc

SEQ ID NO: 294 - DNA, 32 bp, Artificial Sequence
  synthesized nucleic acid probe to detect Enterobacteriaceae_294
ggcccagccactaactttggaagagctgggcc

SEQ ID NO: 295 - DNA, 19 bp, Artificial Sequence
  synthesized nucleic acid probe to detect Enterobacteriaceae_295
atccccaacttacaggcag

SEQ ID NO: 296 - DNA, 31 bp, Artificial Sequence
  synthesized nucleic acid probe to detect Enterobacteriaceae_296
cgcggaatccccaacttacaggcagtccgcg

SEQ ID NO: 297 - DNA, 31 bp, Artificial Sequence
  synthesized nucleic acid probe to detect Enterobacteriaceae_297
ctcgcgatccccaacttacaggcagcgcgag

SEQ ID NO: 298 - DNA, 31 bp, Artificial Sequence
  synthesized nucleic acid probe to detect Enterobacteriaceae_298
ccgcgcatccccaacttacaggcaggcgcgg

SEQ ID NO: 299 - DNA, 31 bp, Artificial Sequence
  synthesized nucleic acid probe to detect Enterobacteriaceae_299
ccgaccatccccaacttacaggcagggtcgg

SEQ ID NO: 300 - DNA, 31 bp, Artificial Sequence
  synthesized nucleic acid probe to detect Enterobacteriaceae_300
cgacgtatccccaacttacaggcagacgtcg

SEQ ID NO: 301 - DNA, 31 bp, Artificial Sequence
  synthesized nucleic acid probe to detect Enterobacteriaceae_301
cccaccatccccaacttacaggcagggtggg

SEQ ID NO: 302 - DNA, 31 bp, Artificial Sequence
  synthesized nucleic acid probe to detect Enterobacteriaceae_302
cgccgaatccccaacttacaggcagtcggcg

SEQ ID NO: 303 - DNA, 31 bp, Artificial Sequence
  synthesized nucleic acid probe to detect Enterobacteriaceae_303
ctggcgatccccaacttacaggcagcgccag

SEQ ID NO: 304 - DNA, 31 bp, Artificial Sequence
  synthesized nucleic acid probe to detect Enterobacteriaceae_304
ctgccgatccccaacttacaggcagcggcag

SEQ ID NO: 305 - DNA, 31 bp, Artificial Sequence
  synthesized nucleic acid probe to detect Enterobacteriaceae_305
ccgggtatccccaacttacaggcagacccgg

SEQ ID NO: 306 - DNA, 31 bp, Artificial Sequence
  synthesized nucleic acid probe to detect Enterobacteriaceae_306
gcgcctatccccaacttacaggcagaggcgc

SEQ ID NO: 307 - DNA, 31 bp, Artificial Sequence
  synthesized nucleic acid probe to detect Enterobacteriaceae_307
gagcgcatccccaacttacaggcaggcgctc

SEQ ID NO: 308 - DNA, 31 bp, Artificial Sequence
  synthesized nucleic acid probe to detect Enterobacteriaceae_308
ggctggatccccaacttacaggcagccagcc

SEQ ID NO: 309 - DNA, 31 bp, Artificial Sequence
  synthesized nucleic acid probe to detect Enterobacteriaceae_309
gctgcaatccccaacttacaggcagtgcagc

SEQ ID NO: 310 - DNA, 31 bp, Artificial Sequence
  synthesized nucleic acid probe to detect Enterobacteriaceae_310
gggtggatccccaacttacaggcagccaccc

SEQ ID NO: 311 - DNA, 31 bp, Artificial Sequence
  synthesized nucleic acid probe to detect Enterobacteriaceae_311
gcggctatccccaacttacaggcagagccgc

SEQ ID NO: 312 - DNA, 31 bp, Artificial Sequence
  synthesized nucleic acid probe to detect Enterobacteriaceae_312
gaccgcatccccaacttacaggcaggcggtc

SEQ ID NO: 313 - DNA, 31 bp, Artificial Sequence
  synthesized nucleic acid probe to detect Enterobacteriaceae_313
gacggcatccccaacttacaggcaggccgtc

SEQ ID NO: 314 - DNA, 31 bp, Artificial Sequence
  synthesized nucleic acid probe to detect Enterobacteriaceae_314
ggcccaatccccaacttacaggcagtgggcc

SEQ ID NO: 315 - DNA, 21 bp, Artificial Sequence
  synthesized nucleic acid probe to detect Enterobacteriaceae_315
ctctatcattcggtattagcc

SEQ ID NO: 316 - DNA, 33 bp, Artificial Sequence
  synthesized nucleic acid probe to detect Enterobacteriaceae_316
cgcggactctatcattcggtattagcctccgcg

SEQ ID NO: 317 - DNA, 33 bp, Artificial Sequence
  synthesized nucleic acid probe to detect Enterobacteriaceae_317
ctcgcgctctatcattcggtattagcccgcgag

SEQ ID NO: 318 - DNA, 33 bp, Artificial Sequence
  synthesized nucleic acid probe to detect Enterobacteriaceae_318
ccgaccctctatcattcggtattagccggtcgg

SEQ ID NO: 319 - DNA, 33 bp, Artificial Sequence
  synthesized nucleic acid probe to detect Enterobacteriaceae_319
cgacgtctctatcattcggtattagccacgtcg

SEQ ID NO: 320 - DNA, 33 bp, Artificial Sequence
  synthesized nucleic acid probe to detect Enterobacteriaceae_320
cccaccctctatcattcggtattagccggtggg

SEQ ID NO: 321 - DNA, 33 bp, Artificial Sequence
  synthesized nucleic acid probe to detect Enterobacteriaceae_321
cgccgactctatcattcggtattagcctcggcg

SEQ ID NO: 322 - DNA, 33 bp, Artificial Sequence
  synthesized nucleic acid probe to detect Enterobacteriaceae_322
ctggcgctctatcattcggtattagcccgccag

SEQ ID NO: 323 - DNA, 33 bp, Artificial Sequence
  synthesized nucleic acid probe to detect Enterobacteriaceae_323
ctgccgctctatcattcggtattagcccggcag

SEQ ID NO: 324 - DNA, 33 bp, Artificial Sequence
  synthesized nucleic acid probe to detect Enterobacteriaceae_324
ccgggtctctatcattcggtattagccacccgg

SEQ ID NO: 325 - DNA, 33 bp, Artificial Sequence
  synthesized nucleic acid probe to detect Enterobacteriaceae_325
gcgcctctctatcattcggtattagccaggcgc

SEQ ID NO: 326 - DNA, 33 bp, Artificial Sequence
  synthesized nucleic acid probe to detect Enterobacteriaceae_326
gagcgcctctatcattcggtattagccgcgctc

SEQ ID NO: 327 - DNA, 33 bp, Artificial Sequence
  synthesized nucleic acid probe to detect Enterobacteriaceae_327
ggctggctctatcattcggtattagccccagcc

SEQ ID NO: 328 - DNA, 33 bp, Artificial Sequence
  synthesized nucleic acid probe to detect Enterobacteriaceae_328
gctgcactctatcattcggtattagcctgcagc

SEQ ID NO: 329 - DNA, 33 bp, Artificial Sequence
  synthesized nucleic acid probe to detect Enterobacteriaceae_329
gggtggctctatcattcggtattagccccaccc

SEQ ID NO: 330 - DNA, 33 bp, Artificial Sequence
  synthesized nucleic acid probe to detect Enterobacteriaceae_330
gcggctctctatcattcggtattagccagccgc

SEQ ID NO: 331 - DNA, 33 bp, Artificial Sequence
  synthesized nucleic acid probe to detect Enterobacteriaceae_331
gaccgcctctatcattcggtattagccgcggtc

SEQ ID NO: 332 - DNA, 33 bp, Artificial Sequence
  synthesized nucleic acid probe to detect Enterobacteriaceae_332
gacggcctctatcattcggtattagccgccgtc

SEQ ID NO: 333 - DNA, 33 bp, Artificial Sequence
  synthesized nucleic acid probe to detect Enterobacteriaceae_333
ggcccactctatcattcggtattagcctgggcc

SEQ ID NO: 334 - DNA, 19 bp, Artificial Sequence
  synthesized nucleic acid probe to detect Enterobacteriaceae_334
gcatgcgccacactttatg

SEQ ID NO: 335 - DNA, 31 bp, Artificial Sequence
  synthesized nucleic acid probe to detect Enterobacteriaceae_335
cgcggagcatgcgccacactttatgtccgcg

SEQ ID NO: 336 - DNA, 31 bp, Artificial Sequence
  synthesized nucleic acid probe to detect Enterobacteriaceae_336
ctcgcggcatgcgccacactttatgcgcgag

SEQ ID NO: 337 - DNA, 31 bp, Artificial Sequence
  synthesized nucleic acid probe to detect Enterobacteriaceae_337
ccgaccgcatgcgccacactttatgggtcgg

SEQ ID NO: 338 - DNA, 31 bp, Artificial Sequence
  synthesized nucleic acid probe to detect Enterobacteriaceae_338
cgacgtgcatgcgccacactttatgacgtcg

SEQ ID NO: 339 - DNA, 31 bp, Artificial Sequence
  synthesized nucleic acid probe to detect Enterobacteriaceae_339
cccaccgcatgcgccacactttatgggtggg

SEQ ID NO: 340 - DNA, 31 bp, Artificial Sequence
  synthesized nucleic acid probe to detect Enterobacteriaceae_340
cgccgagcatgcgccacactttatgtcggcg

SEQ ID NO: 341 - DNA, 31 bp, Artificial Sequence
  synthesized nucleic acid probe to detect Enterobacteriaceae_341
ctggcggcatgcgccacactttatgcgccag

SEQ ID NO: 342 - DNA, 31 bp, Artificial Sequence
  synthesized nucleic acid probe to detect Enterobacteriaceae_342
ctgccggcatgcgccacactttatgcggcag

SEQ ID NO: 343 - DNA, 31 bp, Artificial Sequence
  synthesized nucleic acid probe to detect Enterobacteriaceae_343
ccgggtgcatgcgccacactttatgacccgg

SEQ ID NO: 344 - DNA, 31 bp, Artificial Sequence
  synthesized nucleic acid probe to detect Enterobacteriaceae_344
gcgcctgcatgcgccacactttatgaggcgc

SEQ ID NO: 345 - DNA, 31 bp, Artificial Sequence
  synthesized nucleic acid probe to detect Enterobacteriaceae_345
gagcgcgcatgcgccacactttatggcgctc

SEQ ID NO: 346 - DNA, 31 bp, Artificial Sequence
  synthesized nucleic acid probe to detect Enterobacteriaceae_346
ggctgggcatgcgccacactttatgccagcc

SEQ ID NO: 347 - DNA, 31 bp, Artificial Sequence
  synthesized nucleic acid probe to detect Enterobacteriaceae_347
gctgcagcatgcgccacactttatgtgcagc

SEQ ID NO: 348 - DNA, 31 bp, Artificial Sequence
  synthesized nucleic acid probe to detect Enterobacteriaceae_348
gggtgggcatgcgccacactttatgccaccc

SEQ ID NO: 349 - DNA, 31 bp, Artificial Sequence
  synthesized nucleic acid probe to detect Enterobacteriaceae_349
gcggctgcatgcgccacactttatgagccgc

SEQ ID NO: 350 - DNA, 31 bp, Artificial Sequence
  synthesized nucleic acid probe to detect Enterobacteriaceae_350
gaccgcgcatgcgccacactttatggcggtc

SEQ ID NO: 351 - DNA, 31 bp, Artificial Sequence
  synthesized nucleic acid probe to detect Enterobacteriaceae_351
gacggcgcatgcgccacactttatggccgtc

SEQ ID NO: 352 - DNA, 31 bp, Artificial Sequence
  synthesized nucleic acid probe to detect Enterobacteriaceae_352
ggcccagcatgcgccacactttatgtgggcc

SEQ ID NO: 353 - DNA, 31 bp, Artificial Sequence
  synthesized nucleic acid probe to detect Enterobacteriaceae_353
ccgggcgcatgcgccacactttatcgcccgg

SEQ ID NO: 354 - DNA, 17 bp, Artificial Sequence
  synthesized nucleic acid probe to detect Enterobacteriaceae_354
aagcgtggcatgcgcca

SEQ ID NO: 355 - DNA, 29 bp, Artificial Sequence
  synthesized nucleic acid probe to detect Enterobacteriaceae_355
cgcggaaagcgtggcatgcgccatccgcg

SEQ ID NO: 356 - DNA, 29 bp, Artificial Sequence
  synthesized nucleic acid probe to detect Enterobacteriaceae_356
ctcgcgaagcgtggcatgcgccacgcgag

SEQ ID NO: 357 - DNA, 29 bp, Artificial Sequence
  synthesized nucleic acid probe to detect Enterobacteriaceae_357
ccgaccaagcgtggcatgcgccaggtcgg

SEQ ID NO: 358 - DNA, 29 bp, Artificial Sequence
  synthesized nucleic acid probe to detect Enterobacteriaceae_358
cgacgtaagcgtggcatgcgccaacgtcg

SEQ ID NO: 359 - DNA, 29 bp, Artificial Sequence
  synthesized nucleic acid probe to detect Enterobacteriaceae_359
cccaccaagcgtggcatgcgccaggtggg

SEQ ID NO: 360 - DNA, 29 bp, Artificial Sequence
  synthesized nucleic acid probe to detect Enterobacteriaceae_360
cgccgaaagcgtggcatgcgccatcggcg

SEQ ID NO: 361 - DNA, 29 bp, Artificial Sequence
  synthesized nucleic acid probe to detect Enterobacteriaceae_361
ctggcgaagcgtggcatgcgccacgccag

SEQ ID NO: 362 - DNA, 29 bp, Artificial Sequence
  synthesized nucleic acid probe to detect Enterobacteriaceae_362
ctgccgaagcgtggcatgcgccacggcag

SEQ ID NO: 363 - DNA, 29 bp, Artificial Sequence
  synthesized nucleic acid probe to detect Enterobacteriaceae_363
ccgggtaagcgtggcatgcgccaacccgg

SEQ ID NO: 364 - DNA, 29 bp, Artificial Sequence
  synthesized nucleic acid probe to detect Enterobacteriaceae_364
gcgcctaagcgtggcatgcgccaaggcgc

SEQ ID NO: 365 - DNA, 29 bp, Artificial Sequence
  synthesized nucleic acid probe to detect Enterobacteriaceae_365
gagcgcaagcgtggcatgcgccagcgctc

SEQ ID NO: 366 - DNA, 29 bp, Artificial Sequence
  synthesized nucleic acid probe to detect Enterobacteriaceae_366
ggctggaagcgtggcatgcgccaccagcc

SEQ ID NO: 367 - DNA, 29 bp, Artificial Sequence
  synthesized nucleic acid probe to detect Enterobacteriaceae_367
gctgcaaagcgtggcatgcgccatgcagc

SEQ ID NO: 368 - DNA, 29 bp, Artificial Sequence
  synthesized nucleic acid probe to detect Enterobacteriaceae_368
gggtggaagcgtggcatgcgccaccaccc

SEQ ID NO: 369 - DNA, 29 bp, Artificial Sequence
  synthesized nucleic acid probe to detect Enterobacteriaceae_369
gcggctaagcgtggcatgcgccaagccgc

SEQ ID NO: 370 - DNA, 29 bp, Artificial Sequence
  synthesized nucleic acid probe to detect Enterobacteriaceae_370
gaccgcaagcgtggcatgcgccagcggtc

SEQ ID NO: 371 - DNA, 29 bp, Artificial Sequence
  synthesized nucleic acid probe to detect Enterobacteriaceae_371
gacggcaagcgtggcatgcgccagccgtc

SEQ ID NO: 372 - DNA, 29 bp, Artificial Sequence
  synthesized nucleic acid probe to detect Enterobacteriaceae_372
ggcccaaagcgtggcatgcgccatgggcc

SEQ ID NO: 373 - DNA, 20 bp, Artificial Sequence
  synthesized nucleic acid probe to detect Enterobacteriaceae_373
acaagcagttactcttatcc

SEQ ID NO: 374 - DNA, 32 bp, Artificial Sequence
  synthesized nucleic acid probe to detect Enterobacteriaceae_374
cgcggaacaagcagttactcttatcctccgcg

SEQ ID NO: 375 - DNA, 32 bp, Artificial Sequence
  synthesized nucleic acid probe to detect Enterobacteriaceae_375
ctcgcgacaagcagttactcttatcccgcgag

SEQ ID NO: 376 - DNA, 32 bp, Artificial Sequence
  synthesized nucleic acid probe to detect Enterobacteriaceae_376
ccgaccacaagcagttactcttatccggtcgg

SEQ ID NO: 377 - DNA, 32 bp, Artificial Sequence
  synthesized nucleic acid probe to detect Enterobacteriaceae_377
cgacgtacaagcagttactcttatccacgtcg

SEQ ID NO: 378 - DNA, 32 bp, Artificial Sequence
  synthesized nucleic acid probe to detect Enterobacteriaceae_378
cccaccacaagcagttactcttatccggtggg

SEQ ID NO: 379 - DNA, 32 bp, Artificial Sequence
  synthesized nucleic acid probe to detect Enterobacteriaceae_379
cgccgaacaagcagttactcttatcctcggcg

SEQ ID NO: 380 - DNA, 43 bp, Artificial Sequence
  synthesized nucleic acid probe to detect Enterobacteriaceae_380
agacttcggctgtgattacaagcagttactcttatccccgaag

SEQ ID NO: 381 - DNA, 32 bp, Artificial Sequence
  synthesized nucleic acid probe to detect Enterobacteriaceae_381
ctggcgacaagcagttactcttatcccgccag

SEQ ID NO: 382 - DNA, 32 bp, Artificial Sequence
  synthesized nucleic acid probe to detect Enterobacteriaceae_382
ctgccgacaagcagttactcttatcccggcag

SEQ ID NO: 383 - DNA, 32 bp, Artificial Sequence
  synthesized nucleic acid probe to detect Enterobacteriaceae_383
ccgggtacaagcagttactcttatccacccgg

SEQ ID NO: 384 - DNA, 32 bp, Artificial Sequence
  synthesized nucleic acid probe to detect Enterobacteriaceae_384
gcgcctacaagcagttactcttatccaggcgc

SEQ ID NO: 385 - DNA, 32 bp, Artificial Sequence
  synthesized nucleic acid probe to detect Enterobacteriaceae_385
gagcgcacaagcagttactcttatccgcgctc

SEQ ID NO: 386 - DNA, 32 bp, Artificial Sequence
  synthesized nucleic acid probe to detect Enterobacteriaceae_386
ggctggacaagcagttactcttatccccagcc

SEQ ID NO: 387 - DNA, 32 bp, Artificial Sequence
  synthesized nucleic acid probe to detect Enterobacteriaceae_387
gctgcaacaagcagttactcttatcctgcagc

SEQ ID NO: 388 - DNA, 32 bp, Artificial Sequence
  synthesized nucleic acid probe to detect Enterobacteriaceae_388
gggtggacaagcagttactcttatccccaccc

SEQ ID NO: 389 - DNA, 32 bp, Artificial Sequence
  synthesized nucleic acid probe to detect Enterobacteriaceae_389
gcggctacaagcagttactcttatccagccgc

SEQ ID NO: 390 - DNA, 32 bp, Artificial Sequence
  synthesized nucleic acid probe to detect Enterobacteriaceae_390
gaccgcacaagcagttactcttatccgcggtc

SEQ ID NO: 391 - DNA, 32 bp, Artificial Sequence
  synthesized nucleic acid probe to detect Enterobacteriaceae_391
gacggcacaagcagttactcttatccgccgtc

SEQ ID NO: 392 - DNA, 32 bp, Artificial Sequence
  synthesized nucleic acid probe to detect Enterobacteriaceae_392
ggcccaacaagcagttactcttatcctgggcc

SEQ ID NO: 393 - DNA, 21 bp, Artificial Sequence
  synthesized nucleic acid probe to detect Enterobacteriaceae_393
ggacaagcagttactcttatc

SEQ ID NO: 394 - DNA, 33 bp, Artificial Sequence
  synthesized nucleic acid probe to detect Enterobacteriaceae_394
cgcggaggacaagcagttactcttatctccgcg

SEQ ID NO: 395 - DNA, 33 bp, Artificial Sequence
  synthesized nucleic acid probe to detect Enterobacteriaceae_395
ctcgcgggacaagcagttactcttatccgcgag

SEQ ID NO: 396 - DNA, 33 bp, Artificial Sequence
  synthesized nucleic acid probe to detect Enterobacteriaceae_396
ccgaccggacaagcagttactcttatcggtcgg

SEQ ID NO: 397 - DNA, 33 bp, Artificial Sequence
  synthesized nucleic acid probe to detect Enterobacteriaceae_397
cgacgtggacaagcagttactcttatcacgtcg

SEQ ID NO: 398 - DNA, 33 bp, Artificial Sequence
  synthesized nucleic acid probe to detect Enterobacteriaceae_398
cccaccggacaagcagttactcttatcggtggg

SEQ ID NO: 399 - DNA, 33 bp, Artificial Sequence
  synthesized nucleic acid probe to detect Enterobacteriaceae_399
cgccgaggacaagcagttactcttatctcggcg

SEQ ID NO: 400 - DNA, 33 bp, Artificial Sequence
  synthesized nucleic acid probe to detect Enterobacteriaceae_400
ctggcgggacaagcagttactcttatccgccag

SEQ ID NO: 401 - DNA, 33 bp, Artificial Sequence
  synthesized nucleic acid probe to detect Enterobacteriaceae_401
ctgccgggacaagcagttactcttatccggcag

SEQ ID NO: 402 - DNA, 33 bp, Artificial Sequence
  synthesized nucleic acid probe to detect Enterobacteriaceae_402
ccgggtggacaagcagttactcttatcacccgg

SEQ ID NO: 403 - DNA, 33 bp, Artificial Sequence
  synthesized nucleic acid probe to detect Enterobacteriaceae_403
gcgcctggacaagcagttactcttatcaggcgc

SEQ ID NO: 404 - DNA, 33 bp, Artificial Sequence
  synthesized nucleic acid probe to detect Enterobacteriaceae_404
gagcgcggacaagcagttactcttatcgcgctc

SEQ ID NO: 405 - DNA, 33 bp, Artificial Sequence
  synthesized nucleic acid probe to detect Enterobacteriaceae_405
ggctggggacaagcagttactcttatcccagcc

SEQ ID NO: 406 - DNA, 33 bp, Artificial Sequence
  synthesized nucleic acid probe to detect Enterobacteriaceae_406
gctgcaggacaagcagttactcttatctgcagc

SEQ ID NO: 407 - DNA, 33 bp, Artificial Sequence
  synthesized nucleic acid probe to detect Enterobacteriaceae_407
gggtggggacaagcagttactcttatcccaccc

SEQ ID NO: 408 - DNA, 33 bp, Artificial Sequence
  synthesized nucleic acid probe to detect Enterobacteriaceae_408
gcggctggacaagcagttactcttatcagccgc

SEQ ID NO: 409 - DNA, 33 bp, Artificial Sequence
  synthesized nucleic acid probe to detect Enterobacteriaceae_409
gaccgcggacaagcagttactcttatcgcggtc

SEQ ID NO: 410 - DNA, 33 bp, Artificial Sequence
  synthesized nucleic acid probe to detect Enterobacteriaceae_410
gacggcggacaagcagttactcttatcgccgtc

SEQ ID NO: 411 - DNA, 33 bp, Artificial Sequence
  synthesized nucleic acid probe to detect Enterobacteriaceae_411
ggcccaggacaagcagttactcttatctgggcc

SEQ ID NO: 412 - DNA, 21 bp, Artificial Sequence
  synthesized nucleic acid probe to detect Enterobacteriaceae_412
ctatccgactaaagatagtgg

SEQ ID NO: 413 - DNA, 33 bp, Artificial Sequence
  synthesized nucleic acid probe to detect Enterobacteriaceae_413
cgcggactatccgactaaagatagtggtccgcg

SEQ ID NO: 414 - DNA, 30 bp, Artificial Sequence
  synthesized nucleic acid probe to detect Enterobacteriaceae_414
cgcgcctatccgactaaagatagtggcgcg

SEQ ID NO: 415 - DNA, 33 bp, Artificial Sequence
  synthesized nucleic acid probe to detect Enterobacteriaceae_415
ctcgcgctatccgactaaagatagtggcgcgag

SEQ ID NO: 416 - DNA, 33 bp, Artificial Sequence
  synthesized nucleic acid probe to detect Enterobacteriaceae_416
ccgaccctatccgactaaagatagtggggtcgg

SEQ ID NO: 417 - DNA, 33 bp, Artificial Sequence
  synthesized nucleic acid probe to detect Enterobacteriaceae_417
cgacgtctatccgactaaagatagtggacgtcg

SEQ ID NO: 418 - DNA, 33 bp, Artificial Sequence
  synthesized nucleic acid probe to detect Enterobacteriaceae_418
cccaccctatccgactaaagatagtggggtggg

SEQ ID NO: 419 - DNA, 33 bp, Artificial Sequence
  synthesized nucleic acid probe to detect Enterobacteriaceae_419
cgccgactatccgactaaagatagtggtcggcg

SEQ ID NO: 420 - DNA, 33 bp, Artificial Sequence
  synthesized nucleic acid probe to detect Enterobacteriaceae_420
ctggcgctatccgactaaagatagtggcgccag

SEQ ID NO: 421 - DNA, 33 bp, Artificial Sequence
  synthesized nucleic acid probe to detect Enterobacteriaceae_421
ctgccgctatccgactaaagatagtggcggcag

SEQ ID NO: 422 - DNA, 33 bp, Artificial Sequence
  synthesized nucleic acid probe to detect Enterobacteriaceae_422
ccgggtctatccgactaaagatagtggacccgg

SEQ ID NO: 423 - DNA, 33 bp, Artificial Sequence
  synthesized nucleic acid probe to detect Enterobacteriaceae_423
gcgcctctatccgactaaagatagtggaggcgc

SEQ ID NO: 424 - DNA, 33 bp, Artificial Sequence
  synthesized nucleic acid probe to detect Enterobacteriaceae_424
gagcgcctatccgactaaagatagtgggcgctc

SEQ ID NO: 425 - DNA, 33 bp, Artificial Sequence
  synthesized nucleic acid probe to detect Enterobacteriaceae_425
ggctggctatccgactaaagatagtggccagcc

SEQ ID NO: 426 - DNA, 33 bp, Artificial Sequence
  synthesized nucleic acid probe to detect Enterobacteriaceae_426
gctgcactatccgactaaagatagtggtgcagc

SEQ ID NO: 427 - DNA, 33 bp, Artificial Sequence
  synthesized nucleic acid probe to detect Enterobacteriaceae_427
gggtggctatccgactaaagatagtggccaccc

SEQ ID NO: 428 - DNA, 33 bp, Artificial Sequence
  synthesized nucleic acid probe to detect Enterobacteriaceae_428
gcggctctatccgactaaagatagtggagccgc

SEQ ID NO: 429 - DNA, 33 bp, Artificial Sequence
  synthesized nucleic acid probe to detect Enterobacteriaceae_429
gaccgcctatccgactaaagatagtgggcggtc

SEQ ID NO: 430 - DNA, 33 bp, Artificial Sequence
  synthesized nucleic acid probe to detect Enterobacteriaceae_430
gacggcctatccgactaaagatagtgggccgtc

SEQ ID NO: 431 - DNA, 33 bp, Artificial Sequence
  synthesized nucleic acid probe to detect Enterobacteriaceae_431
ggcccactatccgactaaagatagtggtgggcc

SEQ ID NO: 432 - DNA, 18 bp, Artificial Sequence
  synthesized nucleic acid probe to detect Enterobacteriaceae_432
tccgactaaagatagtgg

SEQ ID NO: 433 - DNA, 30 bp, Artificial Sequence
  synthesized nucleic acid probe to detect Enterobacteriaceae_433
cgcggatccgactaaagatagtggtccgcg

SEQ ID NO: 434 - DNA, 30 bp, Artificial Sequence
  synthesized nucleic acid probe to detect Enterobacteriaceae_434
ctcgcgtccgactaaagatagtggcgcgag

SEQ ID NO: 435 - DNA, 30 bp, Artificial Sequence
  synthesized nucleic acid probe to detect Enterobacteriaceae_435
ccgacctccgactaaagatagtggggtcgg

SEQ ID NO: 436 - DNA, 30 bp, Artificial Sequence
  synthesized nucleic acid probe to detect Enterobacteriaceae_436
cgacgttccgactaaagatagtggacgtcg

SEQ ID NO: 437 - DNA, 30 bp, Artificial Sequence
  synthesized nucleic acid probe to detect Enterobacteriaceae_437
cccacctccgactaaagatagtggggtggg

SEQ ID NO: 438 - DNA, 30 bp, Artificial Sequence
  synthesized nucleic acid probe to detect Enterobacteriaceae_438
cgccgatccgactaaagatagtggtcggcg

SEQ ID NO: 439 - DNA, 30 bp, Artificial Sequence
  synthesized nucleic acid probe to detect Enterobacteriaceae_439
ctggcgtccgactaaagatagtggcgccag

SEQ ID NO: 440 - DNA, 30 bp, Artificial Sequence
  synthesized nucleic acid probe to detect Enterobacteriaceae_440
ctgccgtccgactaaagatagtggcggcag

SEQ ID NO: 441 - DNA, 30 bp, Artificial Sequence
  synthesized nucleic acid probe to detect Enterobacteriaceae_441
ccgggttccgactaaagatagtggacccgg

SEQ ID NO: 442 - DNA, 30 bp, Artificial Sequence
  synthesized nucleic acid probe to detect Enterobacteriaceae_442
gcgccttccgactaaagatagtggaggcgc

SEQ ID NO: 443 - DNA, 30 bp, Artificial Sequence
  synthesized nucleic acid probe to detect Enterobacteriaceae_443
gagcgctccgactaaagatagtgggcgctc

SEQ ID NO: 444 - DNA, 30 bp, Artificial Sequence
  synthesized nucleic acid probe to detect Enterobacteriaceae_444
ggctggtccgactaaagatagtggccagcc

SEQ ID NO: 445 - DNA, 30 bp, Artificial Sequence
  synthesized nucleic acid probe to detect Enterobacteriaceae_445
gctgcatccgactaaagatagtggtgcagc

SEQ ID NO: 446 - DNA, 30 bp, Artificial Sequence
  synthesized nucleic acid probe to detect Enterobacteriaceae_446
gggtggtccgactaaagatagtggccaccc

SEQ ID NO: 447 - DNA, 30 bp, Artificial Sequence
  synthesized nucleic acid probe to detect Enterobacteriaceae_447
gcggcttccgactaaagatagtggagccgc

SEQ ID NO: 448 - DNA, 30 bp, Artificial Sequence
  synthesized nucleic acid probe to detect Enterobacteriaceae_448
gaccgctccgactaaagatagtgggcggtc

SEQ ID NO: 449 - DNA, 30 bp, Artificial Sequence
  synthesized nucleic acid probe to detect Enterobacteriaceae_449
gacggctccgactaaagatagtgggccgtc

SEQ ID NO: 450 - DNA, 30 bp, Artificial Sequence
  synthesized nucleic acid probe to detect Enterobacteriaceae_450
ggcccatccgactaaagatagtggtgggcc

SEQ ID NO: 451 - DNA, 21 bp, Artificial Sequence
  synthesized nucleic acid probe to detect Enterobacteriaceae_451
ctggagggaaagccatttcaa

SEQ ID NO: 452 - DNA, 33 bp, Artificial Sequence
  synthesized nucleic acid probe to detect Enterobacteriaceae_452
cgcggactggagggaaagccatttcaatccgcg

SEQ ID NO: 453 - DNA, 33 bp, Artificial Sequence
  synthesized nucleic acid probe to detect Enterobacteriaceae_453
ctcgcgctggagggaaagccatttcaacgcgag

SEQ ID NO: 454 - DNA, 30 bp, Artificial Sequence
  synthesized nucleic acid probe to detect Enterobacteriaceae_454
cgcgctggagggaaagccatttcaagcgcg

SEQ ID NO: 455 - DNA, 33 bp, Artificial Sequence
  synthesized nucleic acid probe to detect Enterobacteriaceae_455
ccgaccctggagggaaagccatttcaaggtcgg

SEQ ID NO: 456 - DNA, 33 bp, Artificial Sequence
  synthesized nucleic acid probe to detect Enterobacteriaceae_456
cgacgtctggagggaaagccatttcaaacgtcg

SEQ ID NO: 457 - DNA, 33 bp, Artificial Sequence
  synthesized nucleic acid probe to detect Enterobacteriaceae_457
cccaccctggagggaaagccatttcaaggtggg

SEQ ID NO: 458 - DNA, 33 bp, Artificial Sequence
  synthesized nucleic acid probe to detect Enterobacteriaceae_458
cgccgactggagggaaagccatttcaatcggcg

SEQ ID NO: 459 - DNA, 33 bp, Artificial Sequence
  synthesized nucleic acid probe to detect Enterobacteriaceae_459
ctggcgctggagggaaagccatttcaacgccag

SEQ ID NO: 460 - DNA, 33 bp, Artificial Sequence
  synthesized nucleic acid probe to detect Enterobacteriaceae_460
ctgccgctggagggaaagccatttcaacggcag

SEQ ID NO: 461 - DNA, 33 bp, Artificial Sequence
  synthesized nucleic acid probe to detect Enterobacteriaceae_461
ccgggtctggagggaaagccatttcaaacccgg

SEQ ID NO: 462 - DNA, 33 bp, Artificial Sequence
  synthesized nucleic acid probe to detect Enterobacteriaceae_462
gcgcctctggagggaaagccatttcaaaggcgc

SEQ ID NO: 463 - DNA, 33 bp, Artificial Sequence
  synthesized nucleic acid probe to detect Enterobacteriaceae_463
gagcgcctggagggaaagccatttcaagcgctc

SEQ ID NO: 464 - DNA, 33 bp, Artificial Sequence
  synthesized nucleic acid probe to detect Enterobacteriaceae_464
ggctggctggagggaaagccatttcaaccagcc

SEQ ID NO: 465 - DNA, 33 bp, Artificial Sequence
  synthesized nucleic acid probe to detect Enterobacteriaceae_465
gctgcactggagggaaagccatttcaatgcagc

SEQ ID NO: 466 - DNA, 33 bp, Artificial Sequence
  synthesized nucleic acid probe to detect Enterobacteriaceae_466
gggtggctggagggaaagccatttcaaccaccc

SEQ ID NO: 467 - DNA, 33 bp, Artificial Sequence
  synthesized nucleic acid probe to detect Enterobacteriaceae_467
gcggctctggagggaaagccatttcaaagccgc

SEQ ID NO: 468 - DNA, 33 bp, Artificial Sequence
  synthesized nucleic acid probe to detect Enterobacteriaceae_468
gaccgcctggagggaaagccatttcaagcggtc

SEQ ID NO: 469 - DNA, 33 bp, Artificial Sequence
  synthesized nucleic acid probe to detect Enterobacteriaceae_469
gacggcctggagggaaagccatttcaagccgtc

SEQ ID NO: 470 - DNA, 33 bp, Artificial Sequence
  synthesized nucleic acid probe to detect Enterobacteriaceae_470
ggcccactggagggaaagccatttcaatgggcc

SEQ ID NO: 471 - DNA, 24 bp, Artificial Sequence
  synthesized nucleic acid probe to detect Enterobacteriaceae_471
atttcaactaccgggctgttaccg

SEQ ID NO: 472 - DNA, 36 bp, Artificial Sequence
  synthesized nucleic acid probe to detect Enterobacteriaceae_472
cgcggaatttcaactaccgggctgttaccgtccgcg

SEQ ID NO: 473 - DNA, 36 bp, Artificial Sequence
  synthesized nucleic acid probe to detect Enterobacteriaceae_473
ctcgcgatttcaactaccgggctgttaccgcgcgag

SEQ ID NO: 474 - DNA, 36 bp, Artificial Sequence
  synthesized nucleic acid probe to detect Enterobacteriaceae_474
ccgaccatttcaactaccgggctgttaccgggtcgg

SEQ ID NO: 475 - DNA, 36 bp, Artificial Sequence
  synthesized nucleic acid probe to detect Enterobacteriaceae_475
cgacgtatttcaactaccgggctgttaccgacgtcg

SEQ ID NO: 476 - DNA, 36 bp, Artificial Sequence
  synthesized nucleic acid probe to detect Enterobacteriaceae_476
cccaccatttcaactaccgggctgttaccgggtggg

SEQ ID NO: 477 - DNA, 36 bp, Artificial Sequence
  synthesized nucleic acid probe to detect Enterobacteriaceae_477
cgccgaatttcaactaccgggctgttaccgtcggcg

SEQ ID NO: 478 - DNA, 36 bp, Artificial Sequence
  synthesized nucleic acid probe to detect Enterobacteriaceae_478
ctggcgatttcaactaccgggctgttaccgcgccag

SEQ ID NO: 479 - DNA, 36 bp, Artificial Sequence
  synthesized nucleic acid probe to detect Enterobacteriaceae_479
ctgccgatttcaactaccgggctgttaccgcggcag

SEQ ID NO: 480 - DNA, 36 bp, Artificial Sequence
  synthesized nucleic acid probe to detect Enterobacteriaceae_480
ccgggtatttcaactaccgggctgttaccgacccgg

SEQ ID NO: 481 - DNA, 36 bp, Artificial Sequence
  synthesized nucleic acid probe to detect Enterobacteriaceae_481
gcgcctatttcaactaccgggctgttaccgaggcgc

SEQ ID NO: 482 - DNA, 36 bp, Artificial Sequence
  synthesized nucleic acid probe to detect Enterobacteriaceae_482
gagcgcatttcaactaccgggctgttaccggcgctc

SEQ ID NO: 483 - DNA, 36 bp, Artificial Sequence
  synthesized nucleic acid probe to detect Enterobacteriaceae_483
ggctggatttcaactaccgggctgttaccgccagcc

SEQ ID NO: 484 - DNA, 36 bp, Artificial Sequence
  synthesized nucleic acid probe to detect Enterobacteriaceae_484
gctgcaatttcaactaccgggctgttaccgtgcagc

SEQ ID NO: 485 - DNA, 36 bp, Artificial Sequence
  synthesized nucleic acid probe to detect Enterobacteriaceae_485
gggtggatttcaactaccgggctgttaccgccaccc

SEQ ID NO: 486 - DNA, 36 bp, Artificial Sequence
  synthesized nucleic acid probe to detect Enterobacteriaceae_486
gcggctatttcaactaccgggctgttaccgagccgc

SEQ ID NO: 487 - DNA, 36 bp, Artificial Sequence
  synthesized nucleic acid probe to detect Enterobacteriaceae_487
gaccgcatttcaactaccgggctgttaccggcggtc

SEQ ID NO: 488 - DNA, 36 bp, Artificial Sequence
  synthesized nucleic acid probe to detect Enterobacteriaceae_488
gacggcatttcaactaccgggctgttaccggccgtc

SEQ ID NO: 489 - DNA, 36 bp, Artificial Sequence
  synthesized nucleic acid probe to detect Enterobacteriaceae_489
ggcccaatttcaactaccgggctgttaccgtgggcc

SEQ ID NO: 490 - DNA, 19 bp, Artificial Sequence
  synthesized nucleic acid probe to detect Enterobacteriaceae_490
gatccactctggagggaaa

SEQ ID NO: 491 - DNA, 31 bp, Artificial Sequence
  synthesized nucleic acid probe to detect Enterobacteriaceae_491
cgcggagatccactctggagggaaatccgcg

SEQ ID NO: 492 - DNA, 31 bp, Artificial Sequence
  synthesized nucleic acid probe to detect Enterobacteriaceae_492
ctcgcggatccactctggagggaaacgcgag

SEQ ID NO: 493 - DNA, 31 bp, Artificial Sequence
  synthesized nucleic acid probe to detect Enterobacteriaceae_493
ccgaccgatccactctggagggaaaggtcgg

SEQ ID NO: 494 - DNA, 31 bp, Artificial Sequence
  synthesized nucleic acid probe to detect Enterobacteriaceae_494
cgacgtgatccactctggagggaaaacgtcg

SEQ ID NO: 495 - DNA, 31 bp, Artificial Sequence
  synthesized nucleic acid probe to detect Enterobacteriaceae_495
cccaccgatccactctggagggaaaggtggg

SEQ ID NO: 496 - DNA, 31 bp, Artificial Sequence
  synthesized nucleic acid probe to detect Enterobacteriaceae_496
cgccgagatccactctggagggaaatcggcg

SEQ ID NO: 497 - DNA, 31 bp, Artificial Sequence
  synthesized nucleic acid probe to detect Enterobacteriaceae_497
ctggcggatccactctggagggaaacgccag

SEQ ID NO: 498 - DNA, 31 bp, Artificial Sequence
  synthesized nucleic acid probe to detect Enterobacteriaceae_498
ctgccggatccactctggagggaaacggcag

SEQ ID NO: 499 - DNA, 31 bp, Artificial Sequence
  synthesized nucleic acid probe to detect Enterobacteriaceae_499
ccgggtgatccactctggagggaaaacccgg

SEQ ID NO: 500 - DNA, 31 bp, Artificial Sequence
  synthesized nucleic acid probe to detect Enterobacteriaceae_500
gcgcctgatccactctggagggaaaaggcgc

SEQ ID NO: 501 - DNA, 31 bp, Artificial Sequence
  synthesized nucleic acid probe to detect Enterobacteriaceae_501
gagcgcgatccactctggagggaaagcgctc

SEQ ID NO: 502 - DNA, 31 bp, Artificial Sequence
  synthesized nucleic acid probe to detect Enterobacteriaceae_502
ggctgggatccactctggagggaaaccagcc

SEQ ID NO: 503 - DNA, 31 bp, Artificial Sequence
  synthesized nucleic acid probe to detect Enterobacteriaceae_503
gctgcagatccactctggagggaaatgcagc

SEQ ID NO: 504 - DNA, 31 bp, Artificial Sequence
  synthesized nucleic acid probe to detect Enterobacteriaceae_504
gggtgggatccactctggagggaaaccaccc

SEQ ID NO: 505 - DNA, 31 bp, Artificial Sequence
  synthesized nucleic acid probe to detect Enterobacteriaceae_505
gcggctgatccactctggagggaaaagccgc

SEQ ID NO: 506 - DNA, 31 bp, Artificial Sequence
  synthesized nucleic acid probe to detect Enterobacteriaceae_506
gaccgcgatccactctggagggaaagcggtc

SEQ ID NO: 507 - DNA, 31 bp, Artificial Sequence
  synthesized nucleic acid probe to detect Enterobacteriaceae_507
gacggcgatccactctggagggaaagccgtc

SEQ ID NO: 508 - DNA, 31 bp, Artificial Sequence
  synthesized nucleic acid probe to detect Enterobacteriaceae_508
ggcccagatccactctggagggaaatgggcc

SEQ ID NO: 509 - DNA, 23 bp, Artificial Sequence
  synthesized nucleic acid probe to detect Enterobacteriaceae_509
tgactcccgagtataagtacatg

SEQ ID NO: 510 - DNA, 35 bp, Artificial Sequence
  synthesized nucleic acid probe to detect Enterobacteriaceae_510
cgcggatgactcccgagtataagtacatgtccgcg

SEQ ID NO: 511 - DNA, 35 bp, Artificial Sequence
  synthesized nucleic acid probe to detect Enterobacteriaceae_511
ctcgcgtgactcccgagtataagtacatgcgcgag

SEQ ID NO: 512 - DNA, 35 bp, Artificial Sequence
  synthesized nucleic acid probe to detect Enterobacteriaceae_512
ccgacctgactcccgagtataagtacatgggtcgg

SEQ ID NO: 513 - DNA, 35 bp, Artificial Sequence
  synthesized nucleic acid probe to detect Enterobacteriaceae_513
cgacgttgactcccgagtataagtacatgacgtcg

SEQ ID NO: 514 - DNA, 35 bp, Artificial Sequence
  synthesized nucleic acid probe to detect Enterobacteriaceae_514
cccacctgactcccgagtataagtacatgggtggg

SEQ ID NO: 515 - DNA, 35 bp, Artificial Sequence
  synthesized nucleic acid probe to detect Enterobacteriaceae_515
cgccgatgactcccgagtataagtacatgtcggcg

SEQ ID NO: 516 - DNA, 35 bp, Artificial Sequence
  synthesized nucleic acid probe to detect Enterobacteriaceae_516
ctggcgtgactcccgagtataagtacatgcgccag

SEQ ID NO: 517 - DNA, 35 bp, Artificial Sequence
  synthesized nucleic acid probe to detect Enterobacteriaceae_517
ctgccgtgactcccgagtataagtacatgcggcag

SEQ ID NO: 518 - DNA, 35 bp, Artificial Sequence
  synthesized nucleic acid probe to detect Enterobacteriaceae_518
ccgggttgactcccgagtataagtacatgacccgg

SEQ ID NO: 519 - DNA, 35 bp, Artificial Sequence
  synthesized nucleic acid probe to detect Enterobacteriaceae_519
gcgccttgactcccgagtataagtacatgaggcgc

SEQ ID NO: 520 - DNA, 35 bp, Artificial Sequence
  synthesized nucleic acid probe to detect Enterobacteriaceae_520
gagcgctgactcccgagtataagtacatggcgctc

SEQ ID NO: 521 - DNA, 35 bp, Artificial Sequence
  synthesized nucleic acid probe to detect Enterobacteriaceae_521
ggctggtgactcccgagtataagtacatgccagcc

SEQ ID NO: 522 - DNA, 35 bp, Artificial Sequence
  synthesized nucleic acid probe to detect Enterobacteriaceae_522
gctgcatgactcccgagtataagtacatgtgcagc

SEQ ID NO: 523 - DNA, 35 bp, Artificial Sequence
  synthesized nucleic acid probe to detect Enterobacteriaceae_523
gggtggtgactcccgagtataagtacatgccaccc

SEQ ID NO: 524 - DNA, 34 bp, Artificial Sequence
  synthesized nucleic acid probe to detect Enterobacteriaceae_524
ccggtctgactcccgagtataagtacatgaccgg

SEQ ID NO: 525 - DNA, 35 bp, Artificial Sequence
  synthesized nucleic acid probe to detect Enterobacteriaceae_525
gcggcttgactcccgagtataagtacatgagccgc

SEQ ID NO: 526 - DNA, 35 bp, Artificial Sequence
  synthesized nucleic acid probe to detect Enterobacteriaceae_526
gaccgctgactcccgagtataagtacatggcggtc

SEQ ID NO: 527 - DNA, 35 bp, Artificial Sequence
  synthesized nucleic acid probe to detect Enterobacteriaceae_527
gacggctgactcccgagtataagtacatggccgtc

SEQ ID NO: 528 - DNA, 35 bp, Artificial Sequence
  synthesized nucleic acid probe to detect Enterobacteriaceae_528
ggcccatgactcccgagtataagtacatgtgggcc

SEQ ID NO: 529 - DNA, 20 bp, Artificial Sequence
  synthesized nucleic acid probe to detect Enterobacteriaceae_529
cgaacctctaaagaggttca

SEQ ID NO: 530 - DNA, 32 bp, Artificial Sequence
  synthesized nucleic acid probe to detect Enterobacteriaceae_530
cgcggacgaacctctaaagaggttcatccgcg

SEQ ID NO: 531 - DNA, 32 bp, Artificial Sequence
  synthesized nucleic acid probe to detect Enterobacteriaceae_531
ctcgcgcgaacctctaaagaggttcacgcgag

SEQ ID NO: 532 - DNA, 32 bp, Artificial Sequence
  synthesized nucleic acid probe to detect Enterobacteriaceae_532
ccgacccgaacctctaaagaggttcaggtcgg

SEQ ID NO: 533 - DNA, 32 bp, Artificial Sequence
  synthesized nucleic acid probe to detect Enterobacteriaceae_533
cgacgtcgaacctctaaagaggttcaacgtcg

SEQ ID NO: 534 - DNA, 32 bp, Artificial Sequence
  synthesized nucleic acid probe to detect Enterobacteriaceae_534
cccacccgaacctctaaagaggttcaggtggg

SEQ ID NO: 535 - DNA, 32 bp, Artificial Sequence
  synthesized nucleic acid probe to detect Enterobacteriaceae_535
cgccgacgaacctctaaagaggttcatcggcg

SEQ ID NO: 536 - DNA, 32 bp, Artificial Sequence
  synthesized nucleic acid probe to detect Enterobacteriaceae_536
ctggcgcgaacctctaaagaggttcacgccag

SEQ ID NO: 537 - DNA, 32 bp, Artificial Sequence
  synthesized nucleic acid probe to detect Enterobacteriaceae_537
ctgccgcgaacctctaaagaggttcacggcag

SEQ ID NO: 538 - DNA, 32 bp, Artificial Sequence
  synthesized nucleic acid probe to detect Enterobacteriaceae_538
ccgggtcgaacctctaaagaggttcaacccgg

SEQ ID NO: 539 - DNA, 32 bp, Artificial Sequence
  synthesized nucleic acid probe to detect Enterobacteriaceae_539
gcgcctcgaacctctaaagaggttcaaggcgc

SEQ ID NO: 540 - DNA, 32 bp, Artificial Sequence
  synthesized nucleic acid probe to detect Enterobacteriaceae_540
gagcgccgaacctctaaagaggttcagcgctc

SEQ ID NO: 541 - DNA, 32 bp, Artificial Sequence
  synthesized nucleic acid probe to detect Enterobacteriaceae_541
ggctggcgaacctctaaagaggttcaccagcc

SEQ ID NO: 542 - DNA, 32 bp, Artificial Sequence
  synthesized nucleic acid probe to detect Enterobacteriaceae_542
gctgcacgaacctctaaagaggttcatgcagc

SEQ ID NO: 543 - DNA, 32 bp, Artificial Sequence
  synthesized nucleic acid probe to detect Enterobacteriaceae_543
gggtggcgaacctctaaagaggttcaccaccc

SEQ ID NO: 544 - DNA, 32 bp, Artificial Sequence
  synthesized nucleic acid probe to detect Enterobacteriaceae_544
gcggctcgaacctctaaagaggttcaagccgc

SEQ ID NO: 545 - DNA, 32 bp, Artificial Sequence
  synthesized nucleic acid probe to detect Enterobacteriaceae_545
gaccgccgaacctctaaagaggttcagcggtc

SEQ ID NO: 546 - DNA, 32 bp, Artificial Sequence
  synthesized nucleic acid probe to detect Enterobacteriaceae_546
gacggccgaacctctaaagaggttcagccgtc

SEQ ID NO: 547 - DNA, 32 bp, Artificial Sequence
  synthesized nucleic acid probe to detect Enterobacteriaceae_547
ggcccacgaacctctaaagaggttcatgggcc

SEQ ID NO: 548 - DNA, 23 bp, Artificial Sequence
  synthesized nucleic acid probe to detect Enterobacteriaceae_548
catcggttaaacaattagcactg

SEQ ID NO: 549 - DNA, 35 bp, Artificial Sequence
  synthesized nucleic acid probe to detect Enterobacteriaceae_549
cgcggacatcggttaaacaattagcactgtccgcg

SEQ ID NO: 550 - DNA, 35 bp, Artificial Sequence
  synthesized nucleic acid probe to detect Enterobacteriaceae_550
ctcgcgcatcggttaaacaattagcactgcgcgag

SEQ ID NO: 551 - DNA, 35 bp, Artificial Sequence
  synthesized nucleic acid probe to detect Enterobacteriaceae_551
ccgacccatcggttaaacaattagcactgggtcgg

SEQ ID NO: 552 - DNA, 35 bp, Artificial Sequence
  synthesized nucleic acid probe to detect Enterobacteriaceae_552
cgacgtcatcggttaaacaattagcactgacgtcg

SEQ ID NO: 553 - DNA, 35 bp, Artificial Sequence
  synthesized nucleic acid probe to detect Enterobacteriaceae_553
cccacccatcggttaaacaattagcactgggtggg

SEQ ID NO: 554 - DNA, 35 bp, Artificial Sequence
  synthesized nucleic acid probe to detect Enterobacteriaceae_554
cgccgacatcggttaaacaattagcactgtcggcg

SEQ ID NO: 555 - DNA, 35 bp, Artificial Sequence
  synthesized nucleic acid probe to detect Enterobacteriaceae_555
ctggcgcatcggttaaacaattagcactgcgccag

SEQ ID NO: 556 - DNA, 33 bp, Artificial Sequence
  synthesized nucleic acid probe to detect Enterobacteriaceae_556
cgcgccatcggttaaacaattagcactggcgcg

SEQ ID NO: 557 - DNA, 35 bp, Artificial Sequence
  synthesized nucleic acid probe to detect Enterobacteriaceae_557
ctgccgcatcggttaaacaattagcactgcggcag

SEQ ID NO: 558 - DNA, 35 bp, Artificial Sequence
  synthesized nucleic acid probe to detect Enterobacteriaceae_558
ccgggtcatcggttaaacaattagcactgacccgg

SEQ ID NO: 559 - DNA, 35 bp, Artificial Sequence
  synthesized nucleic acid probe to detect Enterobacteriaceae_559
gcgcctcatcggttaaacaattagcactgaggcgc

SEQ ID NO: 560 - DNA, 35 bp, Artificial Sequence
  synthesized nucleic acid probe to detect Enterobacteriaceae_560
gagcgccatcggttaaacaattagcactggcgctc

SEQ ID NO: 561 - DNA, 35 bp, Artificial Sequence
  synthesized nucleic acid probe to detect Enterobacteriaceae_561
ggctggcatcggttaaacaattagcactgccagcc

SEQ ID NO: 562 - DNA, 35 bp, Artificial Sequence
  synthesized nucleic acid probe to detect Enterobacteriaceae_562
gctgcacatcggttaaacaattagcactgtgcagc

SEQ ID NO: 563 - DNA, 35 bp, Artificial Sequence
  synthesized nucleic acid probe to detect Enterobacteriaceae_563
gggtggcatcggttaaacaattagcactgccaccc

SEQ ID NO: 564 - DNA, 35 bp, Artificial Sequence
  synthesized nucleic acid probe to detect Enterobacteriaceae_564
gcggctcatcggttaaacaattagcactgagccgc

SEQ ID NO: 565 - DNA, 35 bp, Artificial Sequence
  synthesized nucleic acid probe to detect Enterobacteriaceae_565
gaccgccatcggttaaacaattagcactggcggtc

SEQ ID NO: 566 - DNA, 35 bp, Artificial Sequence
  synthesized nucleic acid probe to detect Enterobacteriaceae_566
gacggccatcggttaaacaattagcactggccgtc

SEQ ID NO: 567 - DNA, 35 bp, Artificial Sequence
  synthesized nucleic acid probe to detect Enterobacteriaceae_567
ggcccacatcggttaaacaattagcactgtgggcc

SEQ ID NO: 568 - DNA, 23 bp, Artificial Sequence
  synthesized nucleic acid probe to detect Enterobacteriaceae_568
tgcgattccctatccttctgtgt

SEQ ID NO: 569 - DNA, 35 bp, Artificial Sequence
  synthesized nucleic acid probe to detect Enterobacteriaceae_569
cgcggatgcgattccctatccttctgtgttccgcg

SEQ ID NO: 570 - DNA, 35 bp, Artificial Sequence
  synthesized nucleic acid probe to detect Enterobacteriaceae_570
ctcgcgtgcgattccctatccttctgtgtcgcgag

SEQ ID NO: 571 - DNA, 35 bp, Artificial Sequence
  synthesized nucleic acid probe to detect Enterobacteriaceae_571
ccgacctgcgattccctatccttctgtgtggtcgg

SEQ ID NO: 572 - DNA, 35 bp, Artificial Sequence
  synthesized nucleic acid probe to detect Enterobacteriaceae_572
cgacgttgcgattccctatccttctgtgtacgtcg

SEQ ID NO: 573 - DNA, 35 bp, Artificial Sequence
  synthesized nucleic acid probe to detect Enterobacteriaceae_573
cccacctgcgattccctatccttctgtgtggtggg

SEQ ID NO: 574 - DNA, 35 bp, Artificial Sequence
  synthesized nucleic acid probe to detect Enterobacteriaceae_574
cgccgatgcgattccctatccttctgtgttcggcg

SEQ ID NO: 575 - DNA, 35 bp, Artificial Sequence
  synthesized nucleic acid probe to detect Enterobacteriaceae_575
ctggcgtgcgattccctatccttctgtgtcgccag

SEQ ID NO: 576 - DNA, 35 bp, Artificial Sequence
  synthesized nucleic acid probe to detect Enterobacteriaceae_576
ctgccgtgcgattccctatccttctgtgtcggcag

SEQ ID NO: 577 - DNA, 35 bp, Artificial Sequence
  synthesized nucleic acid probe to detect Enterobacteriaceae_577
ccgggttgcgattccctatccttctgtgtacccgg

SEQ ID NO: 578 - DNA, 35 bp, Artificial Sequence
  synthesized nucleic acid probe to detect Enterobacteriaceae_578
gcgccttgcgattccctatccttctgtgtaggcgc

SEQ ID NO: 579 - DNA, 35 bp, Artificial Sequence
  synthesized nucleic acid probe to detect Enterobacteriaceae_579
gagcgctgcgattccctatccttctgtgtgcgctc

SEQ ID NO: 580 - DNA, 35 bp, Artificial Sequence
  synthesized nucleic acid probe to detect Enterobacteriaceae_580
ggctggtgcgattccctatccttctgtgtccagcc

SEQ ID NO: 581 - DNA, 35 bp, Artificial Sequence
  synthesized nucleic acid probe to detect Enterobacteriaceae_581
gctgcatgcgattccctatccttctgtgttgcagc

SEQ ID NO: 582 - DNA, 35 bp, Artificial Sequence
  synthesized nucleic acid probe to detect Enterobacteriaceae_582
gggtggtgcgattccctatccttctgtgtccaccc

SEQ ID NO: 583 - DNA, 35 bp, Artificial Sequence
  synthesized nucleic acid probe to detect Enterobacteriaceae_583
gcggcttgcgattccctatccttctgtgtagccgc

SEQ ID NO: 584 - DNA, 35 bp, Artificial Sequence
  synthesized nucleic acid probe to detect Enterobacteriaceae_584
gaccgctgcgattccctatccttctgtgtgcggtc

SEQ ID NO: 585 - DNA, 35 bp, Artificial Sequence
  synthesized nucleic acid probe to detect Enterobacteriaceae_585
ccagcctgcgattccctatccttctgtgtggctgg

SEQ ID NO: 586 - DNA, 35 bp, Artificial Sequence
  synthesized nucleic acid probe to detect Enterobacteriaceae_586
gacggctgcgattccctatccttctgtgtgccgtc

SEQ ID NO: 587 - DNA, 35 bp, Artificial Sequence
  synthesized nucleic acid probe to detect Enterobacteriaceae_587
ggcccatgcgattccctatccttctgtgttgggcc

SEQ ID NO: 588 - DNA, 24 bp, Artificial Sequence
  synthesized nucleic acid probe to detect Enterobacteriaceae_588
ggatttgcctacttctcacactca

SEQ ID NO: 589 - DNA, 36 bp, Artificial Sequence
  synthesized nucleic acid probe to detect Enterobacteriaceae_589
cgcggaggatttgcctacttctcacactcatccgcg

SEQ ID NO: 590 - DNA, 36 bp, Artificial Sequence
  synthesized nucleic acid probe to detect Enterobacteriaceae_590
ctcgcgggatttgcctacttctcacactcacgcgag

SEQ ID NO: 591 - DNA, 36 bp, Artificial Sequence
  synthesized nucleic acid probe to detect Enterobacteriaceae_591
ccgaccggatttgcctacttctcacactcaggtcgg

SEQ ID NO: 592 - DNA, 36 bp, Artificial Sequence
  synthesized nucleic acid probe to detect Enterobacteriaceae_592
cgacgtggatttgcctacttctcacactcaacgtcg

SEQ ID NO: 593 - DNA, 36 bp, Artificial Sequence
  synthesized nucleic acid probe to detect Enterobacteriaceae_593
cccaccggatttgcctacttctcacactcaggtggg

SEQ ID NO: 594 - DNA, 36 bp, Artificial Sequence
  synthesized nucleic acid probe to detect Enterobacteriaceae_594
cgccgaggatttgcctacttctcacactcatcggcg

SEQ ID NO: 595 - DNA, 36 bp, Artificial Sequence
  synthesized nucleic acid probe to detect Enterobacteriaceae_595
ctggcgggatttgcctacttctcacactcacgccag

SEQ ID NO: 596 - DNA, 36 bp, Artificial Sequence
  synthesized nucleic acid probe to detect Enterobacteriaceae_596
ctgccgggatttgcctacttctcacactcacggcag

SEQ ID NO: 597 - DNA, 36 bp, Artificial Sequence
  synthesized nucleic acid probe to detect Enterobacteriaceae_597
ccgggtggatttgcctacttctcacactcaacccgg

SEQ ID NO: 598 - DNA, 35 bp, Artificial Sequence
  synthesized nucleic acid probe to detect Enterobacteriaceae_598
cccctggatttgcctacttctcacactcacagggg

SEQ ID NO: 599 - DNA, 36 bp, Artificial Sequence
  synthesized nucleic acid probe to detect Enterobacteriaceae_599
gcgcctggatttgcctacttctcacactcaaggcgc

SEQ ID NO: 600 - DNA, 36 bp, Artificial Sequence
  synthesized nucleic acid probe to detect Enterobacteriaceae_600
gagcgcggatttgcctacttctcacactcagcgctc

SEQ ID NO: 601 - DNA, 36 bp, Artificial Sequence
  synthesized nucleic acid probe to detect Enterobacteriaceae_601
ggctggggatttgcctacttctcacactcaccagcc

SEQ ID NO: 602 - DNA, 36 bp, Artificial Sequence
  synthesized nucleic acid probe to detect Enterobacteriaceae_602
gctgcaggatttgcctacttctcacactcatgcagc

SEQ ID NO: 603 - DNA, 36 bp, Artificial Sequence
  synthesized nucleic acid probe to detect Enterobacteriaceae_603
gggtggggatttgcctacttctcacactcaccaccc

SEQ ID NO: 604 - DNA, 36 bp, Artificial Sequence
  synthesized nucleic acid probe to detect Enterobacteriaceae_604
gcggctggatttgcctacttctcacactcaagccgc

SEQ ID NO: 605 - DNA, 36 bp, Artificial Sequence
  synthesized nucleic acid probe to detect Enterobacteriaceae_605
gaccgcggatttgcctacttctcacactcagcggtc

SEQ ID NO: 606 - DNA, 36 bp, Artificial Sequence
  synthesized nucleic acid probe to detect Enterobacteriaceae_606
gacggcggatttgcctacttctcacactcagccgtc

SEQ ID NO: 607 - DNA, 36 bp, Artificial Sequence
  synthesized nucleic acid probe to detect Enterobacteriaceae_607
ggcccaggatttgcctacttctcacactcatgggcc

SEQ ID NO: 608 - DNA, 18 bp, Artificial Sequence
  synthesized nucleic acid probe to detect Enterobacteriaceae_608
gcacacgtctcttcggct

SEQ ID NO: 609 - DNA, 30 bp, Artificial Sequence
  synthesized nucleic acid probe to detect Enterobacteriaceae_609
cgcggagcacacgtctcttcggcttccgcg

SEQ ID NO: 610 - DNA, 30 bp, Artificial Sequence
  synthesized nucleic acid probe to detect Enterobacteriaceae_610
ctcgcggcacacgtctcttcggctcgcgag

SEQ ID NO: 611 - DNA, 30 bp, Artificial Sequence
  synthesized nucleic acid probe to detect Enterobacteriaceae_611
ccgaccgcacacgtctcttcggctggtcgg

SEQ ID NO: 612 - DNA, 30 bp, Artificial Sequence
  synthesized nucleic acid probe to detect Enterobacteriaceae_612
cgacgtgcacacgtctcttcggctacgtcg

SEQ ID NO: 613 - DNA, 30 bp, Artificial Sequence
  synthesized nucleic acid probe to detect Enterobacteriaceae_613
cccaccgcacacgtctcttcggctggtggg

SEQ ID NO: 614 - DNA, 30 bp, Artificial Sequence
  synthesized nucleic acid probe to detect Enterobacteriaceae_614
cgccgagcacacgtctcttcggcttcggcg

SEQ ID NO: 615 - DNA, 30 bp, Artificial Sequence
  synthesized nucleic acid probe to detect Enterobacteriaceae_615
ctggcggcacacgtctcttcggctcgccag

SEQ ID NO: 616 - DNA, 30 bp, Artificial Sequence
  synthesized nucleic acid probe to detect Enterobacteriaceae_616
ctgccggcacacgtctcttcggctcggcag

SEQ ID NO: 617 - DNA, 30 bp, Artificial Sequence
  synthesized nucleic acid probe to detect Enterobacteriaceae_617
ccgggtgcacacgtctcttcggctacccgg

SEQ ID NO: 618 - DNA, 30 bp, Artificial Sequence
  synthesized nucleic acid probe to detect Enterobacteriaceae_618
gcgcctgcacacgtctcttcggctaggcgc

SEQ ID NO: 619 - DNA, 30 bp, Artificial Sequence
  synthesized nucleic acid probe to detect Enterobacteriaceae_619
gagcgcgcacacgtctcttcggctgcgctc

SEQ ID NO: 620 - DNA, 30 bp, Artificial Sequence
  synthesized nucleic acid probe to detect Enterobacteriaceae_620
ggctgggcacacgtctcttcggctccagcc

SEQ ID NO: 621 - DNA, 30 bp, Artificial Sequence
  synthesized nucleic acid probe to detect Enterobacteriaceae_621
gctgcagcacacgtctcttcggcttgcagc

SEQ ID NO: 622 - DNA, 30 bp, Artificial Sequence
  synthesized nucleic acid probe to detect Enterobacteriaceae_622
gggtgggcacacgtctcttcggctccaccc

SEQ ID NO: 623 - DNA, 30 bp, Artificial Sequence
  synthesized nucleic acid probe to detect Enterobacteriaceae_623
gcggctgcacacgtctcttcggctagccgc

SEQ ID NO: 624 - DNA, 30 bp, Artificial Sequence
  synthesized nucleic acid probe to detect Enterobacteriaceae_624
gaccgcgcacacgtctcttcggctgcggtc

SEQ ID NO: 625 - DNA, 30 bp, Artificial Sequence
  synthesized nucleic acid probe to detect Enterobacteriaceae_625
gacggcgcacacgtctcttcggctgccgtc

SEQ ID NO: 626 - DNA, 30 bp, Artificial Sequence
  synthesized nucleic acid probe to detect Enterobacteriaceae_626
ggcccagcacacgtctcttcggcttgggcc

SEQ ID NO: 627 - DNA, 19 bp, Artificial Sequence
  synthesized nucleic acid probe to detect Enterobacteriaceae_627
accattgcctcctcgtatg

SEQ ID NO: 628 - DNA, 31 bp, Artificial Sequence
  synthesized nucleic acid probe to detect Enterobacteriaceae_628
cgcggaaccattgcctcctcgtatgtccgcg

SEQ ID NO: 629 - DNA, 31 bp, Artificial Sequence
  synthesized nucleic acid probe to detect Enterobacteriaceae_629
ctcgcgaccattgcctcctcgtatgcgcgag

SEQ ID NO: 630 - DNA, 31 bp, Artificial Sequence
  synthesized nucleic acid probe to detect Enterobacteriaceae_630
ccgaccaccattgcctcctcgtatgggtcgg

SEQ ID NO: 631 - DNA, 31 bp, Artificial Sequence
  synthesized nucleic acid probe to detect Enterobacteriaceae_631
cgacgtaccattgcctcctcgtatgacgtcg

SEQ ID NO: 632 - DNA, 31 bp, Artificial Sequence
  synthesized nucleic acid probe to detect Enterobacteriaceae_632
cccaccaccattgcctcctcgtatgggtggg

SEQ ID NO: 633 - DNA, 31 bp, Artificial Sequence
  synthesized nucleic acid probe to detect Enterobacteriaceae_633
cgccgaaccattgcctcctcgtatgtcggcg

SEQ ID NO: 634 - DNA, 31 bp, Artificial Sequence
  synthesized nucleic acid probe to detect Enterobacteriaceae_634
ctggcgaccattgcctcctcgtatgcgccag

SEQ ID NO: 635 - DNA, 31 bp, Artificial Sequence
  synthesized nucleic acid probe to detect Enterobacteriaceae_635
ctgccgaccattgcctcctcgtatgcggcag

SEQ ID NO: 636 - DNA, 31 bp, Artificial Sequence
  synthesized nucleic acid probe to detect Enterobacteriaceae_636
ccgggtaccattgcctcctcgtatgacccgg

SEQ ID NO: 637 - DNA, 31 bp, Artificial Sequence
  synthesized nucleic acid probe to detect Enterobacteriaceae_637
gcgcctaccattgcctcctcgtatgaggcgc

SEQ ID NO: 638 - DNA, 31 bp, Artificial Sequence
  synthesized nucleic acid probe to detect Enterobacteriaceae_638
gagcgcaccattgcctcctcgtatggcgctc

SEQ ID NO: 639 - DNA, 31 bp, Artificial Sequence
  synthesized nucleic acid probe to detect Enterobacteriaceae_639
ggctggaccattgcctcctcgtatgccagcc

SEQ ID NO: 640 - DNA, 31 bp, Artificial Sequence
  synthesized nucleic acid probe to detect Enterobacteriaceae_640
gctgcaaccattgcctcctcgtatgtgcagc

SEQ ID NO: 641 - DNA, 31 bp, Artificial Sequence
  synthesized nucleic acid probe to detect Enterobacteriaceae_641
gggtggaccattgcctcctcgtatgccaccc

SEQ ID NO: 642 - DNA, 31 bp, Artificial Sequence
  synthesized nucleic acid probe to detect Enterobacteriaceae_642
gcggctaccattgcctcctcgtatgagccgc

SEQ ID NO: 643 - DNA, 31 bp, Artificial Sequence
  synthesized nucleic acid probe to detect Enterobacteriaceae_643
gaccgcaccattgcctcctcgtatggcggtc

SEQ ID NO: 644 - DNA, 31 bp, Artificial Sequence
  synthesized nucleic acid probe to detect Enterobacteriaceae_644
gacggcaccattgcctcctcgtatggccgtc

SEQ ID NO: 645 - DNA, 31 bp, Artificial Sequence
  synthesized nucleic acid probe to detect Enterobacteriaceae_645
ggcccaaccattgcctcctcgtatgtgggcc

SEQ ID NO: 646 - DNA, 16 bp, Artificial Sequence
  synthesized nucleic acid probe to detect Enterobacteriaceae_646
cacgcgctaagcgtgg

SEQ ID NO: 647 - DNA, 28 bp, Artificial Sequence
  synthesized nucleic acid probe to detect Enterobacteriaceae_647
cgcggacacgcgctaagcgtggtccgcg

SEQ ID NO: 648 - DNA, 28 bp, Artificial Sequence
  synthesized nucleic acid probe to detect Enterobacteriaceae_648
ctcgcgcacgcgctaagcgtggcgcgag

SEQ ID NO: 649 - DNA, 28 bp, Artificial Sequence
  synthesized nucleic acid probe to detect Enterobacteriaceae_649
ccgacccacgcgctaagcgtggggtcgg

SEQ ID NO: 650 - DNA, 28 bp, Artificial Sequence
  synthesized nucleic acid probe to detect Enterobacteriaceae_650
cgacgtcacgcgctaagcgtggacgtcg

SEQ ID NO: 651 - DNA, 28 bp, Artificial Sequence
  synthesized nucleic acid probe to detect Enterobacteriaceae_651
cccacccacgcgctaagcgtggggtggg

SEQ ID NO: 652 - DNA, 28 bp, Artificial Sequence
  synthesized nucleic acid probe to detect Enterobacteriaceae_652
cgccgacacgcgctaagcgtggtcggcg

SEQ ID NO: 653 - DNA, 28 bp, Artificial Sequence
  synthesized nucleic acid probe to detect Enterobacteriaceae_653
ctggcgcacgcgctaagcgtggcgccag

SEQ ID NO: 654 - DNA, 28 bp, Artificial Sequence
  synthesized nucleic acid probe to detect Enterobacteriaceae_654
ctgccgcacgcgctaagcgtggcggcag

SEQ ID NO: 655 - DNA, 28 bp, Artificial Sequence
  synthesized nucleic acid probe to detect Enterobacteriaceae_655
ccgggtcacgcgctaagcgtggacccgg

SEQ ID NO: 656 - DNA, 28 bp, Artificial Sequence
  synthesized nucleic acid probe to detect Enterobacteriaceae_656
gcgcctcacgcgctaagcgtggaggcgc

SEQ ID NO: 657 - DNA, 28 bp, Artificial Sequence
  synthesized nucleic acid probe to detect Enterobacteriaceae_657
gagcgccacgcgctaagcgtgggcgctc

SEQ ID NO: 658 - DNA, 28 bp, Artificial Sequence
  synthesized nucleic acid probe to detect Enterobacteriaceae_658
ggctggcacgcgctaagcgtggccagcc

SEQ ID NO: 659 - DNA, 28 bp, Artificial Sequence
  synthesized nucleic acid probe to detect Enterobacteriaceae_659
gctgcacacgcgctaagcgtggtgcagc

SEQ ID NO: 660 - DNA, 28 bp, Artificial Sequence
  synthesized nucleic acid probe to detect Enterobacteriaceae_660
gggtggcacgcgctaagcgtggccaccc

SEQ ID NO: 661 - DNA, 28 bp, Artificial Sequence
  synthesized nucleic acid probe to detect Enterobacteriaceae_661
gcggctcacgcgctaagcgtggagccgc

SEQ ID NO: 662 - DNA, 28 bp, Artificial Sequence
  synthesized nucleic acid probe to detect Enterobacteriaceae_662
gaccgccacgcgctaagcgtgggcggtc

SEQ ID NO: 663 - DNA, 28 bp, Artificial Sequence
  synthesized nucleic acid probe to detect Enterobacteriaceae_663
gacggccacgcgctaagcgtgggccgtc

SEQ ID NO: 664 - DNA, 28 bp, Artificial Sequence
  synthesized nucleic acid probe to detect Enterobacteriaceae_664
ggcccacacgcgctaagcgtggtgggcc

SEQ ID NO: 665 - DNA, 22 bp, Artificial Sequence
  synthesized nucleic acid probe to detect Enterobacteriaceae_665
atgtcgccatacttccactcca

SEQ ID NO: 666 - DNA, 34 bp, Artificial Sequence
  synthesized nucleic acid probe to detect Enterobacteriaceae_666
cgcggaatgtcgccatacttccactccatccgcg

SEQ ID NO: 667 - DNA, 34 bp, Artificial Sequence
  synthesized nucleic acid probe to detect Enterobacteriaceae_667
ctcgcgatgtcgccatacttccactccacgcgag

SEQ ID NO: 668 - DNA, 34 bp, Artificial Sequence
  synthesized nucleic acid probe to detect Enterobacteriaceae_668
ccgaccatgtcgccatacttccactccaggtcgg

SEQ ID NO: 669 - DNA, 34 bp, Artificial Sequence
  synthesized nucleic acid probe to detect Enterobacteriaceae_669
cgacgtatgtcgccatacttccactccaacgtcg

SEQ ID NO: 670 - DNA, 34 bp, Artificial Sequence
  synthesized nucleic acid probe to detect Enterobacteriaceae_670
cccaccatgtcgccatacttccactccaggtggg

SEQ ID NO: 671 - DNA, 34 bp, Artificial Sequence
  synthesized nucleic acid probe to detect Enterobacteriaceae_671
cgccgaatgtcgccatacttccactccatcggcg

SEQ ID NO: 672 - DNA, 34 bp, Artificial Sequence
  synthesized nucleic acid probe to detect Enterobacteriaceae_672
ctggcgatgtcgccatacttccactccacgccag

SEQ ID NO: 673 - DNA, 34 bp, Artificial Sequence
  synthesized nucleic acid probe to detect Enterobacteriaceae_673
ctgccgatgtcgccatacttccactccacggcag

SEQ ID NO: 674 - DNA, 34 bp, Artificial Sequence
  synthesized nucleic acid probe to detect Enterobacteriaceae_674
ccgggtatgtcgccatacttccactccaacccgg

SEQ ID NO: 675 - DNA, 34 bp, Artificial Sequence
  synthesized nucleic acid probe to detect Enterobacteriaceae_675
cgcgctatgtcgccatacttccactccaagcgcg

SEQ ID NO: 676 - DNA, 34 bp, Artificial Sequence
  synthesized nucleic acid probe to detect Enterobacteriaceae_676
gcgcctatgtcgccatacttccactccaaggcgc

SEQ ID NO: 677 - DNA, 34 bp, Artificial Sequence
  synthesized nucleic acid probe to detect Enterobacteriaceae_677
gagcgcatgtcgccatacttccactccagcgctc

SEQ ID NO: 678 - DNA, 34 bp, Artificial Sequence
  synthesized nucleic acid probe to detect Enterobacteriaceae_678
ggctggatgtcgccatacttccactccaccagcc

SEQ ID NO: 679 - DNA, 34 bp, Artificial Sequence
  synthesized nucleic acid probe to detect Enterobacteriaceae_679
gctgcaatgtcgccatacttccactccatgcagc

SEQ ID NO: 680 - DNA, 34 bp, Artificial Sequence
  synthesized nucleic acid probe to detect Enterobacteriaceae_680
gggtggatgtcgccatacttccactccaccaccc

SEQ ID NO: 681 - DNA, 34 bp, Artificial Sequence
  synthesized nucleic acid probe to detect Enterobacteriaceae_681
gcggctatgtcgccatacttccactccaagccgc

SEQ ID NO: 682 - DNA, 34 bp, Artificial Sequence
  synthesized nucleic acid probe to detect Enterobacteriaceae_682
gaccgcatgtcgccatacttccactccagcggtc

SEQ ID NO: 683 - DNA, 34 bp, Artificial Sequence
  synthesized nucleic acid probe to detect Enterobacteriaceae_683
gacggcatgtcgccatacttccactccagccgtc

SEQ ID NO: 684 - DNA, 34 bp, Artificial Sequence
  synthesized nucleic acid probe to detect Enterobacteriaceae_684
ggcccaatgtcgccatacttccactccatgggcc

SEQ ID NO: 685 - DNA, 21 bp, Artificial Sequence
  synthesized nucleic acid probe to detect Enterobacteriaceae_685
gcctttccagaccgcttcatt

SEQ ID NO: 686 - DNA, 33 bp, Artificial Sequence
  synthesized nucleic acid probe to detect Enterobacteriaceae_686
cgcggagcctttccagaccgcttcatttccgcg

SEQ ID NO: 687 - DNA, 33 bp, Artificial Sequence
  synthesized nucleic acid probe to detect Enterobacteriaceae_687
ctcgcggcctttccagaccgcttcattcgcgag

SEQ ID NO: 688 - DNA, 33 bp, Artificial Sequence
  synthesized nucleic acid probe to detect Enterobacteriaceae_688
ccgaccgcctttccagaccgcttcattggtcgg

SEQ ID NO: 689 - DNA, 33 bp, Artificial Sequence
  synthesized nucleic acid probe to detect Enterobacteriaceae_689
cgacgtgcctttccagaccgcttcattacgtcg

SEQ ID NO: 690 - DNA, 32 bp, Artificial Sequence
  synthesized nucleic acid probe to detect Enterobacteriaceae_690
ctcccgcctttccagaccgcttcattcgggag

SEQ ID NO: 691 - DNA, 33 bp, Artificial Sequence
  synthesized nucleic acid probe to detect Enterobacteriaceae_691
cccaccgcctttccagaccgcttcattggtggg

SEQ ID NO: 692 - DNA, 33 bp, Artificial Sequence
  synthesized nucleic acid probe to detect Enterobacteriaceae_692
cgccgagcctttccagaccgcttcatttcggcg

SEQ ID NO: 693 - DNA, 33 bp, Artificial Sequence
  synthesized nucleic acid probe to detect Enterobacteriaceae_693
ctggcggcctttccagaccgcttcattcgccag

SEQ ID NO: 694 - DNA, 33 bp, Artificial Sequence
  synthesized nucleic acid probe to detect Enterobacteriaceae_694
ctgccggcctttccagaccgcttcattcggcag

SEQ ID NO: 695 - DNA, 33 bp, Artificial Sequence
  synthesized nucleic acid probe to detect Enterobacteriaceae_695
ccgggtgcctttccagaccgcttcattacccgg

SEQ ID NO: 696 - DNA, 33 bp, Artificial Sequence
  synthesized nucleic acid probe to detect Enterobacteriaceae_696
gcgcctgcctttccagaccgcttcattaggcgc

SEQ ID NO: 697 - DNA, 33 bp, Artificial Sequence
  synthesized nucleic acid probe to detect Enterobacteriaceae_697
gagcgcgcctttccagaccgcttcattgcgctc

SEQ ID NO: 698 - DNA, 33 bp, Artificial Sequence
  synthesized nucleic acid probe to detect Enterobacteriaceae_698
ggctgggcctttccagaccgcttcattccagcc

SEQ ID NO: 699 - DNA, 33 bp, Artificial Sequence
  synthesized nucleic acid probe to detect Enterobacteriaceae_699
gctgcagcctttccagaccgcttcatttgcagc

SEQ ID NO: 700 - DNA, 33 bp, Artificial Sequence
  synthesized nucleic acid probe to detect Enterobacteriaceae_700
gggtgggcctttccagaccgcttcattccaccc

SEQ ID NO: 701 - DNA, 33 bp, Artificial Sequence
  synthesized nucleic acid probe to detect Enterobacteriaceae_701
gcggctgcctttccagaccgcttcattagccgc

SEQ ID NO: 702 - DNA, 33 bp, Artificial Sequence
  synthesized nucleic acid probe to detect Enterobacteriaceae_702
gaccgcgcctttccagaccgcttcattgcggtc

SEQ ID NO: 703 - DNA, 33 bp, Artificial Sequence
  synthesized nucleic acid probe to detect Enterobacteriaceae_703
gacggcgcctttccagaccgcttcattgccgtc

SEQ ID NO: 704 - DNA, 33 bp, Artificial Sequence
  synthesized nucleic acid probe to detect Enterobacteriaceae_704
ggcccagcctttccagaccgcttcatttgggcc

SEQ ID NO: 705 - DNA, 22 bp, Artificial Sequence
  synthesized nucleic acid probe to detect Enterobacteriaceae_705
tcgggtctacgacctgttactt

SEQ ID NO: 706 - DNA, 34 bp, Artificial Sequence
  synthesized nucleic acid probe to detect Enterobacteriaceae_706
cgcggatcgggtctacgacctgttactttccgcg

SEQ ID NO: 707 - DNA, 34 bp, Artificial Sequence
  synthesized nucleic acid probe to detect Enterobacteriaceae_707
ctcgcgtcgggtctacgacctgttacttcgcgag

SEQ ID NO: 708 - DNA, 34 bp, Artificial Sequence
  synthesized nucleic acid probe to detect Enterobacteriaceae_708
ccgacctcgggtctacgacctgttacttggtcgg

SEQ ID NO: 709 - DNA, 34 bp, Artificial Sequence
  synthesized nucleic acid probe to detect Enterobacteriaceae_709
cgacgttcgggtctacgacctgttacttacgtcg

SEQ ID NO: 710 - DNA, 34 bp, Artificial Sequence
  synthesized nucleic acid probe to detect Enterobacteriaceae_710
cccacctcgggtctacgacctgttacttggtggg

SEQ ID NO: 711 - DNA, 34 bp, Artificial Sequence
  synthesized nucleic acid probe to detect Enterobacteriaceae_711
cgccgatcgggtctacgacctgttactttcggcg

SEQ ID NO: 712 - DNA, 34 bp, Artificial Sequence
  synthesized nucleic acid probe to detect Enterobacteriaceae_712
ctggcgtcgggtctacgacctgttacttcgccag

SEQ ID NO: 713 - DNA, 34 bp, Artificial Sequence
  synthesized nucleic acid probe to detect Enterobacteriaceae_713
ctgccgtcgggtctacgacctgttacttcggcag

SEQ ID NO: 714 - DNA, 34 bp, Artificial Sequence
  synthesized nucleic acid probe to detect Enterobacteriaceae_714
ccgggttcgggtctacgacctgttacttacccgg

SEQ ID NO: 715 - DNA, 34 bp, Artificial Sequence
  synthesized nucleic acid probe to detect Enterobacteriaceae_715
gcgccttcgggtctacgacctgttacttaggcgc

SEQ ID NO: 716 - DNA, 34 bp, Artificial Sequence
  synthesized nucleic acid probe to detect Enterobacteriaceae_716
gagcgctcgggtctacgacctgttacttgcgctc

SEQ ID NO: 717 - DNA, 34 bp, Artificial Sequence
  synthesized nucleic acid probe to detect Enterobacteriaceae_717
ggctggtcgggtctacgacctgttacttccagcc

SEQ ID NO: 718 - DNA, 34 bp, Artificial Sequence
  synthesized nucleic acid probe to detect Enterobacteriaceae_718
gctgcatcgggtctacgacctgttactttgcagc

SEQ ID NO: 719 - DNA, 34 bp, Artificial Sequence
  synthesized nucleic acid probe to detect Enterobacteriaceae_719
gggtggtcgggtctacgacctgttacttccaccc

SEQ ID NO: 720 - DNA, 34 bp, Artificial Sequence
  synthesized nucleic acid probe to detect Enterobacteriaceae_720
gcggcttcgggtctacgacctgttacttagccgc

SEQ ID NO: 721 - DNA, 34 bp, Artificial Sequence
  synthesized nucleic acid probe to detect Enterobacteriaceae_721
gaccgctcgggtctacgacctgttacttgcggtc

SEQ ID NO: 722 - DNA, 32 bp, Artificial Sequence
  synthesized nucleic acid probe to detect Enterobacteriaceae_722
cccctcgggtctacgacctgttacttgagggg

SEQ ID NO: 723 - DNA, 34 bp, Artificial Sequence
  synthesized nucleic acid probe to detect Enterobacteriaceae_723
gacggctcgggtctacgacctgttacttgccgtc

SEQ ID NO: 724 - DNA, 34 bp, Artificial Sequence
  synthesized nucleic acid probe to detect Enterobacteriaceae_724
ggcccatcgggtctacgacctgttactttgggcc

SEQ ID NO: 725 - DNA, 24 bp, Artificial Sequence
  synthesized nucleic acid probe to detect Enterobacteriaceae_725
accttagatattcggtggaaggga

SEQ ID NO: 726 - DNA, 36 bp, Artificial Sequence
  synthesized nucleic acid probe to detect Enterobacteriaceae_726
cgcggaaccttagatattcggtggaagggatccgcg

SEQ ID NO: 727 - DNA, 36 bp, Artificial Sequence
  synthesized nucleic acid probe to detect Enterobacteriaceae_727
ctcgcgaccttagatattcggtggaagggacgcgag

SEQ ID NO: 728 - DNA, 36 bp, Artificial Sequence
  synthesized nucleic acid probe to detect Enterobacteriaceae_728
ccgaccaccttagatattcggtggaagggaggtcgg

SEQ ID NO: 729 - DNA, 36 bp, Artificial Sequence
  synthesized nucleic acid probe to detect Enterobacteriaceae_729
cgacgtaccttagatattcggtggaagggaacgtcg

SEQ ID NO: 730 - DNA, 36 bp, Artificial Sequence
  synthesized nucleic acid probe to detect Enterobacteriaceae_730
cccaccaccttagatattcggtggaagggaggtggg

SEQ ID NO: 731 - DNA, 36 bp, Artificial Sequence
  synthesized nucleic acid probe to detect Enterobacteriaceae_731
cgccgaaccttagatattcggtggaagggatcggcg

SEQ ID NO: 732 - DNA, 36 bp, Artificial Sequence
  synthesized nucleic acid probe to detect Enterobacteriaceae_732
ctggcgaccttagatattcggtggaagggacgccag

SEQ ID NO: 733 - DNA, 36 bp, Artificial Sequence
  synthesized nucleic acid probe to detect Enterobacteriaceae_733
ctgccgaccttagatattcggtggaagggacggcag

SEQ ID NO: 734 - DNA, 36 bp, Artificial Sequence
  synthesized nucleic acid probe to detect Enterobacteriaceae_734
ccgggtaccttagatattcggtggaagggaacccgg

SEQ ID NO: 735 - DNA, 36 bp, Artificial Sequence
  synthesized nucleic acid probe to detect Enterobacteriaceae_735
gcgcctaccttagatattcggtggaagggaaggcgc

SEQ ID NO: 736 - DNA, 36 bp, Artificial Sequence
  synthesized nucleic acid probe to detect Enterobacteriaceae_736
gagcgcaccttagatattcggtggaagggagcgctc

SEQ ID NO: 737 - DNA, 36 bp, Artificial Sequence
  synthesized nucleic acid probe to detect Enterobacteriaceae_737
ggctggaccttagatattcggtggaagggaccagcc

SEQ ID NO: 738 - DNA, 36 bp, Artificial Sequence
  synthesized nucleic acid probe to detect Enterobacteriaceae_738
gctgcaaccttagatattcggtggaagggatgcagc

SEQ ID NO: 739 - DNA, 36 bp, Artificial Sequence
  synthesized nucleic acid probe to detect Enterobacteriaceae_739
gggtggaccttagatattcggtggaagggaccaccc

SEQ ID NO: 740 - DNA, 36 bp, Artificial Sequence
  synthesized nucleic acid probe to detect Enterobacteriaceae_740
gcggctaccttagatattcggtggaagggaagccgc

SEQ ID NO: 741 - DNA, 36 bp, Artificial Sequence
  synthesized nucleic acid probe to detect Enterobacteriaceae_741
gaccgcaccttagatattcggtggaagggagcggtc

SEQ ID NO: 742 - DNA, 31 bp, Artificial Sequence
  synthesized nucleic acid probe to detect Enterobacteriaceae_742
ccaccttagatattcggtggaagggaggtgg

SEQ ID NO: 743 - DNA, 36 bp, Artificial Sequence
  synthesized nucleic acid probe to detect Enterobacteriaceae_743
gacggcaccttagatattcggtggaagggagccgtc

SEQ ID NO: 744 - DNA, 36 bp, Artificial Sequence
  synthesized nucleic acid probe to detect Enterobacteriaceae_744
ggcccaaccttagatattcggtggaagggatgggcc

SEQ ID NO: 745 - DNA, 24 bp, Artificial Sequence
  synthesized nucleic acid probe to detect Enterobacteriaceae_745
gaacttccgtacttaatttccttc

SEQ ID NO: 746 - DNA, 36 bp, Artificial Sequence
  synthesized nucleic acid probe to detect Enterobacteriaceae_746
cgcggagaacttccgtacttaatttccttctccgcg

SEQ ID NO: 747 - DNA, 36 bp, Artificial Sequence
  synthesized nucleic acid probe to detect Enterobacteriaceae_747
ctcgcggaacttccgtacttaatttccttccgcgag

SEQ ID NO: 748 - DNA, 36 bp, Artificial Sequence
  synthesized nucleic acid probe to detect Enterobacteriaceae_748
ccgaccgaacttccgtacttaatttccttcggtcgg

SEQ ID NO: 749 - DNA, 36 bp, Artificial Sequence
  synthesized nucleic acid probe to detect Enterobacteriaceae_749
cgacgtgaacttccgtacttaatttccttcacgtcg

SEQ ID NO: 750 - DNA, 36 bp, Artificial Sequence
  synthesized nucleic acid probe to detect Enterobacteriaceae_750
cccaccgaacttccgtacttaatttccttcggtggg

SEQ ID NO: 751 - DNA, 36 bp, Artificial Sequence
  synthesized nucleic acid probe to detect Enterobacteriaceae_751
cgccgagaacttccgtacttaatttccttctcggcg

SEQ ID NO: 752 - DNA, 36 bp, Artificial Sequence
  synthesized nucleic acid probe to detect Enterobacteriaceae_752
ctggcggaacttccgtacttaatttccttccgccag

SEQ ID NO: 753 - DNA, 36 bp, Artificial Sequence
  synthesized nucleic acid probe to detect Enterobacteriaceae_753
ctgccggaacttccgtacttaatttccttccggcag

SEQ ID NO: 754 - DNA, 36 bp, Artificial Sequence
  synthesized nucleic acid probe to detect Enterobacteriaceae_754
ccgggtgaacttccgtacttaatttccttcacccgg

SEQ ID NO: 755 - DNA, 36 bp, Artificial Sequence
  synthesized nucleic acid probe to detect Enterobacteriaceae_755
gcgcctgaacttccgtacttaatttccttcaggcgc

SEQ ID NO: 756 - DNA, 36 bp, Artificial Sequence
  synthesized nucleic acid probe to detect Enterobacteriaceae_756
gagcgcgaacttccgtacttaatttccttcgcgctc

SEQ ID NO: 757 - DNA, 36 bp, Artificial Sequence
  synthesized nucleic acid probe to detect Enterobacteriaceae_757
ggctgggaacttccgtacttaatttccttcccagcc

SEQ ID NO: 758 - DNA, 36 bp, Artificial Sequence
  synthesized nucleic acid probe to detect Enterobacteriaceae_758
gctgcagaacttccgtacttaatttccttctgcagc

SEQ ID NO: 759 - DNA, 36 bp, Artificial Sequence
  synthesized nucleic acid probe to detect Enterobacteriaceae_759
gggtgggaacttccgtacttaatttccttcccaccc

SEQ ID NO: 760 - DNA, 36 bp, Artificial Sequence
  synthesized nucleic acid probe to detect Enterobacteriaceae_760
gcggctgaacttccgtacttaatttccttcagccgc

SEQ ID NO: 761 - DNA, 36 bp, Artificial Sequence
  synthesized nucleic acid probe to detect Enterobacteriaceae_761
gaccgcgaacttccgtacttaatttccttcgcggtc

SEQ ID NO: 762 - DNA, 36 bp, Artificial Sequence
  synthesized nucleic acid probe to detect Enterobacteriaceae_762
gacggcgaacttccgtacttaatttccttcgccgtc

SEQ ID NO: 763 - DNA, 36 bp, Artificial Sequence
  synthesized nucleic acid probe to detect Enterobacteriaceae_763
ggcccagaacttccgtacttaatttccttctgggcc

SEQ ID NO: 764 - DNA, 34 bp, Artificial Sequence
  synthesized nucleic acid probe to detect Enterobacteriaceae_764
ccttcgaacttccgtacttaatttccttcgaagg

SEQ ID NO: 765 - DNA, 20 bp, Artificial Sequence
  synthesized nucleic acid probe to detect Enterobacteriaceae_765
tttgcggtacgggcagtact

SEQ ID NO: 766 - DNA, 32 bp, Artificial Sequence
  synthesized nucleic acid probe to detect Enterobacteriaceae_766
cgcggatttgcggtacgggcagtacttccgcg

SEQ ID NO: 767 - DNA, 32 bp, Artificial Sequence
  synthesized nucleic acid probe to detect Enterobacteriaceae_767
ctcgcgtttgcggtacgggcagtactcgcgag

SEQ ID NO: 768 - DNA, 32 bp, Artificial Sequence
  synthesized nucleic acid probe to detect Enterobacteriaceae_768
ccgacctttgcggtacgggcagtactggtcgg

SEQ ID NO: 769 - DNA, 32 bp, Artificial Sequence
  synthesized nucleic acid probe to detect Enterobacteriaceae_769
cgacgttttgcggtacgggcagtactacgtcg

SEQ ID NO: 770 - DNA, 32 bp, Artificial Sequence
  synthesized nucleic acid probe to detect Enterobacteriaceae_770
cccacctttgcggtacgggcagtactggtggg

SEQ ID NO: 771 - DNA, 32 bp, Artificial Sequence
  synthesized nucleic acid probe to detect Enterobacteriaceae_771
cgccgatttgcggtacgggcagtacttcggcg

SEQ ID NO: 772 - DNA, 32 bp, Artificial Sequence
  synthesized nucleic acid probe to detect Enterobacteriaceae_772
ctggcgtttgcggtacgggcagtactcgccag

SEQ ID NO: 773 - DNA, 32 bp, Artificial Sequence
  synthesized nucleic acid probe to detect Enterobacteriaceae_773
ctgccgtttgcggtacgggcagtactcggcag

SEQ ID NO: 774 - DNA, 32 bp, Artificial Sequence
  synthesized nucleic acid probe to detect Enterobacteriaceae_774
ccgggttttgcggtacgggcagtactacccgg

SEQ ID NO: 775 - DNA, 32 bp, Artificial Sequence
  synthesized nucleic acid probe to detect Enterobacteriaceae_775
gcgccttttgcggtacgggcagtactaggcgc

SEQ ID NO: 776 - DNA, 25 bp, Artificial Sequence
  synthesized nucleic acid probe to detect Enterobacteriaceae_776
ccagtagcggtacgggcagtactgg

SEQ ID NO: 777 - DNA, 32 bp, Artificial Sequence
  synthesized nucleic acid probe to detect Enterobacteriaceae_777
gagcgctttgcggtacgggcagtactgcgctc

SEQ ID NO: 778 - DNA, 32 bp, Artificial Sequence
  synthesized nucleic acid probe to detect Enterobacteriaceae_778
ggctggtttgcggtacgggcagtactccagcc

SEQ ID NO: 779 - DNA, 32 bp, Artificial Sequence
  synthesized nucleic acid probe to detect Enterobacteriaceae_779
gctgcatttgcggtacgggcagtacttgcagc

SEQ ID NO: 780 - DNA, 32 bp, Artificial Sequence
  synthesized nucleic acid probe to detect Enterobacteriaceae_780
gggtggtttgcggtacgggcagtactccaccc

SEQ ID NO: 781 - DNA, 32 bp, Artificial Sequence
  synthesized nucleic acid probe to detect Enterobacteriaceae_781
gcggcttttgcggtacgggcagtactagccgc

SEQ ID NO: 782 - DNA, 32 bp, Artificial Sequence
  synthesized nucleic acid probe to detect Enterobacteriaceae_782
gaccgctttgcggtacgggcagtactgcggtc

SEQ ID NO: 783 - DNA, 32 bp, Artificial Sequence
  synthesized nucleic acid probe to detect Enterobacteriaceae_783
gacggctttgcggtacgggcagtactgccgtc

SEQ ID NO: 784 - DNA, 32 bp, Artificial Sequence
  synthesized nucleic acid probe to detect Enterobacteriaceae_784
ggcccatttgcggtacgggcagtacttgggcc

SEQ ID NO: 785 - DNA, 20 bp, Artificial Sequence
  synthesized nucleic acid probe to detect Enterobacteriaceae_785
ggcagtactactcttcctac

SEQ ID NO: 786 - DNA, 32 bp, Artificial Sequence
  synthesized nucleic acid probe to detect Enterobacteriaceae_786
cgcggaggcagtactactcttcctactccgcg

SEQ ID NO: 787 - DNA, 32 bp, Artificial Sequence
  synthesized nucleic acid probe to detect Enterobacteriaceae_787
ctcgcgggcagtactactcttcctaccgcgag

SEQ ID NO: 788 - DNA, 32 bp, Artificial Sequence
  synthesized nucleic acid probe to detect Enterobacteriaceae_788
ccgaccggcagtactactcttcctacggtcgg

SEQ ID NO: 789 - DNA, 32 bp, Artificial Sequence
  synthesized nucleic acid probe to detect Enterobacteriaceae_789
cgacgtggcagtactactcttcctacacgtcg

SEQ ID NO: 790 - DNA, 32 bp, Artificial Sequence
  synthesized nucleic acid probe to detect Enterobacteriaceae_790
cccaccggcagtactactcttcctacggtggg

SEQ ID NO: 791 - DNA, 32 bp, Artificial Sequence
  synthesized nucleic acid probe to detect Enterobacteriaceae_791
cgccgaggcagtactactcttcctactcggcg

SEQ ID NO: 792 - DNA, 32 bp, Artificial Sequence
  synthesized nucleic acid probe to detect Enterobacteriaceae_792
ctggcgggcagtactactcttcctaccgccag

SEQ ID NO: 793 - DNA, 32 bp, Artificial Sequence
  synthesized nucleic acid probe to detect Enterobacteriaceae_793
ctgccgggcagtactactcttcctaccggcag

SEQ ID NO: 794 - DNA, 32 bp, Artificial Sequence
  synthesized nucleic acid probe to detect Enterobacteriaceae_794
ccgggtggcagtactactcttcctacacccgg

SEQ ID NO: 795 - DNA, 32 bp, Artificial Sequence
  synthesized nucleic acid probe to detect Enterobacteriaceae_795
gcgcctggcagtactactcttcctacaggcgc

SEQ ID NO: 796 - DNA, 32 bp, Artificial Sequence
  synthesized nucleic acid probe to detect Enterobacteriaceae_796
gagcgcggcagtactactcttcctacgcgctc

SEQ ID NO: 797 - DNA, 32 bp, Artificial Sequence
  synthesized nucleic acid probe to detect Enterobacteriaceae_797
ggctggggcagtactactcttcctacccagcc

SEQ ID NO: 798 - DNA, 32 bp, Artificial Sequence
  synthesized nucleic acid probe to detect Enterobacteriaceae_798
gctgcaggcagtactactcttcctactgcagc

SEQ ID NO: 799 - DNA, 32 bp, Artificial Sequence
  synthesized nucleic acid probe to detect Enterobacteriaceae_799
gggtggggcagtactactcttcctacccaccc

SEQ ID NO: 800 - DNA, 30 bp, Artificial Sequence
  synthesized nucleic acid probe to detect Enterobacteriaceae_800
cccctggcagtactactcttcctacagggg

SEQ ID NO: 801 - DNA, 32 bp, Artificial Sequence
  synthesized nucleic acid probe to detect Enterobacteriaceae_801
gcggctggcagtactactcttcctacagccgc

SEQ ID NO: 802 - DNA, 32 bp, Artificial Sequence
  synthesized nucleic acid probe to detect Enterobacteriaceae_802
gaccgcggcagtactactcttcctacgcggtc

SEQ ID NO: 803 - DNA, 32 bp, Artificial Sequence
  synthesized nucleic acid probe to detect Enterobacteriaceae_803
gacggcggcagtactactcttcctacgccgtc

SEQ ID NO: 804 - DNA, 32 bp, Artificial Sequence
  synthesized nucleic acid probe to detect Enterobacteriaceae_804
ggcccaggcagtactactcttcctactgggcc

SEQ ID NO: 805 - DNA, 25 bp, Artificial Sequence
  synthesized nucleic acid probe to detect Enterobacteriaceae_805
ggtacaaacattccatatcaggttg

SEQ ID NO: 806 - DNA, 37 bp, Artificial Sequence
  synthesized nucleic acid probe to detect Enterobacteriaceae_806
cgcggaggtacaaacattccatatcaggttgtccgcg

SEQ ID NO: 807 - DNA, 37 bp, Artificial Sequence
  synthesized nucleic acid probe to detect Enterobacteriaceae_807
ctcgcgggtacaaacattccatatcaggttgcgcgag

SEQ ID NO: 808 - DNA, 37 bp, Artificial Sequence
  synthesized nucleic acid probe to detect Enterobacteriaceae_808
ccgaccggtacaaacattccatatcaggttgggtcgg

SEQ ID NO: 809 - DNA, 37 bp, Artificial Sequence
  synthesized nucleic acid probe to detect Enterobacteriaceae_809
cgacgtggtacaaacattccatatcaggttgacgtcg

SEQ ID NO: 810 - DNA, 37 bp, Artificial Sequence
  synthesized nucleic acid probe to detect Enterobacteriaceae_810
cccaccggtacaaacattccatatcaggttgggtggg

SEQ ID NO: 811 - DNA, 37 bp, Artificial Sequence
  synthesized nucleic acid probe to detect Enterobacteriaceae_811
cgccgaggtacaaacattccatatcaggttgtcggcg

SEQ ID NO: 812 - DNA, 37 bp, Artificial Sequence
  synthesized nucleic acid probe to detect Enterobacteriaceae_812
ctggcgggtacaaacattccatatcaggttgcgccag

SEQ ID NO: 813 - DNA, 37 bp, Artificial Sequence
  synthesized nucleic acid probe to detect Enterobacteriaceae_813
ctgccgggtacaaacattccatatcaggttgcggcag

SEQ ID NO: 814 - DNA, 37 bp, Artificial Sequence
  synthesized nucleic acid probe to detect Enterobacteriaceae_814
ccgggtggtacaaacattccatatcaggttgacccgg

SEQ ID NO: 815 - DNA, 37 bp, Artificial Sequence
  synthesized nucleic acid probe to detect Enterobacteriaceae_815
gcgcctggtacaaacattccatatcaggttgaggcgc

SEQ ID NO: 816 - DNA, 37 bp, Artificial Sequence
  synthesized nucleic acid probe to detect Enterobacteriaceae_816
gagcgcggtacaaacattccatatcaggttggcgctc

SEQ ID NO: 817 - DNA, 37 bp, Artificial Sequence
  synthesized nucleic acid probe to detect Enterobacteriaceae_817
ggctggggtacaaacattccatatcaggttgccagcc

SEQ ID NO: 818 - DNA, 37 bp, Artificial Sequence
  synthesized nucleic acid probe to detect Enterobacteriaceae_818
gctgcaggtacaaacattccatatcaggttgtgcagc

SEQ ID NO: 819 - DNA, 37 bp, Artificial Sequence
  synthesized nucleic acid probe to detect Enterobacteriaceae_819
gggtggggtacaaacattccatatcaggttgccaccc

SEQ ID NO: 820 - DNA, 37 bp, Artificial Sequence
  synthesized nucleic acid probe to detect Enterobacteriaceae_820
gcggctggtacaaacattccatatcaggttgagccgc

SEQ ID NO: 821 - DNA, 37 bp, Artificial Sequence
  synthesized nucleic acid probe to detect Enterobacteriaceae_821
gaccgcggtacaaacattccatatcaggttggcggtc

SEQ ID NO: 822 - DNA, 35 bp, Artificial Sequence
  synthesized nucleic acid probe to detect Enterobacteriaceae_822
cccccaggtacaaacattccatatcaggttggggg

SEQ ID NO: 823 - DNA, 37 bp, Artificial Sequence
  synthesized nucleic acid probe to detect Enterobacteriaceae_823
gacggcggtacaaacattccatatcaggttggccgtc

SEQ ID NO: 824 - DNA, 37 bp, Artificial Sequence
  synthesized nucleic acid probe to detect Enterobacteriaceae_824
ggcccaggtacaaacattccatatcaggttgtgggcc

SEQ ID NO: 825 - DNA, 19 bp, Artificial Sequence
  synthesized nucleic acid probe to detect Enterobacteriaceae_825
cgcgaccctttgtactatc

SEQ ID NO: 826 - DNA, 31 bp, Artificial Sequence
  synthesized nucleic acid probe to detect Enterobacteriaceae_826
cgcggacgcgaccctttgtactatctccgcg

SEQ ID NO: 827 - DNA, 31 bp, Artificial Sequence
  synthesized nucleic acid probe to detect Enterobacteriaceae_827
ctcgcgcgcgaccctttgtactatccgcgag

SEQ ID NO: 828 - DNA, 31 bp, Artificial Sequence
  synthesized nucleic acid probe to detect Enterobacteriaceae_828
ccgacccgcgaccctttgtactatcggtcgg

SEQ ID NO: 829 - DNA, 31 bp, Artificial Sequence
  synthesized nucleic acid probe to detect Enterobacteriaceae_829
cgacgtcgcgaccctttgtactatcacgtcg

SEQ ID NO: 830 - DNA, 31 bp, Artificial Sequence
  synthesized nucleic acid probe to detect Enterobacteriaceae_830
cccacccgcgaccctttgtactatcggtggg

SEQ ID NO: 831 - DNA, 31 bp, Artificial Sequence
  synthesized nucleic acid probe to detect Enterobacteriaceae_831
cgccgacgcgaccctttgtactatctcggcg

SEQ ID NO: 832 - DNA, 31 bp, Artificial Sequence
  synthesized nucleic acid probe to detect Enterobacteriaceae_832
ctggcgcgcgaccctttgtactatccgccag

SEQ ID NO: 833 - DNA, 31 bp, Artificial Sequence
  synthesized nucleic acid probe to detect Enterobacteriaceae_833
ctgccgcgcgaccctttgtactatccggcag

SEQ ID NO: 834 - DNA, 31 bp, Artificial Sequence
  synthesized nucleic acid probe to detect Enterobacteriaceae_834
ccgggtcgcgaccctttgtactatcacccgg

SEQ ID NO: 835 - DNA, 31 bp, Artificial Sequence
  synthesized nucleic acid probe to detect Enterobacteriaceae_835
cccccccgcgaccctttgtactatcgggggg

SEQ ID NO: 836 - DNA, 31 bp, Artificial Sequence
  synthesized nucleic acid probe to detect Enterobacteriaceae_836
gcgcctcgcgaccctttgtactatcaggcgc

SEQ ID NO: 837 - DNA, 31 bp, Artificial Sequence
  synthesized nucleic acid probe to detect Enterobacteriaceae_837
gagcgccgcgaccctttgtactatcgcgctc

SEQ ID NO: 838 - DNA, 31 bp, Artificial Sequence
  synthesized nucleic acid probe to detect Enterobacteriaceae_838
ggctggcgcgaccctttgtactatcccagcc

SEQ ID NO: 839 - DNA, 31 bp, Artificial Sequence
  synthesized nucleic acid probe to detect Enterobacteriaceae_839
gctgcacgcgaccctttgtactatctgcagc

SEQ ID NO: 840 - DNA, 31 bp, Artificial Sequence
  synthesized nucleic acid probe to detect Enterobacteriaceae_840
gggtggcgcgaccctttgtactatcccaccc

SEQ ID NO: 841 - DNA, 31 bp, Artificial Sequence
  synthesized nucleic acid probe to detect Enterobacteriaceae_841
gcggctcgcgaccctttgtactatcagccgc

SEQ ID NO: 842 - DNA, 31 bp, Artificial Sequence
  synthesized nucleic acid probe to detect Enterobacteriaceae_842
gaccgccgcgaccctttgtactatcgcggtc

SEQ ID NO: 843 - DNA, 31 bp, Artificial Sequence
  synthesized nucleic acid probe to detect Enterobacteriaceae_843
gacggccgcgaccctttgtactatcgccgtc

SEQ ID NO: 844 - DNA, 31 bp, Artificial Sequence
  synthesized nucleic acid probe to detect Enterobacteriaceae_844
ggcccacgcgaccctttgtactatctgggcc

SEQ ID NO: 845 - DNA, 25 bp, Artificial Sequence
  synthesized nucleic acid probe to detect Enterobacteriaceae_845
aatagttttatgggattagctccac

SEQ ID NO: 846 - DNA, 37 bp, Artificial Sequence
  synthesized nucleic acid probe to detect Enterobacteriaceae_846
cgcggaaatagttttatgggattagctccactccgcg

SEQ ID NO: 847 - DNA, 37 bp, Artificial Sequence
  synthesized nucleic acid probe to detect Enterobacteriaceae_847
ctcgcgaatagttttatgggattagctccaccgcgag

SEQ ID NO: 848 - DNA, 37 bp, Artificial Sequence
  synthesized nucleic acid probe to detect Enterobacteriaceae_848
ccgaccaatagttttatgggattagctccacggtcgg

SEQ ID NO: 849 - DNA, 37 bp, Artificial Sequence
  synthesized nucleic acid probe to detect Enterobacteriaceae_849
cgacgtaatagttttatgggattagctccacacgtcg

SEQ ID NO: 850 - DNA, 37 bp, Artificial Sequence
  synthesized nucleic acid probe to detect Enterobacteriaceae_850
cccaccaatagttttatgggattagctccacggtggg

SEQ ID NO: 851 - DNA, 37 bp, Artificial Sequence
  synthesized nucleic acid probe to detect Enterobacteriaceae_851
cgccgaaatagttttatgggattagctccactcggcg

SEQ ID NO: 852 - DNA, 37 bp, Artificial Sequence
  synthesized nucleic acid probe to detect Enterobacteriaceae_852
ctggcgaatagttttatgggattagctccaccgccag

SEQ ID NO: 853 - DNA, 37 bp, Artificial Sequence
  synthesized nucleic acid probe to detect Enterobacteriaceae_853
ctgccgaatagttttatgggattagctccaccggcag

SEQ ID NO: 854 - DNA, 37 bp, Artificial Sequence
  synthesized nucleic acid probe to detect Enterobacteriaceae_854
ccgggtaatagttttatgggattagctccacacccgg

SEQ ID NO: 855 - DNA, 37 bp, Artificial Sequence
  synthesized nucleic acid probe to detect Enterobacteriaceae_855
ccccccaatagttttatgggattagctccacgggggg

SEQ ID NO: 856 - DNA, 33 bp, Artificial Sequence
  synthesized nucleic acid probe to detect Enterobacteriaceae_856
ccgtggaatagttttatgggattagctccacgg

SEQ ID NO: 857 - DNA, 37 bp, Artificial Sequence
  synthesized nucleic acid probe to detect Enterobacteriaceae_857
gcgcctaatagttttatgggattagctccacaggcgc

SEQ ID NO: 858 - DNA, 37 bp, Artificial Sequence
  synthesized nucleic acid probe to detect Enterobacteriaceae_858
gagcgcaatagttttatgggattagctccacgcgctc

SEQ ID NO: 859 - DNA, 37 bp, Artificial Sequence
  synthesized nucleic acid probe to detect Enterobacteriaceae_859
ggctggaatagttttatgggattagctccacccagcc

SEQ ID NO: 860 - DNA, 37 bp, Artificial Sequence
  synthesized nucleic acid probe to detect Enterobacteriaceae_860
gctgcaaatagttttatgggattagctccactgcagc

SEQ ID NO: 861 - DNA, 37 bp, Artificial Sequence
  synthesized nucleic acid probe to detect Enterobacteriaceae_861
gggtggaatagttttatgggattagctccacccaccc

SEQ ID NO: 862 - DNA, 37 bp, Artificial Sequence
  synthesized nucleic acid probe to detect Enterobacteriaceae_862
gcggctaatagttttatgggattagctccacagccgc

SEQ ID NO: 863 - DNA, 37 bp, Artificial Sequence
  synthesized nucleic acid probe to detect Enterobacteriaceae_863
gaccgcaatagttttatgggattagctccacgcggtc

SEQ ID NO: 864 - DNA, 37 bp, Artificial Sequence
  synthesized nucleic acid probe to detect Enterobacteriaceae_864
gacggcaatagttttatgggattagctccacgccgtc

SEQ ID NO: 865 - DNA, 37 bp, Artificial Sequence
  synthesized nucleic acid probe to detect Enterobacteriaceae_865
ggcccaaatagttttatgggattagctccactgggcc

SEQ ID NO: 866 - DNA, 21 bp, Artificial Sequence
  synthesized nucleic acid probe to detect Enterobacteriaceae_866
gaaaccatctttcaaaagcgt

SEQ ID NO: 867 - DNA, 33 bp, Artificial Sequence
  synthesized nucleic acid probe to detect Enterobacteriaceae_867
cgcggagaaaccatctttcaaaagcgttccgcg

SEQ ID NO: 868 - DNA, 33 bp, Artificial Sequence
  synthesized nucleic acid probe to detect Enterobacteriaceae_868
ctcgcggaaaccatctttcaaaagcgtcgcgag

SEQ ID NO: 869 - DNA, 30 bp, Artificial Sequence
  synthesized nucleic acid probe to detect Enterobacteriaceae_869
ccccgaaaccatctttcaaaagcgtcgggg

SEQ ID NO: 870 - DNA, 33 bp, Artificial Sequence
  synthesized nucleic acid probe to detect Enterobacteriaceae_870
ccgaccgaaaccatctttcaaaagcgtggtcgg

SEQ ID NO: 871 - DNA, 33 bp, Artificial Sequence
  synthesized nucleic acid probe to detect Enterobacteriaceae_871
cgacgtgaaaccatctttcaaaagcgtacgtcg

SEQ ID NO: 872 - DNA, 33 bp, Artificial Sequence
  synthesized nucleic acid probe to detect Enterobacteriaceae_872
cccaccgaaaccatctttcaaaagcgtggtggg

SEQ ID NO: 873 - DNA, 33 bp, Artificial Sequence
  synthesized nucleic acid probe to detect Enterobacteriaceae_873
cgccgagaaaccatctttcaaaagcgttcggcg

SEQ ID NO: 874 - DNA, 33 bp, Artificial Sequence
  synthesized nucleic acid probe to detect Enterobacteriaceae_874
ctggcggaaaccatctttcaaaagcgtcgccag

SEQ ID NO: 875 - DNA, 33 bp, Artificial Sequence
  synthesized nucleic acid probe to detect Enterobacteriaceae_875
ctgccggaaaccatctttcaaaagcgtcggcag

SEQ ID NO: 876 - DNA, 33 bp, Artificial Sequence
  synthesized nucleic acid probe to detect Enterobacteriaceae_876
ccgggtgaaaccatctttcaaaagcgtacccgg

SEQ ID NO: 877 - DNA, 33 bp, Artificial Sequence
  synthesized nucleic acid probe to detect Enterobacteriaceae_877
ccccccgaaaccatctttcaaaagcgtgggggg

SEQ ID NO: 878 - DNA, 33 bp, Artificial Sequence
  synthesized nucleic acid probe to detect Enterobacteriaceae_878
gcgcctgaaaccatctttcaaaagcgtaggcgc

SEQ ID NO: 879 - DNA, 33 bp, Artificial Sequence
  synthesized nucleic acid probe to detect Enterobacteriaceae_879
gagcgcgaaaccatctttcaaaagcgtgcgctc

SEQ ID NO: 880 - DNA, 33 bp, Artificial Sequence
  synthesized nucleic acid probe to detect Enterobacteriaceae_880
ggctgggaaaccatctttcaaaagcgtccagcc

SEQ ID NO: 881 - DNA, 33 bp, Artificial Sequence
  synthesized nucleic acid probe to detect Enterobacteriaceae_881
gctgcagaaaccatctttcaaaagcgttgcagc

SEQ ID NO: 882 - DNA, 33 bp, Artificial Sequence
  synthesized nucleic acid probe to detect Enterobacteriaceae_882
gggtgggaaaccatctttcaaaagcgtccaccc

SEQ ID NO: 883 - DNA, 33 bp, Artificial Sequence
  synthesized nucleic acid probe to detect Enterobacteriaceae_883
gcggctgaaaccatctttcaaaagcgtagccgc

SEQ ID NO: 884 - DNA, 33 bp, Artificial Sequence
  synthesized nucleic acid probe to detect Enterobacteriaceae_884
gaccgcgaaaccatctttcaaaagcgtgcggtc

SEQ ID NO: 885 - DNA, 33 bp, Artificial Sequence
  synthesized nucleic acid probe to detect Enterobacteriaceae_885
gacggcgaaaccatctttcaaaagcgtgccgtc

SEQ ID NO: 886 - DNA, 33 bp, Artificial Sequence
  synthesized nucleic acid probe to detect Enterobacteriaceae_886
ggcccagaaaccatctttcaaaagcgttgggcc

SEQ ID NO: 887 - DNA, 14 bp, Artificial Sequence
  synthesized nucleic acid probe to detect Enterobacteriaceae_887
tggcatgcgccaca

SEQ ID NO: 888 - DNA, 26 bp, Artificial Sequence
  synthesized nucleic acid probe to detect Enterobacteriaceae_888
cgcggatggcatgcgccacatccgcg

SEQ ID NO: 889 - DNA, 26 bp, Artificial Sequence
  synthesized nucleic acid probe to detect Enterobacteriaceae_889
ctcgcgtggcatgcgccacacgcgag

SEQ ID NO: 890 - DNA, 26 bp, Artificial Sequence
  synthesized nucleic acid probe to detect Enterobacteriaceae_890
ccgacctggcatgcgccacaggtcgg

SEQ ID NO: 891 - DNA, 26 bp, Artificial Sequence
  synthesized nucleic acid probe to detect Enterobacteriaceae_891
cgacgttggcatgcgccacaacgtcg

SEQ ID NO: 892 - DNA, 26 bp, Artificial Sequence
  synthesized nucleic acid probe to detect Enterobacteriaceae_892
cccacctggcatgcgccacaggtggg

SEQ ID NO: 893 - DNA, 26 bp, Artificial Sequence
  synthesized nucleic acid probe to detect Enterobacteriaceae_893
cgccgatggcatgcgccacatcggcg

SEQ ID NO: 894 - DNA, 26 bp, Artificial Sequence
  synthesized nucleic acid probe to detect Enterobacteriaceae_894
ctggcgtggcatgcgccacacgccag

SEQ ID NO: 895 - DNA, 26 bp, Artificial Sequence
  synthesized nucleic acid probe to detect Enterobacteriaceae_895
ctgccgtggcatgcgccacacggcag

SEQ ID NO: 896 - DNA, 26 bp, Artificial Sequence
  synthesized nucleic acid probe to detect Enterobacteriaceae_896
ccgggttggcatgcgccacaacccgg

SEQ ID NO: 897 - DNA, 26 bp, Artificial Sequence
  synthesized nucleic acid probe to detect Enterobacteriaceae_897
cccccctggcatgcgccacagggggg

SEQ ID NO: 898 - DNA, 26 bp, Artificial Sequence
  synthesized nucleic acid probe to detect Enterobacteriaceae_898
gcgccttggcatgcgccacaaggcgc

SEQ ID NO: 899 - DNA, 26 bp, Artificial Sequence
  synthesized nucleic acid probe to detect Enterobacteriaceae_899
gagcgctggcatgcgccacagcgctc

SEQ ID NO: 900 - DNA, 26 bp, Artificial Sequence
  synthesized nucleic acid probe to detect Enterobacteriaceae_900
ggctggtggcatgcgccacaccagcc

SEQ ID NO: 901 - DNA, 26 bp, Artificial Sequence
  synthesized nucleic acid probe to detect Enterobacteriaceae_901
gctgcatggcatgcgccacatgcagc

SEQ ID NO: 902 - DNA, 26 bp, Artificial Sequence
  synthesized nucleic acid probe to detect Enterobacteriaceae_902
gggtggtggcatgcgccacaccaccc

SEQ ID NO: 903 - DNA, 26 bp, Artificial Sequence
  synthesized nucleic acid probe to detect Enterobacteriaceae_903
gcggcttggcatgcgccacaagccgc

SEQ ID NO: 904 - DNA, 26 bp, Artificial Sequence
  synthesized nucleic acid probe to detect Enterobacteriaceae_904
gaccgctggcatgcgccacagcggtc

SEQ ID NO: 905 - DNA, 26 bp, Artificial Sequence
  synthesized nucleic acid probe to detect Enterobacteriaceae_905
gacggctggcatgcgccacagccgtc

SEQ ID NO: 906 - DNA, 26 bp, Artificial Sequence
  synthesized nucleic acid probe to detect Enterobacteriaceae_906
ggcccatggcatgcgccacatgggcc

SEQ ID NO: 907 - DNA, 22 bp, Artificial Sequence
  synthesized nucleic acid probe to detect Enterobacteriaceae_907
cctggcatgcgccacagccagg

SEQ ID NO: 908 - DNA, 20 bp, Artificial Sequence
  synthesized nucleic acid probe to detect Enterobacteriaceae_908
catgcgccacactttatcat

SEQ ID NO: 909 - DNA, 32 bp, Artificial Sequence
  synthesized nucleic acid probe to detect Enterobacteriaceae_909
cgcggacatgcgccacactttatcattccgcg

SEQ ID NO: 910 - DNA, 32 bp, Artificial Sequence
  synthesized nucleic acid probe to detect Enterobacteriaceae_910
ctcgcgcatgcgccacactttatcatcgcgag

SEQ ID NO: 911 - DNA, 32 bp, Artificial Sequence
  synthesized nucleic acid probe to detect Enterobacteriaceae_911
ccgacccatgcgccacactttatcatggtcgg

SEQ ID NO: 912 - DNA, 32 bp, Artificial Sequence
  synthesized nucleic acid probe to detect Enterobacteriaceae_912
cgacgtcatgcgccacactttatcatacgtcg

SEQ ID NO: 913 - DNA, 32 bp, Artificial Sequence
  synthesized nucleic acid probe to detect Enterobacteriaceae_913
cccacccatgcgccacactttatcatggtggg

SEQ ID NO: 914 - DNA, 32 bp, Artificial Sequence
  synthesized nucleic acid probe to detect Enterobacteriaceae_914
cgccgacatgcgccacactttatcattcggcg

SEQ ID NO: 915 - DNA, 32 bp, Artificial Sequence
  synthesized nucleic acid probe to detect Enterobacteriaceae_915
ctggcgcatgcgccacactttatcatcgccag

SEQ ID NO: 916 - DNA, 32 bp, Artificial Sequence
  synthesized nucleic acid probe to detect Enterobacteriaceae_916
ctgccgcatgcgccacactttatcatcggcag

SEQ ID NO: 917 - DNA, 32 bp, Artificial Sequence
  synthesized nucleic acid probe to detect Enterobacteriaceae_917
ccgggtcatgcgccacactttatcatacccgg

SEQ ID NO: 918 - DNA, 32 bp, Artificial Sequence
  synthesized nucleic acid probe to detect Enterobacteriaceae_918
cccccccatgcgccacactttatcatgggggg

SEQ ID NO: 919 - DNA, 32 bp, Artificial Sequence
  synthesized nucleic acid probe to detect Enterobacteriaceae_919
gcgcctcatgcgccacactttatcataggcgc

SEQ ID NO: 920 - DNA, 32 bp, Artificial Sequence
  synthesized nucleic acid probe to detect Enterobacteriaceae_920
gagcgccatgcgccacactttatcatgcgctc

SEQ ID NO: 921 - DNA, 32 bp, Artificial Sequence
  synthesized nucleic acid probe to detect Enterobacteriaceae_921
ggctggcatgcgccacactttatcatccagcc

SEQ ID NO: 922 - DNA, 32 bp, Artificial Sequence
  synthesized nucleic acid probe to detect Enterobacteriaceae_922
gctgcacatgcgccacactttatcattgcagc

SEQ ID NO: 923 - DNA, 32 bp, Artificial Sequence
  synthesized nucleic acid probe to detect Enterobacteriaceae_923
gggtggcatgcgccacactttatcatccaccc

SEQ ID NO: 924 - DNA, 32 bp, Artificial Sequence
  synthesized nucleic acid probe to detect Enterobacteriaceae_924
gcggctcatgcgccacactttatcatagccgc

SEQ ID NO: 925 - DNA, 32 bp, Artificial Sequence
  synthesized nucleic acid probe to detect Enterobacteriaceae_925
gaccgccatgcgccacactttatcatgcggtc

SEQ ID NO: 926 - DNA, 32 bp, Artificial Sequence
  synthesized nucleic acid probe to detect Enterobacteriaceae_926
gacggccatgcgccacactttatcatgccgtc

SEQ ID NO: 927 - DNA, 32 bp, Artificial Sequence
  synthesized nucleic acid probe to detect Enterobacteriaceae_927
ggcccacatgcgccacactttatcattgggcc

SEQ ID NO: 928 - DNA, 22 bp, Artificial Sequence
  synthesized nucleic acid probe to detect Enterobacteriaceae_928
aagcagttactcttatccttgt

SEQ ID NO: 929 - DNA, 34 bp, Artificial Sequence
  synthesized nucleic acid probe to detect Enterobacteriaceae_929
cgcggaaagcagttactcttatccttgttccgcg

SEQ ID NO: 930 - DNA, 34 bp, Artificial Sequence
  synthesized nucleic acid probe to detect Enterobacteriaceae_930
ctcgcgaagcagttactcttatccttgtcgcgag

SEQ ID NO: 931 - DNA, 34 bp, Artificial Sequence
  synthesized nucleic acid probe to detect Enterobacteriaceae_931
ccgaccaagcagttactcttatccttgtggtcgg

SEQ ID NO: 932 - DNA, 34 bp, Artificial Sequence
  synthesized nucleic acid probe to detect Enterobacteriaceae_932
cgacgtaagcagttactcttatccttgtacgtcg

SEQ ID NO: 933 - DNA, 34 bp, Artificial Sequence
  synthesized nucleic acid probe to detect Enterobacteriaceae_933
cccaccaagcagttactcttatccttgtggtggg

SEQ ID NO: 934 - DNA, 34 bp, Artificial Sequence
  synthesized nucleic acid probe to detect Enterobacteriaceae_934
cgccgaaagcagttactcttatccttgttcggcg

SEQ ID NO: 935 - DNA, 34 bp, Artificial Sequence
  synthesized nucleic acid probe to detect Enterobacteriaceae_935
ctggcgaagcagttactcttatccttgtcgccag

SEQ ID NO: 936 - DNA, 34 bp, Artificial Sequence
  synthesized nucleic acid probe to detect Enterobacteriaceae_936
ctgccgaagcagttactcttatccttgtcggcag

SEQ ID NO: 937 - DNA, 34 bp, Artificial Sequence
  synthesized nucleic acid probe to detect Enterobacteriaceae_937
ccgggtaagcagttactcttatccttgtacccgg

SEQ ID NO: 938 - DNA, 34 bp, Artificial Sequence
  synthesized nucleic acid probe to detect Enterobacteriaceae_938
ccccccaagcagttactcttatccttgtgggggg

SEQ ID NO: 939 - DNA, 34 bp, Artificial Sequence
  synthesized nucleic acid probe to detect Enterobacteriaceae_939
gcgcctaagcagttactcttatccttgtaggcgc

SEQ ID NO: 940 - DNA, 34 bp, Artificial Sequence
  synthesized nucleic acid probe to detect Enterobacteriaceae_940
gagcgcaagcagttactcttatccttgtgcgctc

SEQ ID NO: 941 - DNA, 34 bp, Artificial Sequence
  synthesized nucleic acid probe to detect Enterobacteriaceae_941
ggctggaagcagttactcttatccttgtccagcc

SEQ ID NO: 942 - DNA, 34 bp, Artificial Sequence
  synthesized nucleic acid probe to detect Enterobacteriaceae_942
gctgcaaagcagttactcttatccttgttgcagc

SEQ ID NO: 943 - DNA, 34 bp, Artificial Sequence
  synthesized nucleic acid probe to detect Enterobacteriaceae_943
gggtggaagcagttactcttatccttgtccaccc

SEQ ID NO: 944 - DNA, 34 bp, Artificial Sequence
  synthesized nucleic acid probe to detect Enterobacteriaceae_944
gcggctaagcagttactcttatccttgtagccgc

SEQ ID NO: 945 - DNA, 34 bp, Artificial Sequence
  synthesized nucleic acid probe to detect Enterobacteriaceae_945
gaccgcaagcagttactcttatccttgtgcggtc

SEQ ID NO: 946 - DNA, 34 bp, Artificial Sequence
  synthesized nucleic acid probe to detect Enterobacteriaceae_946
gacggcaagcagttactcttatccttgtgccgtc

SEQ ID NO: 947 - DNA, 34 bp, Artificial Sequence
  synthesized nucleic acid probe to detect Enterobacteriaceae_947
ggcccaaagcagttactcttatccttgttgggcc

SEQ ID NO: 948 - DNA, 20 bp, Artificial Sequence
  synthesized nucleic acid probe to detect Enterobacteriaceae_948
cggagttatccccaacttac

SEQ ID NO: 949 - DNA, 32 bp, Artificial Sequence
  synthesized nucleic acid probe to detect Enterobacteriaceae_949
cgcggacggagttatccccaacttactccgcg

SEQ ID NO: 950 - DNA, 32 bp, Artificial Sequence
  synthesized nucleic acid probe to detect Enterobacteriaceae_950
ctcgcgcggagttatccccaacttaccgcgag

SEQ ID NO: 951 - DNA, 32 bp, Artificial Sequence
  synthesized nucleic acid probe to detect Enterobacteriaceae_951
ccgacccggagttatccccaacttacggtcgg

SEQ ID NO: 952 - DNA, 32 bp, Artificial Sequence
  synthesized nucleic acid probe to detect Enterobacteriaceae_952
cgacgtcggagttatccccaacttacacgtcg

SEQ ID NO: 953 - DNA, 32 bp, Artificial Sequence
  synthesized nucleic acid probe to detect Enterobacteriaceae_953
cccacccggagttatccccaacttacggtggg

SEQ ID NO: 954 - DNA, 32 bp, Artificial Sequence
  synthesized nucleic acid probe to detect Enterobacteriaceae_954
cgccgacggagttatccccaacttactcggcg

SEQ ID NO: 955 - DNA, 32 bp, Artificial Sequence
  synthesized nucleic acid probe to detect Enterobacteriaceae_955
ctggcgcggagttatccccaacttaccgccag

SEQ ID NO: 956 - DNA, 32 bp, Artificial Sequence
  synthesized nucleic acid probe to detect Enterobacteriaceae_956
ctgccgcggagttatccccaacttaccggcag

SEQ ID NO: 957 - DNA, 32 bp, Artificial Sequence
  synthesized nucleic acid probe to detect Enterobacteriaceae_957
ccgggtcggagttatccccaacttacacccgg

SEQ ID NO: 958 - DNA, 32 bp, Artificial Sequence
  synthesized nucleic acid probe to detect Enterobacteriaceae_958
cccccccggagttatccccaacttacgggggg

SEQ ID NO: 959 - DNA, 32 bp, Artificial Sequence
  synthesized nucleic acid probe to detect Enterobacteriaceae_959
gcgcctcggagttatccccaacttacaggcgc

SEQ ID NO: 960 - DNA, 32 bp, Artificial Sequence
  synthesized nucleic acid probe to detect Enterobacteriaceae_960
gagcgccggagttatccccaacttacgcgctc

SEQ ID NO: 961 - DNA, 32 bp, Artificial Sequence
  synthesized nucleic acid probe to detect Enterobacteriaceae_961
ggctggcggagttatccccaacttacccagcc

SEQ ID NO: 962 - DNA, 32 bp, Artificial Sequence
  synthesized nucleic acid probe to detect Enterobacteriaceae_962
gctgcacggagttatccccaacttactgcagc

SEQ ID NO: 963 - DNA, 32 bp, Artificial Sequence
  synthesized nucleic acid probe to detect Enterobacteriaceae_963
gggtggcggagttatccccaacttacccaccc

SEQ ID NO: 964 - DNA, 32 bp, Artificial Sequence
  synthesized nucleic acid probe to detect Enterobacteriaceae_964
gcggctcggagttatccccaacttacagccgc

SEQ ID NO: 965 - DNA, 32 bp, Artificial Sequence
  synthesized nucleic acid probe to detect Enterobacteriaceae_965
gaccgccggagttatccccaacttacgcggtc

SEQ ID NO: 966 - DNA, 32 bp, Artificial Sequence
  synthesized nucleic acid probe to detect Enterobacteriaceae_966
gacggccggagttatccccaacttacgccgtc

SEQ ID NO: 967 - DNA, 32 bp, Artificial Sequence
  synthesized nucleic acid probe to detect Enterobacteriaceae_967
ggcccacggagttatccccaacttactgggcc

SEQ ID NO: 968 - DNA, 22 bp, Artificial Sequence
  synthesized nucleic acid probe to detect Enterobacteriaceae_968
ctccatgtatcgctacacttcc

SEQ ID NO: 969 - DNA, 34 bp, Artificial Sequence
  synthesized nucleic acid probe to detect Enterobacteriaceae_969
cgcggactccatgtatcgctacacttcctccgcg

SEQ ID NO: 970 - DNA, 34 bp, Artificial Sequence
  synthesized nucleic acid probe to detect Enterobacteriaceae_970
ctcgcgctccatgtatcgctacacttcccgcgag

SEQ ID NO: 971 - DNA, 34 bp, Artificial Sequence
  synthesized nucleic acid probe to detect Enterobacteriaceae_971
ccgaccctccatgtatcgctacacttccggtcgg

SEQ ID NO: 972 - DNA, 34 bp, Artificial Sequence
  synthesized nucleic acid probe to detect Enterobacteriaceae_972
cgacgtctccatgtatcgctacacttccacgtcg

SEQ ID NO: 973 - DNA, 34 bp, Artificial Sequence
  synthesized nucleic acid probe to detect Enterobacteriaceae_973
cccaccctccatgtatcgctacacttccggtggg

SEQ ID NO: 974 - DNA, 34 bp, Artificial Sequence
  synthesized nucleic acid probe to detect Enterobacteriaceae_974
cgccgactccatgtatcgctacacttcctcggcg

SEQ ID NO: 975 - DNA, 34 bp, Artificial Sequence
  synthesized nucleic acid probe to detect Enterobacteriaceae_975
ctggcgctccatgtatcgctacacttcccgccag

SEQ ID NO: 976 - DNA, 34 bp, Artificial Sequence
  synthesized nucleic acid probe to detect Enterobacteriaceae_976
ctgccgctccatgtatcgctacacttcccggcag

SEQ ID NO: 977 - DNA, 34 bp, Artificial Sequence
  synthesized nucleic acid probe to detect Enterobacteriaceae_977
ccgggtctccatgtatcgctacacttccacccgg

SEQ ID NO: 978 - DNA, 34 bp, Artificial Sequence
  synthesized nucleic acid probe to detect Enterobacteriaceae_978
ccccccctccatgtatcgctacacttccgggggg

SEQ ID NO: 979 - DNA, 34 bp, Artificial Sequence
  synthesized nucleic acid probe to detect Enterobacteriaceae_979
gcgcctctccatgtatcgctacacttccaggcgc

SEQ ID NO: 980 - DNA, 34 bp, Artificial Sequence
  synthesized nucleic acid probe to detect Enterobacteriaceae_980
gagcgcctccatgtatcgctacacttccgcgctc

SEQ ID NO: 981 - DNA, 34 bp, Artificial Sequence; identical to SEQ ID NO: 975
  synthesized nucleic acid probe to detect Enterobacteriaceae_981

SEQ ID NO: 982 - DNA, 34 bp, Artificial Sequence
  synthesized nucleic acid probe to detect Enterobacteriaceae_982
ggctggctccatgtatcgctacacttccccagcc

SEQ ID NO: 983 - DNA, 34 bp, Artificial Sequence
  synthesized nucleic acid probe to detect Enterobacteriaceae_983
gctgcactccatgtatcgctacacttcctgcagc

SEQ ID NO: 984 - DNA, 34 bp, Artificial Sequence
  synthesized nucleic acid probe to detect Enterobacteriaceae_984
gggtggctccatgtatcgctacacttccccaccc

SEQ ID NO: 985 - DNA, 34 bp, Artificial Sequence
  synthesized nucleic acid probe to detect Enterobacteriaceae_985
gcggctctccatgtatcgctacacttccagccgc

SEQ ID NO: 986 - DNA, 34 bp, Artificial Sequence
  synthesized nucleic acid probe to detect Enterobacteriaceae_986
gaccgcctccatgtatcgctacacttccgcggtc

SEQ ID NO: 987 - DNA, 34 bp, Artificial Sequence
  synthesized nucleic acid probe to detect Enterobacteriaceae_987
gacggcctccatgtatcgctacacttccgccgtc

SEQ ID NO: 988 - DNA, 34 bp, Artificial Sequence
  synthesized nucleic acid probe to detect Enterobacteriaceae_988
ggcccactccatgtatcgctacacttcctgggcc

SEQ ID NO: 989 - DNA, 21 bp, Artificial Sequence
  synthesized nucleic acid probe to detect Enterobacteriaceae_989
atgctagcgcacacgtctctt

SEQ ID NO: 990 - DNA, 33 bp, Artificial Sequence
  synthesized nucleic acid probe to detect Enterobacteriaceae_990
cgcggaatgctagcgcacacgtctctttccgcg

SEQ ID NO: 991 - DNA, 33 bp, Artificial Sequence
  synthesized nucleic acid probe to detect Enterobacteriaceae_991
ctcgcgatgctagcgcacacgtctcttcgcgag

SEQ ID NO: 992 - DNA, 33 bp, Artificial Sequence
  synthesized nucleic acid probe to detect Enterobacteriaceae_992
ccgaccatgctagcgcacacgtctcttggtcgg

SEQ ID NO: 993 - DNA, 33 bp, Artificial Sequence
  synthesized nucleic acid probe to detect Enterobacteriaceae_993
cgacgtatgctagcgcacacgtctcttacgtcg

SEQ ID NO: 994 - DNA, 33 bp, Artificial Sequence
  synthesized nucleic acid probe to detect Enterobacteriaceae_994
cccaccatgctagcgcacacgtctcttggtggg

SEQ ID NO: 995 - DNA, 33 bp, Artificial Sequence
  synthesized nucleic acid probe to detect Enterobacteriaceae_995
cgccgaatgctagcgcacacgtctctttcggcg

SEQ ID NO: 996 - DNA, 33 bp, Artificial Sequence
  synthesized nucleic acid probe to detect Enterobacteriaceae_996
ctggcgatgctagcgcacacgtctcttcgccag

SEQ ID NO: 997 - DNA, 30 bp, Artificial Sequence
  synthesized nucleic acid probe to detect Enterobacteriaceae_997
cccatgctagcgcacacgtctcttcatggg

SEQ ID NO: 998 - DNA, 33 bp, Artificial Sequence
  synthesized nucleic acid probe to detect Enterobacteriaceae_998
ctgccgatgctagcgcacacgtctcttcggcag

SEQ ID NO: 999 - DNA, 33 bp, Artificial Sequence
  synthesized nucleic acid probe to detect Enterobacteriaceae_999
ccgggtatgctagcgcacacgtctcttacccgg

SEQ ID NO: 1000 - DNA, 33 bp, Artificial Sequence
  synthesized nucleic acid probe to detect Enterobacteriaceae_1000
ccccccatgctagcgcacacgtctcttgggggg

SEQ ID NO: 1001 - DNA, 33 bp, Artificial Sequence
  synthesized nucleic acid probe to detect Enterobacteriaceae_1001
gcgcctatgctagcgcacacgtctcttaggcgc

SEQ ID NO: 1002 - DNA, 33 bp, Artificial Sequence
  synthesized nucleic acid probe to detect Enterobacteriaceae_1002
gagcgcatgctagcgcacacgtctcttgcgctc

SEQ ID NO: 1003 - DNA, 33 bp, Artificial Sequence
  synthesized nucleic acid probe to detect Enterobacteriaceae_1003
ggctggatgctagcgcacacgtctcttccagcc

SEQ ID NO: 1004 - DNA, 33 bp, Artificial Sequence
  synthesized nucleic acid probe to detect Enterobacteriaceae_1004
gctgcaatgctagcgcacacgtctctttgcagc

SEQ ID NO: 1005 - DNA, 33 bp, Artificial Sequence
  synthesized nucleic acid probe to detect Enterobacteriaceae_1005
gggtggatgctagcgcacacgtctcttccaccc

SEQ ID NO: 1006 - DNA, 33 bp, Artificial Sequence
  synthesized nucleic acid probe to detect Enterobacteriaceae_1006
gcggctatgctagcgcacacgtctcttagccgc

SEQ ID NO: 1007 - DNA, 33 bp, Artificial Sequence
  synthesized nucleic acid probe to detect Enterobacteriaceae_1007
gaccgcatgctagcgcacacgtctcttgcggtc

SEQ ID NO: 1008 - DNA, 33 bp, Artificial Sequence
  synthesized nucleic acid probe to detect Enterobacteriaceae_1008
gacggcatgctagcgcacacgtctcttgccgtc

SEQ ID NO: 1009 - DNA, 33 bp, Artificial Sequence
  synthesized nucleic acid probe to detect Enterobacteriaceae_1009
ggcccaatgctagcgcacacgtctctttgggcc

SEQ ID NO: 1010 - DNA, 23 bp, Artificial Sequence
  synthesized nucleic acid probe to detect Enterobacteriaceae_1010
gtaccttcgccgaagctaagcac

SEQ ID NO: 1011 - DNA, 35 bp, Artificial Sequence
  synthesized nucleic acid probe to detect Enterobacteriaceae_1011
cgcggagtaccttcgccgaagctaagcactccgcg

SEQ ID NO: 1012 - DNA, 35 bp, Artificial Sequence
  synthesized nucleic acid probe to detect Enterobacteriaceae_1012
ctcgcggtaccttcgccgaagctaagcaccgcgag

SEQ ID NO: 1013 - DNA, 35 bp, Artificial Sequence
  synthesized nucleic acid probe to detect Enterobacteriaceae_1013
ccgaccgtaccttcgccgaagctaagcacggtcgg

SEQ ID NO: 1014 - DNA, 35 bp, Artificial Sequence
  synthesized nucleic acid probe to detect Enterobacteriaceae_1014
cgacgtgtaccttcgccgaagctaagcacacgtcg

SEQ ID NO: 1015 - DNA, 35 bp, Artificial Sequence
  synthesized nucleic acid probe to detect Enterobacteriaceae_1015
cccaccgtaccttcgccgaagctaagcacggtggg

SEQ ID NO: 1016 - DNA, 35 bp, Artificial Sequence
  synthesized nucleic acid probe to detect Enterobacteriaceae_1016
cgccgagtaccttcgccgaagctaagcactcggcg

SEQ ID NO: 1017 - DNA, 35 bp, Artificial Sequence
  synthesized nucleic acid probe to detect Enterobacteriaceae_1017
ctggcggtaccttcgccgaagctaagcaccgccag

SEQ ID NO: 1018 - DNA, 35 bp, Artificial Sequence
  synthesized nucleic acid probe to detect Enterobacteriaceae_1018
ctgccggtaccttcgccgaagctaagcaccggcag

SEQ ID NO: 1019 - DNA, 35 bp, Artificial Sequence
  synthesized nucleic acid probe to detect Enterobacteriaceae_1019
ccgggtgtaccttcgccgaagctaagcacacccgg

SEQ ID NO: 1020 - DNA, 35 bp, Artificial Sequence
  synthesized nucleic acid probe to detect Enterobacteriaceae_1020
ccccccgtaccttcgccgaagctaagcacgggggg

SEQ ID NO: 1021 - DNA, 35 bp, Artificial Sequence
  synthesized nucleic acid probe to detect Enterobacteriaceae_1021
gcgcctgtaccttcgccgaagctaagcacaggcgc

SEQ ID NO: 1022 - DNA, 35 bp, Artificial Sequence
  synthesized nucleic acid probe to detect Enterobacteriaceae_1022
gagcgcgtaccttcgccgaagctaagcacgcgctc

SEQ ID NO: 1023 - DNA, 35 bp, Artificial Sequence
  synthesized nucleic acid probe to detect Enterobacteriaceae_1023
ggctgggtaccttcgccgaagctaagcacccagcc

SEQ ID NO: 1024 - DNA, 35 bp, Artificial Sequence
  synthesized nucleic acid probe to detect Enterobacteriaceae_1024
gctgcagtaccttcgccgaagctaagcactgcagc

SEQ ID NO: 1025 - DNA, 35 bp, Artificial Sequence
  synthesized nucleic acid probe to detect Enterobacteriaceae_1025
gggtgggtaccttcgccgaagctaagcacccaccc

SEQ ID NO: 1026 - DNA, 35 bp, Artificial Sequence
  synthesized nucleic acid probe to detect Enterobacteriaceae_1026
gcggctgtaccttcgccgaagctaagcacagccgc

SEQ ID NO: 1027 - DNA, 35 bp, Artificial Sequence
  synthesized nucleic acid probe to detect Enterobacteriaceae_1027
gaccgcgtaccttcgccgaagctaagcacgcggtc

SEQ ID NO: 1028 - DNA, 35 bp, Artificial Sequence
  synthesized nucleic acid probe to detect Enterobacteriaceae_1028
gacggcgtaccttcgccgaagctaagcacgccgtc

SEQ ID NO: 1029 - DNA, 35 bp, Artificial Sequence
  synthesized nucleic acid probe to detect Enterobacteriaceae_1029
ggcccagtaccttcgccgaagctaagcactgggcc

SEQ ID NO: 1030 - DNA, 18 bp, Artificial Sequence
  synthesized nucleic acid probe to detect Enterobacteriaceae_1030
tcgccgaagctaagcact

SEQ ID NO: 1031 - DNA, 30 bp, Artificial Sequence
  synthesized nucleic acid probe to detect Enterobacteriaceae_1031
cgcggatcgccgaagctaagcacttccgcg

SEQ ID NO: 1032 - DNA, 30 bp, Artificial Sequence
  synthesized nucleic acid probe to detect Enterobacteriaceae_1032
ctcgcgtcgccgaagctaagcactcgcgag

SEQ ID NO: 1033 - DNA, 30 bp, Artificial Sequence
  synthesized nucleic acid probe to detect Enterobacteriaceae_1033
ccgacctcgccgaagctaagcactggtcgg

SEQ ID NO: 1034 - DNA, 30 bp, Artificial Sequence
  synthesized nucleic acid probe to detect Enterobacteriaceae_1034
cgacgttcgccgaagctaagcactacgtcg

SEQ ID NO: 1035 - DNA, 30 bp, Artificial Sequence
  synthesized nucleic acid probe to detect Enterobacteriaceae_1035
cccacctcgccgaagctaagcactggtggg

SEQ ID NO: 1036 - DNA, 30 bp, Artificial Sequence
  synthesized nucleic acid probe to detect Enterobacteriaceae_1036
cgccgatcgccgaagctaagcacttcggcg

SEQ ID NO: 1037 - DNA, 30 bp, Artificial Sequence
  synthesized nucleic acid probe to detect Enterobacteriaceae_1037
ctggcgtcgccgaagctaagcactcgccag

SEQ ID NO: 1038 - DNA, 30 bp, Artificial Sequence
  synthesized nucleic acid probe to detect Enterobacteriaceae_1038
ctgccgtcgccgaagctaagcactcggcag

SEQ ID NO: 1039 - DNA, 30 bp, Artificial Sequence
  synthesized nucleic acid probe to detect Enterobacteriaceae_1039
ccgggttcgccgaagctaagcactacccgg

SEQ ID NO: 1040 - DNA, 30 bp, Artificial Sequence
  synthesized nucleic acid probe to detect Enterobacteriaceae_1040
cccccctcgccgaagctaagcactgggggg

SEQ ID NO: 1041 - DNA, 29 bp, Artificial Sequence
  synthesized nucleic acid probe to detect Enterobacteriaceae_1041
cccccatcgccgaagctaagcactggggg

SEQ ID NO: 1042 - DNA, 30 bp, Artificial Sequence
  synthesized nucleic acid probe to detect Enterobacteriaceae_1042
gcgccttcgccgaagctaagcactaggcgc

SEQ ID NO: 1043 - DNA, 30 bp, Artificial Sequence
  synthesized nucleic acid probe to detect Enterobacteriaceae_1043
gagcgctcgccgaagctaagcactgcgctc

SEQ ID NO: 1044 - DNA, 30 bp, Artificial Sequence
  synthesized nucleic acid probe to detect Enterobacteriaceae_1044
ggctggtcgccgaagctaagcactccagcc

SEQ ID NO: 1045 - DNA, 30 bp, Artificial Sequence
  synthesized nucleic acid probe to detect Enterobacteriaceae_1045
gctgcatcgccgaagctaagcacttgcagc

SEQ ID NO: 1046 - DNA, 30 bp, Artificial Sequence
  synthesized nucleic acid probe to detect Enterobacteriaceae_1046
gggtggtcgccgaagctaagcactccaccc

SEQ ID NO: 1047 - DNA, 30 bp, Artificial Sequence
  synthesized nucleic acid probe to detect Enterobacteriaceae_1047
gcggcttcgccgaagctaagcactagccgc

SEQ ID NO: 1048 - DNA, 30 bp, Artificial Sequence
  synthesized nucleic acid probe to detect Enterobacteriaceae_1048
gaccgctcgccgaagctaagcactgcggtc

SEQ ID NO: 1049 - DNA, 30 bp, Artificial Sequence
  synthesized nucleic acid probe to detect Enterobacteriaceae_1049
gacggctcgccgaagctaagcactgccgtc

SEQ ID NO: 1050 - DNA, 30 bp, Artificial Sequence
  synthesized nucleic acid probe to detect Enterobacteriaceae_1050
ggcccatcgccgaagctaagcacttgggcc

SEQ ID NO: 1051 - DNA, 17 bp, Artificial Sequence
  synthesized nucleic acid probe to detect Enterobacteriaceae_1051
cgtaccttcgccgaagc

SEQ ID NO: 1052 - DNA, 29 bp, Artificial Sequence
  synthesized nucleic acid probe to detect Enterobacteriaceae_1052
cgcggacgtaccttcgccgaagctccgcg

SEQ ID NO: 1053 - DNA, 29 bp, Artificial Sequence
  synthesized nucleic acid probe to detect Enterobacteriaceae_1053
ctcgcgcgtaccttcgccgaagccgcgag

SEQ ID NO: 1054 - DNA, 29 bp, Artificial Sequence
  synthesized nucleic acid probe to detect Enterobacteriaceae_1054
ccgacccgtaccttcgccgaagcggtcgg

SEQ ID NO: 1055 - DNA, 29 bp, Artificial Sequence
  synthesized nucleic acid probe to detect Enterobacteriaceae_1055
cgacgtcgtaccttcgccgaagcacgtcg

SEQ ID NO: 1056 - DNA, 29 bp, Artificial Sequence
  synthesized nucleic acid probe to detect Enterobacteriaceae_1056
cgcgagcgtaccttcgccgaagcctcgcg

SEQ ID NO: 1057 - DNA, 29 bp, Artificial Sequence
  synthesized nucleic acid probe to detect Enterobacteriaceae_1057
cccacccgtaccttcgccgaagcggtggg

SEQ ID NO: 1058 - DNA, 29 bp, Artificial Sequence
  synthesized nucleic acid probe to detect Enterobacteriaceae_1058
cgccgacgtaccttcgccgaagctcggcg

SEQ ID NO: 1059 - DNA, 29 bp, Artificial Sequence
  synthesized nucleic acid probe to detect Enterobacteriaceae_1059
ctggcgcgtaccttcgccgaagccgccag

SEQ ID NO: 1060 - DNA, 29 bp, Artificial Sequence
  synthesized nucleic acid probe to detect Enterobacteriaceae_1060
ctgccgcgtaccttcgccgaagccggcag

SEQ ID NO: 1061 - DNA, 29 bp, Artificial Sequence
  synthesized nucleic acid probe to detect Enterobacteriaceae_1061
ccgggtcgtaccttcgccgaagcacccgg

SEQ ID NO: 1062 - DNA, 29 bp, Artificial Sequence
  synthesized nucleic acid probe to detect Enterobacteriaceae_1062
cccccccgtaccttcgccgaagcgggggg

SEQ ID NO: 1063 - DNA, 29 bp, Artificial Sequence
  synthesized nucleic acid probe to detect Enterobacteriaceae_1063
gcgcctcgtaccttcgccgaagcaggcgc

SEQ ID NO: 1064 - DNA, 29 bp, Artificial Sequence
  synthesized nucleic acid probe to detect Enterobacteriaceae_1064
gagcgccgtaccttcgccgaagcgcgctc

SEQ ID NO: 1065 - DNA, 29 bp, Artificial Sequence
  synthesized nucleic acid probe to detect Enterobacteriaceae_1065
ggctggcgtaccttcgccgaagcccagcc

SEQ ID NO: 1066 - DNA, 29 bp, Artificial Sequence
  synthesized nucleic acid probe to detect Enterobacteriaceae_1066
gctgcacgtaccttcgccgaagctgcagc

SEQ ID NO: 1067 - DNA, 29 bp, Artificial Sequence
  synthesized nucleic acid probe to detect Enterobacteriaceae_1067
gggtggcgtaccttcgccgaagcccaccc

SEQ ID NO: 1068 - DNA, 29 bp, Artificial Sequence
  synthesized nucleic acid probe to detect Enterobacteriaceae_1068
gcggctcgtaccttcgccgaagcagccgc

SEQ ID NO: 1069 - DNA, 29 bp, Artificial Sequence
  synthesized nucleic acid probe to detect Enterobacteriaceae_1069
gaccgccgtaccttcgccgaagcgcggtc

SEQ ID NO: 1070 - DNA, 29 bp, Artificial Sequence
  synthesized nucleic acid probe to detect Enterobacteriaceae_1070
gacggccgtaccttcgccgaagcgccgtc

SEQ ID NO: 1071 - DNA, 29 bp, Artificial Sequence
  synthesized nucleic acid probe to detect Enterobacteriaceae_1071
ggcccacgtaccttcgccgaagctgggcc

SEQ ID NO: 1072 - DNA, 23 bp, Artificial Sequence
  synthesized nucleic acid probe to detect Enterobacteriaceae_1072
cagtactactcttcctagaggct

SEQ ID NO: 1073 - DNA, 35 bp, Artificial Sequence
  synthesized nucleic acid probe to detect Enterobacteriaceae_1073
cgcggacagtactactcttcctagaggcttccgcg

SEQ ID NO: 1074 - DNA, 35 bp, Artificial Sequence
  synthesized nucleic acid probe to detect Enterobacteriaceae_1074
ctcgcgcagtactactcttcctagaggctcgcgag

SEQ ID NO: 1075 - DNA, 35 bp, Artificial Sequence
  synthesized nucleic acid probe to detect Enterobacteriaceae_1075
ccgacccagtactactcttcctagaggctggtcgg

SEQ ID NO: 1076 - DNA, 35 bp, Artificial Sequence
  synthesized nucleic acid probe to detect Enterobacteriaceae_1076
cgacgtcagtactactcttcctagaggctacgtcg

SEQ ID NO: 1077 - DNA, 35 bp, Artificial Sequence
  synthesized nucleic acid probe to detect Enterobacteriaceae_1077
cccacccagtactactcttcctagaggctggtggg

SEQ ID NO: 1078 - DNA, 35 bp, Artificial Sequence
  synthesized nucleic acid probe to detect Enterobacteriaceae_1078
cgccgacagtactactcttcctagaggcttcggcg

SEQ ID NO: 1079 - DNA, 35 bp, Artificial Sequence
  synthesized nucleic acid probe to detect Enterobacteriaceae_1079
ctggcgcagtactactcttcctagaggctcgccag

SEQ ID NO: 1080 - DNA, 35 bp, Artificial Sequence
  synthesized nucleic acid probe to detect Enterobacteriaceae_1080
ctgccgcagtactactcttcctagaggctcggcag

SEQ ID NO: 1081 - DNA, 35 bp, Artificial Sequence
  synthesized nucleic acid probe to detect Enterobacteriaceae_1081
ccgggtcagtactactcttcctagaggctacccgg

SEQ ID NO: 1082 - DNA, 35 bp, Artificial Sequence
  synthesized nucleic acid probe to detect Enterobacteriaceae_1082
cccccccagtactactcttcctagaggctgggggg

SEQ ID NO: 1083 - DNA, 35 bp, Artificial Sequence
  synthesized nucleic acid probe to detect Enterobacteriaceae_1083
gcgcctcagtactactcttcctagaggctaggcgc

SEQ ID NO: 1084 - DNA, 35 bp, Artificial Sequence
  synthesized nucleic acid probe to detect Enterobacteriaceae_1084
gagcgccagtactactcttcctagaggctgcgctc

SEQ ID NO: 1085 - DNA, 35 bp, Artificial Sequence
  synthesized nucleic acid probe to detect Enterobacteriaceae_1085
ggctggcagtactactcttcctagaggctccagcc

SEQ ID NO: 1086 - DNA, 35 bp, Artificial Sequence
  synthesized nucleic acid probe to detect Enterobacteriaceae_1086
gctgcacagtactactcttcctagaggcttgcagc

SEQ ID NO: 1087 - DNA, 35 bp, Artificial Sequence
  synthesized nucleic acid probe to detect Enterobacteriaceae_1087
gggtggcagtactactcttcctagaggctccaccc

SEQ ID NO: 1088 - DNA, 35 bp, Artificial Sequence
  synthesized nucleic acid probe to detect Enterobacteriaceae_1088
gcggctcagtactactcttcctagaggctagccgc

SEQ ID NO: 1089 - DNA, 35 bp, Artificial Sequence
  synthesized nucleic acid probe to detect Enterobacteriaceae_1089
gaccgccagtactactcttcctagaggctgcggtc

SEQ ID NO: 1090 - DNA, 35 bp, Artificial Sequence
  synthesized nucleic acid probe to detect Enterobacteriaceae_1090
gacggccagtactactcttcctagaggctgccgtc

SEQ ID NO: 1091 - DNA, 35 bp, Artificial Sequence
  synthesized nucleic acid probe to detect Enterobacteriaceae_1091
ggcccacagtactactcttcctagaggcttgggcc

SEQ ID NO: 1092 - DNA, 21 bp, Artificial Sequence
  synthesized nucleic acid probe to detect Enterobacteriaceae_1092
ggcagtactactcttcctaga

SEQ ID NO: 1093 - DNA, 31 bp, Artificial Sequence
  synthesized nucleic acid probe to detect Enterobacteriaceae_1093
ctgcggcagtactactcttcctagaccgcag

SEQ ID NO: 1094 - DNA, 33 bp, Artificial Sequence
  synthesized nucleic acid probe to detect Enterobacteriaceae_1094
cgcggaggcagtactactcttcctagatccgcg

SEQ ID NO: 1095 - DNA, 33 bp, Artificial Sequence
  synthesized nucleic acid probe to detect Enterobacteriaceae_1095
ctcgcgggcagtactactcttcctagacgcgag

SEQ ID NO: 1096 - DNA, 33 bp, Artificial Sequence
  synthesized nucleic acid probe to detect Enterobacteriaceae_1096
ccgaccggcagtactactcttcctagaggtcgg

SEQ ID NO: 1097 - DNA, 33 bp, Artificial Sequence
  synthesized nucleic acid probe to detect Enterobacteriaceae_1097
cgacgtggcagtactactcttcctagaacgtcg

SEQ ID NO: 1098 - DNA, 33 bp, Artificial Sequence
  synthesized nucleic acid probe to detect Enterobacteriaceae_1098
cccaccggcagtactactcttcctagaggtggg

SEQ ID NO: 1099 - DNA, 33 bp, Artificial Sequence
  synthesized nucleic acid probe to detect Enterobacteriaceae_1099
cgccgaggcagtactactcttcctagatcggcg

SEQ ID NO: 1100 - DNA, 33 bp, Artificial Sequence
  synthesized nucleic acid probe to detect Enterobacteriaceae_1100
ctggcgggcagtactactcttcctagacgccag

SEQ ID NO: 1101 - DNA, 33 bp, Artificial Sequence
  synthesized nucleic acid probe to detect Enterobacteriaceae_1101
ctgccgggcagtactactcttcctagacggcag

SEQ ID NO: 1102 - DNA, 33 bp, Artificial Sequence
  synthesized nucleic acid probe to detect Enterobacteriaceae_1102
ccgggtggcagtactactcttcctagaacccgg

SEQ ID NO: 1103 - DNA, 33 bp, Artificial Sequence
  synthesized nucleic acid probe to detect Enterobacteriaceae_1103
ccccccggcagtactactcttcctagagggggg

SEQ ID NO: 1104 - DNA, 33 bp, Artificial Sequence
  synthesized nucleic acid probe to detect Enterobacteriaceae_1104
gcgcctggcagtactactcttcctagaaggcgc

SEQ ID NO: 1105 - DNA, 33 bp, Artificial Sequence
  synthesized nucleic acid probe to detect Enterobacteriaceae_1105
gagcgcggcagtactactcttcctagagcgctc

SEQ ID NO: 1106 - DNA, 33 bp, Artificial Sequence
  synthesized nucleic acid probe to detect Enterobacteriaceae_1106
ggctggggcagtactactcttcctagaccagcc

SEQ ID NO: 1107 - DNA, 33 bp, Artificial Sequence
  synthesized nucleic acid probe to detect Enterobacteriaceae_1107
gctgcaggcagtactactcttcctagatgcagc

SEQ ID NO: 1108 - DNA, 33 bp, Artificial Sequence
  synthesized nucleic acid probe to detect Enterobacteriaceae_1108
gggtggggcagtactactcttcctagaccaccc

SEQ ID NO: 1109 - DNA, 33 bp, Artificial Sequence
  synthesized nucleic acid probe to detect Enterobacteriaceae_1109
gcggctggcagtactactcttcctagaagccgc

SEQ ID NO: 1110 - DNA, 33 bp, Artificial Sequence
  synthesized nucleic acid probe to detect Enterobacteriaceae_1110
gaccgcggcagtactactcttcctagagcggtc

SEQ ID NO: 1111 - DNA, 33 bp, Artificial Sequence
  synthesized nucleic acid probe to detect Enterobacteriaceae_1111
gacggcggcagtactactcttcctagagccgtc

SEQ ID NO: 1112 - DNA, 33 bp, Artificial Sequence
  synthesized nucleic acid probe to detect Enterobacteriaceae_1112
ggcccaggcagtactactcttcctagatgggcc

SEQ ID NO: 1113 - DNA, 23 bp, Artificial Sequence
  synthesized nucleic acid probe to detect Enterobacteriaceae_1113
gggcagtactactcttcctagag

SEQ ID NO: 1114 - DNA, 35 bp, Artificial Sequence
  synthesized nucleic acid probe to detect Enterobacteriaceae_1114
cgcggagggcagtactactcttcctagagtccgcg

SEQ ID NO: 1115 - DNA, 35 bp, Artificial Sequence
  synthesized nucleic acid probe to detect Enterobacteriaceae_1115
ctcgcggggcagtactactcttcctagagcgcgag

SEQ ID NO: 1116 - DNA, 35 bp, Artificial Sequence
  synthesized nucleic acid probe to detect Enterobacteriaceae_1116
ccgaccgggcagtactactcttcctagagggtcgg

SEQ ID NO: 1117 - DNA, 35 bp, Artificial Sequence
  synthesized nucleic acid probe to detect Enterobacteriaceae_1117
cgacgtgggcagtactactcttcctagagacgtcg

SEQ ID NO: 1118 - DNA, 35 bp, Artificial Sequence
  synthesized nucleic acid probe to detect Enterobacteriaceae_1118
cccaccgggcagtactactcttcctagagggtggg

SEQ ID NO: 1119 - DNA, 35 bp, Artificial Sequence
  synthesized nucleic acid probe to detect Enterobacteriaceae_1119
cgccgagggcagtactactcttcctagagtcggcg

SEQ ID NO: 1120 - DNA, 35 bp, Artificial Sequence
  synthesized nucleic acid probe to detect Enterobacteriaceae_1120
ctggcggggcagtactactcttcctagagcgccag

SEQ ID NO: 1121 - DNA, 34 bp, Artificial Sequence
  synthesized nucleic acid probe to detect Enterobacteriaceae_1121
ctcgccgggcagtactactcttcctagaggcgag

SEQ ID NO: 1122 - DNA, 35 bp, Artificial Sequence
  synthesized nucleic acid probe to detect Enterobacteriaceae_1122
ctgccggggcagtactactcttcctagagcggcag

SEQ ID NO: 1123 - DNA, 35 bp, Artificial Sequence
  synthesized nucleic acid probe to detect Enterobacteriaceae_1123
ccgggtgggcagtactactcttcctagagacccgg

SEQ ID NO: 1124 - DNA, 35 bp, Artificial Sequence
  synthesized nucleic acid probe to detect Enterobacteriaceae_1124
ccccccgggcagtactactcttcctagaggggggg

SEQ ID NO: 1125 - DNA, 35 bp, Artificial Sequence
  synthesized nucleic acid probe to detect Enterobacteriaceae_1125
gcgcctgggcagtactactcttcctagagaggcgc

SEQ ID NO: 1126 - DNA, 35 bp, Artificial Sequence
  synthesized nucleic acid probe to detect Enterobacteriaceae_1126
gagcgcgggcagtactactcttcctagaggcgctc

SEQ ID NO: 1127 - DNA, 35 bp, Artificial Sequence
  synthesized nucleic acid probe to detect Enterobacteriaceae_1127
ggctgggggcagtactactcttcctagagccagcc

SEQ ID NO: 1128 - DNA, 35 bp, Artificial Sequence
  synthesized nucleic acid probe to detect Enterobacteriaceae_1128
gctgcagggcagtactactcttcctagagtgcagc

SEQ ID NO: 1129 - DNA, 35 bp, Artificial Sequence
  synthesized nucleic acid probe to detect Enterobacteriaceae_1129
gggtgggggcagtactactcttcctagagccaccc

SEQ ID NO: 1130 - DNA, 35 bp, Artificial Sequence
  synthesized nucleic acid probe to detect Enterobacteriaceae_1130
gcggctgggcagtactactcttcctagagagccgc

SEQ ID NO: 1131 - DNA, 35 bp, Artificial Sequence
  synthesized nucleic acid probe to detect Enterobacteriaceae_1131
gaccgcgggcagtactactcttcctagaggcggtc

SEQ ID NO: 1132 - DNA, 35 bp, Artificial Sequence
  synthesized nucleic acid probe to detect Enterobacteriaceae_1132
gacggcgggcagtactactcttcctagaggccgtc

SEQ ID NO: 1133 - DNA, 35 bp, Artificial Sequence
  synthesized nucleic acid probe to detect Enterobacteriaceae_1133
ggcccagggcagtactactcttcctagagtgggcc

SEQ ID NO: 1134 - DNA, 23 bp, Artificial Sequence
  synthesized nucleic acid probe to detect Enterobacteriaceae_1134
catcggttaaacaattagcactg

SEQ ID NO: 1135 - DNA, 35 bp, Artificial Sequence; identical to SEQ ID NO: 549
  synthesized nucleic acid probe to detect Enterobacteriaceae_1135

SEQ ID NO: 1136 - DNA, 35 bp, Artificial Sequence; identical to SEQ ID NO: 550
  synthesized nucleic acid probe to detect Enterobacteriaceae_1136

SEQ ID NO: 1137 - DNA, 35 bp, Artificial Sequence; identical to SEQ ID NO: 551
  synthesized nucleic acid probe to detect Enterobacteriaceae_1137

SEQ ID NO: 1138 - DNA, 35 bp, Artificial Sequence; identical to SEQ ID NO: 552
  synthesized nucleic acid probe to detect Enterobacteriaceae_1138

SEQ ID NO: 1139 - DNA, 35 bp, Artificial Sequence; identical to SEQ ID NO: 553
  synthesized nucleic acid probe to detect Enterobacteriaceae_1139

SEQ ID NO: 1140 - DNA, 29 bp, Artificial Sequence
  synthesized nucleic acid probe to detect Enterobacteriaceae_1140
cagtgccatcggttaaacaattagcactg

SEQ ID NO: 1141 - DNA, 35 bp, Artificial Sequence; identical to SEQ ID NO: 554
  synthesized nucleic acid probe to detect Enterobacteriaceae_1141

SEQ ID NO: 1142 - DNA, 35 bp, Artificial Sequence; identical to SEQ ID NO: 555
  synthesized nucleic acid probe to detect Enterobacteriaceae_1142

SEQ ID NO: 1143 - DNA, 35 bp, Artificial Sequence; identical to SEQ ID NO: 557
  synthesized nucleic acid probe to detect Enterobacteriaceae_1143

SEQ ID NO: 1144 - DNA, 35 bp, Artificial Sequence; identical to SEQ ID NO: 558
  synthesized nucleic acid probe to detect Enterobacteriaceae_1144

SEQ ID NO: 1145 - DNA, 35 bp, Artificial Sequence
  synthesized nucleic acid probe to detect Enterobacteriaceae_1145
cccccccatcggttaaacaattagcactggggggg

SEQ ID NO: 1146 - DNA, 35 bp, Artificial Sequence; identical to SEQ ID NO: 559
  synthesized nucleic acid probe to detect Enterobacteriaceae_1146

SEQ ID NO: 1147 - DNA, 35 bp, Artificial Sequence; identical to SEQ ID NO: 560
  synthesized nucleic acid probe to detect Enterobacteriaceae_1147

SEQ ID NO: 1148 - DNA, 35 bp, Artificial Sequence; identical to SEQ ID NO: 561
  synthesized nucleic acid probe to detect Enterobacteriaceae_1148

SEQ ID NO: 1149 - DNA, 35 bp, Artificial Sequence; identical to SEQ ID NO: 562
  synthesized nucleic acid probe to detect Enterobacteriaceae_1149

SEQ ID NO: 1150 - DNA, 35 bp, Artificial Sequence; identical to SEQ ID NO: 563
  synthesized nucleic acid probe to detect Enterobacteriaceae_1150

SEQ ID NO: 1151 - DNA, 35 bp, Artificial Sequence; identical to SEQ ID NO: 564
  synthesized nucleic acid probe to detect Enterobacteriaceae_1151

SEQ ID NO: 1152 - DNA, 35 bp, Artificial Sequence; identical to SEQ ID NO: 565
  synthesized nucleic acid probe to detect Enterobacteriaceae_1152

SEQ ID NO: 1153 - DNA, 35 bp, Artificial Sequence; identical to SEQ ID NO: 566
  synthesized nucleic acid probe to detect Enterobacteriaceae_1153

SEQ ID NO: 1154 - DNA, 35 bp, Artificial Sequence; identical to SEQ ID NO: 567
  synthesized nucleic acid probe to detect Enterobacteriaceae_1154

SEQ ID NO: 1155 - DNA, 23 bp, Artificial Sequence
  synthesized nucleic acid probe to detect Enterobacteriaceae_1155
acgaacctctaaagaggttcatc

SEQ ID NO: 1156 - DNA, 35 bp, Artificial Sequence
  synthesized nucleic acid probe to detect Enterobacteriaceae_1156
cgcggaacgaacctctaaagaggttcatctccgcg

SEQ ID NO: 1157 - DNA, 35 bp, Artificial Sequence
  synthesized nucleic acid probe to detect Enterobacteriaceae_1157
ctcgcgacgaacctctaaagaggttcatccgcgag

SEQ ID NO: 1158 - DNA, 35 bp, Artificial Sequence
  synthesized nucleic acid probe to detect Enterobacteriaceae_1158
ccgaccacgaacctctaaagaggttcatcggtcgg

SEQ ID NO: 1159 - DNA, 35 bp, Artificial Sequence
  synthesized nucleic acid probe to detect Enterobacteriaceae_1159
ctcgcggatgaacctctttagaggttcgtcgcgag

SEQ ID NO: 1160 - DNA, 35 bp, Artificial Sequence
  synthesized nucleic acid probe to detect Enterobacteriaceae_1160
cgacgtacgaacctctaaagaggttcatcacgtcg

SEQ ID NO: 1161 - DNA, 35 bp, Artificial Sequence
  synthesized nucleic acid probe to detect Enterobacteriaceae_1161
cccaccacgaacctctaaagaggttcatcggtggg

SEQ ID NO: 1162 - DNA, 35 bp, Artificial Sequence
  synthesized nucleic acid probe to detect Enterobacteriaceae_1162
cgccgaacgaacctctaaagaggttcatctcggcg

SEQ ID NO: 1163 - DNA, 35 bp, Artificial Sequence
  synthesized nucleic acid probe to detect Enterobacteriaceae_1163
ctggcgacgaacctctaaagaggttcatccgccag

SEQ ID NO: 1164 - DNA, 35 bp, Artificial Sequence
  synthesized nucleic acid probe to detect Enterobacteriaceae_1164
ctgccgacgaacctctaaagaggttcatccggcag

SEQ ID NO: 1165 - DNA, 35 bp, Artificial Sequence
  synthesized nucleic acid probe to detect Enterobacteriaceae_1165
ccgggtacgaacctctaaagaggttcatcacccgg

SEQ ID NO: 1166 - DNA, 35 bp, Artificial Sequence
  synthesized nucleic acid probe to detect Enterobacteriaceae_1166
ccccccacgaacctctaaagaggttcatcgggggg

SEQ ID NO: 1167 - DNA, 35 bp, Artificial Sequence
  synthesized nucleic acid probe to detect Enterobacteriaceae_1167
gcgcctacgaacctctaaagaggttcatcaggcgc

SEQ ID NO: 1168 - DNA, 35 bp, Artificial Sequence
  synthesized nucleic acid probe to detect Enterobacteriaceae_1168
gagcgcacgaacctctaaagaggttcatcgcgctc

SEQ ID NO: 1169 - DNA, 35 bp, Artificial Sequence
  synthesized nucleic acid probe to detect Enterobacteriaceae_1169
ggctggacgaacctctaaagaggttcatcccagcc

SEQ ID NO: 1170 - DNA, 35 bp, Artificial Sequence
  synthesized nucleic acid probe to detect Enterobacteriaceae_1170
gctgcaacgaacctctaaagaggttcatctgcagc

SEQ ID NO: 1171 - DNA, 35 bp, Artificial Sequence
  synthesized nucleic acid probe to detect Enterobacteriaceae_1171
gggtggacgaacctctaaagaggttcatcccaccc

SEQ ID NO: 1172 - DNA, 35 bp, Artificial Sequence
  synthesized nucleic acid probe to detect Enterobacteriaceae_1172
gcggctacgaacctctaaagaggttcatcagccgc

SEQ ID NO: 1173 - DNA, 35 bp, Artificial Sequence
  synthesized nucleic acid probe to detect Enterobacteriaceae_1173
gaccgcacgaacctctaaagaggttcatcgcggtc

SEQ ID NO: 1174 - DNA, 35 bp, Artificial Sequence
  synthesized nucleic acid probe to detect Enterobacteriaceae_1174
gacggcacgaacctctaaagaggttcatcgccgtc

SEQ ID NO: 1175 - DNA, 35 bp, Artificial Sequence
  synthesized nucleic acid probe to detect Enterobacteriaceae_1175
ggcccaacgaacctctaaagaggttcatctgggcc

SEQ ID NO: 1176 - DNA, 24 bp, Artificial Sequence
  synthesized nucleic acid probe to detect Enterobacteriaceae_1176
acctctaaagaggttcatccacag

SEQ ID NO: 1177 - DNA, 36 bp, Artificial Sequence
  synthesized nucleic acid probe to detect Enterobacteriaceae_1177
cgcggaacctctaaagaggttcatccacagtccgcg

SEQ ID NO: 1178 - DNA, 36 bp, Artificial Sequence
  synthesized nucleic acid probe to detect Enterobacteriaceae_1178
ctcgcgacctctaaagaggttcatccacagcgcgag

SEQ ID NO: 1179 - DNA, 36 bp, Artificial Sequence
  synthesized nucleic acid probe to detect Enterobacteriaceae_1179
ccgaccacctctaaagaggttcatccacagggtcgg

SEQ ID NO: 1180 - DNA, 36 bp, Artificial Sequence
  synthesized nucleic acid probe to detect Enterobacteriaceae_1180
cgacgtacctctaaagaggttcatccacagacgtcg

SEQ ID NO: 1181 - DNA, 36 bp, Artificial Sequence
  synthesized nucleic acid probe to detect Enterobacteriaceae_1181
cccaccacctctaaagaggttcatccacagggtggg

SEQ ID NO: 1182 - DNA, 36 bp, Artificial Sequence
  synthesized nucleic acid probe to detect Enterobacteriaceae_1182
cgccgaacctctaaagaggttcatccacagtcggcg

SEQ ID NO: 1183 - DNA, 36 bp, Artificial Sequence
  synthesized nucleic acid probe to detect Enterobacteriaceae_1183
ctggcgacctctaaagaggttcatccacagcgccag

SEQ ID NO: 1184 - DNA, 36 bp, Artificial Sequence
  synthesized nucleic acid probe to detect Enterobacteriaceae_1184
ctgccgacctctaaagaggttcatccacagcggcag

SEQ ID NO: 1185 - DNA, 36 bp, Artificial Sequence
  synthesized nucleic acid probe to detect Enterobacteriaceae_1185
ccgggtacctctaaagaggttcatccacagacccgg

SEQ ID NO: 1186 - DNA, 36 bp, Artificial Sequence
  synthesized nucleic acid probe to detect Enterobacteriaceae_1186
ccccccacctctaaagaggttcatccacaggggggg

SEQ ID NO: 1187 - DNA, 36 bp, Artificial Sequence
  synthesized nucleic acid probe to detect Enterobacteriaceae_1187
gcgcctacctctaaagaggttcatccacagaggcgc

SEQ ID NO: 1188 - DNA, 35 bp, Artificial Sequence
  synthesized nucleic acid probe to detect Enterobacteriaceae_1188
cctacctctaaagaggttcatccacaggaggtagg

SEQ ID NO: 1189 - DNA, 36 bp, Artificial Sequence
  synthesized nucleic acid probe to detect Enterobacteriaceae_1189
gagcgcacctctaaagaggttcatccacaggcgctc

SEQ ID NO: 1190 - DNA, 36 bp, Artificial Sequence
  synthesized nucleic acid probe to detect Enterobacteriaceae_1190
ggctggacctctaaagaggttcatccacagccagcc

SEQ ID NO: 1191 - DNA, 36 bp, Artificial Sequence
  synthesized nucleic acid probe to detect Enterobacteriaceae_1191
gctgcaacctctaaagaggttcatccacagtgcagc

SEQ ID NO: 1192 - DNA, 36 bp, Artificial Sequence
  synthesized nucleic acid probe to detect Enterobacteriaceae_1192
gggtggacctctaaagaggttcatccacagccaccc

SEQ ID NO: 1193 - DNA, 36 bp, Artificial Sequence
  synthesized nucleic acid probe to detect Enterobacteriaceae_1193
gcggctacctctaaagaggttcatccacagagccgc

SEQ ID NO: 1194 - DNA, 36 bp, Artificial Sequence
  synthesized nucleic acid probe to detect Enterobacteriaceae_1194
gaccgcacctctaaagaggttcatccacaggcggtc

SEQ ID NO: 1195 - DNA, 36 bp, Artificial Sequence
  synthesized nucleic acid probe to detect Enterobacteriaceae_1195
gacggcacctctaaagaggttcatccacaggccgtc

SEQ ID NO: 1196 - DNA, 36 bp, Artificial Sequence
  synthesized nucleic acid probe to detect Enterobacteriaceae_1196
ggcccaacctctaaagaggttcatccacagtgggcc

SEQ ID NO: 1197 - DNA, 22 bp, Artificial Sequence
  synthesized nucleic acid probe to detect Enterobacteriaceae_1197
ctgttacttatgcgccctattc

SEQ ID NO: 1198 - DNA, 34 bp, Artificial Sequence
  synthesized nucleic acid probe to detect Enterobacteriaceae_1198
cgcggactgttacttatgcgccctattctccgcg

SEQ ID NO: 1199 - DNA, 34 bp, Artificial Sequence
  synthesized nucleic acid probe to detect Enterobacteriaceae_1199
ctcgcgctgttacttatgcgccctattccgcgag

SEQ ID NO: 1200 - DNA, 34 bp, Artificial Sequence
  synthesized nucleic acid probe to detect Enterobacteriaceae_1200
ccgaccctgttacttatgcgccctattcggtcgg

SEQ ID NO: 1201 - DNA, 34 bp, Artificial Sequence
  synthesized nucleic acid probe to detect Enterobacteriaceae_1201
cgacgtctgttacttatgcgccctattcacgtcg

SEQ ID NO: 1202 - DNA, 34 bp, Artificial Sequence
  synthesized nucleic acid probe to detect Enterobacteriaceae_1202
cccaccctgttacttatgcgccctattcggtggg

SEQ ID NO: 1203 - DNA, 34 bp, Artificial Sequence
  synthesized nucleic acid probe to detect Enterobacteriaceae_1203
cgccgactgttacttatgcgccctattctcggcg

SEQ ID NO: 1204 - DNA, 34 bp, Artificial Sequence
  synthesized nucleic acid probe to detect Enterobacteriaceae_1204
ctggcgctgttacttatgcgccctattccgccag

SEQ ID NO: 1205 - DNA, 34 bp, Artificial Sequence
  synthesized nucleic acid probe to detect Enterobacteriaceae_1205
ctgccgctgttacttatgcgccctattccggcag

SEQ ID NO: 1206 - DNA, 34 bp, Artificial Sequence
  synthesized nucleic acid probe to detect Enterobacteriaceae_1206
ccgggtctgttacttatgcgccctattcacccgg

SEQ ID NO: 1207 - DNA, 34 bp, Artificial Sequence
  synthesized nucleic acid probe to detect Enterobacteriaceae_1207
ccccccctgttacttatgcgccctattcgggggg

SEQ ID NO: 1208 - DNA, 34 bp, Artificial Sequence
  synthesized nucleic acid probe to detect Enterobacteriaceae_1208
gcgcctctgttacttatgcgccctattcaggcgc

SEQ ID NO: 1209 - DNA, 34 bp, Artificial Sequence
  synthesized nucleic acid probe to detect Enterobacteriaceae_1209
gagcgcctgttacttatgcgccctattcgcgctc

SEQ ID NO: 1210 - DNA, 34 bp, Artificial Sequence
  synthesized nucleic acid probe to detect Enterobacteriaceae_1210
ggctggctgttacttatgcgccctattcccagcc

SEQ ID NO: 1211 - DNA, 34 bp, Artificial Sequence
  synthesized nucleic acid probe to detect Enterobacteriaceae_1211
gctgcactgttacttatgcgccctattctgcagc

SEQ ID NO: 1212 - DNA, 34 bp, Artificial Sequence
  synthesized nucleic acid probe to detect Enterobacteriaceae_1212
gggtggctgttacttatgcgccctattcccaccc

SEQ ID NO: 1213 - DNA, 34 bp, Artificial Sequence
  synthesized nucleic acid probe to detect Enterobacteriaceae_1213
gcggctctgttacttatgcgccctattcagccgc

SEQ ID NO: 1214 - DNA, 34 bp, Artificial Sequence
  synthesized nucleic acid probe to detect Enterobacteriaceae_1214
gaccgcctgttacttatgcgccctattcgcggtc

SEQ ID NO: 1215 - DNA, 34 bp, Artificial Sequence
  synthesized nucleic acid probe to detect Enterobacteriaceae_1215
gacggcctgttacttatgcgccctattcgccgtc

SEQ ID NO: 1216 - DNA, 34 bp, Artificial Sequence
  synthesized nucleic acid probe to detect Enterobacteriaceae_1216
ggcccactgttacttatgcgccctattctgggcc